(19) United States
(12) Patent Application Publication
(10) Pub. No.: US 2021/0228736 A1
(43) Pub. Date: Jul. 29, 2021

(54) PATHOGEN CONTROL COMPOSITIONS AND USES THEREOF

(71) Applicant: Flagship Pioneering Innovations VI, LLC, Cambridge, MA (US)

(72) Inventors: Maria Helena Christine VAN ROOIJEN, Cambridge, MA (US); Barry Andrew MARTIN, Boston, MA (US); Hok Hei TAM, Newton, MA (US); Ignacio MARTINEZ, Lexington, MA (US); Nataliya Vladimirovna NUKOLOVA, Cambridge, MA (US); Simon SCHWIZER, Boston, MA (US); Daniel Garcia CABANILLAS, Boston, MA (US)

(21) Appl. No.: 17/054,816

(22) PCT Filed: May 15, 2019
(86) PCT No.: PCT/US2019/032473

§ 371 Date: Nov. 12, 2020

Related U.S. Application Data
(60) Provisional application No. 62/672,022, filed on May 15, 2018.

(51) Int. Cl. A61K 47/69; A61K 31/704; A61K 38/14; A61K 38/12; A61K 31/65; A61K 31/4164; A61K 31/43; A61K 31/165; A61K 31/407; A61K 31/42; A61K 31/5377; A01N 25/04; A01N 63/60; A61P 31/04; A61P 31/10

(52) U.S. Cl. CPC A61K 47/6911; A61P 31/10; A61K 38/14; A61K 38/12; A61K 31/65; A61K 31/4164; A61K 31/43; A61K 31/165; A61K 31/407; A61K 31/42; A61K 31/5377; A01N 25/04; A01N 63/60; A61P 31/04; A61K 31/704

(57) ABSTRACT

Disclosed herein are pathogen control compositions including a plurality of plant messenger packs, (e.g., including a plant extracellular vesicle (EV), or segment, portion, or extract thereof), that are useful in methods for treating or preventing an infection in an animal and/or decreasing the fitness of pathogens (e.g., animal pathogens), or vectors thereof.

### BACKGROUND

Pathogens, including animal pathogens (e.g., bacteria, fungi, parasites, or viruses), cause severe disease in humans and animals. Although a multitude of means have been utilized for attempting to control animal pathogens, or vectors thereof, the demand for safe and effective pathogen control strategies is increasing. Thus, there is need in the art for new methods and compositions to control animal pathogens.

### SUMMARY OF THE INVENTION

Disclosed herein are pathogen control compositions including a plurality of plant messenger packs that are useful in methods for treating infections in an animal in need thereof, preventing an infection in an animal at risk thereof, or decreasing the fitness of pathogens (e.g., animal pathogens), or vectors thereof.

In one aspect, the disclosure features a pathogen control composition including a plurality of plant messenger packs (PMPs), wherein the composition is formulated for administration to an animal, and wherein the composition includes at least 5% PMPs as measured by wt/vol, percent PMP protein composition, and/or percent lipid composition (e.g., by measuring fluorescently labelled lipids)

In another aspect, the disclosure features a pathogen control composition including a plurality of PMPs, wherein the composition is formulated for delivery to an animal pathogen, and wherein the composition includes at least 5% PMPs.

In still another aspect, the disclosure features a pathogen control composition including a plurality of PMPs, wherein the composition is formulated for delivery to an animal pathogen vector, and wherein the composition includes at least 5% PMPs.

In yet another aspect, the disclosure features a pathogen control composition including a plurality of PMPs, wherein the composition is stable for at least one day at room temperature, and/or stable for at least one week at 4° C.

In some embodiments of the pathogen control composition, the plurality of PMPs in the composition is at a concentration effective to decrease the fitness of an animal pathogen or an animal pathogen vector. In some embodiments, the plurality of PMPs in the composition is at a concentration effective to treat an infection in an animal infected with a pathogen. In other embodiments, the plurality of PMPs in the composition is at a concentration effective to prevent an infection in an animal at risk of an infection with a pathogen.

In another aspect, the disclosure features a pathogen control composition including a plurality of PMPs, wherein the plurality of PMPs in the composition is at a concentration effective to decrease the fitness of an animal pathogen.

In still another aspect, the disclosure features a pathogen control composition including a plurality of PMPs, wherein the plurality of PMPs in the composition is at a concentration effective to decrease the fitness of an animal pathogen vector.

In yet another aspect, the disclosure features a pathogen control composition including a plurality of PMPs, wherein the plurality of PMPs in the composition is at a concentration effective to treat an infection in an animal infected with a pathogen.

And in yet another aspect, the disclosure features a pathogen control composition including a plurality of PMPs, wherein the plurality of PMPs in the composition is at a concentration effective to prevent an infection in an animal at risk of an infection with a pathogen.

In some embodiments of the pathogen control composition, the plurality of PMPs in the composition is at a concentration of at least 0.01 ng, 0.1 ng, 1 ng, 2 ng, 3 ng, 4 ng, 5 ng, 10 ng, 50 ng, 100 ng, 250 ng, 500 ng, 750 ng, 1 μg, 10 μg, 50 μg, 100 μg, or 250 μg PMP protein/ml. In some embodiments, the plurality of PMPs further includes an additional pathogen control agent.

In another aspect, the disclosure features a pathogen control composition including a plurality of PMPs, wherein each of the plurality of PMPs includes a heterologous pathogen control agent and wherein the composition is formulated for delivery to an agricultural or veterinary animal pathogen or a vector thereof.

In some embodiments of the pathogen control composition, the heterologous pathogen control agent is an antibacterial agent, e.g., doxorubicin, an antifungal agent, a virucidal agent, an anti-viral agent, an insecticidal agent, a nematicidal agent, an antiparasitic agent, or an insect repellent. In some embodiments, the antibacterial agent is an antibiotic, e.g., vancomycin, a penicillin, a cephalosporin, a monobactam, a carbapenem, a macrolide, an aminoglycoside, a quinolone, a sulfonamide, a tetracycline, a glycopeptide, a lipoglycopeptide, an oxazolidinone, a rifamycin, a tuberactinomycin, chloramphenicol, metronidazole, tinidazole, nitrofurantoin, teicoplanin, telavancin, linezolid, cycloserine 2, bacitracin, polymyxin B, viomycin, or capreomycin.

In some embodiments of the pathogen control composition, the antifungal agent is an allylamine, an imidazole, a triazole, a thiazole, a polyene, or an echinocandin.

In some embodiments of the pathogen control composition, the insecticidal agent is a chloronicotinyl, a neonicotinoid, a carbamate, an organophosphate, a pyrethroid, an oxadiazine, a spinosyn, a cyclodiene, an organochlorine, a fiprole, a mectin, a diacylhydrazine, a benzoylurea, an organotin, a pyrrole, a dinitroterpenol, a METI, a tetronic acid, a tetramic acid, or a pthalamide.

In some embodiments of the pathogen control composition, the heterologous pathogen control agent is a small molecule (e.g., an antibiotic or a secondary metabolite), a nucleic acid (e.g., an inhibitory RNA), or a polypeptide.

In some embodiments of the pathogen control composition, the heterologous pathogen control agent is encapsulated by each of the plurality of PMPs; embedded on the surface of each of the plurality of PMPs; or conjugated to the surface of each of the plurality of PMPs. In some embodiments, each of the plurality of PMPs further includes an additional pathogen control agent.

In some embodiments, the pathogen is a bacterium (e.g., a Pseudomonas species (e.g., Pseudomonas aeruginosa), an Escherichia species (e.g., Escherichia coli), a Streptococcus species, a Pneumococcus species, a Shigella species, a Salmonella species, or a Campylobacter species), a fungus (e.g., a Saccharomyces species or a Candida species), a parasitic insect (e.g., a Cimex species), a parasitic nematode (e.g., a Heligmosomoides species), or a parasitic protozoan (e.g., a Trichomonas species).

In some embodiments of the pathogen control composition, the vector is a mosquito, a tick, a mite, or a louse.

In some embodiments of the pathogen control composition, the composition is stable for at least one day at room temperature, and/or stable for at least one week at 4° C.; stable for at least 24 hours, 48 hours, seven days, or 30 days at 4° C.; or stable at a temperature of at least 20° C., 24° C., or 37° C.

In some embodiments of the pathogen control composition, the plurality of PMPs in the composition is at a concentration effective to decrease the fitness of an animal pathogen or an animal pathogen vector; effective to treat an infection in an animal infected with a pathogen; or effective to prevent an infection in an animal at risk of an infection with a pathogen.

In some embodiments, the plurality of PMPs in the composition is at a concentration of at least 0.01 ng, 0.1 ng, 1 ng, 2 ng, 3 ng, 4 ng, 5 ng, 10 ng, 50 ng, 100 ng, 250 ng, 500 ng, 750 ng, 1 μg, 10 μg, 50 μg, 100 μg, or 250 μg PMP protein/mL.

In some embodiments, the composition includes an agriculturally acceptable carrier or a pharmaceutically acceptable carrier. In some embodiments, the composition is formulated to stabilize the PMPs. In some embodiments, the composition is formulated as a liquid, a solid, an aerosol, a paste, a gel, or a gas composition. In some embodiments, the composition includes at least 5% PMPs.

In another aspect, the disclosure features a pathogen control composition including a plurality of PMPs, wherein the PMPs are isolated from a plant by a process which includes the steps of (a) providing an initial sample from a plant, or a part thereof, wherein the plant or part thereof includes EVs; (b) isolating a crude PMP fraction from the initial sample, wherein the crude PMP fraction has a decreased level of at least one contaminant or undesired component from the plant or part thereof relative to the level in the initial sample; (c) purifying the crude PMP fraction, thereby producing a plurality of pure PMPs, wherein the plurality of pure PMPs have a decreased level of at least one contaminant or undesired component from the plant or part thereof relative to the level in the crude EV fraction; (d) loading the plurality of PMPs of step (c) with a pathogen control agent; and (e) formulating the PMPs of step (d) for delivery to an agricultural or veterinary animal pathogen or a vector thereof.

In another aspect, the disclosure features an animal pathogen including any one of the pathogen control compositions described herein.

In another aspect, the disclosure features an animal pathogen vector including any one of the pathogen control compositions described herein.

In still another aspect, the disclosure features a method of delivering a pathogen control composition to an animal including administering to the animal any one of the pathogen control compositions described herein.

In still another aspect, the disclosure features a method of treating an infection in an animal in need thereof, the method including administering to the animal an effective amount of any one of the pathogen control compositions described herein.

In yet another aspect, the disclosure features a method of preventing an infection in an animal at risk thereof, the method including administering to the animal an effective amount of any one of the pathogen control compositions described herein, wherein the method decreases the likelihood of the infection in the animal relative to an untreated animal.

In some embodiments of the above methods, the infection is caused by a pathogen, and the pathogen is a bacterium (e.g., a Pseudomonas species, an Escherichia species, a Streptococcus species, a Pneumococcus species, a Shigella species, a Salmonella species, or a Campylobacter species), a fungus (e.g., a Saccharomyces species or a Candida species), a virus, a parasitic insect (e.g., a Cimex species), a parasitic nematode (e.g., a Heligmosomoides species), or a parasitic protozoan (e.g., a Trichomonas species).

In some embodiments, the pathogen control composition is administered to the animal orally, intravenously, or subcutaneously.

In another aspect, the disclosure features a method of delivering a pathogen control composition to a pathogen including contacting the pathogen with any one of the pathogen control compositions described herein.

In another aspect, the disclosure features a method of decreasing the fitness of a pathogen, the method including delivering to the pathogen any one of the pathogen control compositions described herein, wherein the method decreases the fitness of the pathogen relative to an untreated pathogen.

In some embodiments, the method includes delivering the composition to at least one habitat where the pathogen grows, lives, reproduces, feeds, or infests. In some embodiments, the composition is delivered as a pathogen comestible composition for ingestion by the pathogen.

In some embodiments of the above methods, the pathogen is a bacterium (e.g., a Pseudomonas species, an Escherichia species, a Streptococcus species, a Pneumococcus species, a Shigella species, a Salmonella species, or a Campylobacter species), a fungus (e.g., a Saccharomyces species or a Candida species), a parasitic insect (e.g., a Cimex species), a parasitic nematode (e.g., a Heligmosomoides species), or a parasitic protozoan (e.g., a Trichomonas species).

In some embodiments, the composition is delivered as a liquid, a solid, an aerosol, a paste, a gel, or a gas.

In another aspect, the disclosure features a method of decreasing the fitness of an animal pathogen vector, the method including delivering to the vector an effective amount of any one of the pathogen control compositions described herein, wherein the method decreases the fitness of the vector relative to an untreated vector.

In some embodiments, the method includes delivering the composition to at least one habitat where the vector grows, lives, reproduces, feeds, or infests. In some embodiments, the composition is delivered as a comestible composition for ingestion by the vector. In some embodiments, the vector is an insect, e.g., a mosquito, a tick, a mite, or a louse. In some embodiments, the composition is delivered as a liquid, a solid, an aerosol, a paste, a gel, or a gas.

In another aspect, the disclosure features a method of treating an animal having a fungal infection, wherein the method includes administering to the animal an effective amount of a pathogen control composition including a plurality of PMPs.

In another aspect, the disclosure features a method of treating an animal having a fungal infection, wherein the method includes administering to the animal an effective amount of a pathogen control composition including a plurality of PMPs, and wherein the plurality of PMPs includes an antifungal agent.

In some embodiments, the antifungal agent is a nucleic acid that inhibits expression of a gene in a fungus that causes the fungal infection. In some embodiments, the gene is Enhanced Filamentous Growth Protein (EFG1). In some embodiments, the fungal infection is caused by Candida albicans.

In some embodiments, the composition includes a PMP derived from Arabidopsis.

In some embodiments, the method decreases or substantially eliminates the fungal infection.

In another aspect, the disclosure features a method of treating an animal having a bacterial infection, wherein the method includes administering to the animal an effective amount of a pathogen control composition including a plurality of PMPs.

In another aspect, the disclosure features a method of treating an animal having a bacterial infection, wherein the method includes administering to the animal an effective amount of a pathogen control composition including a plurality of PMPs, and wherein the plurality of PMPs includes an antibacterial agent.

In some embodiments, the antibacterial agent is Amphotericin B.

In some embodiments, the bacterium is a Pseudomonas species, an Escherichia species, a Streptococcus species, a Pneumococcus species, a Shigella species, a Salmonella species, or a Campylobacter species.

In some embodiments, the composition includes a PMP derived from Arabidopsis.

In some embodiments, the method decreases or substantially eliminates the bacterial infection.

In some embodiments, the animal is a veterinary animal, or a livestock animal.

In another aspect, the disclosure features a method of decreasing the fitness of a parasitic insect, wherein the method includes delivering to the parasitic insect a pathogen control composition including a plurality of PMPs.

In another aspect, the disclosure features a method of decreasing the fitness of a parasitic insect, wherein the method includes delivering to the parasitic insect a pathogen control composition including a plurality of PMPs, and wherein the plurality of PMPs include an insecticidal agent.

In some embodiments, the insecticidal agent is a peptide nucleic acid.

In some embodiments, the parasitic insect is a bedbug.

In some embodiments, the method decreases the fitness of the parasitic insect relative to an untreated parasitic insect.

In another aspect, the disclosure features a method of decreasing the fitness of a parasitic nematode, wherein the method includes delivering to the parasitic nematode a pathogen control composition including a plurality of PMPs.

In another aspect, the disclosure features a method of decreasing the fitness of a parasitic nematode, wherein the method includes delivering to the parasitic nematode a pathogen control composition including a plurality of PMPs, and wherein the plurality of PMPs includes a nematicidal agent.

In some embodiments, the parasitic nematode is Heligmosomoides polygyrus.

In some embodiments, the method decreases the fitness of the parasitic nematode relative to an untreated parasitic nematode.

In another aspect, the disclosure features a method of decreasing the fitness of a parasitic protozoan, wherein the method includes delivering to the parasitic protozoan a pathogen control composition including a plurality of PMPs.

In another aspect, the disclosure features a method of decreasing the fitness of a parasitic protozoan, wherein the method includes delivering to the parasitic protozoan a pathogen control composition including a plurality of PMPs, and wherein the plurality of PMPs includes an antiparasitic agent.

In some embodiments, the parasitic protozoan is T. vaginalis.

In some embodiments, the method decreases the fitness of the parasitic protozoan relative to an untreated parasitic protozoan.

In another aspect, the disclosure features a method of decreasing the fitness of an insect vector of an animal pathogen, wherein the method includes delivering to the vector a pathogen control composition including a plurality of PMPs.

In another aspect, the disclosure features a method of decreasing the fitness of an insect vector of an animal pathogen, wherein the method includes delivering to the vector a pathogen control composition including a plurality of PMPs, and wherein the plurality of PMPs includes an insecticidal agent.

In some embodiments, the method decreases the fitness of the vector relative to an untreated vector. In some embodiments, the insect is a mosquito, tick, mite, or louse.

Other features and advantages of the invention will be apparent from the following Detailed Description and the Claims.

### Definitions

As used herein, the term “animal” refers to humans, livestock, farm animals, or mammalian veterinary animals (e.g., including for example, dogs, cats, horses, rabbits, zoo animals, cows, pigs, sheep, chickens, and non-human primates).

As used herein “decreasing the fitness of a pathogen” refers to any disruption to pathogen physiology as a consequence of administration of a pathogen control composition described herein, including, but not limited to, any one or more of the following desired effects: (1) decreasing a population of a pathogen by about 10%, 20%, 30%, 40%, 50%, 60%, 70%, 80%, 90%, 95%, 99%, 100% or more; (2) decreasing the reproductive rate of a pathogen by about 10%, 20%, 30%, 40%, 50%, 60%, 70%, 80%, 90%, 95%, 99%, 100% or more; (3) decreasing the mobility of a pathogen by about 10%, 20%, 30%, 40%, 50%, 60%, 70%, 80%, 90%, 95%, 99%, 100% or more; (4) decreasing the body weight or mass of a pathogen by about 10%, 20%, 30%, 40%, 50%, 60%, 70%, 80%, 90%, 95%, 99%, 100% or more; (5) decreasing the metabolic rate or activity of a pathogen by about 10%, 20%, 30%, 40%, 50%, 60%, 70%, 80%, 90%, 95%, 99%, 100% or more; or (6) decreasing pathogen transmission (e.g., vertical or horizontal transmission of a pathogen from one insect to another) by a pathogen by about 10%, 20%, 30%, 40%, 50%, 60%, 70%, 80%, 90%, 95%, 99%, 100% or more. A decrease in pathogen fitness can be determined, e.g., in comparison to an untreated pathogen.

As used herein “decreasing the fitness of a vector” refers to any disruption to vector physiology, or any activity carried out by said vector, as a consequence of administration of a vector control composition described herein, including, but not limited to, any one or more of the following desired effects: (1) decreasing a population of a vector by about 10%, 20%, 30%, 40%, 50%, 60%, 70%, 80%, 90%, 95%, 99%, 100% or more; (2) decreasing the reproductive rate of a vector (e.g., insect, e.g., mosquito, tick, mite, louse) by about 10%, 20%, 30%, 40%, 50%, 60%, 70%, 80%, 90%, 95%, 99%, 100% or more; (3) decreasing the mobility of a vector (e.g., insect, e.g., mosquito, tick, mite, louse) by about 10%, 20%, 30%, 40%, 50%, 60%, 70%, 80%, 90%, 95%, 99%, 100% or more; (4) decreasing the body weight of a vector (e.g., insect, e.g., mosquito, tick, mite, louse) by about 10%, 20%, 30%, 40%, 50%, 60%, 70%, 80%, 90%, 95%, 99%, 100% or more; (5) increasing the metabolic rate or activity of a vector (e.g., insect, e.g., mosquito, tick, mite, louse) by about 10%, 20%, 30%, 40%, 50%, 60%, 70%, 80%, 90%, 95%, 99%, 100% or more; (6) decreasing vector-vector pathogen transmission (e.g., vertical or horizontal transmission of a vector from one insect to another) by a vector (e.g., insect, e.g., mosquito, tick, mite, louse) by about 10%, 20%, 30%, 40%, 50%, 60%, 70%, 80%, 90%, 95%, 99%, 100% or more; (7) decreasing vector-animal pathogen transmission by about 10%, 20%, 30%, 40%, 50%, 60%, 70%, 80%, 90%, 95%, 99%, 100% or more; (8) decreasing vector (e.g., insect, e.g., mosquito, tick, mite, louse) lifespan by about 10%, 20%, 30%, 40%, 50%, 60%, 70%, 80%, 90%, 95%, 99%, 100% or more; (9) increasing vector (e.g., insect, e.g., mosquito, tick, mite, louse) susceptibility to pesticides (e.g., insecticides) by about 10%, 20%, 30%, 40%, 50%, 60%, 70%, 80%, 90%, 95%, 99%, 100% or more; or (10) decreasing vector competence by a vector (e.g., insect, e.g., mosquito, tick, mite, louse) by about 10%, 20%, 30%, 40%, 50%, 60%, 70%, 80%, 90%, 95%, 99%, 100% or more. A decrease in vector fitness can be determined, e.g., in comparison to an untreated vector.

As used herein, the term “formulated for delivery to an animal” refers to a pathogen control composition that includes a pharmaceutically acceptable carrier.

As used herein, the term “formulated for delivery to a pathogen” refers to a pathogen control composition that includes a pharmaceutically acceptable or agriculturally acceptable carrier.

As used herein, the term “formulated for delivery to a vector” refers to a pathogen control composition that includes an agriculturally acceptable carrier.

As used herein, the term “infection” refers to the presence or colonization of a pathogen in an animal (e.g., in one or more parts of the animal), on an animal (e.g., on one or more parts of the animal), or in the habitat surrounding an animal, particularly where the infection decreases the fitness of the animal, e.g., by causing a disease, disease symptoms, or an immune (e.g., inflammatory) response.

As defined herein, the term “nucleic acid” and “polynucleotide” are interchangeable and refer to RNA or DNA that is linear or branched, single or double stranded, or a hybrid thereof, regardless of length (e.g., at least 2, 3, 4, 5, 6, 7, 8, 9, 10, 20, 30, 40, 50, 100, 150, 200, 250, 500, 1000, or more nucleic acids). The term also encompasses RNA/DNA hybrids. Nucleotides are typically linked in a nucleic acid by phosphodiester bonds, although the term “nucleic acid” also encompasses nucleic acid analogs having other types of linkages or backbones (e.g., phosphoramide, phosphorothioate, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, among others). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases (including, e.g., hypoxanthine, xanthine, 7-methylguanine, 5,6-dihydrouracil, 5-methylcytosine, and 5 hydroxymethylcytosine).

As used herein the term “pathogen” refers to an organism, such as a microorganism or an invertebrate, which causes disease or disease symptoms in an animal by, e.g., (i) directly infecting the animal, (ii) by producing agents that causes disease or disease symptoms in an animal (e.g., bacteria that produce pathogenic toxins and the like), and/or (iii) that elicit an immune (e.g., inflammatory response) in animals (e.g., biting insects, e.g., bedbugs). As used herein, pathogens include, but are not limited to bacteria, protozoa, parasites, fungi, nematodes, insects, viroids and viruses, or any combination thereof, wherein each pathogen is capable, either by itself or in concert with another pathogen, of eliciting disease or symptoms in humans.

As used herein, the term “pathogen control composition” refers to an antibacterial, antifungal, virucidal, anti-viral, anti-parasitic (e.g., antihelminthics), parasiticidal, antiparasitic, insecticidal, nematicidal, or vector repellent composition that includes a plurality of plant messenger (PMP) packs. Each of the plurality of PMPs may comprise a pathogen control agent, e.g., a heterologous pathogen control agent.

As used herein, the term “peptide,” “protein,” or “polypeptide” encompasses any chain of naturally or non-naturally occurring amino acids (either D- or L-amino acids), regardless of length (e.g., at least 2, 3, 4, 5, 6, 7, 10, 12, 14, 16, 18, 20, 25, 30, 40, 50, 100, or more amino acids), the presence or absence of post-translational modifications (e.g., glycosylation or phosphorylation), or the presence of, e.g., one or more non-amino acyl groups (for example, sugar, lipid, etc.) covalently linked to the peptide, and includes, for example, natural proteins, synthetic, or recombinant polypeptides and peptides, hybrid molecules, peptoids, or peptidomimetics.

As used herein, “percent identity” between two sequences is determined by the BLAST 2.0 algorithm, which is described in Altschul et al., (1990) J. Mol. Biol. 215:403-410. Software for performing BLAST analyses is publicly available through the National Center for Biotechnology Information.

As used herein, the term “pathogen control agent” or refers to an agent, composition, or substance therein, that controls or decreases the fitness (e.g., kills or inhibits the growth, proliferation, division, reproduction, or spread) of an agricultural, environmental, or domestic/household pathogen or pathogen vector, such as an insect, mollusk, nematode, fungus, bacterium, or virus. Pathogen control agents are understood to encompass naturally occurring or synthetic insecticides (larvicides or adulticides), insect growth regulators, acaricides (miticides), molluscicides, nematicides, ectoparasiticides, bactericides, fungicides, or herbicides. The term “pathogen control agent” may further encompass other bioactive molecules such as antibiotics, antivirals, pesticides, antifungals, antihelminthics, nutrients, and/or agents that stun or slow pathogen or pathogen vector movement. In some instances, the pathogen control agent is an allelochemical. As used herein, “allelochemical” or “allelochemical agent” is a substance produced by an organism (e.g., a plant) that can effect a physiological function (e.g., the germination, growth, survival, or reproduction) of another organism (e.g., a pathogen or a pathogen vector).

The pathogen control agent may be heterologous. As used herein, the term “heterologous” refers to an agent (e.g., a pathogen control agent) that is either (1) exogenous to the plant (e.g., originating from a source that is not the plant or plant part from which the PMP is produced) (e.g., added the PMP using loading approaches described herein) or (2) endogenous to the plant cell or tissue from which the PMP is produced, but present in the PMP (e.g., added to the PMP using loading approaches described herein, genetic engineering, in vitro or in vivo approaches) at a concentration that is higher than that found in nature (e.g., higher than a concentration found in a naturally-occurring plant extracellular vesicle).

As used herein, the term “plant” refers to whole plants, plant organs, plant tissues, seeds, plant cells, seeds, and progeny of the same. Plant cells include, without limitation, cells from seeds, suspension cultures, embryos, meristematic regions, callus tissue, leaves, roots, shoots, gametophytes, sporophytes, pollen, and microspores. Plant parts include differentiated and undifferentiated tissues including, but not limited to the following: roots, stems, shoots, leaves, pollen, seeds, fruit, harvested produce, tumor tissue, and various forms of cells and culture (e.g., single cells, protoplasts, embryos, and callus tissue). The plant tissue may be in a plant or in a plant organ, tissue, or cell culture. In addition, a plant may be genetically engineered to produce a heterologous protein or RNA, for example, of any of the pathogen control compositions in the methods or compositions described herein.

As used herein, the term “plant extracellular vesicle”, “plant EV”, or “EV” refers to an enclosed lipid-bilayer structure naturally occurring in a plant. Optionally, the plant EV includes one or more plant EV markers. As used herein, the term “plant EV marker” refers to a component that is naturally associated with a plant, such as a plant protein, a plant nucleic acid, a plant small molecule, a plant lipid, or a combination thereof, including but not limited to any of the plant EV markers listed in the Appendix. In some instances, the plant EV marker is an identifying marker of a plant EV but is not a pesticidal agent. In some instances, the plant EV marker is an identifying marker of a plant EV and also a pesticidal agent (e.g., either associated with or encapsulated by the plurality of PMPs, or not directly associated with or encapsulated by the plurality of PMPs).

As used herein, the term “plant messenger pack” or “PMP” refers to a lipid structure (e.g., a lipid bilayer, unilamellar, multilamellar structure; e.g., a vesicular lipid structure), that is about 5-2000 nm (e.g., at least 5-1000 nm, at least 5-500 nm, at least 400-500 nm, at least 25-250 nm, at least 50-150 nm, or at least 70-120 nm) in diameter that is derived from (e.g., enriched, isolated or purified from) a plant source or segment, portion, or extract thereof, including lipid or non-lipid components (e.g., peptides, nucleic acids, or small molecules) associated therewith and that has been enriched, isolated or purified from a plant, a plant part, or a plant cell, the enrichment or isolation removing one or more contaminants or undesired components from the source plant. PMPs may be highly purified preparations of naturally occurring EVs. Preferably, at least 1% of contaminants or undesired components from the source plant are removed (e.g., at least 2%, 5%, 10%, 15%, 20%, 25%, 30%, 40%, 45%, 50%, 55%, 60%, 70%, 80%, 90%, 95%, 96%, 98%, 99%, or 100%) of one or more contaminants or undesired components from the source plant, e.g., plant cell wall components; pectin; plant organelles (e.g., mitochondria; plastids such as chloroplasts, leucoplasts or amyloplasts; and nuclei); plant chromatin (e.g., a plant chromosome); or plant molecular aggregates (e.g., protein aggregates, protein-nucleic acid aggregates, lipoprotein aggregates, or lipido-proteic structures). Preferably, a PMP is at least 30% pure (e.g., at least 40% pure, at least 50% pure, at least 60% pure, at least 70% pure, at least 80% pure, at least 90% pure, at least 99% pure, or 100% pure) relative to the one or more contaminants or undesired components from the source plant as measured by weight (w/w), spectral imaging (% transmittance), or conductivity (S/m).

PMPs may optionally include additional agents, such as heterologous functional agents, e.g., pathogen control agents, repellent agents, polynucleotides, polypeptides, or small molecules. The PMPs can carry or associate with additional agents (e.g., heterologous functional agents) in a variety of ways to enable delivery of the agent to a target plant, e.g., by encapsulating the agent, incorporation of the agent in the lipid bilayer structure, or association of the agent (e.g., by conjugation) with the surface of the lipid bilayer structure. Heterologous functional agents can be incorporated into the PMPs either in vivo (e.g., in planta) or in vitro (e.g., in tissue culture, in cell culture, or synthetically incorporated). As used herein, the term “repellent” refers to an agent, composition, or substance therein, that deters pathogen vectors (e.g., insects, e.g., mosquitos, ticks, mites, or lice) from approaching or remaining on an animal. A repellent may, for example, decrease the number of pathogen vectors on or in the vicinity of an animal, but may not necessarily kill or decreasing the fitness of the pathogen vector.

As used herein, the term “treatment” refers to administering a pharmaceutical composition to an animal for prophylactic and/or therapeutic purposes. To “prevent an infection” refers to prophylactic treatment of an animal who is not yet ill, but who is susceptible to, or otherwise at risk of, a particular disease. To “treat an infection” refers to administering treatment to an animal already suffering from a disease to improve or stabilize the animal's condition.

As used herein, the term “treat an infection” refers to administering treatment to an individual already suffering from a disease to improve or stabilize the individual's condition. This may involve reducing colonization of a pathogen in, on, or around an animal by one or more pathogens (e.g., by about 1%, 2%, 5%, 10%, 20%, 30%, 40%, 50%, 60%, 70%, 80%, 90%, or 100%) relative to a starting amount and/or allow benefit to the individual (e.g., reducing colonization in an amount sufficient to resolve symptoms). In such instances, a treated infection may manifest as a decrease in symptoms (e.g., by about 1%, 2%, 5%, 10%, 20%, 30%, 40%, 50%, 60%, 70%, 80%, 90%, or 100%). In some instances, a treated infection is effective to increase the likelihood of survival of an individual (e.g., an increase in likelihood of survival by about 1%, 2%, 5%, 10%, 20%, 30%, 40%, 50%, 60%, 70%, 80%, 90%, or 100%) or increase the overall survival of a population (e.g., an increase in likelihood of survival by about 1%, 2%, 5%, 10%, 20%, 30%, 40%, 50%, 60%, 70%, 80%, 90%, or 100%). For example, the compositions and methods may be effective to “substantially eliminate” an infection, which refers to a decrease in the infection in an amount sufficient to sustainably resolve symptoms (e.g., for at least 1, 2, 3, 4, 5, 6, 7, 8, 9, 10, 11, or 12 months) in the animal.

As used herein, the term “prevent an infection” refers to preventing an increase in colonization in, on, or around an animal by one or more pathogens (e.g., by about 1%, 2%, 5%, 10%, 20%, 30%, 40%, 50%, 60%, 70%, 80%, 90%, 100%, or more than 100% relative to an untreated animal) in an amount sufficient to maintain an initial pathogen population (e.g., approximately the amount found in a healthy individual), prevent the onset of an infection, and/or prevent symptoms or conditions associated with infection. For example, individuals may receive prophylaxis treatment to prevent a fungal infection while being prepared for an invasive medical procedure (e.g., preparing for surgery, such as receiving a transplant, stem cell therapy, a graft, a prosthesis, receiving long-term or frequent intravenous catheterization, or receiving treatment in an intensive care unit), in immunocompromised individuals (e.g., individuals with cancer, with HIV/AIDS, or taking immunosuppressive agents), or in individuals undergoing long term antibiotic therapy.

As used herein, the term “stable PMP composition” (e.g., a composition including loaded or non-loaded PMPs) refers to a PMP composition that over a period of time (e.g., at least 24 hours, at least 48 hours, at least 1 week, at least 2 weeks, at least 3 weeks, at least 4 weeks, at least 30 days, at least 60 days, or at least 90 days) retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of the initial number of PMPs (e.g., PMPs per mL of solution) relative to the number of PMPs in the PMP composition (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)); or retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of its activity (e.g., pathogen control or repellent activity) relative to the initial activity of the PMP (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

As used herein, the term “untreated” refers to an animal or pathogen vector that has not been contacted with or delivered a pathogen control composition, including a separate animal that has not been delivered the pathogen control composition, the same animal undergoing treatment assessed at a time point prior to delivery of the pathogen control compositions, or the same animal undergoing treatment assessed at an untreated part of the animal.

As used herein, the term “vector” refers to an insect that can carry or transmit an animal pathogen from a reservoir to an animal. Exemplary vectors include insects, such as those with piercing-sucking mouthparts, as found in Hemiptera and some Hymenoptera and Diptera such as mosquitoes, bees, wasps, midges, lice, tsetse fly, fleas and ants, as well as members of the Arachnidae such as ticks and mites.

As used herein, the term “juice sac” or “juice vesicle” refers to a juice-containing membrane-bound component of the endocarp (carpel) of a hesperidium, e.g., a citrus fruit. In some aspects, the juice sacs are separated from other portions of the fruit, e.g., the rind (exocarp or flavedo), the inner rind (mesocarp, albedo, or pith), the central column (placenta), the segment walls, or the seeds. In some aspects, the juice sacs are juice sacs of a grapefruit, a lemon, a lime, or an orange.

### BRIEF DESCRIPTION OF THE DRAWINGS

FIG. 1A is a schematic diagram showing a protocol for grapefruit PMP production using a destructive juicing step involving the use of a blender, followed by ultracentrifugation and sucrose gradient purification. Images are included of the grapefruit juice after centrifugation at 1000×g for 10 min and the sucrose gradient band pattern after ultracentrifugation at 150,000×g for 2 hours.

FIG. 1B is a plot of the PMP particle distribution measured by the Spectradyne NCS1.

FIG. 2 is a schematic diagram showing a protocol for grapefruit PMP production using a mild juicing step involving use of a mesh filter, followed by ultracentrifugation and sucrose gradient purification. Images are included of the grapefruit juice after centrifugation at 1000×g for 10 min and the sucrose gradient band pattern after ultracentrifugation at 150,000×g for 2 hours.

FIG. 3A is a schematic diagram showing a protocol for grapefruit PMP production using ultracentrifugation, followed by size exclusion chromatography (SEC) to isolate the PMP-containing fractions. The eluted SEC fractions are analyzed for particle concentration (NanoFCM), median particle size (NanoFCM), and protein concentration (BCA).

FIG. 3B is a graph showing particle concentration per mL in eluted size exclusion chromatography (SEC) fractions (NanoFCM). The fractions containing the majority of PMPs (“PMP fraction”) are indicated with an arrow. PMPs are eluted in fractions 2-4.

FIG. 3C is a set of graphs and a table showing particle size in nm for selected SEC fractions, as measured using NanoFCM. The graphs show PMP size distribution in fractions 1, 3, 5, and 8.

FIG. 3D is a graph showing protein concentration in μg/mL in SEC fractions, as measured using a BCA assay. The fraction containing the majority of PMPs (“PMP fraction”) is labeled, and an arrow indicates a fraction containing contaminants.

FIG. 4A is a schematic diagram showing a protocol for scaled PMP production from 1 liter of grapefruit juice (˜7 grapefruits) using a juice press, followed by differential centrifugation to remove large debris, 100× concentration of the juice using TFF, and size exclusion chromatography (SEC) to isolate the PMP containing fractions. The SEC elution fractions are analyzed for particle concentration (NanoFCM), median particle size (NanoFCM) and protein concentration (BCA).

FIG. 4B is a pair of graphs showing protein concentration (BCA assay, top panel) and particle concentration (NanoFCM, bottom panel) of SEC eluate volume (ml) from a scaled starting material of 1000 ml of grapefruit juice, showing a high amount of contaminants in the late SEC elution volumes.

FIG. 4C is a graph showing that incubation of the crude grapefruit PMP fraction with a final concentration of 50 mM EDTA, pH 7.15 followed by overnight dialysis using a 300 kDa membrane, successfully removed contaminants present in the late SEC elution fractions, as shown by absorbance at 280 nm. There was no difference in the dialysis buffers used (PBS without calcium/magnesium pH 7.4, MES pH 6, Tris pH 8.6).

FIG. 4D is a graph showing that incubation of the crude grapefruit PMP fraction with a final concentration of 50 mM EDTA, pH 7.15, followed by overnight dialysis using a 300 kDa membrane, successfully removed contaminants present in the late elution fractions after SEC, as shown by BCA protein analysis, which, besides detecting protein, is sensitive to the presence of sugars and pectins. There was no difference in the dialysis buffers used (PBS without calcium/magnesium pH 7.4, MES pH 6, Tris pH 8.6).

FIG. 5A is a schematic diagram showing a protocol for PMP production from grapefruit juice using a juice press, followed by differential centrifugation to remove large debris, incubation with EDTA to reduce the formation of pectin macromolecules, sequential filtration to remove large particles, 5× concentration/wash by TFF, dialysis overnight to remove contaminants, further concentration by TFF (20× final), and SEC to isolate the PMP-containing fractions.

FIG. 5B is a graph showing the absorbance at 280 nm (A.U.) of eluted grapefruit SEC fractions using multiple SEC columns. PMPs are eluted in early fractions 4-6, and contaminants are eluted in late fractions.

FIG. 5C is a graph showing the protein concentration (μg/ml) of eluted grapefruit SEC fractions using multiple SEC columns. PMPs are eluted in early fractions 4-6, and contaminants are eluted in late fractions.

FIG. 5D is a graph showing the absorbance at 280 nm (A.U.) of eluted lemon SEC fractions using multiple SEC columns. PMPs are eluted in early fractions 4-6, and contaminants are eluted in late fractions.

FIG. 5E is a graph showing the protein concentration (μg/ml) of eluted lemon SEC fractions using multiple SEC columns. PMPs were eluted in early fractions 4-6, and contaminants were eluted in late fractions.

FIG. 5F is a scatter plot and a graph showing particle size in grapefruit PMP-containing SEC fractions after 0.22 um filter sterilization. The top panel is a scatter plot of particles in the combined SEC fractions, as measured by nano-flow cytometry (NanoFCM). The bottom panel is a size (nm) distribution graph of the gated particles (background subtracted). PMP concentration (particles/ml) and median size (nm) were determined using bead standards according to NanoFCM's instructions.

FIG. 5G is a scatter plot and a graph showing particle size in lemon PMP-containing SEC fractions after 0.22 um filter sterilization. The top panel is a scatter plot of particles in the combined SEC fractions, as measured by nano-flow cytometry (NanoFCM). The bottom panel is a size (nm) distribution graph of the gated particles (background subtracted). PMP concentration (particles/ml) and median size (nm) were determined using bead standards according to NanoFCM's instructions.

FIG. 5H is a graph showing grapefruit and lemon PMP stability at 4° Celsius, determined by the PMP concentration (PMP particles/ml) at different time points (days after production), as measured by NanoFCM.

FIG. 5I is a bar graph showing the stability of lemon (LM) PMPs after one freeze-thaw cycle at −20° Celsius and −20° Celsius compared to lemon PMPs stored at 4° Celsius, as determined by the PMP concentration (PMP particles/ml) after one week storage at the indicated temperatures, as measured by NanoFCM.

FIG. 6A is a graph showing particle concentration (particles/ml) in eluted BMS plant cell culture SEC fractions, as measured by nano-flow cytometry (NanoFCM). PMPs were eluted in SEC fractions 4-6.

FIG. 6B is a graph showing absorbance at 280 nm (A.U.) in eluted BMS SEC fractions, measured on a SpectraMax® spectrophotometer. PMPs were eluted in fractions 4-6; fractions 9-13 contained contaminants.

FIG. 6C is a graph showing protein concentration (μg/ml) in eluted BMS SEC fractions, as determined by BCA analysis. PMPs were eluted in fractions 4-6; fractions 9-13 contained contaminants.

FIG. 6D is a scatter plot showing particles in the combined BMS PMP-containing SEC fractions as measured by nano-flow cytometry (NanoFCM). PMP concentration (particles/ml) was determined using a bead standard according to NanoFCM's instructions.

FIG. 6E is a graph showing the size distribution of BMS PMPs (nm) for the gated particles (background subtracted) of FIG. 6D. Median PMP size (nm) was determined using Exo bead standards according to NanoFCM's instructions.

FIG. 7A is a scatter plot and a graph showing DyLight800 nm-labeled grapefruit PMPs as measured by Nano flow cytometry (NanoFCM). The top panel is a scatter plot of particles in the combined SEC fractions. The PMP concentration (4.44×10¹² PMPs/ml) was determined using a bead standard according to NanoFCM's instructions. The bottom panel is a size (nm) distribution graph of grapefruit DyLight800-PMPs. The median PMP size was determined using Exo bead standards according to NanoFCM's instructions. The median grapefruit DyLight800-PMPs size was 72.6 nm+/−14.6 nm (SD).

FIG. 7B is a scatter plot and a graph showing DyLight800 nm-labeled lemon PMPs as measured by Nano flow cytometry (NanoFCM). The median PMP concentration (5.18Ex10¹² PMPs/ml) was determined using a bead standard according to NanoFCM's instructions. The bottom panel is a size (nm) distribution graph of grapefruit DyLight800-PMPs. The PMP size was determined using Exo bead standards according to NanoFCM's instructions. The median lemon DyLight800-PMPs size was 68.5 nm+/−14 nm (SD).

FIG. 7C is a bar graph showing the uptake of grapefruit and lemon-derived DyL800 nm-labeled PMPs by bacteria (E. coli, and P. aeruginosa) and yeast (S. cerevisiae) 2 hours post-treatment. Uptake is defined in relative fluorescence intensity (A.U.), normalized to the relative fluorescence intensity of dye-only treated microbe controls.

FIG. 8A is a scatter plot and a graph showing purified lemon PMPs (combined and pelleted PMP SEC fractions), as measured by nano flow cytometry (NanoFCM). The top panel is a scatter plot of particles in the combined SEC fractions. The final lemon PMP concentration (1.53×10¹³ PMPs/ml) was determined using a bead standard according to NanoFCM's instructions. The bottom panel is a size (nm) distribution graph of purified lemon PMPs. The bottom panel is a size (nm) distribution graph of the gated particles. The median PMP size was determined using Exo bead standards according to NanoFCM's instructions. The median lemon PMP size was 72.4 nm+/−19.8 nm (SD).

FIG. 8B is a scatter plot and a graph showing Alexa Fluor® 488-(AF488)-labeled lemon PMPs as measured by nano flow cytometry (NanoFCM). The top panel is a scatter plot. Particles were gated on the FITC fluorescence signal, relative to unlabeled particles and background signal. The labeling efficiency was 99%, as determined by the number of fluorescent particles relative to the total number of particles detected. The final AF488-PMP concentration (1.34×10¹³ PMPs/ml) was determined from the number of fluorescent particles and using a bead standard with a known concentration according to NanoFCM's instructions. The bottom panel is a size (nm) distribution graph of AF488-labeled lemon PMPs. The median PMP size was determined using Exo bead standards according to NanoFCM's instructions. The median lemon PMPs size was 72.1 nm+/−15.9 nm (SD).

FIG. 9A is a graph showing the absorbance at 280 nm (A.U.) in eluted grapefruit SEC fractions produced from different SEC columns (Columns A, B, C, D, and E) measured on a SpectraMax® spectrophotometer. PMPs were eluted in fractions 4-6.

FIG. 9B is a scatter plot showing purified grapefruit PMPs (combined and pelleted PMP SEC fractions), as measured by nano flow cytometry (NanoFCM). The final grapefruit PMP concentration (6.34×10¹² PMPs/ml) was determined using a bead standard according to NanoFCM's instructions.

FIG. 9C is a graph showing size distribution (nm) of purified grapefruit PMPs. The median PMP size was determined using Exo bead standards according to NanoFCM's instructions. The median grapefruit PMPs size was 63.7 nm+/−11.5 nm (SD).

FIG. 9D is a graph showing the absorbance at 280 nm (A.U.) in eluted lemon SEC fractions of different SEC columns used, measured on a SpectraMax® spectrophotometer. PMPs were eluted in fractions 4-6.

FIG. 9E is a scatter plot showing purified lemon PMPs (combined and pelleted PMP SEC fractions), as measured by nano flow cytometry (NanoFCM). The final lemon PMP concentration (7.42×10¹² PMPs/ml) was determined using a bead standard according to NanoFCM's instructions.

FIG. 9F is a graph showing size distribution (nm) of purified lemon PMPs. The median PMP size was determined using Exo bead standards according to NanoFCM's instructions. The median lemon PMPs size was 68 nm+/−17.5 nm (SD).

FIG. 9G is a bar graph showing the DOX loading capacity (pg DOX per 1000 PMPs) of lemon (LM) and grapefruit (GF) PMPs that were actively (sonication/extrusion) or passively (incubation) loaded with doxorubicin. The loading capacity was calculated by dividing the total concentration of DOX (pg/mL) in the PMP-DOX sample (assessed by fluorescence intensity measurement (Ex/Em=485/550 nm) using a SpectraMax® spectrophotometer) by the total PMP concentration (PMPs/mL) in the sample.

FIG. 9H is a graph showing the stability of grapefruit and lemon DOX-loaded PMP at 4° Celsius, as determined by the PMP concentration (PMP particles/ml) at different time points (days after loading), as measured by NanoFCM.

FIG. 10A is a schematic diagram showing a protocol production of PMPs from 4 liters of grapefruit juice treated with pectinase and EDTA, concentrated 5× using a 300 kDa TFF, washed by 6 volume exchanges of PBS, and concentrated to a final concentration of 20×. Size exclusion chromatography was used to elute the PMP-containing fractions.

FIG. 10B is a graph showing the absorbance at 280 nm (A.U.) of eluted SEC fractions across 9 different SEC columns used (SEC column A-J). PMPs are eluted in SEC fractions 3-7.

FIG. 10C is a graph showing the protein concentration (μg/ml) of eluted SEC fractions across 9 different SEC columns used (SEC column A-J). PMPs are eluted in SEC fractions 3-7. An arrow indicates a fraction containing contaminants.

FIG. 10D is a scatter plot showing purified grapefruit PMPs (combined and pelleted PMP SEC fractions), as measured by nano flow cytometry (NanoFCM). The final grapefruit PMP concentration (7.56×10¹² PMPs/ml) was determined using a bead standard according to NanoFCM's instructions.

FIG. 10E is a graph showing size distribution (nm) of purified grapefruit PMPs. The median PMP size was determined using Exo bead standards according to NanoFCM's instructions. The median grapefruit PMPs size was 70.3 nm+/−12.4 nm (SD).

FIG. 10F is a graph showing the cytotoxic effect of doxorubicin (DOX)-loaded grapefruit PMP treatment of P. aeruginosa. Bacteria were treated in duplicate with PMP-DOX to an effective DOX concentration of 0 (negative control), 5 μM, 10 μM, 25 μM, 50 μM and 100 μM. A kinetic Absorbance measurement at 600 nm was performed (SpectraMax® spectrophotometer) to monitor the OD of the cultures at the indicated time points. All OD values per treatment dose were first normalized to the OD of the first time point at that dose, to normalize for DOX fluorescence bleed-through at 600 nm at high concentration. To determine the cytotoxic effect of PMP-DOX on bacteria, the relative OD was determined within each treatment group as compared to the untreated control (set to 100%).

FIG. 10G is a graph showing the cytotoxic effect of doxorubicin (DOX)-loaded grapefruit PMP treatment of E. coli. Bacteria were treated in duplicate with PMP-DOX to an effective DOX concentration of 0 (negative control), 5 μM, 10 μM, 25 μM, 50 μM and 100 μM. A kinetic Absorbance measurement at 600 nm was performed (SpectraMax® spectrophotometer) to monitor the OD of the cultures at the indicated time points. All OD values per treatment dose were first normalized to the OD of the first time point at that dose, to normalize for DOX fluorescence bleed-through at 600 nm at high concentration. To determine the cytotoxic effect of PMP-DOX on bacteria, the relative OD was determined within each treatment group as compared to the untreated control (set to 100%).

FIG. 10H is a graph showing the cytotoxic effect of doxorubicin (DOX)-loaded grapefruit PMP treatment of S.cerevisiae. Yeast cells were treated in duplicate with PMP-DOX to an effective DOX concentration of 0 (negative control), 5 μM, 10 μM, 25 μM, 50 μM and 100 μM. A kinetic Absorbance measurement at 600 nm was performed (SpectraMax® spectrophotometer) to monitor the OD of the cultures at the indicated time points. All OD values per treatment dose were first normalized to the OD of the first time point at that dose, to normalize for DOX fluorescence bleed-through at 600 nm at high concentration. To determine the cytotoxic effect of PMP-DOX on yeast, the relative OD was determined within each treatment group as compared to the untreated control (set to 100%).

FIG. 11 is a graph showing the luminescence (R.L.U., relative luminescence unit) of Pseudomonas aeruginosa bacteria that were treated with Ultrapure water (negative control), 3 ng free luciferase protein (protein only control) or with an effective luciferase protein dose of 3 ng by luciferase protein-loaded PMPs (PMP-Luc) in duplicate samples for 2 hrs at RT. Luciferase protein in the supernatant and pelleted bacteria was measured by luminescence using the ONE-Glo™ luciferase assay kit (Promega) and measured on a SpectraMax® spectrophotometer.

### DETAILED DESCRIPTION

Featured herein are compositions and related methods for controlling pathogens based on pathogen control compositions that include plant messenger packs (PMPs), lipid assemblies produced wholly or in part from plant extracellular vesicles (EVs), or segments, portions, or extracts thereof. The PMPs can have antipathogen (e.g., an agent suitable for administration to animals to treat infection, e.g., an antibacterial agent, virucidal agent, antiviral agent, antiparasitic agent, or a nematicidal agent), pesticidal, or insect repellant activity without the inclusion of additional agents, but may be optionally modified to include additional antipathogen, pesticidal, or pest repellent agents. Also included are formulations in which the PMPs are provided in substantially pure form or concentrated forms. The pathogen control compositions and formulations described herein can be delivered directly to an animal to treat or prevent pathogen infections. Additionally, or alternatively, the pathogen control compositions can be delivered to a variety of animal pathogens or vectors of animal pathogens to decrease the fitness of the pathogen, or vector thereof, and thereby control the spread of harmful pathogens.

I. Pathogen Control Compositions

The pathogen control compositions described herein include a plurality of plant messenger packs (PMPs). A PMP is a lipid (e.g., lipid bilayer, unilamellar, or multilamellar structure) structure that includes a plant EV, or segment, portion, or extract (e.g., lipid extract) thereof. Plant EVs refer to an enclosed lipid-bilayer structure that naturally occurs in a plant. PMPs may be about 5-2000 nm in diameter. Plant EVs can originate from a variety of plant biogenesis pathways. In nature, plant EVs can be found in the intracellular and extracellular compartments of plants, such as the plant apoplast, the compartment located outside the plasma membrane and formed by a continuum of cell walls and the extracellular space. Alternatively, PMPs can be enriched plant EVs found in cell culture media upon secretion from plant cells. Plant EVs can be separated from plants (e.g., from the apoplastic fluid), thereby providing PMPs by a variety of methods, further described herein.

The pathogen control compositions can include PMPs that have antipathogen activity (e.g., antibacterial, antifungal, antinematicidal, antiparasitic, or antiviral activity), pesticidal activity, or repellent activity against pathogens, without the further inclusion of additional antipathogen, pesticidal, or repellent agents. However, PMPs can additionally include a heterologous pathogen control agent, e.g., antipathogen agent (e.g., antibacterial, antifungal, antinematicidal, antiparasitic, or antiviral), pesticidal agent, or repellent agent, which can be introduced in vivo or in vitro. As such, the PMPs can include a substance with antipathogen, pesticidal activity that is loaded into or onto the PMP by the plant from which the PMP is produced. For example, a heterologous functional agent loaded into the PMP in vivo may be a factor endogenous to a plant or a factor exogenous to a plant (e.g., as expressed by a heterologous genetic construct in a genetically engineered plant). Alternatively, the PMPs may be loaded with a heterologous functional agent in vitro (e.g., following production by a variety of methods further described herein).

PMPs can include plant EVs, or segments, portions, or extracts, thereof, in which the plant EVs are about 5-2000 nm in diameter. For example, the PMP can include a plant EV, or segment, portion, or extract thereof, that has a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1250 nm, about 1250-1500 nm, about 1500-1750 nm, or about 1750-2000 nm. In some instances, the PMP includes a plant EV, or segment, portion, or extract thereof, that has a mean diameter of about 5-950 nm, about 5-900 nm, about 5-850 nm, about 5-800 nm, about 5-750 nm, about 5-700 nm, about 5-650 nm, about 5-600 nm, about 5-550 nm, about 5-500 nm, about 5-450 nm, about 5-400 nm, about 5-350 nm, about 5-300 nm, about 5-250 nm, about 5-200 nm, about 5-150 nm, about 5-100 nm, about 5-50 nm, or about 5-25 nm. In certain instances, the plant EV, or segment, portion, or extract thereof, has a mean diameter of about 50-200 nm. In certain instances, the plant EV, or segment, portion, or extract thereof, has a mean diameter of about 50-300 nm. In certain instances, the plant EV, or segment, portion, or extract thereof, has a mean diameter of about 200-500 nm. In certain instances, the plant EV, or segment, portion, or extract thereof, has a mean diameter of about 30-150 nm.

In some instances, the PMP may include a plant EV, or segment, portion, or extract thereof, that has a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, or at least 1000 nm. In some instances, the PMP includes a plant EV, or segment, portion, or extract thereof, that has a mean diameter less than 1000 nm, less than 950 nm, less than 900 nm, less than 850 nm, less than 800 nm, less than 750 nm, less than 700 nm, less than 650 nm, less than 600 nm, less than 550 nm, less than 500 nm, less than 450 nm, less than 400 nm, less than 350 nm, less than 300 nm, less than 250 nm, less than 200 nm, less than 150 nm, less than 100 nm, or less than 50 nm. A variety of methods (e.g., a dynamic light scattering method) standard in the art can be used to measure the particle diameter of the plant EVs, or segment, portion, or extract thereof.

In some instances, the PMP may include a plant EV, or segment, portion, or extract thereof, that has a mean surface area of 77 nm² to 3.2×10⁶ nm² (e.g., 77-100 nm², 100-1000 nm², 1000-1×10⁴ nm², 1×10⁴-1×10⁵ nm², 1×10⁵-1×10⁶ nm², or 1×10⁶-3.2×10⁶ nm²). In some instances, the PMP may include a plant EV, or segment, portion, or extract thereof, that has a mean volume of 65 nm³ to 5.3×10⁸ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-5.3×10⁸ nm³). In some instances, the PMP may include a plant EV, or segment, portion, or extract thereof, that has a mean surface area of at least 77 nm², (e.g., at least 77 nm², at least 100 nm², at least 1000 nm², at least 1×10⁴ nm², at least 1×10⁵ nm², at least 1×10⁶ nm², or at least 2×10⁶ nm²). In some instances, the PMP may include a plant EV, or segment, portion, or extract thereof, that has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 2×10⁸ nm³, at least 3×10⁸ nm³, at least 4×10⁸ nm³, or at least 5×10⁸ nm³.

In some instances, the PMP can have the same size as the plant EV or segment, extract, or portion thereof. Alternatively, the PMP may have a different size than the initial plant EV from which the PMP is produced. For example, the PMP may have a diameter of about 5-2000 nm in diameter. For example, the PMP can have a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1200 nm, about 1200-1400 nm, about 1400-1600 nm, about 1600-1800 nm, or about 1800-2000 nm. In some instances, the PMP may have a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1000 nm, at least 1200 nm, at least 1400 nm, at least 1600 nm, at least 1800 nm, or about 2000 nm. A variety of methods (e.g., a dynamic light scattering method) standard in the art can be used to measure the particle diameter of the PMPs. In some instances, the size of the PMP is determined following loading of heterologous functional agents, or following other modifications to the PMPs.

In some instances, the PMP may have a mean surface area of 77 nm² to 1.3×10⁷ nm² (e.g., 77-100 nm², 100-1000 nm², 1000-1×10⁴ nm², 1×10⁴-1×10⁵ nm², 1×10⁵-1×10⁶ nm², or 1×10⁶-1.3×10⁷ nm²). In some instances, the PMP may have a mean volume of 65 nm³ to 4.2×10⁹ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-1×10⁹ nm³, or 1×10⁹-4.2×10⁹ nm³). In some instances, the PMP has a mean surface area of at least 77 nm², (e.g., at least 77 nm², at least 100 nm², at least 1000 nm², at least 1×10⁴ nm², at least 1×10⁵ nm², at least 1×10⁶ nm², or at least 1×10′ nm²). In some instances, the PMP has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 1×10⁹ nm³, at least 2×10⁹ nm³, at least 3×10⁹ nm³, or at least 4×10⁹ nm³).

In some instances, the PMP may include an intact plant EV. Alternatively, the PMP may include a segment, portion, or extract of the full surface area of the vesicle (e.g., a segment, portion, or extract including less than 100% (e.g., less than 90%, less than 80%, less than 70%, less than 60%, less than 50%, less than 40%, less than 30%, less than 20%, less than 10%, less than 10%, less than 5%, or less than 1%) of the full surface area of the vesicle) of a plant EV. The segment, portion, or extract may be any shape, such as a circumferential segment, spherical segment (e.g., hemisphere), curvilinear segment, linear segment, or flat segment. In instances where the segment is a spherical segment of the vesicle, the spherical segment may represent one that arises from the splitting of a spherical vesicle along a pair of parallel lines, or one that arises from the splitting of a spherical vesicle along a pair of non-parallel lines. Accordingly, the plurality of PMPs can include a plurality of intact plant EVs, a plurality of plant EV segments, portions, or extracts, or a mixture of intact and segments of plant EVs. One skilled in the art will appreciate that the ratio of intact to segmented plant EVs will depend on the particular isolation method used. For example, grinding or blending a plant, or part thereof, may produce PMPs that contain a higher percentage of plant EV segments, portions, or extracts than a non-destructive extraction method, such as vacuum-infiltration.

In instances where, the PMP includes a segment, portion, or extract of a plant EV, the EV segment, portion, or extract may have a mean surface area less than that of an intact vesicle, e.g., a mean surface area less than 77 nm², 100 nm², 1000 nm², 1×10⁴ nm², 1×10⁵ nm², 1×10⁶ nm², or 3.2×10⁶ nm²). In some instances, the EV segment, portion, or extract has a surface area of less than 70 nm², 60 nm², 50 nm², 40 nm², 30 nm², 20 nm², or 10 nm²). In some instances, the PMP may include a plant EV, or segment, portion, or extract thereof, that has a mean volume less than that of an intact vesicle, e.g., a mean volume of less than 65 nm³, 100 nm³, 1000 nm³, 1×10⁴ nm³, 1×10⁵ nm³, 1×10⁶ nm³, 1×10⁷ nm³, 1×10⁸ nm³, or 5.3×10⁸ nm³).

In instances where the PMP includes an extract of a plant EV, e.g., in instances where the PMP includes lipids extracted (e.g., with chloroform) from a plant EV, the PMP may include at least 1%, 2%, 5%, 10%, 20%, 30%, 40%, 50%, 60% or more, of lipids extracted (e.g., with chloroform) from a plant EV. The PMPs in the plurality may include plant EV segments and/or plant EV-extracted lipids or a mixture thereof.

Further outlined herein are details regarding methods of producing PMPs, plant EV markers that can be associated with PMPs, and formulations for compositions including PMPs.

### A. Production Methods

PMPs may be produced from plant EVs, or a segment, portion or extract (e.g., lipid extract) thereof, that occur naturally in plants, or parts thereof, including plant tissues or plant cells. An exemplary method for producing PMPs includes (a) providing an initial sample from a plant, or a part thereof, wherein the plant or part thereof comprises EVs; and (b) isolating a crude PMP fraction from the initial sample, wherein the crude PMP fraction has a decreased level of at least one contaminant or undesired component from the plant or part thereof relative to the level in the initial sample. The method can further include an additional step (c) comprising purifying the crude PMP fraction, thereby producing a plurality of pure PMPs, wherein the plurality of pure PMPs have a decreased level of at least one contaminant or undesired component from the plant or part thereof relative to the level in the crude EV fraction. Each production step is discussed in further detail, below. Exemplary methods regarding the isolation and purification of PMPs is found, for example, in Rutter and Innes, Plant Physiol. 173(1): 728-741, 2017; Rutter et al, Bio. Protoc. 7(17): e2533, 2017; Regente et al, J of Exp. Biol. 68(20): 5485-5496, 2017; Mu et al, Mol. Nutr. Food Res., 58, 1561-1573, 2014, and Regente et al, FEBS Letters. 583: 3363-3366, 2009, each of which is herein incorporated by reference.

For example, a plurality of PMPs may be isolated from a plant by a process which includes the steps of: (a) providing an initial sample from a plant, or a part thereof, wherein the plant or part thereof comprises EVs; (b) isolating a crude PMP fraction from the initial sample, wherein the crude PMP fraction has a decreased level of at least one contaminant or undesired component from the plant or part thereof relative to the level in the initial sample (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%); and (c) purifying the crude PMP fraction, thereby producing a plurality of pure PMPs, wherein the plurality of pure PMPs have a decreased level of at least one contaminant or undesired component from the plant or part thereof relative to the level in the crude EV fraction (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%).

The PMPs provided herein can include a plant EV, or segment, portion, or extract thereof, isolated from a variety of plants. PMPs may be isolated from any genera of plants (vascular or nonvascular), including but not limited to angiosperms (monocotyledonous and dicotyledonous plants), gymnosperms, ferns, selaginellas, horsetails, psilophytes, lycophytes, algae (e.g., unicellular or multicellular, e.g., archaeplastida), or bryophytes. In certain instances, PMPs can be produced from a vascular plant, for example monocotyledons or dicotyledons or gymnosperms. For example, PMPs can be produced from alfalfa, apple, Arabidopsis, banana, barley, canola, castor bean, chicory, chrysanthemum, clover, cocoa, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat or vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes, kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, or wheat.

PMPs may be produced from a whole plant (e.g., a whole rosettes or seedlings) or alternatively from one or more plant parts (e.g., leaf, seed, root, fruit, vegetable, pollen, phloem sap, or xylem sap). For example, PMPs can be produced from shoot vegetative organs/structures (e.g., leaves, stems, or tubers), roots, flowers and floral organs/structures (e.g., pollen, bracts, sepals, petals, stamens, carpels, anthers, or ovules), seed (including embryo, endosperm, or seed coat), fruit (the mature ovary), sap (e.g., phloem or xylem sap), plant tissue (e.g., vascular tissue, ground tissue, tumor tissue, or the like), and cells (e.g., single cells, protoplasts, embryos, callus tissue, guard cells, egg cells, or the like), or progeny of same. For instance, the isolation step may involve (a) providing a plant, or a part thereof. In some examples, the plant part is an Arabidopsis leaf. The plant may be at any stage of development. For example, the PMP can be produced from seedlings, e.g., 1 week, 2 week, 3 week, 4 week, 5 week, 6 week, 7 week, or 8 week old seedlings (e.g., Arabidopsis seedlings). Other exemplary PMPs can include PMPs produced from roots (e.g., ginger roots), fruit juice (e.g., grapefruit juice), vegetables (e.g., broccoli), pollen (e.g., olive pollen), phloem sap (e.g., Arabidopsis phloem sap), or xylem sap (e.g., tomato plant xylem sap).

PMPs can be produced from a plant, or part thereof, by a variety of methods. Any method that allows release of the EV-containing apoplastic fraction of a plant, or an otherwise extracellular fraction that contains PMPs comprising secreted EVs (e.g., cell culture media) is suitable in the present methods. EVs can be released by either destructive (e.g., grinding or blending of a plant, or any plant part) or non-destructive (washing or vacuum infiltration of a plant or any plant part) methods. For instance, the plant, or part thereof, can be vacuum-infiltrated, ground, blended, or a combination thereof to isolate EVs from the plant or plant part, thereby producing PMPs. For instance, the isolating step may involve (b) isolating a crude PMP fraction from the initial sample (e.g., a plant, a plant part, or a sample derived from a plant or plant part), wherein the isolating step involves vacuum infiltrating the plant (e.g., with a vesicle isolation buffer) to release and collect the apoplastic fraction. Alternatively, the isolating step may involve (b) providing a plant, or a part thereof, wherein the releasing step involves grinding or blending the plant to release the EVs, thereby producing PMPs.

Upon isolating the plant EVs, thereby producing PMPs, the PMPs can be separated or collected into a crude PMP fraction (e.g., an apoplastic fraction). For instance, the separating step may involve separating the plurality of PMPs into a crude PMP fraction using centrifugation (e.g., differential centrifugation or ultracentrifugation) and/or filtration to separate the PMP-containing fraction from large contaminants, including plant tissue debris, plant cells, or plant cell organelles (e.g., nuclei, mitochondria, or chloroplasts). As such, the crude plant EV fraction will have a decreased number of large contaminants, including, for example, plant tissue debris, plant cells, or plant cell organelles (e.g., nuclei, mitochondria or chloroplast), as compared to the initial sample from the source plant or plant part.

The crude PMP fraction can be further purified by additional purification methods to produce a plurality of pure PMPs. For example, the crude PMP fraction can be separated from other plant components by ultracentrifugation, e.g., using a density gradient (iodixanol or sucrose), size-exclusion, and/or use of other approaches to remove aggregated components (e.g., precipitation or size-exclusion chromatography). The resulting pure PMPs may have a decreased level of contaminants (e.g., one or more non-PMP components, such as protein aggregates, nucleic acid aggregates, protein-nucleic acid aggregates, free lipoproteins, lipido-proteic structures), nuclei, cell wall components, cell organelles, or a combination thereof) relative to one or more fractions generated during the earlier separation steps, or relative to a pre-established threshold level, e.g., a commercial release specification. For example, the pure PMPs may have a decreased level (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) of plant organelles or cell wall components relative to the level in the initial sample. In some instances, the pure PMPs are is substantially free (e.g., have undetectable levels) of one or more non-PMP components, such as protein aggregates, nucleic acid aggregates, protein-nucleic acid aggregates, free lipoproteins, lipido-proteic structures), nuclei, cell wall components, cell organelles, or a combination thereof. Further examples of the releasing and separation steps can be found in Example 1. The PMPs may be at a concentration of, e.g., 1×10⁹, 5×10⁹, 1×10¹⁰ 5×10¹⁰, 5×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, or more than 1×10¹³ PMPs/mL.

For example, protein aggregates may be removed from isolated PMPs. For example, the isolated PMP solution can be taken through a range of pHs (e.g., as measured using a pH probe) to precipitate out protein aggregates in solution. The pH can be adjusted to, e.g., pH 3, pH 5, pH 7, pH 9, or pH 11 with the addition of, e.g., sodium hydroxide or hydrochloric acid. Once the solution is at the specified pH, it can be filtered to remove particulates. Alternatively, the isolated PMP solution can be flocculated using the addition of charged polymers, such as Polymin-P or Praestol 2640. Briefly, Polymin-P or Praestol 2640 is added to the solution and mixed with an impeller. The solution can then be filtered to remove particulates. Alternatively, aggregates can be solubilized by increasing salt concentration. For example NaCl can be added to the isolated PMP solution until it is at, e.g., 1 mol/L. The solution can then be filtered to isolate the PMPs. Alternatively, aggregates are solubilized by increasing the temperature. For example, the isolated PMPs can be heated under mixing until the solution has reached a uniform temperature of, e.g., 50° C. for 5 minutes. The PMP mixture can then be filtered to isolate the PMPs. Alternatively, soluble contaminants from PMP solutions can be separated by size-exclusion chromatography column according to standard procedures, where PMPs elute in the first fractions, whereas proteins and ribonucleoproteins and some lipoproteins are eluted later. The efficiency of protein aggregate removal can be determined by measuring and comparing the protein concentration before and after removal of protein aggregates via BCA/Bradford protein quantification.

Any of the production methods described herein can be supplemented with any quantitative or qualitative methods known in the art to characterize or identify the PMPs at any step of the production process. PMPs may be characterized by a variety of analysis methods to estimate PMP yield, PMP concentration, PMP purity, PMP composition, or PMP sizes. PMPs can be evaluated by a number of methods known in the art that enable visualization, quantitation, or qualitative characterization (e.g., identification of the composition) of the PMPs, such as microscopy (e.g., transmission electron microscopy), dynamic light scattering, nanoparticle tracking, spectroscopy (e.g., Fourier transform infrared analysis), or mass spectrometry (protein and lipid analysis). In certain instances, methods (e.g., mass spectroscopy) may be used to identify plant EV markers present on the PMP, such as markers disclosed in the Appendix. To aid in analysis and characterization, of the PMP fraction, the PMPs can additionally be labelled or stained. For example, the PMPs can be stained with 3,3′-dihexyloxacarbocyanine iodide (DIOC₆), a fluorescent lipophilic dye, PKH67 (Sigma Aldrich); Alexa Fluor® 488 (Thermo Fisher Scientific), or DyLight™ 800 (Thermo Fisher). In the absence of sophisticated forms of nanoparticle tracking, this relatively simple approach quantifies the total membrane content and can be used to indirectly measure the concentration of PMPs (Rutter and Innes, Plant Physiol. 173(1): 728-741, 2017; Rutter et al, Bio. Protoc. 7(17): e2533, 2017). For more precise measurements, and to assess the size distributions of PMPs, nanoparticle tracking or Tunable Resistive Pulse Sensing can be used.

During the production process, the PMPs can optionally be prepared such that the PMPs are at an increased concentration (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) relative to the EV level in a control or initial sample. The isolated PMPs may make up about 0.1% to about 100% of the pathogen control composition, such as any one of about 0.01% to about 100%, about 1% to about 99.9%, about 0.1% to about 10%, about 1% to about 25%, about 10% to about 50%, about 50% to about 99%, or about 75% to about 100%. In some instances, the composition includes at least any of 0.1%, 0.5%, 1%, 5%, 10%, 20%, 30%, 40%, 50%, 60%, 70%, 80%, 90%, 95%, or more PMPs, e.g., as measured by wt/vol, percent PMP protein composition, and/or percent lipid composition (e.g., by measuring fluorescently labelled lipids); See, e.g., Example 3). In some instances, the concentrated agents are used as commercial products, e.g., the final user may use diluted agents, which have a substantially lower concentration of active ingredient. In some embodiments, the composition is formulated as a pathogen control concentrate formulation, e.g., an ultra-low-volume concentrate formulation.

As illustrated by Example 1, PMPs can be produced from a variety of plants, or parts thereof (e.g., the leaf apoplast, seed apoplast, root, fruit, vegetable, pollen, phloem, or xylem sap). For example, PMPs can be isolated from the apoplastic fraction of a plant, such as the apoplast of a leaf (e.g., apoplast Arabidopsis thaliana leaves) or the apoplast of seeds (e.g., apoplast of sunflower seeds). Other exemplary PMPs are produced from roots (e.g., ginger roots), fruit juice (e.g., grapefruit juice), vegetables (e.g., broccoli), pollen (e.g., olive pollen), phloem sap (e.g., Arabidopsis phloem sap), xylem sap (e.g., tomato plant xylem sap), or cell culture supernatant (e.g. BY2 tobacco cell culture supernatant). This example further demonstrates the production of PMPs from these various plant sources.

As illustrated by Example 2, PMPs can be purified by a variety of methods, for example, by using a density gradient (iodixanol or sucrose) in conjunction with ultracentrifugation and/or methods to remove aggregated contaminants, e.g., precipitation or size-exclusion chromatography. For instance, Example 2 illustrates purification of PMPs that have been obtained via the separation steps outlined in Example 1. Further, PMPs can be characterized in accordance with the methods illustrated in Example 3.

In some instances, the PMPs of the present compositions and methods can be isolated from a plant, or part thereof, and used without further modification to the PMP. In other instances, the PMP can be modified prior to use, as outlined further herein.

### B. Plant EV-Markers

The PMPs of the present compositions and methods may have a range of markers that identify the PMP as being produced from a plant EV, and/or including a segment, portion, or extract thereof. As used herein, the term “plant EV-marker” refers to a component that is naturally associated with a plant and incorporated into or onto the plant EV in planta, such as a plant protein, a plant nucleic acid, a plant small molecule, a plant lipid, or a combination thereof. Examples of plant EV-markers can be found, for example, in Rutter and Innes, Plant Physiol. 173(1): 728-741, 2017; Raimondo et al., Oncotarget. 6(23): 19514, 2015; Ju et al., Mol. Therapy. 21(7):1345-1357, 2013; Wang et al., Molecular Therapy. 22(3): 522-534, 2014; and Regente et al, J of Exp. Biol. 68(20): 5485-5496, 2017; each of which is incorporated herein by reference. Additional examples of plant EV-markers are listed in the Appendix, and are further outlined herein.

The plant EV marker can include a plant lipid. Examples of plant lipid markers that may be found in the PMP include phytosterol, campesterol, β-sitosterol, stigmasterol, avenasterol, glycosyl inositol phosphoryl ceramides (GIPCs), glycolipids (e.g., monogalactosyldiacylglycerol (MGDG) or digalactosyldiacylglycerol (DGDG)), or a combination thereof. For instance, the PMP may include GIPCs, which represent the main sphingolipid class in plants and are one of the most abundant membrane lipids in plants. Other plant EV markers may include lipids that accumulate in plants in response to abiotic or biotic stressors (e.g., bacterial or fungal infection), such as phosphatidic acid (PA) or phosphatidylinositol-4-phosphate (PI4P).

Alternatively, the plant EV marker may include a plant protein. In some instances, the protein plant EV marker may be an antimicrobial protein naturally produced by plants, including defense proteins that plants secrete in response to abiotic or biotic stressors (e.g., bacterial or fungal infection). Plant pathogen defense proteins include soluble N-ethylmalemide-sensitive factor association protein receptor protein (SNARE) proteins (e.g., Syntaxin-121 (SYP121; GenBank Accession No.: NP_187788.1 or NP_974288.1), Penetration1 (PEN1; GenBank Accession No: NP_567462.1)) or ABC transporter Penetration3 (PENS; GenBank Accession No: NP_191283.2). Other examples of plant EV markers includes proteins that facilitate the long-distance transport of RNA in plants, including phloem proteins (e.g., Phloem protein2-A1 (PP2-A1), GenBank Accession No: NP_193719.1), calcium-dependent lipid-binding proteins, or lectins (e.g., Jacalin-related lectins, e.g., Helianthus annuus jacalin (Helja; GenBank: AHZ86978.1). For example, the RNA binding protein may be Glycine-Rich RNA Binding Protein-7 (GRP7; GenBank Accession Number: NP_179760.1). Additionally, proteins that regulate plasmodesmata function can in some instances be found in plant EVs, including proteins such as Synap-Totgamin A A (GenBank Accession No: NP_565495.1). In some instances, the plant EV marker can include a protein involved in lipid metabolism, such as phospholipase C or phospholipase D. In some instances, the plant protein EV marker is a cellular trafficking protein in plants. In certain instances where the plant EV marker is a protein, the protein marker may lack a signal peptide that is typically associated with secreted proteins. Unconventional secretory proteins seem to share several common features like (i) lack of a leader sequence, (ii) absence of PTMs specific for ER or Golgi apparatus, and/or (iii) secretion not affected by brefeldin A which blocks the classical ER/Golgi-dependent secretion pathway. One skilled in the art can use a variety of tools freely accessible to the public (e.g., SecretomeP Database; SUBA3 (SUBcellular localization database for Arabidopsis proteins)) to evaluate a protein for a signal sequence, or lack thereof.

In instances where the plant EV marker is a protein, the protein may have an amino acid sequence having at least 35%, 40%, 45%, 50%, 55%, 60%, 65%, 70%, 75%, 80%, 85%, 90%, 95%, 98%, 99%, or 100% sequence identity to a plant EV marker, such as any of the plant EV markers listed in the Appendix. For example, the protein may have an amino acid sequence having at least 35%, 40%, 45%, 50%, 55%, 60%, 65%, 70%, 75%, 80%, 85%, 90%, 95%, 98%, 99%, or 100% sequence identity to PEN1 from Arabidopsis thaliana (GenBank Accession Number: NP_567462.1).

In some instances, the plant EV marker includes a nucleic acid encoded in plants, e.g., a plant RNA, a plant DNA, or a plant PNA. For example, the PMP may include dsRNA, mRNA, a viral RNA, a microRNA (miRNA), or a small interfering RNA (siRNA) encoded by a plant. In some instances, the nucleic acid may be one that is associated with a protein that facilitates the long-distance transport of RNA in plants, as discussed herein. In some instances, the nucleic acid plant EV marker may be one involved in host-induced gene silencing (HIGS), which is the process by which plants silence foreign transcripts of plant pests (e.g., pathogens such as fungi). For example, the nucleic acid may be one that silences bacterial or fungal genes. In some instances, the nucleic acid may be a microRNA, such as miR159 or miR166, which target genes in a fungal pathogen (e.g., Verticillium dahliae). In some instances, the protein may be one involved in carrying plant defense compounds, such as proteins involved in glucosinolate (GSL) transport and metabolism, including Glucosinolate Transporter-1-1 (GTR1; GenBank Accesion No: NP_566896.2), Glucosinolate Transporter-2 (GTR2; NP_201074.1), orEpithiospecific Modifier 1 (ESM1; NP_188037.1).

In instances where the plant EV marker is a nucleic acid, the nucleic acid may have a nucleotide sequence having at least 35%, 40%, 45%, 50%, 55%, 60%, 65%, 70%, 75%, 80%, 85%, 90%, 95%, 98%, 99%, or 100% sequence identity to a plant EV marker, e.g., such as those encoding the plant EV markers listed in the Appendix. For example, the nucleic acid may have a polynucleotide sequence having at least 35%, 40%, 45%, 50%, 55%, 60%, 65%, 70%, 75%, 80%, 85%, 90%, 95%, 98%, 99%, or 100% sequence identity to miR159 or miR166.

In some instances, the plant EV marker includes a compound produced by plants. For example, the compound may be a defense compound produced in response to abiotic or biotic stressors, such as secondary metabolites. One such secondary metabolite that be found in PMPs are glucosinolates (GSLs), which are nitrogen and sulfur-containing secondary metabolites found mainly in Brassicaceae plants. Other secondary metabolites may include allelochemicals.

In some instances, the PMP may also be identified as being produced from a plant EV based on the lack of certain markers (e.g., lipids, polypeptides, or polynucleotides) that are not typically produced by plants, but are generally associated with other organisms (e.g., markers of animal EVs, bacterial EVs, or fungal EVs). For example, in some instances, the PMP lacks lipids typically found in animal EVs, bacterial EVs, or fungal EVs. In some instances, the PMP lacks lipids typical of animal EVs (e.g., sphingomyelin). In some instances, the PMP does not contain lipids typical of bacterial EVs or bacterial membranes (e.g., LPS). In some instances, the PMP lacks lipids typical of fungal membranes (e.g., ergosterol).

Plant EV markers can be identified using any approaches known in the art that enable identification of small molecules (e.g., mass spectroscopy, mass spectrometry), lipds (e.g., mass spectroscopy, mass spectrometry), proteins (e.g., mass spectroscopy, immunoblotting), or nucleic acids (e.g., PCR analysis). In some instances, a PMP composition described herein includes a detectable amount, e.g., a pre-determined threshold amount, of a plant EV marker described herein.

### C. Loading of Agents

The PMP can be modified to include a heterologous functional agent, e.g., a pathogen control agent or repellent agent, such as those described herein. The PMP can carry or associate with such agents by a variety of means to enable delivery of the agent to a target plant or plant pest, e.g., by encapsulating the agent, incorporation of the component in the lipid bilayer structure, or association of the component (e.g., by conjugation) with the surface of the lipid bilayer structure of the PMP.

The heterologous functional agent can be incorporated or loaded into or onto the PMP by any methods known in the art that allow association, directly or indirectly, between the PMP and agent. Heterologous functional agent agents can be incorporated into the PMP by an in vivo method (e.g., in planta, e.g., through production of PMPs from a transgenic plant that comprises the heterologous agent), or in vitro (e.g., in tissue culture, or in cell culture), or both in vivo and in vitro methods.

In instances where the PMPs are loaded with a heterologous functional agent (e.g., a pathogen control agent or repellent) in vivo, the PMP may be produced from an EV, or segment, portion, or extract thereof, that has been loaded in planta, in tissue culture, or in cell culture. In planta methods include expression of the heterologous functional agent (e.g., pathogen control agent or repellent agent) in a plant that has been genetically modified to express the heterologous functional agent. In some instances, the heterologous functional agent is exogenous to the plant. Alternatively, the heterologous functional agent may be naturally found in the plant, but expressed at an elevated level relative to level of that found in a non-genetically modified plant.

In some instances, the PMP can be loaded in vitro. The substance may be loaded onto or into (e.g., may be encapsulated by) the PMPs using, but not limited to, physical, chemical, and/or biological methods. For example, the heterologous functional agent may be introduced into PMP by one or more of electroporation, sonication, passive diffusion, stirring, lipid extraction, or extrusion. Loaded PMPs can be assessed to confirm the presence or level of the loaded agent using a variety methods, such as HPLC (e.g., to assess small molecules); immunoblotting (e.g., to assess proteins); and quantitative PCR (e.g., to assess nucleotides). However, it should be appreciated by those skilled in the art that the loading of a substance of interest into PMPs is not limited to the above-illustrated methods.

In some instances, the heterologous functional agent can be conjugated to the PMP, in which the heterologous functional agent is connected or joined, indirectly or directly, to the PMP. For instance, one or more pathogen control agents can be chemically-linked to a PMP, such that the one or more pathogen control agents are joined (e.g., by covalent or ionic bonds) directly to the lipid bilayer of the PMP. In some instances, the conjugation of various pathogen control agents to the PMPs can be achieved by first mixing the one or more heterologous functional agents with an appropriate cross-linking agent (e.g., N-ethylcarbo-diimide (“EDC”), which is generally utilized as a carboxyl activating agent for amide bonding with primary amines and also reacts with phosphate groups) in a suitable solvent. After a period of incubation sufficient to allow the heterologous functional agent to attach to the cross-linking agent, the cross-linking agent/heterologous functional agent mixture can then be combined with the PMPs and, after another period of incubation, subjected to a sucrose gradient (e.g., and 8, 30, 45, and 60% sucrose gradient) to separate the free heterologous functional agent and free PMPs from the pathogen control agents conjugated to the PMPs. As part of combining the mixture with a sucrose gradient, and an accompanying centrifugation step, the PMPs conjugated to the pathogen control agents are then seen as a band in the sucrose gradient, such that the conjugated PMPs can then be collected, washed, and dissolved in a suitable solution for use as described herein.

In some instances, the PMP is stably associated with the heterologous functional agent prior to and following delivery of the PMP, e.g., to a plant or to a pest. In other instances, the PMP is associated with the heterologous functional agent such that the heterologous functional agent becomes dissociated from the PMP following delivery of the PMP, e.g., to a plant or to a pest.

The PMP can be further modified with other components (e.g., lipids, e.g., sterols, e.g., cholesterol; or small molecules) to further alter the functional and structural characteristics of the PMP. For example, the PMPs can be further modified with stabilizing molecules that increase the stability of the PMP (e.g., for at least one day at room temperature, and/or stable for at least one week at 4° C.).

The PMPs can be loaded with various concentrations of the heterologous functional agent, depending on the particular agent or use. For example, in some instances, the PMPs are loaded such that the pathogen control composition disclosed herein includes about 0.001, 0.01, 0.1, 1.0, 2, 3, 4, 5, 6, 7, 8, 9, 10, 15, 20, 30, 40, 50, 60, 70, 80, 90, or 95 (or any range between about 0.001 and 95) or more wt % of a pathogen control agent and/or a repellent agent. In some instances, the PMPs are loaded such that the pathogen control composition includes about 95, 90, 80, 70, 60, 50, 40, 30, 20, 15, 10, 9, 8, 7, 6, 5, 4, 3, 2, 1.0, 0.1, 0.01, 0.001 (or any range between about 95 and 0.001) or less wt % of a pathogen control agent and/or a repellent agent. For example, the pathogen control composition can include about 0.001 to about 0.01 wt %, about 0.01 to about 0.1 wt %, about 0.1 to about 1 wt %, about 1 to about 5 wt %, or about 5 to about 10 wt %, about 10 to about 20 wt % of the pathogen control agent and/or a repellent agent. In some instances, the PMP can be loaded with about 1, 5, 10, 50, 100, 200, or 500, 1,000, 2,000 (or any range between about 1 and 2,000) or more pg/ml of a pathogen control agent and/or a repellent agent. A liposome of the invention can be loaded with about 2,000, 1,000, 500, 200, 100, 50, 10, 5, 1 (or any range between about 2,000 and 1) or less pg/ml of a pathogen control agent and/or a repellent agent.

in some instances, the PMPs are loaded such that the pathogen control composition disclosed herein includes at least 0.001 wt %, at least 0.01 wt %, at least 0.1 wt %, at least 1.0 wt %, at least 2 wt %, at least 3 wt %, at least 4 wt %, at least 5 wt %, at least 6 wt %, at least 7 wt %, at least 8 wt %, at least 9 wt %, at least 10 wt %, at least 15 wt %, at least 20 wt %, at least 30 wt %, at least 40 wt %, at least 50 wt %, at least 60 wt %, at least 70 wt %, at least 80 wt %, at least 90 wt %, or at least 95 wt % of a pathogen control agent and/or a repellent agent. In some instances, the PMP can be loaded with at least 1 pg/ml, at least 5 pg/ml, at least 10 pg/ml, at least 50 pg/ml, at least 100 pg/ml, at least 200 pg/ml, at least 500 pg/ml, at least 1,000 pg/ml, at least 2,000 pg/ml of a pathogen control agent and/or a repellent agent.

Examples of particular pathogen control agents or repellent agents that can be loaded into the PMP are further outlined in the section entitled “Heterologous Functional Agents.”

### D. Pharmaceutical Formulations

Included herein are pathogen control compositions that can be formulated into pharmaceutical compositions, e.g., for administration to an animal. The pharmaceutical composition may be administered to an animal with a pharmaceutically acceptable diluent, carrier, and/or excipient. Depending on the mode of administration and the dosage, the pharmaceutical composition of the methods described herein will be formulated into suitable pharmaceutical compositions to permit facile delivery. The single dose may be in a unit dose form as needed.

A pathogen control composition may be formulated for e.g., oral administration, intravenous administration (e.g., injection or infusion), or subcutaneous administration to an animal. For injectable formulations, various effective pharmaceutical carriers are known in the art (See, e.g., Remington: The Science and Practice of Pharmacy, 22^{nd} ed., (2012) and ASHP Handbook on Injectable Drugs, 18th ed., (2014)).

Pharmaceutically acceptable carriers and excipients in the present compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. The compositions may be formulated according to conventional pharmaceutical practice. The concentration of the compound in the formulation will vary depending upon a number of factors, including the dosage of the active agent (e.g., PMP) to be administered, and the route of administration.

For oral administration to an animal, the pathogen control composition can be prepared in the form of an oral formulation. Formulations for oral use can include tablets, caplets, capsules, syrups, or oral liquid dosage forms containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Formulations for oral use may also be provided in unit dosage form as chewable tablets, non-chewable tablets, caplets, capsules (e.g., as hard gelatin capsules wherein the active ingredient is mixed with an inert solid diluent, or as soft gelatin capsules wherein the active ingredient is mixed with water or an oil medium). The compositions disclosed herein may also further include an immediate-release, extended release or delayed-release formulation.

For parenteral administration to an animal, the pathogen control compositions may be formulated in the form of liquid solutions or suspensions and administered by a parenteral route (e.g., subcutaneous, intravenous, or intramuscular). The pharmaceutical composition can be formulated for injection or infusion. Pharmaceutical compositions for parenteral administration can be formulated using a sterile solution or any pharmaceutically acceptable liquid as a vehicle. Pharmaceutically acceptable vehicles include, but are not limited to, sterile water, physiological saline, or cell culture media (e.g., Dulbecco's Modified Eagle Medium (DMEM), α-Modified Eagles Medium (α-MEM), F-12 medium). Formulation methods are known in the art, see e.g., Gibson (ed.) Pharmaceutical Preformulation and Formulation (2nd ed.) Taylor & Francis Group, CRC Press (2009).

### E. Agricultural Formulations

Included herein are pathogen control compositions that can be formulated into agricultural compositions, e.g., for administration to pathogen or pathogen vector (e.g., an insect). The pharmaceutical composition may be administered to a pathogen or pathogen vector (e.g., an insect) with an agriculturally acceptable diluent, carrier, and/or excipient. Further examples of agricultural formulations useful in the present compositions and methods are further outlined herein.

To allow ease of application, handling, transportation, storage, and maximum activity, the active agent, here PMPs, can be formulated with other substances. PMPs can be formulated into, for example, baits, concentrated emulsions, dusts, emulsifiable concentrates, fumigants, gels, granules, microencapsulations, seed treatments, suspension concentrates, suspoemulsions, tablets, water soluble liquids, water dispersible granules or dry flowables, wettable powders, and ultra-low volume solutions. For further information on formulation types see “Catalogue of Pesticide Formulation Types and International Coding System” Technical Monograph n° 2, 5th Edition by CropLife International (2002).

Active agents (e.g., PMPs with or without heterologous functional agents, e.g., antipathogen agents, pesticidal agents, or repellent agents) can be applied most often as aqueous suspensions or emulsions prepared from concentrated formulations of such agents. Such water-soluble, water-suspendable, or emulsifiable formulations are either solids, usually known as wettable powders, or water dispersible granules, or liquids usually known as emulsifiable concentrates, or aqueous suspensions. Wettable powders, which may be compacted to form water dispersible granules, comprise an intimate mixture of the pesticide, a carrier, and surfactants. The carrier is usually selected from among the attapulgite clays, the montmorillonite clays, the diatomaceous earths, or the purified silicates. Effective surfactants, including from about 0.5% to about 10% of the wettable powder, are found among sulfonated lignins, condensed naphthalenesulfonates, naphthalenesulfonates, alkylbenzenesulfonates, alkyl sulfates, and non-ionic surfactants such as ethylene oxide adducts of alkyl phenols.

Emulsifiable concentrates can comprise a suitable concentration of PMPs, such as from about 50 to about 500 grams per liter of liquid dissolved in a carrier that is either a water miscible solvent or a mixture of water-immiscible organic solvent and emulsifiers. Useful organic solvents include aromatics, especially xylenes and petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are selected from conventional anionic and non-ionic surfactants.

Aqueous suspensions comprise suspensions of water-insoluble pesticides dispersed in an aqueous carrier at a concentration in the range from about 5% to about 50% by weight. Suspensions are prepared by finely grinding the pesticide and vigorously mixing it into a carrier comprised of water and surfactants. Ingredients, such as inorganic salts and synthetic or natural gums may also be added, to increase the density and viscosity of the aqueous carrier.

PMPs may also be applied as granular compositions that are particularly useful for applications to the soil. Granular compositions usually contain from about 0.5% to about 10% by weight of the pesticide, dispersed in a carrier that includes clay or a similar substance. Such compositions are usually prepared by dissolving the formulation in a suitable solvent and applying it to a granular carrier which has been pre-formed to the appropriate particle size, in the range of from about 0.5 to about 3 mm. Such compositions may also be formulated by making a dough or paste of the carrier and compound and crushing and drying to obtain the desired granular particle size.

Dusts containing the present PMP formulation are prepared by intimately mixing PMPs in powdered form with a suitable dusty agricultural carrier, such as kaolin clay, ground volcanic rock, and the like. Dusts can suitably contain from about 1% to about 10% of the packets. They can be applied as a seed dressing or as a foliage application with a dust blower machine.

It is equally practical to apply the present formulation in the form of a solution in an appropriate organic solvent, usually petroleum oil, such as the spray oils, which are widely used in agricultural chemistry.

PMPs can also be applied in the form of an aerosol composition. In such compositions the packets are dissolved or dispersed in a carrier, which is a pressure-generating propellant mixture. The aerosol composition is packaged in a container from which the mixture is dispensed through an atomizing valve.

Another embodiment is an oil-in-water emulsion, wherein the emulsion includes oily globules which are each provided with a lamellar liquid crystal coating and are dispersed in an aqueous phase, wherein each oily globule includes at least one compound which is agriculturally active, and is individually coated with a monolamellar or oligolamellar layer including: (1) at least one non-ionic lipophilic surface-active agent, (2) at least one non-ionic hydrophilic surface-active agent and (3) at least one ionic surface-active agent, wherein the globules having a mean particle diameter of less than 800 nanometers. Further information on the embodiment is disclosed in U.S. patent publication 20070027034 published Feb. 1, 2007. For ease of use, this embodiment will be referred to as “OIWE.”

Additionally, generally, when the molecules disclosed above are used in a formulation, such formulation can also contain other components. These components include, but are not limited to, (this is a non-exhaustive and non-mutually exclusive list) wetters, spreaders, stickers, penetrants, buffers, sequestering agents, drift reduction agents, compatibility agents, anti-foam agents, cleaning agents, and emulsifiers. A few components are described forthwith.

A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank to reduce the wetting time of wettable powders and to improve the penetration of water into water-dispersible granules. Examples of wetting agents used in wettable powder, suspension concentrate, and water-dispersible granule formulations are: sodium lauryl sulfate; sodium dioctyl sulfosuccinate; alkyl phenol ethoxylates; and aliphatic alcohol ethoxylates.

A dispersing agent is a substance which adsorbs onto the surface of particles and helps to preserve the state of dispersion of the particles and prevents them from reaggregating. Dispersing agents are added to agrochemical formulations to facilitate dispersion and suspension during manufacture, and to ensure the particles redisperse into water in a spray tank. They are widely used in wettable powders, suspension concentrates and water-dispersible granules. Surfactants that are used as dispersing agents have the ability to adsorb strongly onto a particle surface and provide a charged or steric barrier to reaggregation of particles. The most commonly used surfactants are anionic, non-ionic, or mixtures of the two types. For wettable powder formulations, the most common dispersing agents are sodium lignosulfonates. For suspension concentrates, very good adsorption and stabilization are obtained using polyelectrolytes, such as sodium naphthalene sulfonate formaldehyde condensates. Tristyrylphenol ethoxylate phosphate esters are also used. Non-ionics such as alkylarylethylene oxide condensates and EO-PO block copolymers are sometimes combined with anionics as dispersing agents for suspension concentrates. In recent years, new types of very high molecular weight polymeric surfactants have been developed as dispersing agents. These have very long hydrophobic ‘backbones’ and a large number of ethylene oxide chains forming the ‘teeth’ of a ‘comb’ surfactant. These high molecular weight polymers can give very good long-term stability to suspension concentrates because the hydrophobic backbones have many anchoring points onto the particle surfaces. Examples of dispersing agents used in agrochemical formulations are: sodium lignosulfonates; sodium naphthalene sulfonate formaldehyde condensates; tristyrylphenol ethoxylate phosphate esters; aliphatic alcohol ethoxylates; alkyl ethoxylates; EO-PO (ethylene oxide-propylene oxide) block copolymers; and graft copolymers.

An emulsifying agent is a substance which stabilizes a suspension of droplets of one liquid phase in another liquid phase. Without the emulsifying agent the two liquids would separate into two immiscible liquid phases. The most commonly used emulsifier blends contain alkylphenol or aliphatic alcohol with twelve or more ethylene oxide units and the oil-soluble calcium salt of dodecylbenzenesulfonic acid. A range of hydrophile-lipophile balance (“HLB”) values from 8 to 18 will normally provide good stable emulsions. Emulsion stability can sometimes be improved by the addition of a small amount of an E0-PO block copolymer surfactant.

A solubilizing agent is a surfactant which will form micelles in water at concentrations above the critical micelle concentration. The micelles are then able to dissolve or solubilize water-insoluble materials inside the hydrophobic part of the micelle. The types of surfactants usually used for solubilization are non-ionics, sorbitan monooleates, sorbitan monooleate ethoxylates, and methyl oleate esters.

Surfactants are sometimes used, either alone or with other additives such as mineral or vegetable oils as adjuvants to spray-tank mixes to improve the biological performance of the pesticide on the target. The types of surfactants used for bioenhancement depend generally on the nature and mode of action of the pesticide. However, they are often non-ionics such as: alkyl ethoxylates; linear aliphatic alcohol ethoxylates; aliphatic amine ethoxylates.

A carrier or diluent in an agricultural formulation is a material added to the pesticide to give a product of the required strength. Carriers are usually materials with high absorptive capacities, while diluents are usually materials with low absorptive capacities. Carriers and diluents are used in the formulation of dusts, wettable powders, granules, and water-dispersible granules.

Organic solvents are used mainly in the formulation of emulsifiable concentrates, oil-in-water emulsions, suspoemulsions, and ultra low volume formulations, and to a lesser extent, granular formulations. Sometimes mixtures of solvents are used. The first main groups of solvents are aliphatic paraffinic oils such as kerosene or refined paraffins. The second main group (and the most common) includes the aromatic solvents such as xylene and higher molecular weight fractions of C9 and C10 aromatic solvents. Chlorinated hydrocarbons are useful as cosolvents to prevent crystallization of pesticides when the formulation is emulsified into water. Alcohols are sometimes used as cosolvents to increase solvent power. Other solvents may include vegetable oils, seed oils, and esters of vegetable and seed oils.

Thickeners or gelling agents are used mainly in the formulation of suspension concentrates, emulsions, and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate formulations using clays and silicas. Examples of these types of materials, include, but are not limited to, montmorillonite, bentonite, magnesium aluminum silicate, and attapulgite. Water-soluble polysaccharides have been used as thickening-gelling agents for many years. The types of polysaccharides most commonly used are natural extracts of seeds and seaweeds or are synthetic derivatives of cellulose. Examples of these types of materials include, but are not limited to, guar gum; locust bean gum; carrageenam; alginates; methyl cellulose; sodium carboxymethyl cellulose (SCMC); hydroxyethyl cellulose (HEC). Other types of anti-settling agents are based on modified starches, polyacrylates, polyvinyl alcohol, and polyethylene oxide. Another good anti-settling agent is xanthan gum.

Microorganisms can cause spoilage of formulated products. Therefore preservation agents are used to eliminate or reduce their effect. Examples of such agents include, but are not limited to: propionic acid and its sodium salt; sorbic acid and its sodium or potassium salts; benzoic acid and its sodium salt; p-hydroxybenzoic acid sodium salt; methyl p-hydroxybenzoate; and 1,2-benzisothiazolin-3-one (BIT).

The presence of surfactants often causes water-based formulations to foam during mixing operations in production and in application through a spray tank. In order to reduce the tendency to foam, anti-foam agents are often added either during the production stage or before filling into bottles. Generally, there are two types of anti-foam agents, namely silicones and non-silicones. Silicones are usually aqueous emulsions of dimethyl polysiloxane, while the non-silicone anti-foam agents are water-insoluble oils, such as octanol and nonanol, or silica. In both cases, the function of the anti-foam agent is to displace the surfactant from the air-water interface.

“Green” agents (e.g., adjuvants, surfactants, solvents) can reduce the overall environmental footprint of crop protection formulations. Green agents are biodegradable and generally derived from natural and/or sustainable sources, e.g., plant and animal sources. Specific examples are: vegetable oils, seed oils, and esters thereof, also alkoxylated alkyl polyglucosides.

In some instances, PMPs can be freeze-dried or lyophilized. See U.S. Pat. No. 4,311,712. The PMPs can later be reconstituted on contact with water or another liquid. Other components can be added to the lyophilized or reconstituted liposomes, for example, other antipathogen agents, pesticidal agents, repellent agents, agriculturally acceptable carriers, or other materials in accordance with the formulations described herein.

Other optional features of the composition include carriers or delivery vehicles that protect the pathogen control composition against UV and/or acidic conditions. In some instances, the delivery vehicle contains a pH buffer. In some instances, the composition is formulated to have a pH in the range of about 4.5 to about 9.0, including for example pH ranges of about any one of 5.0 to about 8.0, about 6.5 to about 7.5, or about 6.5 to about 7.0.

The composition may additionally be formulated with an attractant (e.g., a chemoattractant) that attracts a pest, such as a pathogen vector (e.g., an insect), to the vicinity of the composition. Attractants include pheromones, a chemical that is secreted by an animal, especially a pest, or chemoattractants which influences the behavior or development of others of the same species. Other attractants include sugar and protein hydrolysate syrups, yeasts, and rotting meat. Attractants also can be combined with an active ingredient and sprayed onto foliage or other items in the treatment area. Various attractants are known which influence a pest's behavior as a pest's search for food, oviposition, or mating sites, or mates. Attractants useful in the methods and compositions described herein include, for example, eugenol, phenethyl propionate, ethyl dimethylisobutyl-cyclopropane carboxylate, propyl benszodioxancarboxylate, cis-7,8-epoxy-2-methyloctadecane, trans-8,trans-0-dodecadienol, cis-9-tetradecenal (with cis-11-hexadecenal), trans-11-tetradecenal, cis-11-hexadecenal, (Z)-11,12-hexadecadienal, cis-7-dodecenyl acetate, cis-8-dodecenyul acetate, cis-9-dodecenyl acetate, cis-9-tetradecenyl acetate, cis-11-tetradecenyl acetate, trans-11-tetradecenyl acetate (with cis-11), cis-9,trans-11-tetradecadienyl acetate (with cis-9,trans-12), cis-9,trans-1 2-tetradecadienyl acetate, cis-7,cis-11-hexadecadienyl acetate (with cis-7,trans-11), cis-3,cis-13-octadecadienyl acetate, trans-3,cis-13-octadecadienyl acetate, anethole and isoamyl salicylate.

For further information on agricultural formulations, see “Chemistry and Technology of Agrochemical Formulations” edited by D. A. Knowles, copyright 1998 by Kluwer Academic Publishers. Also see “Insecticides in Agriculture and Environment—Retrospects and Prospects” by A. S. Perry, I. Yamamoto, I. Ishaaya, and R. Perry, copyright 1998 by Springer-Verlag.

II. Therapeutic Methods

The pathogen control compositions described herein are useful in a variety of therapeutic methods, particularly for the prevention or treatment of pathogen infections in animals. The present methods involve delivering the pathogen control compositions described herein to an animal.

Provided herein are methods of administering to a plant a pathogen control composition disclosed herein. The methods can be useful for treating or preventing a pathogen infection in an animal.

For example, provided herein is a method of treating an animal having a fungal infection, wherein the method includes administering to the animal an effective amount of a pathogen control composition including a plurality of PMPs. In some instances, the method includes administering to the animal an effective amount of a pathogen control composition including a plurality of PMPs, wherein the plurality of PMPs includes an antifungal agent. In some instances, the antifungal agent is a nucleic acid that inhibits expression of a gene in a fungus that causes the fungal infection (e.g., Enhanced Filamentous Growth Protein (EFG1)). In some instances, the fungal infection is caused by Candida albicans. In some instances, composition includes a PMP produced from an Arabidopsis apoplast EV. In some instances, the method decreases or substantially eliminates the fungal infection.

In another aspect, provided herein is a method of treating an animal having a bacterial infection, wherein the method includes administering to the animal an effective amount of a pathogen control composition including a plurality of PMPs. In some instances, the method includes administering to the animal an effective amount of a pathogen control composition including a plurality of PMPs, and wherein the plurality of PMPs includes an antibacterial agent (e.g., Amphotericin B). In some instances, the bacterium is a Streptococcus spp., Pneumococcus spp., Pseudomonas spp., Shigella spp, Salmonella spp., Campylobacter spp., or an Escherichia spp. In some instances, the composition includes a PMP produced from an Arabidopsis apoplast EV. In some instances, the method decreases or substantially eliminates the bacterial infection. In some instances, the animal is a human, a veterinary animal, or a livestock animal.

The present methods are useful to treat an infection (e.g., as caused by an animal pathogen) in an animal, which refers to administering treatment to an animal already suffering from a disease to improve or stabilize the animal's condition. This may involve reducing colonization of a pathogen in, on, or around an animal by one or more pathogens (e.g., by about 1%, 2%, 5%, 10%, 20%, 30%, 40%, 50%, 60%, 70%, 80%, 90%, or 100%) relative to a starting amount and/or allow benefit to the individual (e.g., reducing colonization in an amount sufficient to resolve symptoms). In such instances, a treated infection may manifest as a decrease in symptoms (e.g., by about 1%, 2%, 5%, 10%, 20%, 30%, 40%, 50%, 60%, 70%, 80%, 90%, or 100%). In some instances, a treated infection is effective to increase the likelihood of survival of an individual (e.g., an increase in likelihood of survival by about 1%, 2%, 5%, 10%, 20%, 30%, 40%, 50%, 60%, 70%, 80%, 90%, or 100%) or increase the overall survival of a population (e.g., an increase in likelihood of survival by about 1%, 2%, 5%, 10%, 20%, 30%, 40%, 50%, 60%, 70%, 80%, 90%, or 100%). For example, the compositions and methods may be effective to “substantially eliminate” an infection, which refers to a decrease in the infection in an amount sufficient to sustainably resolve symptoms (e.g., for at least 1, 2, 3, 4, 5, 6, 7, 8, 9, 10, 11, or 12 months) in the animal.

The present methods are useful to prevent an infection (e.g., as caused by an animal pathogen), which refers to preventing an increase in colonization in, on, or around an animal by one or more pathogens (e.g., by about 1%, 2%, 5%, 10%, 20%, 30%, 40%, 50%, 60%, 70%, 80%, 90%, 100%, or more than 100% relative to an untreated animal) in an amount sufficient to maintain an initial pathogen population (e.g., approximately the amount found in a healthy individual), prevent the onset of an infection, and/or prevent symptoms or conditions associated with infection. For example, individuals may receive prophylaxis treatment to prevent a fungal infection while being prepared for an invasive medical procedure (e.g., preparing for surgery, such as receiving a transplant, stem cell therapy, a graft, a prosthesis, receiving long-term or frequent intravenous catheterization, or receiving treatment in an intensive care unit), in immunocompromised individuals (e.g., individuals with cancer, with HIV/AIDS, or taking immunosuppressive agents), or in individuals undergoing long term antibiotic therapy.

The pathogen control composition can be formulated for administration or administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some instances, pathogen control composition is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

For the prevention or treatment of an infection described herein (when used alone or in combination with one or more other additional therapeutic agents) will depend on the type of disease to be treated, the severity and course of the disease, whether the is administered for preventive or therapeutic purposes, previous therapy, the patient's clinical history and response to the pathogen control composition. The pathogen control composition can be, e.g., administered to the patient at one time or over a series of treatments. For repeated administrations over several days or longer, depending on the condition, the treatment would generally be sustained until a desired suppression of disease symptoms occurs or the infection is no longer detectable. Such doses may be administered intermittently, e.g., every week or every two weeks (e.g., such that the patient receives, for example, from about two to about twenty, doses of the pathogen control composition. An initial higher loading dose, followed by one or more lower doses may be administered. However, other dosage regimens may be useful. The progress of this therapy is easily monitored by conventional techniques and assays.

In some instances, the amount of the pathogen control composition administered to individual (e.g., human) may be in the range of about 0.01 mg/kg to about 5 g/kg (e.g., about 0.01 mg/kg-0.1 mg/kg, about 0.1 mg/kg-1 mg/kg, about 1 mg/kg-10 mg/kg, about 10 mg/kg-100 mg/kg, about 100 mg/kg-1 g/kg, or about 1 g/kg-5 g/kg), of the individual's body weight. In some instances, the amount of the pathogen control composition administered to individual (e.g., human) is at least 0.01 mg/kg (e.g., at least 0.01 mg/kg, at least 0.1 mg/kg, at least 1 mg/kg, at least 10 mg/kg, at least 100 mg/kg, at least 1 g/kg, or at least 5 g/kg), of the individual's body weight. The dose may be administered as a single dose or as multiple doses (e.g., 2, 3, 4, 5, 6, 7, or more than 7 doses). In some instances, the pathogen control composition administered to the animal may be administered alone or in combination with an additional therapeutic agent or pathogen control agent. The dose of the antibody administered in a combination treatment may be reduced as compared to a single treatment. The progress of this therapy is easily monitored by conventional techniques.

III. Agricultural Methods

The pathogen control compositions described herein are useful in a variety of agricultural methods, particularly for the prevention or treatment of pathogen infections in animals and for the control of the spread of such pathogens, e.g., by pathogen vectors. The present methods involve delivering the pathogen control compositions described herein to a pathogen or a pathogen vector.

The compositions and related methods can be used to prevent infestation by or reduce the numbers of pathogens or pathogen vectors in any habitats in which they reside (e.g., outside of animals, e.g., on plants, plant parts (e.g., roots, fruits and seeds), in or on soil, water, or on another pathogen or pathogen vector habitat. Accordingly, the compositions and methods can reduce the damaging effect of pathogen vectors by for example, killing, injuring, or slowing the activity of the vector, and can thereby control the spread of the pathogen to animals. Compositions disclosed herein can be used to control, kill, injure, paralyze, or reduce the activity of one or more of any pathogens or pathogen vectors in any developmental stage, e.g., their egg, nymph, instar, larvae, adult, juvenile, or desiccated forms. The details of each of these methods are described further below.

A. Delivery to a Pathogen

Provided herein are methods of delivering a pathogen control composition to a pathogen, such as one disclosed herein, by contacting the pathogen with a pathogen control composition. The methods can be useful for decreasing the fitness of a pathogen, e.g., to prevent or treat a pathogen infection or control the spread of a pathogen as a consequence of delivery of the pathogen control composition. Examples of pathogens that can be targeted in accordance with the methods described herein include bacteria (e.g., Streptococcus spp., Pneumococcus spp., Pseudomonas spp., Shigella spp, Salmonella spp., Campylobacter spp., or an Escherichia spp), fungi (Saccharomyces spp. or a Candida spp), parasitic insects (e.g., Cimex spp), parasitic nematodes (e.g., Heligmosomoides spp), or parasitic protozoa (e.g., Trichomoniasis spp).

For example, provided herein is a method of decreasing the fitness of a pathogen, the method including delivering to the pathogen any of the compositions described herein, wherein the method decreases the fitness of the pathogen relative to an untreated pathogen. In some embodiments, the method includes delivering the composition to at least one habitat where the pathogen grows, lives, reproduces, feeds, or infests. In some instances of the methods described herein, the composition is delivered as a pathogen comestible composition for ingestion by the pathogen. In some instances of the methods described herein, the composition is delivered (e.g., to a pathogen) as a liquid, a solid, an aerosol, a paste, a gel, or a gas.

Also provided herein is a method of decreasing the fitness of a parasitic insect, wherein the method includes delivering to the parasitic insect a pathogen control composition including a plurality of PMPs. In some instances, the method includes delivering to the parasitic insect a pathogen control composition including a plurality of PMPs, wherein the plurality of PMPs includes an insecticidal agent. For example, the parasitic insect may be a bedbug. Other non-limiting examples of parasitic insects are provided herein. In some instances, the method decreases the fitness of the parasitic insect relative to an untreated parasitic insect

Additionally provided herein is a method of decreasing the fitness of a parasitic nematode, wherein the method includes delivering to the parasitic nematode a pathogen control composition including a plurality of PMPs. In some instances, the method includes delivering to the parasitic nematode a pathogen control composition including a plurality of PMPs, wherein the plurality of PMPs includes a nematicidal agent. For example, the parasitic nematode is Heligmosomoides polygyrus. Other non-limiting examples of parasitic nematodes are provided herein. In some instances, the method decreases the fitness of the parasitic nematode relative to an untreated parasitic nematode.

Further provided herein is a method of decreasing the fitness of a parasitic protozoan, wherein the method includes delivering to the parasitic protozoan a pathogen control composition including a plurality of PMPs. In some instances, the method includes delivering to the parasitic protozoan a pathogen control composition including a plurality of PMPs, wherein the plurality of PMPs includes an antiparasitic agent. For example, the parasitic protozoan may be T. vaginalis. Other non-limiting examples of parasitic protozoans are provided herein. In some instances, the method decreases the fitness of the parasitic protozoan relative to an untreated parasitic protozoan.

A decrease in the fitness of the pathogen as a consequence of delivery of a pathogen control composition can manifest in a number of ways. In some instances, the decrease in fitness of the pathogen may manifest as a deterioration or decline in the physiology of the pathogen (e.g., reduced health or survival) as a consequence of delivery of the pathogen control composition. In some instances, the fitness of an organism may be measured by one or more parameters, including, but not limited to, reproductive rate, fertility, lifespan, viability, mobility, fecundity, pathogen development, body weight, metabolic rate or activity, or survival in comparison to a pathogen to which the pathogen control composition has not been administered. For example, the methods or compositions provided herein may be effective to decrease the overall health of the pathogen or to decrease the overall survival of the pathogen. In some instances, the decreased survival of the pathogen is about 2%, 5%, 10%, 20%, 30%, 40%, 50%, 60%, 70%, 80%, 90%, 100%, or greater than 100% greater relative to a reference level (e.g., a level found in a pathogen that does not receive a pathogen control. In some instances, the methods and compositions are effective to decrease pathogen reproduction (e.g., reproductive rate, fertility) in comparison to a pathogen to which the pathogen control composition has not been administered. In some instances, the methods and compositions are effective to decrease other physiological parameters, such as mobility, body weight, life span, fecundity, or metabolic rate, by about 2%, 5%, 10%, 20%, 30%, 40%, 50%, 60%, 70%, 80%, 90%, 100%, or greater than 100% relative to a reference level (e.g., a level found in a pathogen that does not receive a pathogen control composition).

In some instances, the decrease in pest fitness may manifest as an increase in the pathogen's sensitivity to an antipathogen agent and/or a decrease in the pathogen's resistance to an antipathogen agent in comparison to a pathogen to which the pathogen control composition has not been delivered. In some instances, the methods or compositions provided herein may be effective to increase the pathogen's sensitivity to a pathogen control agent by about 2%, 5%, 10%, 20%, 30%, 40%, 50%, 60%, 70%, 80%, 90%, 100%, or greater than 100% relative to a reference level (e.g., a level found in a pest that does not receive a pathogen control composition).

In some instances, the decrease in pathogen fitness may manifest as other fitness disadvantages, such as a decreased tolerance to certain environmental factors (e.g., a high or low temperature tolerance), a decreased ability to survive in certain habitats, or a decreased ability to sustain a certain diet in comparison to a pathogen to which the pathogen control (composition has not been delivered. In some instances, the methods or compositions provided herein may be effective to decrease pathogen fitness in any plurality of ways described herein. Further, the pathogen control composition may decrease pathogen fitness in any number of pathogen classes, orders, families, genera, or species (e.g., 1 pathogen species, 2, 3, 4, 5, 6, 7, 8, 9, 10, 15, 20, 30, 40, 50, 60, 70, 80, 90, 100, 150, 200, 200, 250, 500, or more pathogen species). In some instances, the pathogen control composition acts on a single pest class, order, family, genus, or species.

Pathogen fitness may be evaluated using any standard methods in the art. In some instances, pest fitness may be evaluated by assessing an individual pathogen. Alternatively, pest fitness may be evaluated by assessing a pathogen population. For example, a decrease in pathogen fitness may manifest as a decrease in successful competition against other pathogens, thereby leading to a decrease in the size of the pathogen population.

B. Delivery to a Pathogen Vector

Provided herein are methods of delivering a pathogen control composition to a pathogen vector, such as one disclosed herein, by contacting the pathogen with a pathogen control composition. The methods can be useful for decreasing the fitness of a pathogen vector, e.g., to control the spread of a pathogen as a consequence of delivery of the pathogen control composition. Examples of pathogen vectors that can be targeted in accordance with the methods described herein include insects, such as those described in Section IV.G.

For example, provided herein is a method of decreasing the fitness of an animal pathogen vector, the method including delivering to the vector an effective amount of any of the compositions described herein, wherein the method decreases the fitness of the vector relative to an untreated vector. In some instances, the method includes delivering the composition to at least one habitat where the vector grows, lives, reproduces, feeds, or infests. In some instances, the composition is delivered as a comestible composition for ingestion by the vector. In some instances, the vector is an insect. In some instances, the insect is a mosquito, a tick, a mite, or a louse. In some instances, the composition is delivered (e.g., to the pathogen vector) as a liquid, a solid, an aerosol, a paste, a gel, or a gas.

For example, provided herein is a method of decreasing the fitness of an insect vector of an animal pathogen, wherein the method includes delivering to the vector a pathogen control composition including a plurality of PMPs. In some instances, the method includes delivering to the vector a pathogen control composition including a plurality of PMPs, wherein the plurality of PMPs includes an insecticidal agent. For example, the insect vector may be a mosquito, tick, mite, or louse. Other non-limiting examples of pathogen vectors are provided herein. In some instances, the method decreases the fitness of the vector relative to an untreated vector.

In some instances, the decrease in vector fitness may manifest as a deterioration or decline in the physiology of the vector (e.g., reduced health or survival) as a consequence of administration of a composition. In some instances, the fitness of an organism may be measured by one or more parameters, including, but not limited to, reproductive rate, lifespan, mobility, fecundity, body weight, metabolic rate or activity, or survival in comparison to a vector organism to which the composition has not been delivered. For example, the methods or compositions provided herein may be effective to decrease the overall health of the vector or to decrease the overall survival of the vector. In some instances, the decreased survival of the vector is about 2%, 5%, 10%, 20%, 30%, 40%, 50%, 60%, 70%, 80%, 90%, 100%, or greater than 100% greater relative to a reference level (e.g., a level found in a vector that does not receive a composition). In some instances, the methods and compositions are effective to decrease vector reproduction (e.g., reproductive rate) in comparison to a vector organism to which the composition has not been delivered. In some instances, the methods and compositions are effective to decrease other physiological parameters, such as mobility, body weight, life span, fecundity, or metabolic rate, by about 2%, 5%, 10%, 20%, 30%, 40%, 50%, 60%, 70%, 80%, 90%, 100%, or greater than 100% relative to a reference level (e.g., a level found in a vector that is not delivered the composition).

In some instances, the decrease in vector fitness may manifest as an increase in the vector's sensitivity to a pesticidal agent and/or a decrease in the vector's resistance to a pesticidal agent in comparison to a vector organism to which the composition has not been delivered. In some instances, the methods or compositions provided herein may be effective to increase the vector's sensitivity to a pesticidal agent by about 2%, 5%, 10%, 20%, 30%, 40%, 50%, 60%, 70%, 80%, 90%, 100%, or greater than 100% relative to a reference level (e.g., a level found in a vector that does not receive a composition). The pesticidal agent may be any pesticidal agent known in the art, including insecticidal agents. In some instances, the methods or compositions provided herein may increase the vector's sensitivity to a pesticidal agent by decreasing the vector's ability to metabolize or degrade the pesticidal agent into usable substrates in comparison to a vector to which the composition has not been delivered.

In some instances, the decrease in vector fitness may manifest as other fitness disadvantages, such as decreased tolerance to certain environmental factors (e.g., a high or low temperature tolerance), decreased ability to survive in certain habitats, or a decreased ability to sustain a certain diet in comparison to a vector organism to which the composition has not been delivered. In some instances, the methods or compositions provided herein may be effective to decrease vector fitness in any plurality of ways described herein. Further, the composition may decrease vector fitness in any number of vector classes, orders, families, genera, or species (e.g., 1 vector species, 2, 3, 4, 5, 6, 7, 8, 9, 10, 15, 20, 30, 40, 50, 60, 70, 80, 90, 100, 150, 200, 200, 250, 500, or more vector species). In some instances, the composition acts on a single vector class, order, family, genus, or species.

Vector fitness may be evaluated using any standard methods in the art. In some instances, vector fitness may be evaluated by assessing an individual vector. Alternatively, vector fitness may be evaluated by assessing a vector population. For example, a decrease in vector fitness may manifest as a decrease in successful competition against other vectors, thereby leading to a decrease in the size of the vector population.

By decreasing the fitness of vectors that carry animal pathogens, the compositions provided herein are effective to reduce the spread of vector-borne diseases. The composition may be delivered to the insects using any of the formulations and delivery methods described herein, in an amount and for a duration effective to reduce transmission of the disease, e.g., reduce vertical or horizontal transmission between vectors and/or reduce transmission to animals. For example, the composition described herein may reduce vertical or horizontal transmission of a vector-borne pathogen by about 2%, 5%, 10%, 20%, 30%, 40%, 50%, 60%, 70%, 80%, 90%, 100%, or more in comparison to a vector organism to which the composition has not been delivered. As another example, the composition described herein may reduce vectorial competence of an insect vector by about 2%, 5%, 10%, 20%, 30%, 40%, 50%, 60%, 70%, 80%, 90%, 100%, or more in comparison to a vector organism to which the composition has not been delivered.

Non-limiting examples of diseases that may be controlled by the compositions and methods provided herein include diseases caused by Togaviridae viruses (e.g., Chikungunya, Ross River fever, Mayaro, Onyon-nyong fever, Sindbis fever, Eastern equine enchephalomyeltis, Wesetern equine encephalomyelitis, Venezualan equine encephalomyelitis, or Barmah forest); diseases caused by Flavivirdae viruses (e.g., Dengue fever, Yellow fever, Kyasanur Forest disease, Omsk haemorrhagic fever, Japaenese encephalitis, Murray Valley encephalitis, Rocio, St. Louis encephalitis, West Nile encephalitis, or Tick-borne encephalitis); diseases caused by Bunyaviridae viruses (e.g., Sandly fever, Rift Valley fever, La Crosse encephalitis, California encephalitis, Crimean-Congo haemorrhagic fever, or Oropouche fever); disease caused by Rhabdoviridae viruses (e.g., Vesicular stomatitis); disease caused by Orbiviridae (e.g., Bluetongue); diseases caused by bacteria (e.g., Plague, Tularaemia, Q fever, Rocky Mountain spotted fever, Murine typhus, Boutonneuse fever, Queensland tick typhus, Siberian tick typhus, Scrub typhus, Relapsing fever, or Lyme disease); or diseases caused by protozoa (e.g., Malaria, African trypanosomiasis, Nagana, Chagas disease, Leishmaniasis, Piroplasmosis, Bancroftian filariasis, or Brugian filariasis).

C. Application Methods

A pathogen or pathogen vector described herein can be exposed to any of the compositions described herein in any suitable manner that permits delivering or administering the composition to the pathogen or pathogen vector. The pathogen control composition may be delivered either alone or in combination with other active (e.g., pesticidal agents) or inactive substances and may be applied by, for example, spraying, microinjection, through plants, pouring, dipping, in the form of concentrated liquids, gels, solutions, suspensions, sprays, powders, pellets, briquettes, bricks and the like, formulated to deliver an effective concentration of the pathogen control composition. Amounts and locations for application of the compositions described herein are generally determined by the habits of the pathogen or pathogen vector, the lifecycle stage at which the pathogen or pathogen vector can be targeted by the pathogen control composition, the site where the application is to be made, and the physical and functional characteristics of the pathogen control composition. The pathogen control compositions described herein may be administered to the pathogen or pathogen vector by oral ingestion, but may also be administered by means which permit penetration through the cuticle or penetration of the pathogen or pathogen vector respiratory system.

In some instances, the pathogen or pathogen vector can be simply “soaked” or “sprayed” with a solution including the pathogen control composition. Alternatively, the pathogen control composition can be linked to a food component (e.g., comestible) of the pathogen or pathogen vector for ease of delivery and/or in order to increase uptake of the pathogen control composition by the pest. Methods for oral introduction include, for example, directly mixing a pathogen control composition with the pathogen's or pathogen vector's food, spraying the pathogen control composition in the pathogen's or pathogen vector's habitat or field, as well as engineered approaches in which a species that is used as food is engineered to express a pathogen control composition, then fed to the pathogen or pathogen vector to be affected. In some instances, for example, the pathogen control composition can be incorporated into, or overlaid on the top of, the pathogen or pathogen vector's diet. For example, the pathogen control composition can be sprayed onto a field of crops which a pathogen or pathogen vector inhabits.

In some instances, the composition is sprayed directly onto a plant e.g., crops, by e.g., backpack spraying, aerial spraying, crop spraying/dusting etc. In instances where the pathogen control composition is delivered to a plant, the plant receiving the pathogen control composition may be at any stage of plant growth. For example, formulated pathogen control compositions can be applied as a seed-coating or root treatment in early stages of plant growth or as a total plant treatment at later stages of the crop cycle. In some instances, the pathogen control composition may be applied as a topical agent to a plant, such that the pathogen or pathogen vector ingests or otherwise comes in contact with the plant upon interacting with the plant.

Further, the pathogen control composition may be applied (e.g., in the soil in which a plant grows, or in the water that is used to water the plant) as a systemic agent that is absorbed and distributed through the tissues of a plant or animal pathogen or pathogen vector, such that a pathogen or pathogen vector feeding thereon will obtain an effective dose of the pathogen control composition. In some instances, plants or food organisms may be genetically transformed to express the pathogen control composition such that a pathogen or pathogen vector feeding upon the plant or food organism will ingest the pathogen control composition.

Delayed or continuous release can also be accomplished by coating the pathogen control composition or a composition with the pathogen control composition(s) with a dissolvable or bioerodable coating layer, such as gelatin, which coating dissolves or erodes in the environment of use, to then make the pathogen control composition available, or by dispersing the agent in a dissolvable or erodable matrix. Such continuous release and/or dispensing means devices may be advantageously employed to consistently maintain an effective concentration of one or more of the pathogen control compositions described herein in a specific pathogen or pathogen vector habitat.

The pathogen control composition can also be incorporated into the medium in which the pathogen or pathogen vector grows, lives, reproduces, feeds, or infests. For example, a pathogen control composition can be incorporated into a food container, feeding station, protective wrapping, or a hive. For some applications the pathogen control composition may be bound to a solid support for application in powder form or in a trap or feeding station. As an example, for applications where the composition is to be used in a trap or as bait for a particular pathogen or pathogen vector, the compositions may also be bound to a solid support or encapsulated in a time-release material. For example, the compositions described herein can be administered by delivering the composition to at least one habitat where an agricultural pathogen or pathogen vector grows, lives, reproduces, or feeds.

Pesticides are often recommended for field application as an amount of pesticide per hectare (g/ha or kg/ha) or the amount of active ingredient or acid equivalent per hectare (kg a.i./ha or g a.i./ha). In some instances, a lower amount of pesticide in the present compositions may be required to be applied to soil, plant media, seeds plant tissue, or plants to achieve the same results as where the pesticide is applied in a composition lacking PMPs. For example, the amount of pesticidal agent may be applied at levels about 2, 3, 4, 5, 6, 7, 8, 9, 10, 15, 20, 30, 50, or 100-fold (or any range between about 2 and about 100-fold, for example about 2- to 10-fold; about 5- to 15-fold, about 10- to 20-fold; about 10- to 50-fold) less than the same pesticidal agent applied in a non-PMP composition, e.g., direct application of the same pesticidal agent. Pathogen control compositions disclosed herein can be applied at a variety of amounts per hectare, for example at about 0.0001, 0.001, 0.005, 0.01, 0.1, 1, 2, 10, 100, 1,000, 2,000, 5,000 (or any range between about 0.0001 and 5,000) kg/ha. For example, about 0.0001 to about 0.01, about 0.01 to about 10, about 10 to about 1,000, about 1,000 to about 5,000 kg/ha.

IV. Pathogens or Vectors Thereof

The pathogen control compositions and related methods described herein are useful to decrease the fitness of an animal pathogen and thereby treat or prevent infections in animals. Examples of animal pathogens, or vectors thereof, that can be treated with the present compositions or related methods are further described herein.

### A. Fungi

The pathogen control compositions and related methods can be useful for decreasing the fitness of a fungus, e.g., to prevent or treat a fungal infection in an animal. Included are methods for delivering a pathogen control composition to a fungus by contacting the fungus with the pathogen control composition. Additionally or alternatively, the methods include preventing or treating a fungal infection (e.g., caused by a fungus described herein) in an animal at risk of or in need thereof, by administering to the animal a pathogen control composition.

The pathogen control compositions and related methods are suitable for treatment or preventing of fungal infections in animals, including infections caused by fungi belonging to Ascomycota (Fusarium oxysporum, Pneumocystis jirovecii, Aspergillus spp., Coccidioides immitis/posadasii, Candida albicans), Basidiomycota (Filobasidiella neoformans, Trichosporon), Microsporidia (Encephalitozoon cuniculi, Enterocytozoon bieneusi), Mucoromycotina (Mucor circinelloides, Rhizopus oryzae, Lichtheimia corymbifera).

In some instances, the fungal infection is one caused by a belonging to the phylum Ascomycota, Basidomycota, Chytridiomycota, Microsporidia, or Zygomycota. The fungal infection or overgrowth can include one or more fungal species, e.g., Candida albicans, C. tropicalis, C. parapsilosis, C. glabrata, C. auris, C. krusei, Saccharomyces cerevisiae, Malassezia globose, M. restricta, or Debaryomyces hansenii, Gibberella moniliformis, Alternaria brassicicola, Cryptococcus neoformans, Pneumocystis carinii, P. jirovecii, P. murina, P. oryctolagi, P. wakefieldiae, and Aspergillus clavatus. The fungal species may be considered a pathogen or an opportunistic pathogen.

In some instances, the fungal infection is caused by a fungus in the genus Candida (i.e., a Candida infection). For example, a Candida infection can be caused by a fungus in the genus Candida that is selected from the group consisting of C. albicans, C. glabrata, C. dubliniensis, C. krusei, C. auris, C. parapsilosis, C. tropicalis, C. orthopsilosis, C. guilliermondii, C. rugose, and C. lusitaniae. Candida infections that can be treated by the methods disclosed herein include, but are not limited to candidemia, oropharyngeal candidiasis, esophageal candidiasis, mucosal candidiasis, genital candidiasis, vulvovaginal candidiasis, rectal candidiasis, hepatic candidiasis, renal candidiasis, pulmonary candidiasis, splenic candidiasis, otomycosis, osteomyelitis, septic arthritis, cardiovascular candidiasis (e.g., endocarditis), and invasive candidiasis.

### B. Bacteria

The pathogen control compositions and related methods can be useful for decreasing the fitness of a bacterium, e.g., to prevent or treat a bacterial infection in an animal. Included are methods for administering a pathogen control composition to a bacterium by contacting the bacteria with the pathogen control composition. Additionally or alternatively, the methods include preventing or treating a bacterial infection (e.g., caused by a bacteria described herein) in an animal at risk of or in need thereof, by administering to the animal a pathogen control composition.

The pathogen control compositions and related methods are suitable for preventing or treating a bacterial infection in animals caused by any bacteria described further below. For example, the bacteria may be one belonging to BaciHales (B. anthracis, B. cereus, S. aureus, L. monocytogenes), Lactobacillales (S. pneumoniae, S. pyogenes), Clostridiales (C. botulinum, C. difficile, C. perfringens, C. tetani), Spirochaetales (Borrelia burgdorferi, Treponema pallidum), Chlamydiales (Chlamydia trachomatis, Chlamydophila psittaci), Actinomycetales (C. diphtheriae, Mycobacterium tuberculosis, M. avium), Rickettsiales (a prowazekii, R. rickettsii, R. typhi, A. phagocytophilum, E. chaffeensis), Rhizobiales (Brucella melitensis), Burkholderiales (Bordetella pertussis, Burkholderia mallei, B. pseudomallei), Neisseriales (Neisseria gonorrhoeae, N. meningitidis), Campylobacterales (Campylobacter jejuni, Helicobacter pylori), Legionellales (Legionella pneumophila), Pseudomonadales (A. baumannii, Moraxella catarrhalis, P. aeruginosa), Aeromonadales (Aeromonas sp.), Vibrionales (Vibrio cholerae, V. parahaemolyticus), Thiotrichales, Pasteurellales (Haemophilus influenzae), Enterobacteriales (Klebsiella pneumoniae, Proteus mirabilis, Yersinia pestis, Y. enterocolitica, Shigella flexneri, Salmonella enterica, E. coli).

In some instances, the bacteria is Pseudomonas aeruginosa or Escherichia coli.

### C. Parasitic Insects

The pathogen control compositions and related methods can be useful for decreasing the fitness of a parasitic insect, e.g., to prevent or treat a parasitic insect infection in an animal. The term “insect” includes any organism belonging to the phylum Arthropoda and to the class Insecta or the class Arachnida, in any stage of development, i.e., immature and adult insects. Included are methods for delivering a pathogen control composition to an insect by contacting the insect with the pathogen control composition. Additionally or alternatively, the methods include preventing or treating a parasitic insect infection (e.g., caused by a parasitic insect described herein) in an animal at risk of or in need thereof, by administering to the animal a pathogen control composition.

The pathogen control compositions and related methods are suitable for preventing or treating infection in animals by a parasitic insect, including infections by insects belonging to Phthiraptera: Anoplura (Sucking lice), Ischnocera (Chewing lice), Amblycera (Chewing lice). Siphonaptera: Pulicidae (Cat fleas), Ceratophyllidae (Chicken-fleas). Diptera: Culicidae (Mosquitoes), Ceratopogonidae (Midges), Psychodidae (Sandflies), Simuliidae (Blackflies), Tabanidae (Horse-flies), Muscidae (House-flies, etc.), Calliphoridae (Blowflies), Glossinidae (Tsetse-flies), Oestridae (Bot-flies), Hippoboscidae (Louse-flies). Hemiptera: Reduviidae (Assassin-bugs), Cimicidae (Bed-bugs). Arachnida: Sarcoptidae (Sarcoptic mites), Psoroptidae (Psoroptic mites), Cytoditidae (Air-sac mites), Laminosioptes (Cyst-mites), Analgidae (Feather-mites), Acaridae (Grain-mites), Demodicidae (Hair-follicle mites), Cheyletiellidae (Fur-mites), Trombiculidae (Trombiculids), Dermanyssidae (Bird mites), Macronyssidae (Bird mites), Argasidae (Soft-ticks), Ixodidae (Hard-ticks).

### D. Protozoa

The pathogen control compositions and related methods can be useful for decreasing the fitness of a parasitic protozoa, e.g., to prevent or treat a parasitic protozoa infection in an animal. The term “protozoa” includes any organism belonging to the phylum Protozoa. Included are methods for delivering a pathogen control composition to a parasitic protozoa by contacting the parasitic protozoa with the pathogen control composition. Additionally or alternatively, the methods include preventing or treating a protozoal infection (e.g., caused by a protozoan described herein) in an animal at risk of or in need thereof, by administering to the animal a pathogen control composition.

The pathogen control compositions and related methods are suitable for preventing or treating infection by parasitic protozoa in animals, including protozoa belonging to Euglenozoa (Trypanosoma cruzi, Trypanosoma brucei, Leishmania spp.), Heterolobosea (Naegleria fowleri), Diplomonadida (Giardia intestinalis), Amoebozoa (Acanthamoeba castellanii, Balamuthia mandrillaris, Entamoeba histolytica), Blastocystis (Blastocystis hominis), Apicomplexa (Babesia microti, Cryptosporidium parvum, Cyclospora cayetanensis, Plasmodium spp., Toxoplasma gondii).

### E. Nematodes

The pathogen control compositions and related methods can be useful for decreasing the fitness of a parasitic nematode, e.g., to prevent or treat a parasitic nematode infection in an animal. Included are methods for delivering a pathogen control composition to a parasitic nematode by contacting the parasitic nematode with the pathogen control composition. Additionally or alternatively, the methods include preventing or treating a parasitic nematode infection (e.g., caused by a parasitic nematode described herein) in an animal at risk of or in need thereof, by administering to the animal a pathogen control composition.

The pathogen control compositions and related methods are suitable for preventing or treating infection by parasitic nematodes in animals, including nematodes belonging to Nematoda (roundworms): Angiostrongylus cantonensis (rat lungworm), Ascaris lumbricoides (human roundworm), Baylisascaris procyonis (raccoon roundworm), Trichuris trichiura (human whipworm), Trichinella spiralis, Strongyloides stercoralis, Wuchereria bancrofti, Brugia malayi, Ancylostoma duodenale and Necator americanus (human hookworms), Cestoda (tapeworms): Echinococcus granulosus, Echinococcus multilocularis, Taenia solium (pork tapeworm).

### F. Viruses

The pathogen control compositions and related methods can be useful for decreasing the fitness of a virus, e.g., to prevent or treat a viral infection in an animal. Included are methods for delivering a pathogen control composition to a virus by contacting the virus with the pathogen control composition. Additionally or alternatively, the methods include preventing or treating a viral infection (e.g., caused by a virus described herein) in an animal at risk of or in need thereof, by administering to the animal a pathogen control composition.

The pathogen control compositions and related methods are suitable for preventing or treating a viral infection in animals, including infections by viruses belonging to DNA viruses: Parvoviridae, Papillomaviridae, Polyomaviridae, Poxviridae, Herpesviridae; Single-stranded negative strand RNA viruses: Arenaviridae, Paramyxoviridae (Rubulavirus, Respirovirus, Pneumovirus, Moribillivirus), Filoviridae (Marburgvirus, Ebolavirus), Bornaoviridae, Rhabdoviridae, Orthomyxoviridae, Bunyaviridae, Nairovirus, Hantaviruses, Orthobunyavirus, Phlebovirus. Single-stranded positive strand RNA viruses: Astroviridae, Coronaviridae, Caliciviridae, Togaviridae (Rubivirus, Alphavirus), Flaviviridae (Hepacivirus, Flavivirus), Picornaviridae (Hepatovirus, Rhinovirus, Enterovirus); or dsRNA and Retro-transcribed Viruses: Reoviridae (Rotavirus, Coltivirus, Seadornavirus), Retroviridae (Deltaretrovirus, Lentivirus), Hepadnaviridae (Orthohepadnavirus).

### G. Pathogen Vectors

The methods and compositions provided herein may be usesful for decreasing the fitness of a vector for an animal pathogen. In some instances, the vector may be an insect. For example, the insect vectormay include, but is not limited to those with piercing-sucking mouthparts, as found in Hemiptera and some Hymenoptera and Diptera such as mosquitoes, bees, wasps, midges, lice, tsetse fly, fleas and ants, as well as members of the Arachnidae such as ticks and mites; order, class or family of Acarina (ticks and mites) e.g. representatives of the families Argasidae, Dermanyssidae, Ixodidae, Psoroptidae or Sarcoptidae and representatives of the species Amblyomma spp., Anocenton spp., Argas spp., Boophilus spp., Cheyletiella spp., Chorioptes spp., Demodex spp., Dermacentor spp., Denmanyssus spp., Haemophysalis spp., Hyalomma spp., Ixodes spp., Lynxacarus spp., Mesostigmata spp., Notoednes spp., Ornithodoros spp., Ornithonyssus spp., Otobius spp., Otodectes spp., Pneumonyssus spp., Psoroptes spp., Rhipicephalus spp., Sancoptes spp., or Trombicula spp.; Anoplura (sucking and biting lice) e.g. representatives of the species Bovicola spp., Haematopinus spp., Linognathus spp., Menopon spp., Pediculus spp., Pemphigus spp., Phylloxera spp., or Solenopotes spp.; Diptera (flies) e.g. representatives of the species Aedes spp., Anopheles spp., Calliphora spp., Chrysomyia spp., Chrysops spp., Cochliomyia spp., Cw/ex spp., Culicoides spp., Cuterebra spp., Dermatobia spp., Gastrophilus spp., Glossina spp., Haematobia spp., Haematopota spp., Hippobosca spp., Hypoderma spp., Lucilia spp., Lyperosia spp., Melophagus spp., Oestrus spp., Phaenicia spp., Phlebotomus spp., Phormia spp., Acari (sarcoptic mange) e.g., Sarcoptidae spp., Sarcophaga spp., Simulium spp., Stomoxys spp., Tabanus spp., Tannia spp. or Zzpu/alpha spp.; Mallophaga (biting lice) e.g. representatives of the species Damalina spp., Felicola spp., Heterodoxus spp. or Trichodectes spp.; or Siphonaptera (wingless insects) e.g. representatives of the species Ceratophyllus spp., Xenopsylla spp; Cimicidae (true bugs) e.g. representatives of the species Cimex spp., Tritominae spp., Rhodinius spp., or Triatoma spp.

In some instances, the insect is a blood-sucking insect from the order Diptera (e.g., suborder Nematocera, e.g., family Colicidae). In some instances, the insect is from the subfamilies Culicinae, Corethrinae, Ceratopogonidae, or Simuliidae. In some instances, the insect is of a Culex spp., Theobaldia spp., Aedes spp., Anopheles spp., Aedes spp., Forciponiyia spp., Culicoides spp., or Helea spp.

In certain instances, the insect is a mosquito. In certain instances, the insect is a tick. In certain instances, the insect is a mite. In certain instances, the insect is a biting louse.

V. Heterologous Functional Agents

The pathogen control compositions described herein can further include an additional agent, such as a heterologous functional agent (e.g., antifungal agent, an antibacterial agent, a virucidal agent, an anti-viral agent, an insecticidal agent, a nematicidal agent, an antiparasitic agent, or an insect repellent). In some instances, the heterologous functional agent (e.g., antifungal agent, an antibacterial agent, a virucidal agent, an anti-viral agent, an insecticidal agent, a nematicidal agent, an antiparasitic agent, or an insect repellent) is included in the PMP. For example, the PMP may encapsulate the heterologous functional agent (e.g., antifungal agent, an antibacterial agent, a virucidal agent, an anti-viral agent, an insecticidal agent, a nematicidal agent, an antiparasitic agent, or an insect repellent). Alternatively, the heterologous functional agent (e.g., antifungal agent, an antibacterial agent, a virucidal agent, an anti-viral agent, an insecticidal agent, a nematicidal agent, an antiparasitic agent, or an insect repellent) can be embedded on or conjugated to the surface of the PMP. In some instances, the pathogen control composition includes two or more (e.g., 2, 3, 4, 5, 6, 7, 8, 9, 10, or more than 10) different heterologous functional agents.

In other instances, the pathogen control composition can be formulated to include the heterologous functional agent (e.g., antifungal agent, an antibacterial agent, a virucidal agent, an anti-viral agent, an insecticidal agent, a nematicidal agent, an antiparasitic agent, or an insect repellent), without it necessarily being associated with the PMP. In formulations and in the use forms prepared from these formulations, the pest control composition may include additional active compounds, such as antibactierals, insecticides, sterilants, acaricides, nematicides, molluscicides, bactericides, fungicides, virucides, attractants, or repellents.

The pesticidal agent can include an agent suitable for delivery to a vector of an animal pathogen, e.g., a pesticidal agent, such as an antifungal agent, an antibacterial agent, an insecticidal agent, a molluscicidal agent, a nematicidal agent, a virucidal agent, or a combination thereof. The pesticidal agent can be a chemical agent, such as those well known in the art. The pesticidal agent may be an agent that can decrease the fitness of a variety of animal pathogens, or vectors thereof, or can be one that targets one or more specific animal pathogens, or vectors thereof, (e.g., a specific species or genus of pathogens, or vectors thereof).

Alternatively or additionally, the heterologous functional agent (e.g., antifungal agent, an antibacterial agent, a virucidal agent, an anti-viral agent, an insecticidal agent, a nematicidal agent, an antiparasitic agent, or an insect repellent) can be a peptide, a polypeptide, a nucleic acid, a polynucleotide, or a small molecule. In some instances, the heterologous functional agent can be modified. For example, the modification can be a chemical modification, e.g., conjugation to a marker, e.g., fluorescent marker or a radioactive marker. In other examples, the modification can include conjugation or operational linkage to a moiety that enhances the stability, delivery, targeting, bioavailability, or half-life of the agent, e.g., a lipid, a glycan, a polymer (e.g., PEG), a cation moiety.

Examples of additional heterologous functional agents (e.g., antifungal agent, an antibacterial agent, a virucidal agent, an anti-viral agent, an insecticidal agent, a nematicidal agent, an antiparasitic agent, or an insect repellent) that can be used in the presently disclosed pathogen control compositions and methods are outlined below.

### A. Antibacterial Agents

The pathogen control compositions described herein can further include an antibacterial agent. For example, a pathogen control composition including an antibiotic as described herein can be administered to an animal in an amount and for a time sufficient to: reach a target level (e.g., a predetermined or threshold level) of antibiotic concentration inside or on the animal; and/or treat or prevent a bacterial infection in the animal. The antibacterials described herein may be formulated in a pathogen control composition for any of the methods described herein, and in certain instances, may be associated with the PMP thereof. In some instances, the pathogen control compositions includes two or more (e.g., 2, 3, 4, 5, 6, 7, 8, 9, 10, or more than 10) different antibacterial agents.

As used herein, the term “antibacterial agent” refers to a material that kills or inhibits the growth, proliferation, division, reproduction, or spread of bacteria, such as phytopathogenic bacteria, and includes bactericidal (e.g., disinfectant compounds, antiseptic compounds, or antibiotics) or bacteriostatic agents (e.g., compounds or antibiotics). Bactericidal antibiotics kill bacteria, while bacteriostatic antibiotics only slow their growth or reproduction.

Bactericides can include disinfectants, antiseptics, or antibiotics. The most used disinfectants can comprise: active chlorine (i.e., hypochlorites (e.g., sodium hypochlorite), chloramines, dichloroisocyanurate and trichloroisocyanurate, wet chlorine, chlorine dioxide etc.), active oxygen (peroxides, such as peracetic acid, potassium persulfate, sodium perborate, sodium percarbonate and urea perhydrate), iodine (iodpovidone (povidone-iodine, Betadine), Lugol's solution, iodine tincture, iodinated nonionic surfactants), concentrated alcohols (mainly ethanol, 1-propanol, called also n-propanol and 2-propanol, called isopropanol and mixtures thereof; further, 2-phenoxyethanol and 1- and 2-phenoxypropanols are used), phenolic substances (such as phenol (also called carbolic acid), cresols (called Lysole in combination with liquid potassium soaps), halogenated (chlorinated, brominated) phenols, such as hexachlorophene, triclosan, trichlorophenol, tribromophenol, pentachlorophenol, Dibromol and salts thereof), cationic surfactants, such as some quaternary ammonium cations (such as benzalkonium chloride, cetyl trimethylammonium bromide or chloride, didecyldimethylammonium chloride, cetylpyridinium chloride, benzethonium chloride) and others, non-quaternary compounds, such as chlorhexidine, glucoprotamine, octenidine dihydrochloride etc.), strong oxidizers, such as ozone and permanganate solutions; heavy metals and their salts, such as colloidal silver, silver nitrate, mercury chloride, phenylmercury salts, copper sulfate, copper oxide-chloride, copper hydroxide, copper octanoate, copper oxychloride sulfate, copper sulfate, copper sulfate pentahydrate, etc. Heavy metals and their salts are the most toxic, and environment-hazardous bactericides and therefore, their use is strongly oppressed or canceled; further, also properly concentrated strong acids (phosphoric, nitric, sulfuric, amidosulfuric, toluenesulfonic acids) and alkalis (sodium, potassium, calcium hydroxides). As antiseptics (i.e., germicide agents that can be used on human or animal body, skin, mucoses, wounds and the like), few of the above mentioned disinfectants can be used, under proper conditions (mainly concentration, pH, temperature and toxicity toward man/animal). Among them, important are: properly diluted chlorine preparations (i.e. Daquin's solution, 0.5% sodium or potassium hypochlorite solution, pH-adjusted to pH 7-8, or 0.5-1% solution of sodium benzenesulfochloramide (chloramine B)), some iodine preparations, such as iodopovidone in various galenics (ointment, solutions, wound plasters), in the past also Lugol's solution, peroxides as urea perhydrate solutions and pH-buffered 0.1-0.25% peracetic acid solutions, alcohols with or without antiseptic additives, used mainly for skin antisepsis, weak organic acids such as sorbic acid, benzoic acid, lactic acid and salicylic acid some phenolic compounds, such as hexachlorophene, triclosan and Dibromol, and cation-active compounds, such as 0.05-0.5% benzalkonium, 0.5-4% chlorhexidine, 0.1-2% octenidine solutions.

The pathogen control composition described herein may include an antibiotic. Any antibiotic known in the art may be used. Antibiotics are commonly classified based on their mechanism of action, chemical structure, or spectrum of activity.

The antibiotic described herein may target any bacterial function or growth processes and may be either bacteriostatic (e.g., slow or prevent bacterial growth) or bactericidal (e.g., kill bacteria). In some instances, the antibiotic is a bactericidal antibiotic. In some instances, the bactericidal antibiotic is one that targets the bacterial cell wall (e.g., penicillins and cephalosporins); one that targets the cell membrane (e.g., polymyxins); or one that inhibits essential bacterial enzymes (e.g., rifamycins, lipiarmycins, quinolones, and sulfonamides). In some instances, the bactericidal antibiotic is an aminoglycoside (e.g., kasugamycin). In some instances, the antibiotic is a bacteriostatic antibiotic. In some instances the bacteriostatic antibiotic targets protein synthesis (e.g., macrolides, lincosamides, and tetracyclines). Additional classes of antibiotics that may be used herein include cyclic lipopeptides (such as daptomycin), glycylcyclines (such as tigecycline), oxazolidinones (such as linezolid), or lipiarmycins (such as fidaxomicin). Examples of antibiotics include rifampicin, ciprofloxacin, doxycycline, ampicillin, and polymyxin B. The antibiotic described herein may have any level of target specificity (e.g., narrow- or broad-spectrum). In some instances, the antibiotic is a narrow-spectrum antibiotic, and thus targets specific types of bacteria, such as gram-negative or gram-positive bacteria. Alternatively, the antibiotic may be a broad-spectrum antibiotic that targets a wide range of bacteria. In some instances, the antibiotic is doxorubicin or vancomycin.

Examples of antibacterial agents suitable for the treatment of animals include Penicillins (Amoxicillin, Ampicillin, Bacampicillin, Carbenicillin, Cloxacillin, Dicloxacillin, Flucloxacillin, Mezlocillin, Nafcillin, Oxacillin, Penicillin G, Crysticillin 300 A.S., Pentids, Permapen, Pfizerpen, Pfizerpen-AS, Wycillin, Penicillin V, Piperacillin, Pivampicillin, Pivmecillinam, Ticarcillin), Cephalosporins (Cefacetrile (cephacetrile), Cefadroxil (cefadroxyl), Cefalexin (cephalexin), Cefaloglycin (cephaloglycin), Cefalonium (cephalonium), Cefaloridine (cephaloradine), Cefalotin (cephalothin), Cefapirin (cephapirin), Cefatrizine, Cefazaflur, Cefazedone, Cefazolin (cephazolin), Cefradine (cephradine), Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Ceftobiprole, Ceftaroline, Cefaclomezine, Cefaloram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Cefuracetime, Ceftioxide, Combinations, Ceftazidime/Avibactam, Ceftolozane/Tazobactam), Monobactams (Aztreonam), Carbapenems (Imipenem, Imipenem/cilastatin, Doripenem, Ertapenem, Meropenem, Meropenem/vaborbactam), Macrolide (Azithromycin, Erythromycin, Clarithromycin, Dirithromycin, Roxithromycin, Telithromycin), Lincosamides (Clindamycin, Lincomycin), Streptogramins (Pristinamycin, Quinupristin/dalfopristin), Aminoglycoside (Amikacin, Gentamicin, Kanamycin, Neomycin, Netilmicin, Paromomycin, Streptomycin, Tobramycin), Quinolone (Flumequine, Nalidixic acid, Oxolinic acid, Piromidic acid, Pipemidic acid, Rosoxacin, Second Generation, Ciprofloxacin, Enoxacin, Lomefloxacin, Nadifloxacin, Norfloxacin, Ofloxacin, Pefloxacin, Rufloxacin, Balofloxacin, Gatifloxacin, Grepafloxacin, Levofloxacin, Moxifloxacin, Pazufloxacin, Sparfloxacin, Temafloxacin, Tosufloxacin, Besifloxacin, Delafloxacin, Clinafloxacin, Gemifloxacin, Prulifloxacin, Sitafloxacin, Trovafloxacin), Sulfonamides (Sulfamethizole, Sulfamethoxazole, Sulfisoxazole, Trimethoprim-Sulfamethoxazole), Tetracycline (Demeclocycline, Doxycycline, Minocycline, Oxytetracycline, Tetracycline, Tigecycline), Other (Lipopeptides, Fluoroquinolone, Lipoglycopeptides, Cephalosporin, Macrocyclics, Chloramphenicol, Metronidazole, Tinidazole, Nitrofurantoin, Glycopeptides, Vancomycin, Teicoplanin, Lipoglycopeptides, Telavancin, Oxazolidinones, Linezolid, Cycloserine 2, Rifamycins, Rifampin, Rifabutin, Rifapentine, Rifalazil, Polypeptides, Bacitracin, Polymyxin B, Tuberactinomycins, Viomycin, Capreomycin).

One skilled in the art will appreciate that a suitable concentration of each antibiotic in the composition depends on factors such as efficacy, stability of the antibiotic, number of distinct antibiotics, the formulation, and methods of application of the composition.

### B. Antifungal Agents

The pathogen control compositions described herein can further include an antifungal agent. For example, a pathogen control composition including an antifungal as described herein can be administered to an animal in an amount and for a time sufficient to reach a target level (e.g., a predetermined or threshold level) of antifungal concentration inside or on the animal; and/or treat or prevent a fungal infection in the animal. The antifungals described herein may be formulated in a pathogen control composition for any of the methods described herein, and in certain instances, may be associated with the PMP thereof. In some instances, the pathogen control compositions includes two or more (e.g., 2, 3, 4, 5, 6, 7, 8, 9, 10, or more than 10) different antifungal agents.

As used herein, the term “fungicide” or “antifungal agent” refers to a substance that kills or inhibits the growth, proliferation, division, reproduction, or spread of fungi, such as fungi that are pathogenic to animals. Many different types of antifungal agent have been produced commercially. Non limiting examples of antifungal agents include: Allylamines (Amorolfin, Butenafine, Naftifine, Terbinafine), Imidazoles ((Bifonazole, Butoconazole, Clotrimazole, Econazole, Fenticonazole, Ketoconazole, Isoconazole, Luliconazole, Miconazole, Omoconazole, Oxiconazole, Sertaconazole, Sulconazole, Tioconazole, Terconazole); Triazoles (Albaconazole, Efinaconazole, Fluconazole, Isavuconazole, Itraconazole, Posaconazole, Ravuconazole, Terconazole, Voriconazole), Thiazoles (Abafungin), Polyenes (Amphotericin B, Nystatin, Natamycin, Trichomycin), Echinocandins (Anidulafungin, Caspofungin, Micafungin), Other (Tolnaftate, Flucytosine, Butenafine, Griseofulvin, Ciclopirox, Selenium sulfide, Tavaborole). One skilled in the art will appreciate that a suitable concentration of each antifungal in the composition depends on factors such as efficacy, stability of the antifungal, number of distinct antifungals, the formulation, and methods of application of the composition.

### C. Insecticides

The pathogen control compositions described herein can further include an insecticide. For example, the insecticide can decrease the fitness of (e.g., decrease growth or kill) an insect vector of an animal pathogen. A pathogen control composition including an insecticide as described herein can be contacted with an insect, in an amount and for a time sufficient to: (a) reach a target level (e.g., a predetermined or threshold level) of insecticide concentration inside or on the insect; and (b) decrease fitness of the insect. In some instances, the insecticide can decrease the fitness of (e.g., decrease growth or kill) a parasitic insect. A pathogen control composition including an insecticide as described herein can be contacted with a parasitic insect, or an animal infected therewith, in an amount and for a time sufficient to: (a) reach a target level (e.g., a predetermined or threshold level) of insecticide concentration inside or on the parasitic insect; and (b) decrease the fitness of the parasitic insect. The insecticides described herein may be formulated in a pathogen control composition for any of the methods described herein, and in certain instances, may be associated with the PMP thereof. In some instances, the pathogen control compositions include two or more (e.g., 2, 3, 4, 5, 6, 7, 8, 9, 10, or more than 10) different insecticide agents.

As used herein, the term “insecticide” or “insecticidal agent” refers to a substance that kills or inhibits the growth, proliferation, reproduction, or spread of insects, such as insect vectors of animal pathogens or parasitic insects. Non limiting examples of insecticides are shown in Table 1. Additional non-limiting examples of suitable insecticides include biologics, hormones or pheromones such as azadirachtin, Bacillus species, Beauveria species, codlemone, Metarrhizium species, Paecilomyces species, thuringiensis, and Verticillium species, and active compounds having unknown or non-specified mechanisms of action such as fumigants (such as aluminium phosphide, methyl bromide and sulphuryl fluoride) and selective feeding inhibitors (such as cryolite, flonicamid and pymetrozine). One skilled in the art will appreciate that a suitable concentration of each insecticide in the composition depends on factors such as efficacy, stability of the insecticide, number of distinct insecticides, the formulation, and methods of application of the composition.

TABLE 1
Examples of insecticides

| Class | Compounds |
|---|---|
| chloronicotinyls/ | acetamiprid, clothianidin, dinotefuran, imidacloprid, nitenpyram, |
| neonicotinoids | nithiazine, thiacloprid, thiamethoxam, imidaclothiz, (2E)-1-[(2- |
| | chloro-1,3-thiazol-5-yl)methyl]-3,5-dimethyl-N-nitro-1,3,5-tri-azinan- |
| | 2-imine, acetylcholinesterase (AChE) inhibitors (such as |
| | carbamates and organophosphates) |
| carbamates | alanycarb, aldicarb, aldoxycarb, allyxycarb, aminocarb, bendiocarb, |
| | benfuracarb, bufencarb, butacarb, butocarboxim, butoxycarboxim, |
| | carbaryl, carbofuran, carbosulfan, chloethocarb, dimetilan, |
| | ethiofencarb, fenobucarb, fenothiocarb, formetanate, furathiocarb, |
| | isoprocarb, metam-sodium, methiocarb, methomyl, metolcarb, |
| | oxamyl, phosphocarb, pirimicarb, promecarb, propoxur, thiodicarb, |
| | thiofanox, triazamate, trimethacarb, XMC, xylylcarb |
| organophosphates | acephate, azamethiphos, azinphos (-methyl, -ethyl), bromophos- |
| | ethyl, bromfenvinfos (-methyl), butathiofos, cadusafos, |
| | carbophenothion, chlorethoxyfos, chlorfenvinphos, chlormephos, |
| | chlorpyrifos (-methyl/-ethyl), coumaphos, cyanofenphos, |
| | cyanophos, demeton-S-methyl, demeton-S-methylsulphon, dialifos, |
| | diazinon, dichlofenthion, dichlorvos/DDVP, dicrotophos, |
| | dimethoate, dimethylvinphos, dioxabenzofos, disulfoton, EPN, |
| | ethion, ethoprophos, etrimfos, famphur, fenamiphos, fenitrothion, |
| | fensulfothion, fenthion, flupyrazofos, fonofos, formothion, |
| | fosmethilan, fosthiazate, heptenophos, iodofenphos, iprobenfos, |
| | isazofos, isofenphos, isopropyl O-salicylate, isoxathion, malathion, |
| | mecarbam, methacrifos, methamidophos, methidathion, |
| | mevinphos, monocrotophos, naled, omethoate, oxydemeton- |
| | methyl, parathion (-methyl/-ethyl), phenthoate, phorate, phosalone, |
| | phosmet, phosphamidon, phosphocarb, phoxim, pirimiphos |
| | (-methyl/-ethyl), profenofos, propaphos, propetamphos, prothiofos, |
| | prothoate, pyraclofos, pyridaphenthion, pyridathion, quinalphos, |
| | sebufos, sulfotep, sulprofos, tebupirimfos, temephos, terbufos, |
| | tetrachlorvinphos, thiometon, triazophos, triclorfon, vamidothion |
| pyrethroids | acrinathrin, allethrin (d-cis-trans, d-trans), cypermethrin (alpha-, |
| | beta-, theta-, zeta-), permethrin (cis-, trans-), beta-cyfluthrin, |
| | bifenthrin, bioallethrin, bioallethrin-S-cyclopentyl-isomer, |
| | bioethanomethrin, biopermethrin, bioresmethrin, chlovaporthrin, |
| | cis-cypermethrin, cis-resmethrin, cis-permethrin, clocythrin, |
| | cycloprothrin, cyfluthrin, cyhalothrin, cyphenothrin, DDT, |
| | deltamethrin, empenthrin (1R-isomer), esfenvalerate, etofenprox, |
| | fenfluthrin, fenpropathrin, fenpyrithrin, fenvalerate, flubrocythrinate, |
| | flucythrinate, flufenprox, flumethrin, fluvalinate, fubfenprox, gamma- |
| | cyhalothrin, imiprothrin, kadethrin, lambda, cyhalothrin, |
| | metofluthrin, phenothrin (1R-trans isomer), prallethrin, profluthrin, |
| | protrifenbute, pyresmethrin, resmethrin, RU 15525, silafluofen, tau- |
| | fluvalinate, tefluthrin, terallethrin, tetramethrin (1R-isomer), |
| | tralocythrin, tralomethrin, transfluthrin, ZXI 8901, pyrethrins |
| | (pyrethrum) |
| oxadiazines | indoxacarb, acetylcholine receptor modulators (such as spinosyns) |
| spinosyns | spinosad |
| cyclodiene | camphechlor, chlordane, endosulfan, gamma-HCH, HCH, |
| | heptachlor, |
| organochlorines | lindane, methoxychlor |
| fiproles | acetoprole, ethiprole, vaniliprole, fipronil |
| mectins | abamectin, avermectin, emamectin, emamectin-benzoate, |
| | fenoxycarb, hydroprene, kinoprene, methoprene, ivermectin, |
| | lepimectin, epofenonane, pyriproxifen, milbemectin, milbemycin, |
| | triprene |
| diacylhydrazines | chromafenozide, halofenozide, methoxyfenozide, tebufenozide |
| benzoylureas | bistrifluoron, chlorfluazuron, diflubenzuron, fluazuron, flucycloxuron, |
| | flufenoxuron, hexaflumuron, lufenuron, novaluron, noviflumuron, |
| | penfluoron, teflubenzuron, triflumuron |
| organotins | azocyclotin, cyhexatin, fenbutatin oxide |
| pyrroles | chlorfenapyr |
| dinitrophenols | binapacyrl, dinobuton, dinocap, DNOC |
| METIs | fenazaquin, fenpyroximate, pyrimidifen, pyridaben, tebufenpyrad, |
| | tolfenpyrad, rotenone, acequinocyl, fluacrypyrim, microbial |
| | disrupters of the intestinal membrane of insects (such as Bacillus |
| | thuringiensis strains), inhibitors of lipid synthesis (such as tetronic |
| | acids and tetramic acids) |
| tetronic acids | spirodiclofen, spiromesifen, spirotetramat |
| tetramic acids | cis-3-(2,5-dimethylphenyl)-8-methoxy-2-oxo-1-azaspiro[4.5]dec-3- |
| | en-4-yl ethyl carbonate (alias: carbonic acid, 3-(2,5- |
| | dimethylphenyl)-8-methoxy-2-oxo-1-azaspiro[4.5]dec-3-en-4-yl |
| | ethyl ester; CAS Reg. No.: 382608-10-8), carboxamides (such as |
| | flonicamid), octopaminergic agonists (such as amitraz), inhibitors of |
| | the magnesium-stimulated ATPase (such as propargite), ryanodin |
| | receptor agonists (such as phthalamides or rynaxapyr) |
| phthalamides | N2-[1,1-dimethyl-2-(methylsulphonyl)ethyl]-3-iodo-N1-[2-methyl--4- |
| | [1,2,2,2-tetrafluoro-1-(trifluoromethyl)ethyl]phenyl]-1,2-benzenedi- |
| | carboxamide (i.e., flubendiamide; CAS reg. No.: 272451-65-7) |

### D. Nematicides

The pathogen control compositions described herein can further include a nematicide. In some instances, the pathogen control composition includes two or more (e.g., 2, 3, 4, 5, 6, 7, 8, 9, 10, or more than 10) different nematicides. For example, the nematicide can decrease the fitness of (e.g., decrease growth or kill) a parasitic nematode. A pathogen control composition including a nematicide as described herein can be contacted with a parasitic nematode, or an animal infected therewith, in an amount and for a time sufficient to: (a) reach a target level (e.g., a predetermined or threshold level) of nematicide concentration inside or on the target nematode; and (b) decrease fitness of the parasitic nematode. The nematicides described herein may be formulated in a pathogen control composition for any of the methods described herein, and in certain instances, may be associated with the PMP thereof.

As used herein, the term “nematicide” or “nematicidal agent” refers to a substance that kills or inhibits the growth, proliferation, reproduction, or spread of nematodes, such as a parasitic nematode. Non limiting examples of nematicides are shown in Table 2. One skilled in the art will appreciate that a suitable concentration of each nematicide in the composition depends on factors such as efficacy, stability of the nematicide, number of distinct nematicides, the formulation, and methods of application of the composition.

TABLE 2
Examples of Nematicides

| | |
|---|---|
| FUMIGANTS | D-D, 1,3-Dichloropropene, Ethylene Dibromide, 1,2-Dibromo-3- |
| | Chloropropane, Methyl Bromide, Chloropicrin, Metam Sodium, Dazomet, |
| | Methyl Isothiocyanate (MITC), Sodium Tetrathiocarbonate, Chloropicrin, |
| CARBAMATES | Aldicarb, Aldoxycarb, Carbofuran, Oxamyl, Cleothocarb |
| ORGANOPHOSPHATES | Ethoprophos, Fenamiphos, Cadusafos, Fosthiazate, Fensulfothion, |
| | Thionazin, Isazofos, |
| BIOCHEMICALS | DITERA ®, CLANDOSAN ®, SINCOCIN ® |

### E. Antiparasitic Agent

The pathogen control compositions described herein can further include an antiparasitic agent. For example, the antiparasitic can decrease the fitness of (e.g., decrease growth or kill) a parasitic protozoan. A pathogen control composition including an antiparasitic as described herein can be contacted with a protozoan in an amount and for a time sufficient to: (a) reach a target level (e.g., a predetermined or threshold level) of antiparasitic concentration inside or on the protozoan, or animal infected therewith; and (b) decrease fitness of the protozoan. This can be useful in the treatment or prevention of parasites in animals. For example, a pathogen control composition including an antiparasitic agent as described herein can be administered to an animal in an amount and for a time sufficient to: reach a target level (e.g., a predetermined or threshold level) of antiparasitic concentration inside or on the animal; and/or treat or prevent a parasite (e.g., parasitic nematode, parasitic insect, or protozoan) infection in the animal. The antiparasitic described herein may be formulated in a pathogen control composition for any of the methods described herein, and in certain instances, may be associated with the PMP thereof. In some instances, the pathogen control composition includes two or more (e.g., 2, 3, 4, 5, 6, 7, 8, 9, 10, or more than 10) different antiparasitic agents.

As used herein, the term “antiparasitic” or “antiparasitic agent” refers to a substance that kills or inhibits the growth, proliferation, reproduction, or spread of parasites, such as parasitic protozoa, parasitic nematodes, or parasitic insects. Examples of antiparasitic agents include Antihelmintics (Bephenium, Diethylcarbamazine, Ivermectin, Niclosamide, Piperazine, Praziquantel, Pyrantel, Pyrvinium, Benzimidazoles, Albendazole, Flubendazole, Mebendazole, Thiabendazole, Levamisole, Nitazoxanide, Monopantel, Emodepside, Spiroindoles), Scabicides (Benzyl benzoate, Benzyl benzoate/disulfiram, Lindane, Malathion, Permethrin), Pediculicides (Piperonyl butoxide/pyrethrins, Spinosad, Moxidectin), Scabicides (Crotamiton), Anticestodes (Niclosamide, Pranziquantel, Albendazole), Antiamoebics (Rifampin, Apmphotericin B); or Antiprotozoals (Melarsoprol, Eflornithine, Metronidazole, Tinidazole, Miltefosine, Artemisinin). In certain instances, the antiparasitic agent may be use for treating or prevening infections in livestock animals, e.g., Levamisole, Fenbendazole, Oxfendazole, Albendazole, Moxidectin, Eprinomectin, Doramectin, Ivermectin, or Clorsulon. One skilled in the art will appreciate that a suitable concentration of each antiparasitic in the composition depends on factors such as efficacy, stability of the antiparasitic, number of distinct antiparasitics, the formulation, and methods of application of the composition.

### F. Antiviral Agent

The pathogen control compositions described herein can further include an antiviral agent. A pathogen control composition including an antivirual agent as described herein can be administered to an animal in an amount and for a time sufficient to reach a target level (e.g., a predetermined or threshold level) of antiviral concentration inside or on the animal; and/or to treat or prevent a viral infection in the animal. The antivirals described herein may be formulated in a pathogen control composition for any of the methods described herein, and in certain instances, may be associated with the PMP thereof. In some instances, the pathogen control composition includes two or more (e.g., 2, 3, 4, 5, 6, 7, 8, 9, 10, or more than 10) different antivirals.

As used herein, the term “antiviral” or “virucide” refers to a substance that kills or inhibits the growth, proliferation, reproduction, development, or spread of viruses, such as viral pathogens that infect animals. A number of agents can be employed as an antiviral, including chemicals or biological agents (e.g., nucleic acids, e.g., dsRNA). Examples of antiviral agents useful herein include Abacavir, Acyclovir (Aciclovir), Adefovir, Amantadine, Amprenavir (Agenerase), Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Nucleoside analogues, Norvir, Oseltamivir (Tamiflu), Peginterferon alfa-2a, Penciclovir, Peramivir, Pleconaril, Podophyllotoxin, Raltegravir, Ribavirin, Rimantadine, Ritonavir, Pyramidine, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), or Zidovudine. One skilled in the art will appreciate that a suitable concentration of each antiviral in the composition depends on factors such as efficacy, stability of the antivirals, number of distinct antivirals, the formulation, and methods of application of the composition.

### G. Repellents

The pathogen control compositions described herein can further include a repellent. For example, the repellent can repel a vector of animal pathogens, such as insects. The repellent described herein may be formulated in a pathogen control composition for any of the methods described herein, and in certain instances, may be associated with the PMP thereof. In some instances, the pathogen control composition includes two or more (e.g., 2, 3, 4, 5, 6, 7, 8, 9, 10, or more than 10) different repellents.

For example, a pathogen control composition including a repellent as described herein can be contacted with an insect vector or a habitat of the vector in an amount and for a time sufficient to: (a) reach a target level (e.g., a predetermined or threshold level) of repellent concentration; and/or (b) decrease the levels of the insect near or on nearby animals relative to a control. Alternatively, a pathogen control composition including a repellent as described herein can be contacted with an animal in an amount and for a time sufficient to: (a) reach a target level (e.g., a predetermined or threshold level) of repellent concentration; and/or (b) decrease the levels of the insect near or on the animal relative to an untreated animal.

Some examples of well-known insect repellents include: benzil; benzyl benzoate; 2,3,4,5-bis(butyl-2-ene)tetrahydrofurfural (MGK Repellent 11); butoxypolypropylene glycol; N-butylacetanilide; normal-butyl-6,6-dimethyl-5,6-dihydro-1,4-pyrone-2-carboxylate (Indalone); dibutyl adipate; dibutyl phthalate; di-normal-butyl succinate (Tabatrex); N,N-diethyl-meta-toluamide (DEET); dimethyl carbate (endo,endo)-dimethyl bicyclo[2.2.1] hept-5-ene-2,3-dicarboxylate); dimethyl phthalate; 2-ethyl-2-butyl-1,3-propanediol; 2-ethyl-1,3-hexanediol (Rutgers 612); di-normal-propyl isocinchomeronate (MGK Repellent 326); 2-phenylcyclohexanol; p-methane-3,8-diol, and normal-propyl N,N-diethylsuccinamate. Other repellents include citronella oil, dimethyl phthalate, normal-butylmesityl oxide oxalate and 2-ethyl hexanediol-1,3 (See, Kirk-Othmer Encyclopedia of Chemical Technology, 2nd Ed., Vol. 11: 724-728; and The Condensed Chemical Dictionary, 8th Ed., p 756).

In some instances, the repellent is an insect repellent, including synthetic or nonsynthetic insect repellents. Examples of synthetic insect repellents include methyl anthranilate and other anthranilate-based insect repellents, benzaldehyde, DEET (N,N-diethyl-m-toluamide), dimethyl carbate, dimethyl phthalate, icaridin (i.e., picaridin, Bayrepel, and KBR 3023), indalone (e.g., as used in a “6-2-2” mixture (60% Dimethyl phthalate, 20% Indalone, 20% Ethylhexanediol), IR3535 (3-[N-Butyl-N-acetyl]-aminopropionic acid, ethyl ester), metofluthrin, permethrin, SS220, or tricyclodecenyl allyl ether. Examples of natural insect repellents include beautyberry (Callicarpa) leaves, birch tree bark, bog myrtle (Myrica Gale), catnip oil (e.g., nepetalactone), citronella oil, essential oil of the lemon eucalyptus (Corymbia citriodora; e.g., p-menthane-3,8-diol (PMD)), neem oil, lemongrass, tea tree oil from the leaves of Melaleuca alternifolia, tobacco, or extracts thereof.

### H. Biological Agents

i. Polypeptides

The pathogen control composition (e.g., PMPs) described herein may include a polypeptide, e.g., a polypeptide that is an antibacterial, antifungal, insecticidal, nematicidal, antiparasitic, or virucidal. In some instances, the pathogen control composition described herein includes a polypeptide or functional fragments or derivative thereof, that targets pathways in the pathogen. A pathogen control composition including a polypeptide as described herein can be administered to a pathogen, a vector thereof, in an amount and for a time sufficient to: (a) reach a target level (e.g., a predetermined or threshold level) of polypeptide concentration; and (b) decrease or eliminate the pathogen. In some instances, a pathogen control composition including a polypeptide as described herein can be administered to an animal having or at risk of an infection by a pathogen in an amount and for a time sufficient to: (a) reach a target level (e.g., a predetermined or threshold level) of polypeptide concentration in the animal; and (b) decrease or eliminate the pathogen. The polypeptides described herein may be formulated in a pathogen control composition for any of the methods described herein, and in certain instances, may be associated with the PMP thereof.

Examples of polypeptides that can be used herein can include an enzyme (e.g., a metabolic recombinase, a helicase, an integrase, a RNAse, a DNAse, or an ubiquitination protein), a pore-forming protein, a signaling ligand, a cell penetrating peptide, a transcription factor, a receptor, an antibody, a nanobody, a gene editing protein (e.g., CRISPR-Cas system, TALEN, or zinc finger), riboprotein, a protein aptamer, or a chaperone.

Polypeptides included herein may include naturally occurring polypeptides or recombinantly produced variants. In some instances, the polypeptide may be a functional fragments or variants thereof (e.g., an enzymatically active fragment or variant thereof). For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide may have at least 50% (e.g., at least 50%, 60%, 70%, 80%, 90%, 95%, 97%, 99%, or greater) identity to a protein of interest.

The polypeptides described herein may be formulated in a composition for any of the uses described herein. The compositions disclosed herein may include any number or type (e.g., classes) of polypeptides, such as at least about any one of 1 polypeptide, 2, 3, 4, 5, 10, 15, 20, or more polypeptides. A suitable concentration of each polypeptide in the composition depends on factors such as efficacy, stability of the polypeptide, number of distinct polypeptides in the composition, the formulation, and methods of application of the composition. In some instances, each polypeptide in a liquid composition is from about 0.1 ng/mL to about 100 mg/mL. In some instances, each polypeptide in a solid composition is from about 0.1 ng/g to about 100 mg/g.

Methods of making a polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Methods for producing a polypeptide involve expression in plant cells, although recombinant proteins can also be produced using insect cells, yeast, bacteria, mammalian cells, or other cells under the control of appropriate promoters. Mammalian expression vectors may comprise nontranscribed elements such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture a recombinant polypeptide agent. Examples of mammalian expression systems include CHO cells, COS cells, HeLA and BHK cell lines. Processes of host cell culture for production of protein therapeutics are described in, e.g., Zhou and Kantardjieff (Eds.), Mammalian Cell Cultures for Biologics Manufacturing (Advances in Biochemical Engineering/Biotechnology), Springer (2014). Purification of proteins is described in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012).

In some instances, the pathogen control composition includes an antibody or antigen binding fragment thereof. For example, an agent described herein may be an antibody that blocks or potentiates activity and/or function of a component of the pathogen. The antibody may act as an antagonist or agonist of a polypeptide (e.g., enzyme or cell receptor) in the pathogen. The making and use of antibodies against a target antigen in a pathogen is known in the art. See, for example, Zhiqiang An (Ed.), Therapeutic Monoclonal Antibodies: From Bench to Clinic, 1st Edition, Wiley, 2009 and also Greenfield (Ed.), Antibodies: A Laboratory Manual, 2nd Edition, Cold Spring Harbor Laboratory Press, 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

The pathogen control composition described herein may include a bacteriocin. In some instances, the bacteriocin is naturally produced by Gram-positive bacteria, such as Pseudomonas, Streptomyces, Bacillus, Staphylococcus, or lactic acid bacteria (LAB, such as Lactococcus lactis). In some instances, the bacteriocin is naturally produced by Gram-negative bacteria, such as Hafnia alvei, Citrobacter freundii, Klebsiella oxytoca, Klebsiella pneumonia, Enterobacter cloacae, Serratia plymithicum, Xanthomonas campestris, Erwinia carotovora, Ralstonia solanacearum, or Escherichia coli. Exemplary bacteriocins include, but are not limited to, Class I-IV LAB antibiotics (such as lantibiotics), colicins, microcins, and pyocins.

The pathogen control composition described herein may include an antimicrobial peptide (AMP). Any AMP suitable for inhibiting a microorganism may be used. AMPs are a diverse group of molecules, which are divided into subgroups on the basis of their amino acid composition and structure. The AMP may be derived or produced from any organism that naturally produces AMPs, including AMPs derived from plants (e.g., copsin), insects (e.g., mastoparan, poneratoxin, cecropin, moricin, melittin), frogs (e.g., magainin, dermaseptin, aurein), and mammals (e.g., cathelicidins, defensins and protegrins).

ii. Nucleic Acids

Numerous nucleic acids are useful in the compositions and methods described herein. The compositions disclosed herein may include any number or type (e.g., classes) of nucleic acids (e.g., DNA molecule or RNA molecule, e.g., mRNA, guide RNA (gRNA), or inhibitory RNA molecule (e.g., siRNA, shRNA, or miRNA), or a hybrid DNA-RNA molecule), such as at least about 1 class or variant of a nucleic acid, 2, 3, 4, 5, 10, 15, 20, or more classes or variants of nucleic acids. A suitable concentration of each nucleic acid in the composition depends on factors such as efficacy, stability of the nucleic acid, number of distinct nucleic acids, the formulation, and methods of application of the composition. Examples of nucleic acids useful herein include a Dicer substrate small interfering RNA (dsiRNA), an antisense RNA, a short interfering RNA (siRNA), a short hairpin (shRNA), a microRNA (miRNA), an (asymmetric interfering RNA) aiRNA, a peptide nucleic acid (PNA), a morpholino, a locked nucleic acid (LNA), a piwi-interacting RNA (piRNA), a ribozyme, a deoxyribozymes (DNAzyme), an aptamer (DNA, RNA), a circular RNA (circRNA), a guide RNA (gRNA), or a DNA molecule

A pathogen control composition including a nucleic acid as described herein can be contacted with a pathogen, or vector thereof, in an amount and for a time sufficient to: (a) reach a target level (e.g., a predetermined or threshold level) of nucleic acid concentration; and (b) decrease or eliminate the pathogen. In some instances, a pathogen control composition including a nucleic acid as described herein can be administered to an animal having or at risk of an infection by a pathogen in an amount and for a time sufficient to: (a) reach a target level (e.g., a predetermined or threshold level) of nucleic acid concentration in the animal; and (b) decrease or eliminate the pathogen. The nucleic acids described herein may be formulated in a pathogen control composition for any of the methods described herein, and in certain instances, may be associated with the PMP thereof.

### (a) Nucleic Acid Encoding Peptides

In some instances, the pathogen control composition includes a nucleic acid encoding a polypeptide. Nucleic acids encoding a polypeptide may have a length from about 10 to about 50,000 nucleotides (nts), about 25 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, about 5000 to about 6000 nts, about 6000 to about 7000 nts, about 7000 to about 8000 nts, about 8000 to about 9000 nts, about 9000 to about 10,000 nts, about 10,000 to about 15,000 nts, about 10,000 to about 20,000 nts, about 10,000 to about 25,000 nts, about 10,000 to about 30,000 nts, about 10,000 to about 40,000 nts, about 10,000 to about 45,000 nts, about 10,000 to about 50,000 nts, or any range therebetween.

The pathogen control composition may also include functionally active variants of a nucleic acid sequence of interest. In some instances, the variant of the nucleic acids has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a nucleic acid of interest. In some instances, the invention includes a functionally active polypeptide encoded by a nucleic acid variant as described herein. In some instances, the functionally active polypeptide encoded by the nucleic acid variant has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire amino acid sequence, to a sequence of a polypeptide of interest or the naturally derived polypeptide sequence.

Some methods for expressing a nucleic acid encoding a protein may involve expression in cells, including insect, yeast, plant, bacteria, or other cells under the control of appropriate promoters. Expression vectors may include nontranscribed elements, such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green et al., Molecular Cloning: A Laboratory Manual, Fourth Edition, Cold Spring Harbor Laboratory Press, 2012.

Genetic modification using recombinant methods is generally known in the art. A nucleic acid sequence coding for a desired gene can be obtained using recombinant methods known in the art, such as, for example by screening libraries from cells expressing the gene, by deriving the gene from a vector known to include the same, or by isolating directly from cells and tissues containing the same, using standard techniques. Alternatively, a gene of interest can be produced synthetically, rather than cloned.

Expression of natural or synthetic nucleic acids is typically achieved by operably linking a nucleic acid encoding the gene of interest to a promoter, and incorporating the construct into an expression vector. Expression vectors can be suitable for replication and expression in bacteria. Expression vectors can also be suitable for replication and integration in eukaryotes. Typical cloning vectors contain transcription and translation terminators, initiation sequences, and promoters useful for expression of the desired nucleic acid sequence.

Additional promoter elements, e.g., enhancers, regulate the frequency of transcriptional initiation. Typically, these are located in the region 30-110 basepairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Alternatively, the promoter may be an inducible promoter. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes may be used for identifying potentially transformed cells and for evaluating the functionality of regulatory sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., FEBS Letters 479:79-82, 2000). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some instances, an organism may be genetically modified to alter expression of one or more proteins. Expression of the one or more proteins may be modified for a specific time, e.g., development or differentiation state of the organism. In one instances, the invention includes a composition to alter expression of one or more proteins, e.g., proteins that affect activity, structure, or function. Expression of the one or more proteins may be restricted to a specific location(s) or widespread throughout the organism.

(b) Synthetic mRNA

The pathogen control composition may include a synthetic mRNA molecule, e.g., a synthetic mRNA molecule encoding a polypeptide. The synthetic mRNA molecule can be modified, e.g., chemically. The mRNA molecule can be chemically synthesized or transcribed in vitro. The mRNA molecule can be disposed on a plasmid, e.g., a viral vector, bacterial vector, or eukaryotic expression vector. In some examples, the mRNA molecule can be delivered to cells by transfection, electroporation, or transduction (e.g., adenoviral or lentiviral transduction).

In some instances, the modified RNA agent of interest described herein has modified nucleosides or nucleotides. Such modifications are known and are described, e.g., in WO 2012/019168. Additional modifications are described, e.g., in WO 2015/038892; WO 2015/038892; WO 2015/089511; WO 2015/196130; WO 2015/196118 and WO 2015/196128 A2.

In some instances, the modified RNA encoding a polypeptide of interest has one or more terminal modification, e.g., a 5′ cap structure and/or a poly-A tail (e.g., of between 100-200 nucleotides in length). The 5′ cap structure may be selected from the group consisting of CapO, CapI, ARCA, inosine, NI-methyl-guanosine, 2′fluoro-guanosine, 7-deaza-guanosine, 8-oxo-guanosine, 2-amino-guanosine, LNA-guanosine, and 2-azido-guanosine. In some cases, the modified RNAs also contain a 5 UTR including at least one Kozak sequence, and a 3 UTR. Such modifications are known and are described, e.g., in WO 2012/135805 and WO 2013/052523. Additional terminal modifications are described, e.g., in WO 2014/164253 and WO 2016/011306, WO 2012/045075, and WO 2014/093924. Chimeric enzymes for synthesizing capped RNA molecules (e.g., modified mRNA) which may include at least one chemical modification are described in WO 2014/028429.

In some instances, a modified mRNA may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, and 3) ribozyme catalyzed. The newly formed 5′-/3′-linkage may be intramolecular or intermolecular. Such modifications are described, e.g., in WO 2013/151736.

Methods of making and purifying modified RNAs are known and disclosed in the art. For example, modified RNAs are made using only in vitro transcription (IVT) enzymatic synthesis. Methods of making IVT polynucleotides are known in the art and are described in WO 2013/151666, WO 2013/151668, WO 2013/151663, WO 2013/151669, WO 2013/151670, WO 2013/151664, WO 2013/151665, WO 2013/151671, WO 2013/151672, WO 2013/151667 and WO 2013/151736.S Methods of purification include purifying an RNA transcript including a polyA tail by contacting the sample with a surface linked to a plurality of thymidines or derivatives thereof and/or a plurality of uracils or derivatives thereof (polyT/U) under conditions such that the RNA transcript binds to the surface and eluting the purified RNA transcript from the surface (WO 2014/152031); using ion (e.g., anion) exchange chromatography that allows for separation of longer RNAs up to 10,000 nucleotides in length via a scalable method (WO 2014/144767); and subjecting a modified mRNA sample to DNAse treatment (WO 2014/152030).

Formulations of modified RNAs are known and are described, e.g., in WO 2013/090648. For example, the formulation may be, but is not limited to, nanoparticles, poly(lactic-co-glycolic acid)(PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs) and combinations thereof.

Modified RNAs encoding polypeptides in the fields of human disease, antibodies, viruses, and a variety of in vivo settings are known and are disclosed in for example, Table 6 of International Publication Nos. WO 2013/151666, WO 2013/151668, WO 2013/151663, WO 2013/151669, WO 2013/151670, WO 2013/151664, WO 2013/151665, WO 2013/151736; Tables 6 and 7 International Publication No. WO 2013/151672; Tables 6, 178 and 179 of International Publication No. WO 2013/151671; Tables 6, 185 and 186 of International Publication No WO 2013/151667. Any of the foregoing may be synthesized as an IVT polynucleotide, chimeric polynucleotide or a circular polynucleotide, and each may include one or more modified nucleotides or terminal modifications.

### (c) Inhibitory RNA

In some instances, the pathogen control composition includes an inhibitory RNA molecule, e.g., that acts via the RNA interference (RNAi) pathway. In some instances, the inhibitory RNA molecule decreases the level of gene expression in a pathogen, or vector thereof. In some instances, the inhibitory RNA molecule decreases the level of a protein in the pathogen, or vector thereof. In some instances, the inhibitory RNA molecule inhibits expression of a pathogen gene. In some instances, the inhibitory RNA molecule inhibits expression of a gene in a vector of a pathogen. For example, an inhibitory RNA molecule may include a short interfering RNA, short hairpin RNA, and/or a microRNA that targets a gene in the pathogen. Certain RNA molecules can inhibit gene expression through the biological process of RNA interference (RNAi). RNAi molecules include RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: Dicer substrate small interfering RNAs (dsiRNA), short interfering RNAs (siRNAs), double-strand RNAs (dsRNA), short hairpin RNAs (shRNA), meroduplexes, dicer substrates, and multivalent RNA interference (U.S. Pat. Nos. 8,084,599 8,349,809, 8,513,207 and 9,200,276). A shRNA is a RNA molecule including a hairpin turn that decreases expression of target genes via RNAi. shRNAs can be delivered to cells in the form of plasmids, e.g., viral or bacterial vectors, e.g., by transfection, electroporation, or transduction). A microRNA is a non-coding RNA molecule that typically has a length of about 22 nucleotides. MiRNAs bind to target sites on mRNA molecules and silence the mRNA, e.g., by causing cleavage of the mRNA, destabilization of the mRNA, or inhibition of translation of the mRNA. In some instances, the inhibitory RNA molecule decreases the level and/or activity of a negative regulator of function. In other instances, the inhibitor RNA molecule decreases the level and/or activity of an inhibitor of a positive regulator of function. The inhibitory RNA molecule can be chemically synthesized or transcribed in vitro.

In some instances, the nucleic acid is a DNA, a RNA, or a PNA. In some instances, the RNA is an inhibitory RNA. In some instances, the inhibitory RNA inhibits gene expression in a pathogen. In some instances, the nucleic acid is an mRNA, a modified mRNA, or a DNA molecule that increases expression in the pathogen of an enzyme (e.g., a metabolic recombinase, a helicase, an integrase, a RNAse, a DNAse, or an ubiquitination protein), a pore-forming protein, a signaling ligand, a cell penetrating peptide, a transcription factor, a receptor, an antibody, a nanobody, a gene editing protein (e.g., CRISPR-Cas system, TALEN, or zinc finger), riboprotein, a protein aptamer, or a chaperone. In some instances, the nucleic acid is an mRNA, a modified mRNA, or a DNA molecule that increases the expression of an enzyme (e.g., a metabolic enzyme, a recombinase enzyme, a helicase enzyme, an integrase enzyme, a RNAse enzyme, a DNAse enzyme, or an ubiquitination protein), a pore-forming protein, a signaling ligand, a cell penetrating peptide, a transcription factor, a receptor, an antibody, a nanobody, a gene editing protein (e.g., a CRISPR-Cas system, a TALEN, or a zinc finger), a riboprotein, a protein aptamer, or a chaperone. In some instances, the increase in expression in the pathogen is an increase in expression of about 5%, 10%, 15%, 20%, 30%, 40%, 50%, 60%, 70%, 80%, 90%, 100%, or more than 100% relative to a reference level (e.g., the expression in an untreated pathogen). In some instances, the increase in expression in the pathogen is an increase in expression of about 2× fold, about 4× fold, about 5× fold, about 10× fold, about 20× fold, about 25× fold, about 50× fold, about 75× fold, or about 100× fold or more, relative to a reference level (e.g., the expression in an untreated pathogen).

In some instances, the nucleic acid is an antisense RNA, a siRNA, a shRNA, a miRNA, an aiRNA, a PNA, a morpholino, a LNA, a piRNA, a ribozyme, a DNAzyme, an aptamer (DNA, RNA), a circRNA, a gRNA, or a DNA molecules (e.g., an antisense polynucleotide) to reduces expression in the pathogen of, e.g., an enzyme (a metabolic enzyme, a recombinase enzyme, a helicase enzyme, an integrase enzyme, a RNAse enzyme, a DNAse enzyme, a polymerase enzyme, a ubiquitination protein, a superoxide management enzyme, or an energy production enzyme), a transcription factor, a secretory protein, a structural factor (actin, kinesin, or tubulin), a riboprotein, a protein aptamer, a chaperone, a receptor, a signaling ligand, or a transporter. In some instances, the decrease in expression in the pathogen is a decrease in expression of about 5%, 10%, 15%, 20%, 30%, 40%, 50%, 60%, 70%, 80%, 90%, 100%, or more than 100% relative to a reference level (e.g., the expression in an untreated pathogen). In some instances, the decrease in expression in the pathogen is a decrease in expression of about 2× fold, about 4× fold, about 5× fold, about 10× fold, about 20× fold, about 25× fold, about 50× fold, about 75× fold, or about 100× fold or more, relative to a reference level (e.g., the expression in an untreated pathogen).

RNAi molecules include a sequence substantially complementary, or fully complementary, to all or a fragment of a target gene. RNAi molecules may complement sequences at the boundary between introns and exons to prevent the maturation of newly-generated nuclear RNA transcripts of specific genes into mRNA for transcription. RNAi molecules complementary to specific genes can hybridize with the mRNA for a target gene and prevent its translation. The antisense molecule can be DNA, RNA, or a derivative or hybrid thereof. Examples of such derivative molecules include, but are not limited to, peptide nucleic acid (PNA) and phosphorothioate-based molecules such as deoxyribonucleic guanidine (DNG) or ribonucleic guanidine (RNG).

RNAi molecules can be provided as ready-to-use RNA synthesized in vitro or as an antisense gene transfected into cells which will yield RNAi molecules upon transcription. Hybridization with mRNA results in degradation of the hybridized molecule by RNAse H and/or inhibition of the formation of translation complexes. Both result in a failure to produce the product of the original gene.

The length of the RNAi molecule that hybridizes to the transcript of interest may be around 10 nucleotides, between about 15 or 30 nucleotides, or about 15, 16, 17, 18, 19, 20, 21, 22, 23, 24, 25, 26, 27, 28, 29, 30 or more nucleotides. The degree of identity of the antisense sequence to the targeted transcript may be at least 75%, at least 80%, at least 85%, at least 90%, or at least 95.

RNAi molecules may also include overhangs, i.e., typically unpaired, overhanging nucleotides which are not directly involved in the double helical structure normally formed by the core sequences of the herein defined pair of sense strand and antisense strand. RNAi molecules may contain 3′ and/or 5′ overhangs of about 1-5 bases independently on each of the sense strands and antisense strands. In some instances, both the sense strand and the antisense strand contain 3′ and 5′ overhangs. In some instances, one or more of the 3′ overhang nucleotides of one strand base pairs with one or more 5′ overhang nucleotides of the other strand. In other instances, the one or more of the 3′ overhang nucleotides of one strand base do not pair with the one or more 5′ overhang nucleotides of the other strand. The sense and antisense strands of an RNAi molecule may or may not contain the same number of nucleotide bases. The antisense and sense strands may form a duplex wherein the 5′ end only has a blunt end, the 3′ end only has a blunt end, both the 5′ and 3′ ends are blunt ended, or neither the 5′ end nor the 3′ end are blunt ended. In another instance, one or more of the nucleotides in the overhang contains a thiophosphate, phosphorothioate, deoxynucleotide inverted (3′ to 3′ linked) nucleotide or is a modified ribonucleotide or deoxynucleotide.

Small interfering RNA (siRNA) molecules include a nucleotide sequence that is identical to about 15 to about 25 contiguous nucleotides of the target mRNA. In some instances, the siRNA sequence commences with the dinucleotide AA, includes a GC-content of about 30-70% (about 30-60%, about 40-60%, or about 45%-55%), and does not have a high percentage identity to any nucleotide sequence other than the target in the genome in which it is to be introduced, for example as determined by standard BLAST search.

siRNAs and shRNAs resemble intermediates in the processing pathway of the endogenous microRNA (miRNA) genes (Bartel, Cell 116:281-297, 2004). In some instances, siRNAs can function as miRNAs and vice versa (Zeng et al., Mol. Cell 9:1327-1333, 2002; Doench et al., Genes Dev. 17:438-442, 2003). Exogenous siRNAs downregulate mRNAs with seed complementarity to the siRNA (Birmingham et al., Nat. Methods 3:199-204, 2006). Multiple target sites within a 3′ UTR give stronger downregulation (Doench et al., Genes Dev. 17:438-442, 2003).

Known effective siRNA sequences and cognate binding sites are also well represented in the relevant literature. RNAi molecules are readily designed and produced by technologies known in the art. In addition, there are computational tools that increase the chance of finding effective and specific sequence motifs (Pei et al., Nat. Methods 3(9):670-676, 2006; Reynolds et al., Nat. Biotechnol. 22(3):326-330, 2004; Khvorova et al., Nat. Struct. Biol. 10(9):708-712, 2003; Schwarz et al., Cell 115(2):199-208, 2003; Ui-Tei et al., Nucleic Acids Res. 32(3):936-948, 2004; Heale et al., Nucleic Acids Res. 33(3):e30, 2005; Chalk et al., Biochem. Biophys. Res. Commun. 319(1):264-274, 2004; and Amarzguioui et al., Biochem. Biophys. Res. Commun. 316(4):1050-1058, 2004).

The RNAi molecule modulates expression of RNA encoded by a gene. Because multiple genes can share some degree of sequence homology with each other, in some instances, the RNAi molecule can be designed to target a class of genes with sufficient sequence homology. In some instances, the RNAi molecule can contain a sequence that has complementarity to sequences that are shared amongst different gene targets or are unique for a specific gene target. In some instances, the RNAi molecule can be designed to target conserved regions of an RNA sequence having homology between several genes thereby targeting several genes in a gene family (e.g., different gene isoforms, splice variants, mutant genes, etc.). In some instances, the RNAi molecule can be designed to target a sequence that is unique to a specific RNA sequence of a single gene.

An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that such modifications can increase nuclease resistance and/or serum stability, or decrease immunogenicity.

In some instances, the RNAi molecule is linked to a delivery polymer via a physiologically labile bond or linker. The physiologically labile linker is selected such that it undergoes a chemical transformation (e.g., cleavage) when present in certain physiological conditions, (e.g., disulfide bond cleaved in the reducing environment of the cell cytoplasm). Release of the molecule from the polymer, by cleavage of the physiologically labile linkage, facilitates interaction of the molecule with the appropriate cellular components for activity.

The RNAi molecule-polymer conjugate may be formed by covalently linking the molecule to the polymer. The polymer is polymerized or modified such that it contains a reactive group A. The RNAi molecule is also polymerized or modified such that it contains a reactive group B. Reactive groups A and B are chosen such that they can be linked via a reversible covalent linkage using methods known in the art.

Conjugation of the RNAi molecule to the polymer can be performed in the presence of an excess of polymer. Because the RNAi molecule and the polymer may be of opposite charge during conjugation, the presence of excess polymer can reduce or eliminate aggregation of the conjugate. Alternatively, an excess of a carrier polymer, such as a polycation, can be used. The excess polymer can be removed from the conjugated polymer prior to administration of the conjugate. Alternatively, the excess polymer can be co-administered with the conjugate.

Injection of double-stranded RNA (dsRNA) into mother insects efficiently suppresses their offspring's gene expression during embryogenesis, see for example, Khila et al., PLoS Genet. 5(7):e1000583, 2009; and Liu et al., Development 131(7):1515-1527, 2004. Matsuura et al. (PNAS 112(30):9376-9381, 2015) has shown that suppression of Ubx eliminates bacteriocytes and the symbiont localization of bacteriocytes.

The making and use of inhibitory agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press (2010).

### (d) Gene Editing

The pathogen control compositions described herein may include a component of a gene editing system. For example, the agent may introduce an alteration (e.g., insertion, deletion (e.g., knockout), translocation, inversion, single point mutation, or other mutation) in a gene in the pathogen. Exemplary gene editing systems include the zinc finger nucleases (ZFNs), Transcription Activator-Like Effector-based Nucleases (TALEN), and the clustered regulatory interspaced short palindromic repeat (CRISPR) system. ZFNs, TALENs, and CRISPR-based methods are described, e.g., in Gaj et al., Trends Biotechnol. 31(7):397-405, 2013.

In a typical CRISPR/Cas system, an endonuclease is directed to a target nucleotide sequence (e.g., a site in the genome that is to be sequence-edited) by sequence-specific, non-coding guide RNAs that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (crRNA), and a trans-activating crRNA (tracrRNA). The crRNA contains a guide RNA, i.e., typically an about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The RNAs serve as guides to direct Cas proteins to silence specific DNA/RNA sequences, depending on the spacer sequence. See, e.g., Horvath et al., Science 327:167-170, 2010; Makarova et al., Biology Direct 1:7, 2006; Pennisi, Science 341:833-836, 2013. The target DNA sequence must generally be adjacent to a protospacer adjacent motif (PAM) that is specific for a given Cas endonuclease; however, PAM sequences appear throughout a given genome. CRISPR endonucleases identified from various prokaryotic species have unique PAM sequence requirements; examples of PAM sequences include 5′-NGG (SEQ ID NO: 78) (Streptococcus pyogenes), 5′-NNAGAA (SEQ ID NO: 79) (Streptococcus thermophilus CRISPR1), 5′-NGGNG (SEQ ID NO: 80) (Streptococcus thermophilus CRISPR3), and 5′-NNNGATT (SEQ ID NO: 81) (Neisseria meningiditis). Some endonucleases, e.g., Cas9 endonucleases, are associated with G-rich PAM sites, e.g., 5′-NGG (SEQ ID NO: 78), and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; in other words a Cpf1 system requires only the Cpf1 nuclease and a crRNA to cleave the target DNA sequence. Cpf1 endonucleases, are associated with T-rich PAM sites, e.g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al., Cell 163:759-771, 2015.

For the purposes of gene editing, CRISPR arrays can be designed to contain one or multiple guide RNA sequences corresponding to a desired target DNA sequence; see, for example, Cong et al., Science 339:819-823, 2013; Ran et al., Nature Protocols 8:2281-2308, 2013. At least about 16 or 17 nucleotides of gRNA sequence are required by Cas9 for DNA cleavage to occur; for Cpf1 at least about 16 nucleotides of gRNA sequence is needed to achieve detectable DNA cleavage. In practice, guide RNA sequences are generally designed to have a length of between 17-24 nucleotides (e.g., 19, 20, or 21 nucleotides) and complementarity to the targeted gene or nucleic acid sequence. Custom gRNA generators and algorithms are available commercially for use in the design of effective guide RNAs. Gene editing has also been achieved using a chimeric single guide RNA (sgRNA), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al., Nature Biotechnol. 985-991, 2015.

Whereas wild-type Cas9 generates double-strand breaks (DSBs) at specific DNA sequences targeted by a gRNA, a number of CRISPR endonucleases having modified functionalities are available, for example: a nickase version of Cas9 generates only a single-strand break; a catalytically inactive Cas9 (dCas9) does not cut the target DNA but interferes with transcription by steric hindrance. dCas9 can further be fused with an effector to repress (CRISPRi) or activate (CRISPRa) expression of a target gene. For example, Cas9 can be fused to a transcriptional repressor (e.g., a KRAB domain) or a transcriptional activator (e.g., a dCas9-VP64 fusion). A catalytically inactive Cas9 (dCas9) fused to Fokl nuclease (dCas9-Fokl) can be used to generate DSBs at target sequences homologous to two gRNAs. See, e.g., the numerous CRISPR/Cas9 plasmids disclosed in and publicly available from the Addgene repository (Addgene, 75 Sidney St., Suite 550A, Cambridge, Mass. 02139; addgene.org/crispr/). A double nickase Cas9 that introduces two separate double-strand breaks, each directed by a separate guide RNA, is described as achieving more accurate genome editing by Ran et al., Cell 154:1380-1389, 2013.

CRISPR technology for editing the genes of eukaryotes is disclosed in US Patent Application Publications US 2016/0138008 A1 and US 2015/0344912 A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some instances, the desired genome modification involves homologous recombination, wherein one or more double-stranded DNA breaks in the target nucleotide sequence is generated by the RNA-guided nuclease and guide RNA(s), followed by repair of the break(s) using a homologous recombination mechanism (homology-directed repair). In such instances, a donor template that encodes the desired nucleotide sequence to be inserted or knocked-in at the double-stranded break is provided to the cell or subject; examples of suitable templates include single-stranded DNA templates and double-stranded DNA templates (e.g., linked to the polypeptide described herein). In general, a donor template encoding a nucleotide change over a region of less than about 50 nucleotides is provided in the form of single-stranded DNA; larger donor templates (e.g., more than 100 nucleotides) are often provided as double-stranded DNA plasmids. In some instances, the donor template is provided to the cell or subject in a quantity that is sufficient to achieve the desired homology-directed repair but that does not persist in the cell or subject after a given period of time (e.g., after one or more cell division cycles). In some instances, a donor template has a core nucleotide sequence that differs from the target nucleotide sequence (e.g., a homologous endogenous genomic region) by at least 1, at least 5, at least 10, at least 20, at least 30, at least 40, at least 50, or more nucleotides. This core sequence is flanked by homology arms or regions of high sequence identity with the targeted nucleotide sequence; in some instances, the regions of high identity include at least 10, at least 50, at least 100, at least 150, at least 200, at least 300, at least 400, at least 500, at least 600, at least 750, or at least 1000 nucleotides on each side of the core sequence. In some instances where the donor template is in the form of a single-stranded DNA, the core sequence is flanked by homology arms including at least 10, at least 20, at least 30, at least 40, at least 50, at least 60, at least 70, at least 80, or at least 100 nucleotides on each side of the core sequence. In instances, where the donor template is in the form of a double-stranded DNA, the core sequence is flanked by homology arms including at least 500, at least 600, at least 700, at least 800, at least 900, or at least 1000 nucleotides on each side of the core sequence. In one instance, two separate double-strand breaks are introduced into the cell or subject's target nucleotide sequence with a double nickase Cas9 (see Ran et al., Cell 154:1380-1389, 2013), followed by delivery of the donor template.

In some instances, the composition includes a gRNA and a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. The choice of nuclease and gRNA(s) is determined by whether the targeted mutation is a deletion, substitution, or addition of nucleotides, e.g., a deletion, substitution, or addition of nucleotides to a targeted sequence. Fusions of a catalytically inactive endonuclease e.g., a dead Cas9 (dCas9, e.g., D10A; H840A) tethered with all or a portion of (e.g., biologically active portion of) an (one or more) effector domain create chimeric proteins that can be linked to the polypeptide to guide the composition to specific DNA sites by one or more RNA sequences (sgRNA) to modulate activity and/or expression of one or more target nucleic acids sequences.

In instances, the agent includes a guide RNA (gRNA) for use in a CRISPR system for gene editing. In some instances, the agent includes a zinc finger nuclease (ZFN), or a mRNA encoding a ZFN, that targets (e.g., cleaves) a nucleic acid sequence (e.g., DNA sequence) of a gene in the pathogen. In some instances, the agent includes a TALEN, or an mRNA encoding a TALEN, that targets (e.g., cleaves) a nucleic acid sequence (e.g., DNA sequence) in a gene in the pathogen.

For example, the gRNA can be used in a CRISPR system to engineer an alteration in a gene in the pathogen. In other examples, the ZFN and/or TALEN can be used to engineer an alteration in a gene in the pathogen. Exemplary alterations include insertions, deletions (e.g., knockouts), translocations, inversions, single point mutations, or other mutations. The alteration can be introduced in the gene in a cell, e.g., in vitro, ex vivo, or in vivo. In some examples, the alteration increases the level and/or activity of a gene in the pathogen. In other examples, the alteration decreases the level and/or activity of (e.g., knocks down or knocks out) a gene in the pathogen. In yet another example, the alteration corrects a defect (e.g., a mutation causing a defect), in a gene in the pathogen.

In some instances, the CRISPR system is used to edit (e.g., to add or delete a base pair) a target gene in the pathogen. In other instances, the CRISPR system is used to introduce a premature stop codon, e.g., thereby decreasing the expression of a target gene. In yet other instances, the CRISPR system is used to turn off a target gene in a reversible manner, e.g., similarly to RNA interference. In some instances, the CRISPR system is used to direct Cas to a promoter of a gene, thereby blocking an RNA polymerase sterically.

In some instances, a CRISPR system can be generated to edit a gene in the pathogen, using technology described in, e.g., U.S. Publication No. 20140068797, Cong, Science 339: 819-823, 2013; Tsai, Nature Biotechnol. 32:6 569-576, 2014; U.S. Pat. Nos. 8,871,445; 8,865,406; 8,795,965; 8,771,945; and 8,697,359.

In some instances, the CRISPR interference (CRISPRi) technique can be used for transcriptional repression of specific genes in the pathogen. In CRISPRi, an engineered Cas9 protein (e.g., nuclease-null dCas9, or dCas9 fusion protein, e.g., dCas9-KRAB or dCas9-SID4X fusion) can pair with a sequence specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

In some instances, CRISPR-mediated gene activation (CRISPRa) can be used for transcriptional activation of a gene in the pathogen. In the CRISPRa technique, dCas9 fusion proteins recruit transcriptional activators. For example, dCas9 can be fused to polypeptides (e.g., activation domains) such as VP64 or the p65 activation domain (p65D) and used with sgRNA (e.g., a single sgRNA or multiple sgRNAs), to activate a gene or genes in the pathogen. Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains) such as VP64. In some examples, the synergistic activation mediator (SAM) system can be used for transcriptional activation. In SAM, MS2 aptamers are added to the sgRNA. MS2 recruits the MS2 coat protein (MCP) fused to p65AD and heat shock factor 1 (HSF1).

The CRISPRi and CRISPRa techniques are described in greater detail, e.g., in Dominguez et al., Nat. Rev. Mol. Cell Biol. 17:5-15, 2016, incorporated herein by reference. In addition, dCas9-mediated epigenetic modifications and simultaneous activation and repression using CRISPR systems, as described in Dominguez et al., can be used to modulate a gene in the pathogen.

iii. Small Molecules

In some instances, the pathogen control composition includes a small molecule, e.g., a biological small molecule. Numerous small molecule agents are useful in the methods and compositions described herein.

Small molecules include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds.

The small molecule described herein may be formulated in a composition or associated with the PMP for any of the pathogen control compositions or related methods described herein. The compositions disclosed herein may include any number or type (e.g., classes) of small molecules, such as at least about any one of 1 small molecule, 2, 3, 4, 5, 10, 15, 20, or more small molecules. A suitable concentration of each small molecule in the composition depends on factors such as efficacy, stability of the small molecule, number of distinct small molecules, the formulation, and methods of application of the composition. In some instances, wherein the composition includes at least two types of small molecules, the concentration of each type of small molecule may be the same or different.

A pathogen control composition including a small molecule as described herein can be contacted with the pathogen, or vector thereof, in an amount and for a time sufficient to: (a) reach a target level (e.g., a predetermined or threshold level) of small molecule concentration inside or on a pathogen, or vector thereof, and (b) decrease the fitness of the pathogen.

In some instances, the pathogen control composition including a small molecule as described herein can be administered to an animal having or at risk of an infection by a pathogen in an amount and for a time sufficient to: (a) reach a target level (e.g., a predetermined or threshold level) of small molecule concentration in the animal; and (b) decrease or eliminate the pathogen.

In some instances, the pathogen control composition of the compositions and methods described herein includes a secondary metabolite. Secondary metabolites are derived from organic molecules produced by an organism. Secondary metabolites may act (i) as competitive agents used against bacteria, fungi, amoebae, plants, insects, and large animals; (ii) as metal transporting agents; (iii) as agents of symbiosis between microbes and plants, insects, and higher animals; (iv) as sexual hormones; and (v) as differentiation effectors.

The secondary metabolite used herein may include a metabolite from any known group of secondary metabolites. For example, secondary metabolites can be categorized into the following groups: alkaloids, terpenoids, flavonoids, glycosides, natural phenols (e.g., gossypol acetic acid), enals (e.g., trans-cinnamaldehyde), phenazines, biphenols and dibenzofurans, polyketides, fatty acid synthase peptides, nonribosomal peptides, ribosomally synthesized and post-translationally modified peptides, polyphenols, polysaccharides (e.g., chitosan), and biopolymers. For an in-depth review of secondary metabolites see, for example, Vining, Annu. Rev. Microbiol. 44:395-427, 1990.

VI. Kits

The present invention also provides a kit for the control, prevention, or treatment of diseases caused by animal pathogens, or to control vectors of such pathogens, where the kit includes a container having a pathogen control composition described herein. The kit may further include instructional material for applying or deliverying (e.g., to an animal, to an animal pathogen, or to a vector of an animal pathogen) the pathogen control composition to control, prevent, or treat an infection in accordance with a method of the present invention. The skilled artisan will appreciate that the instructions for applying the pathogen control composition in the methods of the present invention can be any form of instruction. Such instructions include, but are not limited to, written instruction material (such as, a label, a booklet, a pamphlet), oral instructional material (such as on an audio cassette or CD) or video instructions (such as on a video tape or DVD).

### EXAMPLES

The following is an example of the methods of the invention. It is understood that various other embodiments may be practiced, given the general description provided above.

### Example 1: Isolation of Plant Messenger Packs from Plants

This example demonstrates the isolation of crude plant messenger packs (PMPs) from various plant sources, including the leaf apoplast, seed apoplast, root, fruit, vegetable, pollen, phloem, xylem sap, and plant cell culture medium.

### Experimental Design:

a) PMP Isolation from the Apoplast of Arabidopsis thaliana Leaves

Arabidopsis (Arabidopsis thaliana Col-0) seeds are surface sterilized with 50% bleach and plated on 0.53 Murashige and Skoog medium containing 0.8% agar. The seeds are vernalized for 2 d at 4° C. before being moved to short-day conditions (9-h days, 22° C., 150 ρEm⁻²). After 1 week, the seedlings are transferred to Pro-Mix PGX. Plants are grown for 4-6 weeks before harvest.

PMPs are isolated from the apoplastic wash of 4-6-week old Arabidopsis rosettes, as described by Rutter and Innes, Plant Physiol. 173(1): 728-741, 2017. Briefly, whole rosettes are harvested at the root and vacuum infiltrated with vesicle isolation buffer (20 mM MES, 2 mM CaCl₂), and 0.1 M NaCl, pH6). Infiltrated plants are carefully blotted to remove excess fluid, placed inside 30-mL syringes, and centrifuged in 50 mL conical tubes at 700 g for 20 min at 2° C. to collect the apoplast extracellular fluid containing EVs. Next, the apoplast extracellular fluid is filtered through a 0.85 pm filter to remove large particles, and PMPs are purified as described in Example 2.

b) PMP Isolation from the Apoplast of Sunflower Seeds

Intact sunflower seeds (H. annuus L.), and are imbibed in water for 2 hours, peeled to remove the pericarp, and the apoplastic extracellular fluid is extracted by a modified vacuum infiltration-centrifugation procedure, adapted from Regente et al, FEBS Letters. 583: 3363-3366, 2009. Briefly, seeds are immersed in vesicle isolation buffer (20 mM MES, 2 mM CaCl₂), and 0.1 M NaCl, pH6) and subjected to three vacuum pulses of 10s, separated by 30s intervals at a pressure of 45 kPa. The infiltrated seeds are recovered, dried on filter paper, placed in fritted glass filters and centrifuged for 20 min at 400 g at 4° C. The apoplast extracellular fluid is recovered, filtered through a 0.85 pm filter to remove large particles, and PMPs are purified as described in Example 2.

c) PMP Isolation from Ginger Roots

Fresh ginger (Zingiber officinale) rhizome roots are purchased from a local supplier and washed 3× with PBS. A total of 200 grams of washed roots is ground in a mixer (Osterizer 12-speed blender) at the highest speed for 10 min (pause 1 min for every 1 min of blending), and PMPs are isolated as described in Zhuang et al., J Extracellular Vesicles. 4(1):28713, 2015. Briefly, ginger juice is sequentially centrifuged at 1,000 g for 10 min, 3,000 g for 20 min and 10,000 g for 40 min to remove large particles from the PMP-containing supernatant. PMPs are purified as described in Example 2.

d) PMP Isolation from Grapefruit Juice

Fresh grapefruits (Citrus×paradise) are purchased from a local supplier, their skins are removed, and the fruit is manually pressed, or ground in a mixer (Osterizer 12-speed blender) at the highest speed for 10 min (pause 1 min for every minute of blending) to collect the juice, as described by Wang et al., Molecular Therapy. 22(3): 522-534, 2014 with minor modifications. Briefly, juice/juice pulp is sequentially centrifuged at 1,000 g for 10 min, 3,000 g for 20 min, and 10,000 g for 40 min to remove large particles from the PMP-containing supernatant. PMPs are purified as described in Example 2.

e) PMP Isolation from Broccoli Heads

Broccoli (Brassica oleracea var. italica) PMPs are isolated as previously described (Deng et al., Molecular Therapy, 25(7): 1641-1654, 2017). Briefly, fresh broccoli is purchased from a local supplier, washed three times with PBS, and ground in a mixer (Osterizer 12-speed blender) at the highest speed for 10 min (pause 1 min for every minute of blending). Broccoli juice is then sequentially centrifuged at 1,000 g for 10 min, 3,000 g for 20 min, and 10,000 g for 40 min to remove large particles from the PMP-containing supernatant. PMPs are purified as described in Example 2.

f) PMP Isolation from Olive Pollen

Olive (Olea europaea) pollen PMPs are isolated as previously described in Prado et al., Molecular Plant. 7(3):573-577, 2014. Briefly, olive pollen (0.1 g) is hydrated in a humid chamber at room temperature for 30 min before transferring to petri dishes (15 cm in diameter) containing 20 ml germination medium: 10% sucrose, 0.03% Ca(NO₃)₂, 0.01% KNO₃, 0.02% MgSO₄, and 0.03% H₃B₃. Pollen is germinated at 30° C. in the dark for 16 h. Pollen grains are considered germinated only when the tube is longer than the diameter of the pollen grain. Cultured medium containing PMPs is collected and cleared of pollen debris by two successive filtrations on 0.85 um filters by centrifugation. PMPs are purified as described in Example 2.

d) PMP Isolation from Arabidopsis Phloem Sap

Arabidopsis (Arabidopsis thaliana Col-0) seeds are surface sterilized with 50% bleach and plated on 0.53 Murashige and Skoog medium containing 0.8% agar. The seeds are vernalized for 2 d at 4° C. before being moved to short-day conditions (9-h days, 22° C., 150 μEm⁻²). After 1 week, the seedlings are transferred to Pro-Mix PGX. Plants are grown for 4-6 weeks before harvest.

Phloem sap from 4-6-week old Arabidopsis rosette leaves is collected as described by Tetyuk et al., JoVE. 80, 2013. Briefly, leaves are cut at the base of the petiole, stacked, and placed in a reaction tube containing 20 mM K2-EDTA for one hour in the dark to prevent sealing of the wound. Leaves are gently removed from the container, washed thoroughly with distilled water to remove all EDTA, put in a clean tube, and phloem sap is collected for 5-8 hours in the dark. Leaves are discarded, phloem sap is filtered through a 0.85 pm filter to remove large particles, and PMPs are purified as described in Example 2.

h) PMP Isolation from Tomato Plant Xylem Sap

Tomato (Solanum lycopersicum) seeds are planted in a single pot in an organic-rich soil, such as Sunshine Mix (Sun Gro Horticulture, Agawam, Mass.) and maintained in a greenhouse between 22° C. and 28° C. About two weeks after germination, at the two true-leaf stage, the seedlings are transplanted individually into pots (10 cm diameter and 17 cm deep) filled with sterile sandy soil containing 90% sand and 10% organic mix. Plants are maintained in a greenhouse at 22-28° C. for four weeks.

Xylem sap from 4-week old tomato plants is collected as described by Kohlen et al., Plant Physiology. 155(2):721-734, 2011. Briefly, tomato plants are decapitated above the hypocotyl, and a plastic ring is placed around the stem. The accumulating xylem sap is collected for 90 min after decapitation. Xylem sap is filtered through a 0.85 pm filter to remove large particles, and PMPs are purified as described in Example 2.

i) PMP Isolation from Tobacco BY-2 Cell Culture Medium

Tobacco BY-2 (Nicotiana tabacum L cv. Bright Yellow 2) cells are cultured in the dark at 26° C., on a shaker at 180 rpm in MS (Murashige and Skoog, 1962) BY-2 cultivation medium (pH 5.8) comprised MS salts (Duchefa, Haarlem, Netherlands, at #M0221) supplemented with 30 g/L sucrose, 2.0 mg/L potassium dihydrogen phosphate, 0.1 g/L myo-inositol, 0.2 mg/L 2,4-dichlorophenoxyacetic acid, and 1 mg/L thiamine HCl. The BY-2 cells are subcultured weekly by transferring 5% (v/v) of a 7-day-old cell culture into 100 mL fresh liquid medium. After 72-96 hours, BY-2 cultured medium is collected and centrifuged at 300 g at 4° C. for 10 minutes to remove cells. The supernatant containing PMPs is collected and cleared of debris by filtration on 0.85 um filter. PMPs are purified as described in Example 2.

### Example 2: Production of Purified Plant Messenger Packs (PMPs)

This example demonstrates the production of purified PMPs from crude PMP fractions as described in Example 1, using ultrafiltration combined with size-exclusion chromatography, a density gradient (iodixanol or sucrose), and the removal of aggregates by precipitation or size-exclusion chromatography.

### Experimental Design:

a) Production of Purified Grapefruit PMPs Using Ultrafiltration Combined with Size-Exclusion Chromatography

The crude grapefruit PMP fraction from Example 1a is concentrated using 100-kDA molecular weight cut-off (MWCO) Amicon spin filter (Merck Millipore). Subsequently, the concentrated crude PMP solution is loaded onto a PURE-EV size exclusion chromatography column (HansaBioMed Life Sciences Ltd) and isolated according to the manufacturer's instructions. The purified PMP-containing fractions are pooled after elution. Optionally, PMPs can be further concentrated using a 100-kDa MWCO Amicon spin filter, or by Tangential Flow Filtration (TFF). The purified PMPs are analyzed as described in Example 3.

b) Production of Purified Arabidopsis Apoplast PMPs Using an Iodixanol Gradient

Crude Arabidopsis leaf apoplast PMPs are isolated as described in Example 1a, and PMPs are purified by using an iodixanol gradient as described in Rutter and Innes, Plant Physiol. 173(1): 728-741, 2017. To prepare discontinuous iodixanol gradients (OptiPrep; Sigma-Aldrich), solutions of 40% (v/v), 20% (v/v), 10% (v/v), and 5% (v/v) iodixanol are created by diluting an aqueous 60% OptiPrep stock solution in vesicle isolation buffer (VIB; 20 mM MES, 2 mM CaCl₂), and 0.1 M NaCl, pH6). The gradient is formed by layering 3 ml of 40% solution, 3 mL of 20% solution, 3 mL of 10% solution, and 2 mL of 5% solution. The crude apoplast PMP solution from Example 1a is centrifuged at 40,000 g for 60 min at 4° C. The pellet is resuspended in 0.5 ml of VIB and layered on top of the gradient. Centrifugation is performed at 100,000 g for 17 h at 4° C. The first 4.5 ml at the top of the gradient is discarded, and subsequently 3 volumes of 0.7 ml that contain the apoplast PMPs are collected, brought up to 3.5 mL with VIB and centrifuged at 100,000 g for 60 min at 4° C. The pellets are washed with 3.5 ml of VIB and repelleted using the same centrifugation conditions. The purified PMP pellets are combined for subsequent analysis, as described in Example 3.

c) Production of Purified Grapefruit PMPs Using a Sucrose Gradient

Crude grapefruit juice PMPs are isolated as described in Example 1d, centrifuged at 150,000 g for 90 min, and the PMP-containing pellet is resuspended in 1 ml PBS as described (Mu et al., Molecular Nutrition & Food Research. 58(7):1561-1573, 20141. The resuspended pellet is transferred to a sucrose step gradient (8%/15%/30%/45%/60%) and centrifuged at 150,000 g for 120 min to produce purified PMPs. Purified grapefruit PMPs are harvested from the 30%/45% interface, and subsequently analyzed, as described in Example 3.

d) Removal of Aggregates from Grapefruit PMPs

In order to remove protein aggregates from produced grapefruit PMPs as described in Example 1d or purified PMPs from Example 2a-c, an additional purification step can be included. The produced PMP solution is taken through a range of pHs to precipitate protein aggregates in solution. The pH is adjusted to 3, 5, 7, 9, or 11 with the addition of sodium hydroxide or hydrochloric acid. pH is measured using a calibrated pH probe. Once the solution is at the specified pH, it is filtered to remove particulates. Alternatively, the isolated PMP solution can be flocculated using the addition of charged polymers, such as Polymin-P or Praestol 2640. Briefly, 2-5 g per L of Polymin-P or Praestol 2640 is added to the solution and mixed with an impeller. The solution is then filtered to remove particulates. Alternatively, aggregates are solubilized by increasing salt concentration. NaCl is added to the PMP solution until it is at 1 mol/L. The solution is then filtered to purify the PMPs. Alternatively, aggregates are solubilized by increasing the temperature. The isolated PMP mixture is heated under mixing until it has reached a uniform temperature of 50° C. for 5 minutes. The PMP mixture is then filtered to isolate the PMPs. Alternatively, soluble contaminants from PMP solutions are separated by size-exclusion chromatography column according to standard procedures, where PMPs elute in the first fractions, whereas proteins and ribonucleoproteins and some lipoproteins are eluted later. The efficiency of protein aggregate removal is determined by measuring and comparing the protein concentration before and after removal of protein aggregates via BCA/Bradford protein quantification. The produced PMPs are analyzed as described in Example 3

### Example 3: Plant Messenger Pack Characterization

This example demonstrates the characterization of PMPs produced as described in Example 1 or Example 2.

### Experimental Design:

a) Determining PMP Concentration

PMP particle concentration is determined by Nanoparticle Tracking Analysis (NTA) using a Malvern NanoSight, or by Tunable Resistive Pulse Sensing (TRPS) using an iZon qNano, following the manufacturer's instructions. The protein concentration of purified PMPs is determined by using the DC Protein assay (Bio-Rad). The lipid concentration of purified PMPs is determined using a fluorescent lipophilic dye, such as DiOC6 (ICN Biomedicals) as described by Rutter and Innes, Plant Physiol. 173(1): 728-741, 2017. Briefly, purified PMP pellets from Example 2 are resuspended in 100 ml of 10 mM DiOC6 (ICN Biomedicals) diluted with MES buffer (20 mM MES, pH 6) plus 1% plant protease inhibitor cocktail (Sigma-Aldrich) and 2 mM 2,29-dipyridyl disulfide. The resuspended PMPs are incubated at 37° C. for 10 min, washed with 3 mL of MES buffer, repelleted (40,000 g, 60 min, at 4° C.), and resuspended in fresh MES buffer. DiOC6 fluorescence intensity is measured at 485 nm excitation and 535 nm emission.

b) Biophysical and Molecular Characterization of PMPs

PMPs are characterized by electron and cryo-electron microscopy on a JEOL 1010 transmission electron microscope, following the protocol from Wu et al., Analyst. 140(2):386-406, 2015. The size and zeta potential of the PMPs are also measured using a Malvern Zetasizer or iZon qNano, following the manufacturer's instructions. Lipids are isolated from PMPs using chloroform extraction and characterized with LC-MS/MS as demonstrated in Xiao et al. Plant Cell. 22(10): 3193-3205, 2010. Glycosyl inositol phosphorylceramides (GIPCs) lipids are extracted and purified as described by Cacas et al., Plant Physiology. 170: 367-384, 2016, and analyzed by LC-MS/MS as described above. Total RNA, DNA, and protein are characterized using Quant-It kits from Thermo Fisher according to instructions. Proteins on the PMPs are characterized by LC-MS/MS following the protocol in Rutter and Innes, Plant Physiol. 173(1): 728-741, 2017. RNA and DNA are extracted using Trizol, prepared into libraries with the TruSeq Total RNA with Ribo-Zero Plant kit and the Nextera Mate Pair Library Prep Kit from Illumina, and sequenced on an Illumina MiSeq following manufacturer's instructions.

### Example 4: Characterization of Plant Messenger Pack Stability

This example demonstrates measuring the stability of PMPs under a wide variety of storage and physiological conditions.

### Experimental Design:

PMPs produced as described in Examples 1 and 2 are subjected to various conditions. PMPs are suspended in water, 5% sucrose, or PBS and left for 1, 7, 30, and 180 days at −20° C., 4° C., 20° C., and 37° C. PMPs are also suspended in water and dried using a rotary evaporator system and left for 1, 7, and 30, and 180 days at 4° C., 20° C., and 37° C. PMPs are also suspended in water or 5% sucrose solution, flash-frozen in liquid nitrogen and lyophilized. After 1, 7, 30, and 180 days, dried and lyophilized PMPs are then resuspended in water. The previous three experiments with conditions at temperatures above 0° C. are also exposed to an artificial sunlight simulator in order to determine content stability in simulated outdoor UV conditions. PMPs are also subjected to temperatures of 37° C., 40° C., 45° C., 50° C., and 55° C. for 1, 6, and 24 hours in buffered solutions with a pH of 1, 3, 5, 7, and 9 with or without the addition of 1 unit of trypsin or in other simulated gastric fluids.

After each of these treatments, PMPs are bought back to 20° C., neutralized to pH 7.4, and characterized using some or all of the methods described in Example 3.

### Example 5: Treatment of a Fungus with Plant Messenger Packs

This example demonstrates the ability of PMPs produced from Arabidopsis thaliana rosettes to decrease fitness of a pathogenic fungus. In this example, the yeast Saccharomyces cerevisiae as a model pathogenic fungus.

Pathogenic fungi like Candida species represent the main cause of opportunistic fungal infections worldwide, Saccharomyces cerevisiae (also known as “baker's yeast”) is mostly considered to be an occasional digestive commensal. However, since the 1990s, there have been a growing number of reports about its implication as an etiologic agent of invasive infection. Infections with pathogenic fungi are typically associated with high morbidity and mortality, mainly due to the limited efficacy of current antifungal drugs.

Therapeutic Design:

The Arabidopsis apoplast PMP solution was formulated with 0 (negative control), 1, 10, or 50, 100 and 250 μg PMP protein/ml from Example 1a, in 10 ml of PBS.

Experimental Design:

a) Labeling Apoplast PMPs with a Lipophilic Membrane Dye

Arabidopsis thaliana apoplast PMPs are isolated and purified as described in Examples 1-2, and are labeled with PKH26 (Sigma), according to the manufacturer's protocol, with some modifications. Briefly, 50 mg apoplast PMPs in 1 mL dilute C or the PKH26 kit are mixed with 2 ml of 1 mM PKH26 and incubated at 37° C. for 5 min. Labelling is stopped by adding 1 mL of 1% BSA. All unlabeled dye is washed away by centrifugation at 150,000 g for 90 min, and labelled PMP pellets are resuspended in sterile water.

b) Apoplast PMP Uptake by Saccharomyces cerevisiae

Saccharomyces cerevisiae is obtainedfrom the ATCC (#9763) and maintained at 30° C. in yeast extract peptone dextrose broth (YPD) as indicated by the manufacturer. To determine the PMP uptake by S. cerevisiae, yeast cells are grown to an OD₆₀₀ of 0.4-0.6 in selection media, and incubated with 0 (negative control), 1, 10, 50, 100, or 250 pg/ml of PKH26-labeled apoplast-derived PMPs directly on glass slides. In addition to a PBS control, S. cerevisiae cells are incubated in the presence of PKH26 dye (final concentration 5 pg/ml). After incubation of 5 min, 30 min and 1 h at room temperature, images are acquired on a high-resolution fluorescence microscope. Apoplast-derived PMPs are taken up by yeast cells when red PMPs are observed in the cytoplasm or if the cytoplasm of the yeast cell turns red, versus exclusive staining of the cell membrane by PKH26 dye. To assess PMP uptake, the percentage of yeast cells with a red cytoplasm/red PMPs in the cytoplasm, versus membrane only staining are compared between PMP-treated cells and the PBS and PKH26 dye only controls.

c) Treatment of S. cerevisiae with an Arabidopsis Apoplast PMP Solution In Vitro

To determine the effect of Arabidopsis apoplast PMP treatment on the fitness of yeast cells, a modified drug susceptibility test is performed. S. cerevisiae cells (10⁵ cells/ml) are mixed with molten YPD agar (approximately 40° C.) and poured in a petri dish. After agar solidification, 5 μl of 0 (PBS, negative control), 1, 10, or 50, 100 and 250 pg PMP protein/ml solutions are spotted onto the plate. The plates are incubated at 30° C., and zones of inhibition (dark circles) are scored after 2 and 3 days.

Additionally, a spot test is performed to assess the effect of PMPs on yeast growth. S. cerevisiae cells are grown overnight on YPD medium. The cells are then suspended in normal saline to an OD600 of 0.1 (A₆₀₀). Five microliters of fivefold serial dilutions of each yeast culture are spotted onto YPD plates in the absence (PBS control) and presence of 1, 10, 50, 100, or 250 pg PMP protein/ml. Growth differences are recorded following incubation of the plates for 48 h at 30° C.

The overall effect of Arabidopsis apoplast PMPs on fungal fitness is determined by comparing the inhibition zones and growth differences between the PBS control and PMP-treated fungal cells.

### Example 6: Treatment of a Bacterium with Plant Messenger Packs

This example demonstrates the ability of purified apoplast PMPs from Arabidopsis thaliana rosettes to be uptaken by bacteria, and to decrease the fitness of the pathogenic bacterium Escherichia coli. In this example, E. coli is used as a model bacterial pathogen.

Human and animal diseases triggered by bacterial pathogens, like Staphylococcus aureus, Salmonella, and E. coli, cause significant morbidity and mortality, due to the limited efficacy and increasing resistance to current antimicrobial drugs.

Therapeutic Design:

The Arabidopsis apoplast PMP solution is formulated with 0 (negative control), 1, 10, 50, 100, or 250 μg PMP protein/ml in 10 ml sterile water.

a) Labeling Apoplast PMPs with a Lipophilic Membrane Dye

Arabidopsis thaliana apoplast PMPs are PMPs produced from as described in Examples 1-2, and are labeled with PKH26 (Sigma) according to the manufacturer's protocol with some modifications. Briefly, 50 mg PMPs are diluted in 1 mL dilute C, and are mixed with 2 ml of 1 mM PKH26 and incubated at 37° C. for 5 min. Labelling is stopped by adding 1 mL 1% BSA. All unlabeled dye is washed away by centrifugation at 150,000 g for 90 min, and labelled PMP pellets are resuspended in sterile water, and analyzed as described in Example 3.

b) Apoplast PMP Uptake by E. coli

E. coli are acquired from ATCC (#25922) and grown on Trypticase Soy Agar/broth at 37° C. according to the manufacturer's instructions. To determine the PMP uptake by E. coli, 10 ul of a 1 ml overnight bacterial suspension is incubated with 0 (negative control), 1, 10, 50, 100, or 250 μg/ml of PKH26-labeled apoplast PMPs directly on a glass slides. In addition to a water control, E. coli bacteria are incubated in the presence of PKH26 dye (final concentration 5 μg/ml). After incubation of 5 min, 30 min, and 1 h at room temperature, images are acquired on a high-resolution fluorescence microscope. Apoplast PMPs are taken up by bacteria when the cytoplasm of the bacteria turns red versus exclusive staining of the cell membrane by PKH26 dye. The percentage of PKH26-PMP treated bacteria with a red cytoplasm compared to control treatments with PBS and PKH26 dye only are recorded to determine PMP uptake.

c) Treatment of E. coli with an Arabidopsis Apoplast PMP Solution In Vitro

The ability of Arabidopsis apoplast PMPs to affect the growth of E. coli is determined using a modified standard disk diffusion susceptibility method. Briefly, an E. coli inoculum suspension is prepared by selecting several morphologically similar colonies from an overnight growth (16-24 h of incubation) on a non-selective medium with a sterile loop or a cotton swab and suspending the colonies in sterile saline (0.85% NaCl w/v in water) to the density of a McFarland 0.5 standard, approximately corresponding to 1-2×10⁸ CFU/ml. Mueller-Hinton agar plates (150 mm diameter) are inoculated with the E. coli suspension, by dipping a sterile cotton swab into the inoculum suspension, removing the excess fluid from the swab, and spreading bacteria evenly over the entire surface of the agar plate by swabbing in three directions. Next, 3 uL of water (negative control), 1, 10, 50, 100, or 250 μg PMP protein/ml are spotted onto the plate and allowed to dry. The plates are incubated for 16-18 hours at 35° C., photographed, and scanned. The diameter of the lytic zone (area without bacteria) around the spotted area is measured. Control (water) and PMP treated lytic zones are compared to determine the bactericidal effect of Arabidopsis apoplast PMPs.

### Example 7: Treatment of a Parasitic Insect with PMPs

This example demonstrates the ability to kill or decrease the fitness of a parasitic insect, such as bed bugs, by treating them with a solution of PMPs produced from a plant, such as ginger roots. In this example, bed bugs are used as a model organism for parasitic insects.

Bed bugs (Cimex lectularius) are hematophagous ectoparasites that are an important emerging public health pest worldwide. The unavailability of effective residual insecticides and greater resistance to pyrethroid insecticides in bed bug populations warrants the development of effective and environmentally safe treatment options.

### Therapeutic Design:

The ginger root PMP solution is formulated with 0 (negative control), 1, 10, 50, 100, and 250 μg PMP protein/ml in 10 ml of PBS.

### Experimental Design:

a) Cultivation of Bed Bugs (Cimex lectularius)

Cimex lectularius are obtained from Sierra Research Laboratories (Modesto, Calif.). Bed bug colonies are maintained in glass enclosures containing cardboard harborages and kept on a 12:12 photoperiod at 25° C. and 40-45% (ambient) humidity. Colonies are blood-fed once per week with a parafilm-membrane feeder containing defibrinated rabbit blood (Hemostat Laboratories, Dixon, Calif.).

b) Treatment of Cimex lectularius with a Ginger Root PMP Solution

Ginger root PMPs are isolated as described in Example 1, and the effect of PMP treatment on bed bug survival, fecundity, and development are determined. Prior to treatment, 0-2 week old bed bug adults which have not blood-fed for four days are isolated, and placed in glass jars to allow mating for two days. Males are sorted out, and female bed bugs are separated into experimental cohorts of 10-15 insects which are housed together. Female bed bugs are treated by allowing them to feed on defibrinated rabbit blood spiked with a final concentration 0 (PBS, negative control), 1, 10, 50, 100, or 250 μg PMP protein/ml for 15 min until fully engorged. After PMP treatment, cohorts of 10-15 bed bugs are maintained at 25° C. and 40-45% (ambient) humidity in a petri dish containing a sterile pad, which provides a suitable substrate for oviposition (Advantec MFS, Inc., Dublin, Calif.). For survival assays, dead insects are counted, recorded, and removed from their enclosure each day for 10 days, and the mean percent survival of PMP treated bed bugs is calculated compared to PBS controls.

Thereafter, bed bugs are fed every 10 days with PMP-spiked blood as indicated above, and transferred to a new petri dish. Petri dishes with eggs are kept inside a growth chamber for 2 wks to allow sufficient hatching time. The eggs laid are observed under a stereomicroscope with a 16× magnification, and the average number of eggs laid by female bed bugs per feeding interval is calculated for 30 d, the average number of nymphs that emerge from the eggs are assessed, and the mean percent survival of bed bugs is calculated. The effect of ginger root PMPs on bed bug survival, fecundity, and development are determined by comparing the ginger root PMP-treated cohorts to the PBS-treated control cohorts.

### Example 8: Treatment of a Parasitic Nematode with PMPs

This example demonstrates the ability to kill or decrease the fitness of a parasitic nematode, such as Heligmosomoides polygyrus, by treating them with a solution of PMPs produced from a plant, such as ginger roots.

Chronic helminth infections remain a huge global health problem, causing extensive morbidity in both humans and livestock. Many of the most prevalent helminth parasites are difficult to study in the laboratory, as they have co-evolved with, and are closely adapted to, their definitive host species. In this example, we use the model pathogenic helminth H. polygyrus, a natural mouse parasite, to show the effect of ginger root PMPs on its fitness.

### Therapeutic Design:

The ginger root PMP solution is formulated with 0 (negative control), 1, 10, 50, 100, or 250 μg PMP protein/ml from Example 1a in 10 ml of sterile water.

### Experimental Design:

a) Cultivation of Parasitic Nematode Heligmosomoides Polygyrus

Cultivation of H. polygyrus is performed as described Keiser et al., Parasites & Vectors. 9(1):376, 2016. Four week-old female NMRI mice and H. polygyrus L3 are purchased from a local supplier. Female NMRI mice are infected with 80 H. polygyrus L3 nematodes per os. H. polygyrus eggs are obtained from infected feces.

b) Treatment of H. polygyrus Eggs with a Ginger Root PMP Solution In Vitro

To assess the nematocidal activity of the ginger root PMP solution on egg hatching, H. polygyrus eggs are obtained from infected mouse feces, cleaned and soaked in a solution containing 0 (negative control), 1, 10, 50, 100, or 250 μg PMP protein/ml ginger root PMPs for 30 min, 1 hour, or 2 hours. Next, eggs are placed on agar for 14 days in the dark at 24° C., and from 6 days the number of hatched L3 larvae are recorded. The effect of ginger root PMPs on egg hatching is determined by comparing the percentage of hatched H. polygyrus eggs with and without PMP treatment.

c) Treatment of H. polygyrus L3 Larvae with a Ginger Root PMP Solution In Vitro

To assess the nematocidal activity of the PMP solution on H. polygyrus L3 larvae, H. polygyrus eggs are obtained from infected feces, placed on agar and, after 9 days in the dark at 24° C., the L3 larvae hatch. For PMP treatment, 40 L3 larvae are placed in each well of a 96-well plate. Worms are incubated in the presence of 100 μl RPMI 1640 medium, supplemented with 0.63 μg/ml amphotericin B, 500 U/ml penicillin, 500 μg/ml streptomycin, and 0 (negative control), 1, 10, 50, 100, or 250 μg PMP protein/ml. Each treatment is tested in duplicate. Worms incubated with 100 pM levamisole (Sigma-Aldrich) serve as a positive control. The plates are kept at room temperature for up to 72 h. To assess the effect of the PMP treatment on L3 fitness, the total number of L3 larvae per well is counted, and the moving larvae after stimulation with 100 μl hot water 80° C.) is recorded. The relative percentage of moving L3 larvae between PMP-treatment and the positive and negative controls are compared to determine the larval nematocidal effect of ginger root PMPs.

d) Treatment of H. polygyrus Adults with a Ginger Root PMP Solution In Vitro

Female NMRI mice are infected with 80 H. polygyrus L3 per os. Two weeks post-infection, mice are dissected and three adult worms are placed in each well of a 24-well plate. Worms are incubated with culture medium and 0 (negative control), 1, 10, 50, 100, or 250 μg ginger root PMP protein/ml. Each treatment is tested in triplicate. Adult worms incubated with medium only and 50 μM levamisole serve as negative and positive control, respectively. Worms are kept in an incubator at 37° C. and 5% CO₂ for 72 h and, subsequently, are microscopically evaluated using a viability scale from 3 (active) to 0 (not moving). The average viability scores of H. polygyrus adults between PMP-treated and the positive and negative controls are compared to determine the adult nematocidal effect of ginger root PMPs.

e) Treatment of H. polygyrus In Vivo with a Ginger Root PMP Solution in Mouse

To test the nematocidal in vivo effect of ginger root PMP treatment, NMRI mice are infected with 80 H. polygyrus L3 per os. Fourteen days post-infection, mice are treated orally with the test drugs at dosages of 10, 100, 300, or 400 mg PMP protein/kg or a levamisole control. Four to six untreated mice serve as controls. Ten days posttreatment, animals are killed by the CO2 method, and the gastrointestinal tract is collected. The intestine is dissected, and adult worms are collected and counted. The nematocidal activity of orally administered ginger root PMPs is determined by comparing the average number of adult worms in PMP-treated versus negative and positive control treated mice cohorts.

### Example 9: Treatment of a Parasitic Protozoan with PMPs

This example demonstrates the ability to kill or decrease the fitness of a parasitic protozoan, such as Trichomonas vaginalis, by treatment with a solution of PMPs produced from a plant, such as ginger roots. In this example, T. vaginalis is used as a model parasitic protozoan.

Trichomonas vaginalis is one of the most common non-viral sexually transmitted diseases (STD) worldwide. This anaerobic protozoan, motile by means of anterior flagella and an undulating membrane, infects an estimated 180 million women worldwide with conservative estimates indicating that 6 million are infected annually in the United States. In view of increased resistance of the parasite to classical drugs of the metronidazole family, the need for new unrelated agents is increasing.

Therapeutic Design:

The ginger root PMP solution is formulated with 0 (negative control), 1, 10, 50, 100, or 250 μg PMP protein/ml in 10 ml of sterile water

Experimental Design:

a) Cultivation of Parasitic Protozoan T. vaginalis

Trichomonas vaginalis is obtained from the ATCC (#50167) and cultured according to the manufacturer's instruction, and as described by Tiwarti et al., Journal of Antimicrobial Chemotherapy, 62(3): 526-534, 2008. Protozoans are grown in standard TYI-S33 medium (pH 6.8) supplemented with 10% FCS, vitamin mixture and 100 U/mL penicillin/streptomycin at 37° C. in 15 mL screw-stoppered glass tubes. The cultures routinely attain a concentration of 2×10⁷ cells/mL in 48 h. An inoculum of 1×10⁴ cells per tube is used for maintenance of the culture.

b) Treatment of T. vaginalis with a Ginger Root PMP Solution

Ginger root PMPs are produced as described in Example 1. To determine the effect of ginger root PMPs on T. vaginalis fitness, a drug susceptibility assay is performed as previously described (Tiwarti et al., Journal of Antimicrobial Chemotherapy, 62(3): 526-534, 2008). Briefly, 5×10³ Trichomonas trophozoites per mL are incubated in the presence 0 (sterile water, negative control), 1, 10, or 50, 100 and 250 μg PMP protein/ml or 1-12 mM Metronidazole (Sigma-Aldrich), as positive control, in the TYI-S33 culture medium in 24-well culture plates at 37° C. Cells are checked for viability at different time intervals from 3 h to 48 h under the microscope at a 20× magnification. The viability of T. vaginalis cells is determined by Trypan Blue exclusion assay. Cells are counted using a haemocytometer. The minimum concentration of the PMP solution at which all cells are found dead is considered as its Minimal Inhibitory Concentration (MIC). The experiment is repeated three times to confirm the MIC. The effect of ginger root PMPs on T. vaginalis fitness is determined by comparing the mean MIC of PMP-treated versus negative and positive controls.

### Example 10: Treatment of a Fungus with Short Nucleic Acid-Loaded Plant Messenger Packs

This example demonstrates the ability of PMPs to deliver short nucleic acid, by isolating PMP lipids and synthesizing them into vesicles containing short nucleic acids. In this example, short double-stranded RNAs (dsRNA)-loaded PMPs are used to knock down a virulence factor in a pathogenic fungus, Candida albicans. It also demonstrates that short nucleic-acid loaded-PMPs are stable and retain their activity over a range of processing and environmental conditions. In this example, dsRNA is used as a model nucleic acid, and Candida albicans is used as a model pathogenic fungus.

Candida species represent the main cause of opportunistic fungal infections worldwide, and Candida albicans remains the most common etiological agent of candidiasis, now the third to fourth most common nosocomial infection. These infections are typically associated with high morbidity and mortality, mainly due to the limited efficacy of current antifungal drugs. In C. albicans morphogenetic conversions between yeast and filamentous forms and biofilm formation represent two important biological processes that are intimately associated with the biology of this fungus, and also play important roles during the pathogenesis of candidiasis.

Therapeutic Dose:

PMPs loaded with dsRNA, formulated in water to a concentration that delivers an equivalent of an effective siRNA dose of 0, 50, 500, or 1000 nM in sterile water.

Experimental Protocol:

a) Synthesis of EFG1 dsRNA-Loaded Grapefruit PMPs from Isolated Grapefruit PMP Lipids

Short nucleic acids are loaded in PMPs according to a modified protocol from Wang et al, Nature Comm., 4:1867, 2013. Briefly, purified PMPs are produced from grapefruit according to Example 1-2, and grapefruit PMP lipids are isolated, adapted from Xiao et al. Plant Cell. 22(10): 3193-3205, 2010. Briefly, 3.75 ml 2:1 (v/v) MeOH:CHCl3 is added to 1 ml of PMPs in PBS and vortexed. CHCl3 (1.25 ml) and ddH2O (1.25 ml) are added sequentially and vortexed. The mixture is then centrifuged at 2,000 r.p.m. for 10 min at 22° C. in glass tubes to separate the mixture into two phases (aqueous phase and organic phase). For collection of the organic phase, a glass pipette is inserted through the aqueous phase with gentle positive pressure, and the bottom phase (organic phase) is aspirated and dispensed into fresh glass tubes. The organic phase samples are aliquoted and dried by heating under nitrogen (2 psi).

Short Double stranded RNA (dsRNA) targeting Candida albicans EFG1 siRNA with sequences antisense: 5′ACAUUGAGCAAUUUGGUUC-3′ and sense: 5′-GAACCAAAUUGCUCAAUGU-3′, and a scrambled siRNA control 5′-AUAUGCGCAACAUUGACA-3′ as specified in Moazeni et al., Mycopathologia. 174(3):177-185, 2012, are obtained from IDT. Sense/antisense annealing is performed in annealing buffer (30 mM HEPES-KOH pH 7.4, 100 mM KCl, 2 mM MgCl2, and 50 mM NH4 Ac as described (Moazeni et al., Mycopathologia. 174(3):177-185, 20121 to generate siRNA duplex (dsRNA). dsRNA loaded-PMPs are synthesized from both targeted and control siRNA, by mixing the lipids and short nucleic acids, which are dried to form a thin film. The film is dispersed in PBS and sonicated to form loaded liposomal formulations. PMPs are purified using a sucrose gradient as described in Example 2 and washed via ultracentrifugation before use to remove unbound nucleic acid. A small portion of both samples are characterized using the methods in Example 3, RNA content is measured using the Quant-It RiboGreen RNA assay kit, and their stability is tested as described in Example 4.

To determine the efficiency of fungal blockade using siRNA-loaded PMPs from Exampe 10a, Candida albicans fungi are treated with a PMP solution with an effective siRNA dose of 0, 50, 500 and 1000 nM in sterile water. C. albicans wild-type strain (ATCC #14053) is cultured on yeast extract peptone/dextrose (YPD) medium plates, incubated at 37° C. for 24 h, and maintained at 4° C. until use. The effect and efficiency of treatment with EFG1 dsRNA-loaded PMPs are compared to scrambled and negative controls.

b) Treatment of Candida albicans with EFG1 siRNA-Loaded Grapefruit PMPs for Reducing Fungal Biofilm

To measure the effect of siRNA-loaded PMPs on C. albicans biofilm formation, an overnight culture of C. albicans is grown by inoculating in 20 mL of yeast peptone dextrose (YPD) (1% [wt/vol] yeast extract, 2% [wt/vol] peptone, 2% [wt/vol] dextrose) liquid media in 150 mL flasks and incubating in an orbital shaker (150-180 rpm) at 30° C. Under these conditions, C. albicans grow as budding-yeast. Biofilms are formed using the 96-well microtiter plate model as described by Pierce et al., Pathog Dis. April; 70(3): 423-431, 2014. Briefly, cells are harvested from overnight YPD cultures and after washings they were resuspended in RPMI-1640 supplemented with L-glutamine (Cellgro) and buffered with 165 mM morpholinepropanesulfonic acid (MOPS) at a final concentration of 1.0×106 cells/mL. C. albicans biofilms are formed on commercially available pre-sterilized, polystyrene, flat-bottom, 96-well microtiter plates (Corning Incorporated, Corning, N.Y.). Per well, 250 ul of the 1.0×10⁶ cells/mL C. albicans cells are dispensed, and EFGR1 siRNA-loaded PMPs or a scrabbled control were added to a final concentration of 0 (water, negative control), 50, 500, or 1000 nM. Treatments are done in triplicate and plates are incubated at 37° C. for 24 h. Following biofilm formation, the wells are washed twice to remove non-adherent cells, visualized by light microscopy and processed using semi-quantitative colorimetric assay based on the reduction of 2,3-bis(2-methoxy-4-nitro-5-sulfo-phenyl)-2H-tetra-zolium-5-carboxanilide (XTT, Sigma). The OD of control biofilms formed (in the absence of PMPs) was arbitrarily set at 100% and the inhibitory effects of siRNA-loaded PMPs were determined by the percent reduction in absorbance in relation to the controls. Data is calculated as percent biofilm inhibition relative to the average of the control wells.

To quantify changes in EFGR1 expression, the level of EFG1 mRNA in C. albicans is measured by quantitative real-time RT-PCR. Total RNA is extracted using the Fisher BioReagents™ SurePrep™ Plant/Fungi Total RNA Purification Kit (Fisher scientific, Waltham, Mass.), cDNA synthesis using SuperScript III Reverse Transcriptase (Invitrogen Carlsbad, Calif.), and quantitative RT-PCR quantification. The expression of EFG1 (XM_709104.1) and housekeeping gene beta actin ACT1 (XM_717232.1) are determined in C. albicans after treatment of synthesized EFGR1-dsRNA and scrambled control is measured using the following primers: EFG1-Fw:TGCCAATAATGTGTCGGTTG, EFG1-Rev: CCCATCTCTTCTACCACGTGTC, ACT1-Fw: ACGGTATTGTTTCCAACTGGGACG, ACT1-Rev:TGGAGCTTCGGTCAACAAAACTGG (Moazeni et al., Mycopathologia. 174(3):177-185, 20121. RT-qPCR is performed using SsoAdvanced™ Universal SYBR® Green Supermix (BioRad) with three technical replicates according to the following protocol: denaturation at 95° C. for 3 min, 40 repeats of 95° C. for 20 s, 61° C. for 20 s and 72° C. for 15 s.

The abundance of EFG1 is normalized to the ACT1 abundance of the plant derived PCR product to determine the knock down efficiency is determined by calculating the ΔΔCt value, comparing the normalized fungal growth in the negative PBS control to the normalized fungal growth in the ds-RNA loaded PMP treatment samples.

c) Treatment of Candida albicans with EFG1 siRNA-Loaded Grapefruit PMPs for Reducing Fungal Fitness

To assess the effect of EFG1 siRNA-loaded PMPs on fungal growth, a PMP activity assay using yeast embedded in agar was performed, as described by Beaumont et al., Cell Death and Disease. 4(5): e619, 2013. Overnight cultures of transformants in minimal media containing glucose (2%, w/v) are washed twice in 10 mM Tris-HCl (pH 8.0), 1 mM EDTA (TE) then resuspended in TE. OD600 is measured and used to introduce 5×10⁷ colony-forming units of yeast into 7.5 ml of minimal media containing galactose, which is equilibrated to 37° C. Each yeast suspension is mixed with 7.5 ml of minimal media agar containing galactose (2%, w/v) that is pre-equilibrated to 50° C., quickly mixed by inversion, then poured onto previously made 10 cm plates containing 15 ml of galactose-containing minimal media agar. The plates are set at room temperature for an hour. Five microliters of EFGR1 siRNA-loaded PMPs or a scrabbled control with a concentration of 0 (water, negative control), 50, 500, or 1000 nM are pipetted onto plates containing embedded yeast, allowed to dry at room temperature, incubated at 30° C. for 3 days, then photographed. Dark circles reveal PMP-mediated suppression of yeast growth.

### Example 11: Treatment of an Insect with Peptide Nucleic Acid-Loaded PMPs

This example demonstrates loading of PMPs with a peptide nucleic acid construct for the purpose of reducing insect fitness by knocking down vATPase-E in bed bugs (Cimex lectularius), which has been demonstrated by siRNA to affect survival and reproduction (Basnet and Kamble, Journal of Medical Entomology, 55(3): 540-546. 2018). This example also demonstrates that PNA-loaded PMPs are stable and retain their activity over a range of processing and environmental conditions. In this example, PNA is used as a model protein, and Cimex lectularius is used as a model pathogenic insect.

Therapeutic Dose:

PMPs loaded with PNA, formulated in water to a concentration that delivers an equivalent of an effective PNA dose of 0, 0.1, 1, 5, or 10 pM in sterile water

Experimental Protocol:

a) Loading of Grapefruit PMPs with a Peptide Nucleic Acid

PNAs against Cimex lectularius vATPase-E (NCBI GenBank accession #LOCI 06667865) are designed and synthesized by an appropriate vendor. PMPs from grapefruit are isolated according to Example 1. PMPs are placed in solution with the PNA in PBS. The solution is then sonicated to induce poration and diffusion into the PMPs according to the protocol from Wang et al, Nature Comm., 4:1867, 2013. Alternatively, the solution can be passed through a lipid extruder according to the protocol from Haney et al., J Contr. Rel., 207: 18-30, 2015. Alternatively, they can be electroporated according to the protocol from Wahlgren et al, Nucl. Acids. Res. 40(17):e130, 2012. After 1 hour, the PMPs are purified using a sucrose gradient and washed via ultracentrifugation as described in Example 2 before use to remove unbound nucleic acid.

Size, zeta potential, and particle count are measured using the methods in Example 3, and their stability is tested as described in Example 4. PNAs in the PMPs are quantified using an electrophoretic gel shift assay following the protocol in Nikravesh et al, Mol. Ther., 15(8): 1537-1542, 2007. Briefly, DNA antisense to the PNAs are mixed with PNA-PMPs treated with detergent to release the PNAs. PNA-DNA complexes are run on a gel and visualized with an ssDNA dye. The duplexes are then quantified by fluorescent imaging. Loaded and unloaded PMPs are compared to determine loading efficiency.

b) Treatment of Cimex lectularius with vATPase-E PNA-Loaded Grapefruit PMPs for Reducing Insect Fitness

PMPs loaded with the vATPase-E PNAs identified above and a scrambled PNA control are loaded into PMPs according to the method described above. Cimex lectularius are obtained from Sierra Research Laboratories (Modesto, Calif.). Bed bug colonies are maintained in glass enclosures containing cardboard harborages and kept on a 12:12 photoperiod at 25° C. and 40-45% (ambient) humidity. Colonies are blood-fed once per week with a parafilm-membrane feeder containing defibrinated rabbit blood (Hemostat Laboratories, Dixon, Calif.).

Prior to PNA-loaded PMP treatments, 0-2 week old adults which have not blood-fed for four days are isolated and placed in glass jars to allow mating for two days. Males are sorted out, and female bed bugs are separated into experimental cohorts of 10-15 insects which are housed together. Female bed bugs are treated by allowing them to feed on defibrinated rabbit blood spiked with a final concentration of 0, 0.1, 1, 5, or 10 μM vATPase-E PNA-loaded PMPs or 0, 0.1, 1, 5, or 10 pM of a scrambled PNA-loaded PMPs for 15 min until fully engorged. Bed bugs fed defibrinated rabbit blood only serve as controls for feeding experiments. After PNA-loaded PMP treatment, cohorts of 10-15 bed bugs are maintained at 25° C. and 40-45% (ambient) humidity in a petri dish containing a sterile pad, which provides a suitable substrate for oviposition (Advantec MFS, Inc., Dublin, Calif.). For survival assays, dead insects are counted, recorded, and removed from their enclosure each day for 10 days, and the mean percent survival of vATPase-E PNA-loaded PMPs bed bugs is calculated compared to scrambled PNA-loaded PMP and water controls.

Thereafter, bed bugs are fed every 10 days with PNA-loaded PMP spiked blood, and transferred to a new petri dish. Petri dishes with eggs are kept inside a growth chamber for 2 wks to allow sufficient hatching time. The eggs laid are observed under a stereomicroscope with a 16× magnification, and the average number of eggs laid by female bed bugs per feeding interval is calculated for 30 d, the average number of nymphs that emerge from the eggs are assessed, and the mean percent survival of bed bugs is calculated. The effect of ginger root PMPs on bed bug survival, fecundity, and development are determined by comparing the vATPase-E PNA PMP-treated cohorts to the scrambled PNA-loaded PMP and PBS-treated control cohorts.

At day 3 and 30 post treatment, three bed bugs per treatment are snap-frozen in liquid nitrogen and stored at −80° C. to assess PNA vATPase-E mRNA knockdown by Real-Time Quantitative PCR RT-qPCR. Total RNA is extracted using a RNeasy Mini Kit (Qiagen), and cDNA is synthesized using SuperScript III Reverse Transcriptase (Invitrogen Carlsbad, Calif.). RT-qPCR is performed using SsoAdvanced™ Universal SYBR® Green Supermix (BioRad) using previously reported primers: v-A TPase-E-Forward: AGGTCGCCTTGTCCAAAAC, v-ATPase-E-Reverse: GCTTTTAGTCTCGCCTGGTTC, and housekeeping gene rpL8-Forward: AGGCACGGTTACATCAAAGG, rpL8-Reverse: TCGGGAGCAATGAAGAGTTC (Basnet and Kamble, Journal of Medical Entomology, 55(3): 540-546. 20181. The abundance of v-ATPase-E is normalized to the ribosomal protein L8 abundance, and the relative v-ATPase-E knock down efficiency is determined by calculating the ΔΔCt value, comparing normalized v-ATPase-E expression in v-ATPase-E PNA-loaded PMP treated samples compared to scrambled PNA-loaded PMP treated controls.

### Example 12: Treatment of a Bacterium with Small Molecule-Loaded PMPs

This example demonstrates methods of loading PMPs with small molecules, in this embodiment, streptomycin, for the purpose of reducing the fitness of E. coli. It also demonstrates that small molecule loaded-PMPs are stable and retain their activity over a range of processing and environmental conditions. In this example, streptomycin is used as a model small molecule, and E. coli is used as a model pathogenic bacterium.

Therapeutic Dose:

PMPs loaded with small molecule, formulated in water to a concentration that delivers an equivalent of an effective Streptomycin sulfate dose of 0, 2.5, 10, 50, 100, or 200 mg/ml

a) Loading of Grapefruit PMPs with Streptomycin

PMPs are produced as described above are placed in PBS solution with solubilized Streptomycin. The solution is left for 1 hour at 22° C., according to the protocol in Sun et al., Mol Ther. September; 18(9):1606-14, 2010. Alternatively, the solution is sonicated to induce poration and diffusion into the exosomes according to the protocol from Wang et al, Nature Comm., 4:1867, 2013. Alternatively, the solution can be passed through a lipid extruder according to the protocol from Haney et al., J Contr. Rel., 207: 18-30, 2015. Alternatively, they can be electroporated according to the protocol from Wahlgren et al, Nucl. Acids. Res. 40(17):e130, 2012. After 1 hour, the loaded PMPs are purified using a sucrose gradient and washed via ultracentrifugation as described in Example 2 before use to remove unbound small molecules. Streptomycin-loaded PMPs are characterized for size and zeta potential using the methods in Example 3. A small amount of the PMPs are Streptomycin content is assessed using UV-Vis at 195 nm using a standard curve. Briefly, stock solutions of streptomycin at various concentrations of interest are made and 100 microliters of the solution are placed in a flat-bottom clear 96 well plate. The absorbance at 195 nm is measured using a UV-V plate reader. Samples are also put on the plate, and a regression is used to determine what the concentration could be according to the standard. For insufficiently high concentrations, the protocol from Kurosawa et al., J. Chromatogr., 343:379-385, 1985 is used to measure the streptomycin content by HPLC. Streptomycin-loaded PMP stability is tested as described in Example 4.

b) Treatment of E. coli with Streptomycin-Loaded Grapefruit PMPs for Reducing Bacterial Fitness

E. coli are acquired from ATCC (#25922) and grown on Trypticase Soy Agar/broth at 37° C. according to the manufacturer's instructions. Effective concentrations of streptomycin, PMPs, and streptomycin-loaded PMPs are tested for the ability to prevent growth of E. coli according to a modified standard disk diffusion susceptibility method.

An E. coli inoculum suspension is prepared by selecting several morphologically similar colonies from an overnight growth (16-24 h of incubation) on a non-selective medium with a sterile loop or a cotton swab and suspending the colonies in sterile saline (0.85% NaCl w/v in water) to the density of a McFarland 0.5 standard, approximately corresponding to 1-2×10⁸ CFU/ml. Mueller-Hinton agar plates (150 mm diameter) are inoculated with the E. coli suspension, by dipping a sterile cotton swab into the inoculum suspension, removing the excess fluid from the swab, and spreading bacteria evenly over the entire surface of the agar plate by swabbing in three directions. Next, 3 uL of PBS (negative control), 0 (PMP control), 2.5, 10, 50, 100, or 200 mg/ml effective dose of Streptomycin-loaded PMPs, and 200 mg/ml streptomycin alone (+control) are spotted onto the plate and allowed to dry. The plates are incubated for 16-18 hours at 35° C., photographed, and scanned. The diameter of the lytic zone (area without bacteria) around the spotted area is measured. Control (PBS), streptomycin, PMP, and streptomycin-loaded PMP-treated lytic zones are compared to determine the bactericidal effect.

### Example 13: Treatment of a Nematode with Protein/Peptide-Loaded Plant Messenger Packs

This example demonstrates loading of PMPs with a peptide construct for the purpose of reducing fitness in parasitic nematodes. This example demonstrates PMPs loaded with GFP are taken up in the digestive tract of C. elegans, and it demonstrates that peptide-loaded PMPs are stable and retain their activity over a range of processing and environmental conditions. In this example, GFP is used as a model peptide, and C. elegans is used as model nematodes.

Therapeutic Dose:

PMPs loaded with GFP, formulated in water to a concentration that delivers 0 (unloaded PMP control), 10, 100, or 1000 μg/ml GFP-protein loaded in PMPs

Experimental Protocol:

a) Loading Grapefruit PMPs with a Protein or Peptide

PMPs are produced from grapefruit juice according to Example 1. Green fluorescent protein is synthesized commercially and solubilized in PBS. PMPs are placed in solution with the protein in PBS. If the protein or peptide is insoluble, pH is adjusted until it is soluble. If the protein or peptide is still insoluble, the insoluble protein or peptide is used. The solution is then sonicated to induce poration and diffusion into the exosomes according to the protocol from Wang et al., Molecular Therapy. 22(3): 522-534, 2014. Alternatively, the solution can be passed through a lipid extruder according to the protocol from Haney et al., J Contr. Rel., 207: 18-30, 2015. Alternatively, PMPs can be electroporated according to the protocol from Wahlgren et al, Nucl. Acids. Res. 40(17):e130, 2012. After 1 hour, the PMPs are purified using a sucrose gradient and washed via ultracentrifugation as described in Example 1 before use to remove unbound protein. PMP-derived liposomes are characterized as described in Example 3, and their stability is tested as described in Example 4. GFP encapsulation of PMPs is measured by Western blot or fluorescence.

b) Delivery of a Model Protein to a Nematode

C. elegans wild-type N2 Bristol strain (C. elegans Genomics Center) are maintained on an Escherichia coli (strain 0P50) lawn on nematode growth medium (NOM) agar plates (3 g/l NaCl, 17 g/l agar, 2.5 g/l peptone, 5 mg/I cholesterol, 25 mM KH₂PO₄ (pH 6.0), 1 mM CaCl₂, 1 mM MgSO₄) at 20° C., from L1 until the L4 stage.

One-day old C. elegans are transferred to a new plate and are fed 0 (unloaded PMP control), 10, 100, or 1000 ug/ml GFP-loaded PMPs in a liquid solution following the feeding protocol in Conte et al., Curr. Protoc. Mol. Bio., 109:26.3.1-30 2015. Worms are next examined for GFP-loaded PMP uptake in the digestive tract by using a fluorescent microscope for green fluorescence, compared to unloaded PMP-treatment and a sterile water control.

### Example 14: PMP Production from Blended Fruit Juice Using Ultracentrifugation and Sucrose Gradient Purification

This example demonstrates that PMPs can be produced from fruit by blending the fruit and using a combination of sequential centrifugation to remove debris, ultracentrifugation to pellet crude PMPs, and using a sucrose density gradient to purify PMPs. In this example, grapefruit was used as a model fruit.

a) Production of Grapefruit PMPs by Ultracentrifugation and Sucrose Density Gradient Purification

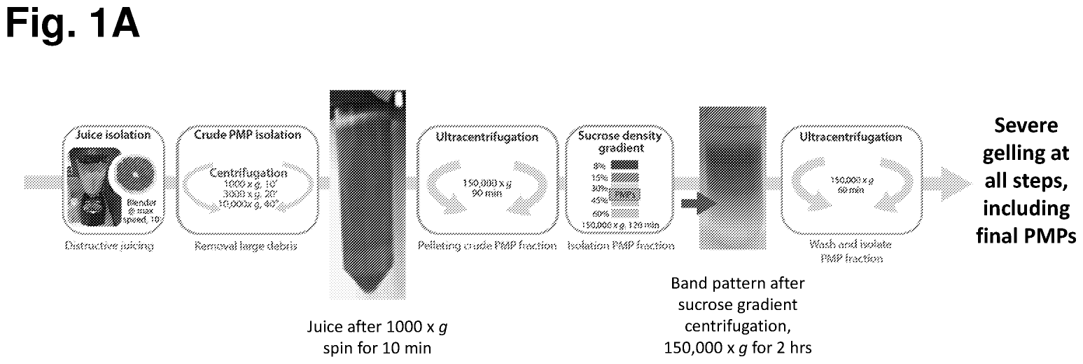

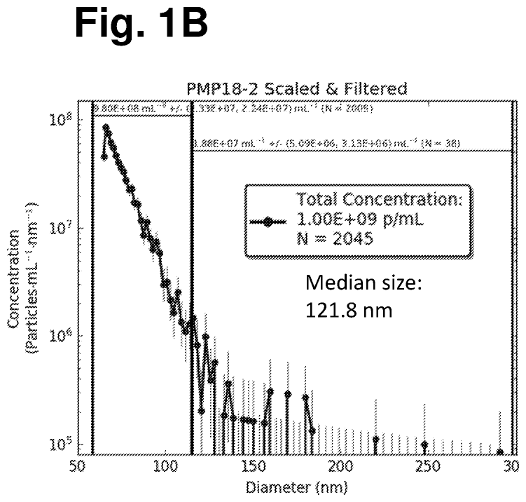

A workflow for grapefruit PMP production using a blender, ultracentrifugation and sucrose gradient purification is shown in FIG. 1A. One red grapefruit was purchased from a local Whole Foods Market®, and the albedo, flavedo, and segment membranes were removed to collect juice sacs, which were homogenized using a blender at maximum speed for 10 minutes. One hundred mL juice was diluted 5× with PBS, followed by subsequent centrifugation at 1000×g for 10 minutes, 3000×g for 20 minutes, and 10,000×g for 40 minutes to remove large debris. 28 mL of cleared juice was ultracentrifuged on a Sorvall™ MX 120 Plus Micro-Ultracentrifuge at 150,000×g for 90 minutes at 4° C. using a S50-ST (4×7 mL) swing bucket rotor to obtain a crude PMP pellet which was resuspended in PBS pH 7.4. Next, a sucrose gradient was prepared in Tris-HCL pH7.2, crude PMPs were layered on top of the sucrose gradient (from top to bottom: 8, 15. 30. 45 and 60% sucrose), and spun down by ultracentrifugation at 150,000×g for 120 minutes at 4° C. using a S50-ST (4×7 mL) swing bucket rotor. One mL fractions were collected and PMPs were isolated at the 30-45% interface. The fractions were washed with PBS by ultracentrifugation at 150,000×g for 120 minutes at 4° C. and pellets were dissolved in a minimal amount of PBS.

PMP concentration (1×10⁹ PMPs/mL) and median PMP size (121.8 nm) were determined using a Spectradyne nCS1™ particle analyzer, using a TS-400 cartridge (FIG. 1B). The zeta potential was determined using a Malvern Zetasizer Ultra and was −11.5+/−0.357 mV.

This example demonstrates that grapefruit PMPs can be isolated using ultracentrifugation combined with sucrose gradient purification methods. However, this method induced severe gelling of the samples at all PMP production steps and in the final PMP solution.

### Example 15: PMP Production from Mesh-Pressed Fruit Juice Using Ultracentrifugation and Sucrose Gradient Purification

This example demonstrates that cell wall and cell membrane contaminants can be reduced during the PMP production process by using a milder juicing process (mesh strainer). In this example, grapefruit was used as a model fruit.

a) Mild Juicing Reduces Gelling During PMP Production from Grapefruit PMPs

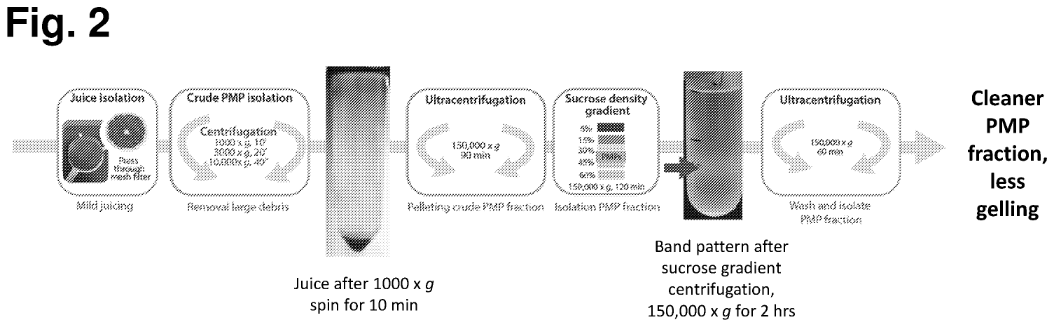

Juice sacs were isolated from a red grapefruit as described in Example 14. To reduce gelling during PMP production, instead of using a destructive blending method, juice sacs were gently pressed against a tea strainer mesh to collect the juice and to reduce cell wall and cell membrane contaminants. After differential centrifugation, the juice was more clear than after using a blender, and one clean PMP-containing sucrose band at the 30-45% intersection was observed after sucrose density gradient centrifugation (FIG. 2). There was overall less gelling during and after PMP production.

Our data shows that use of a mild juicing step reduces gelling caused by contaminants during PMP production when compared to a method comprising blending.

### Example 16: PMP Production Using Ultracentrifugation and Size Exclusion Chromatography

This example describes the production of PMPs from fruits by using Ultracentrifugation (UC) and Size Exclusion Chromatography (SEC). In this example, grapefruit is used as a model fruit.

a) Production of Grapefruit PMPs Using UC and SEC

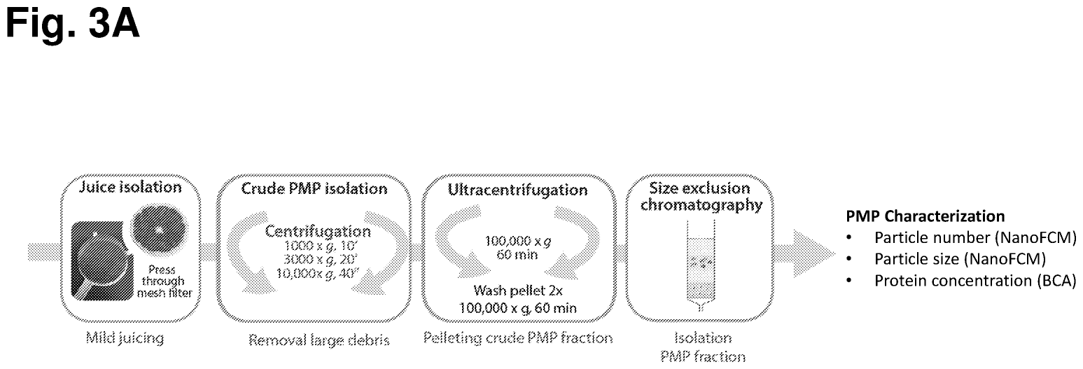

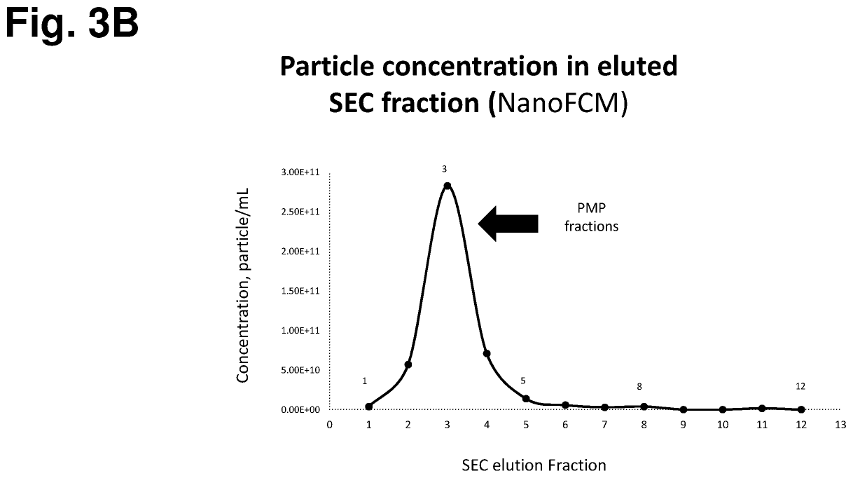

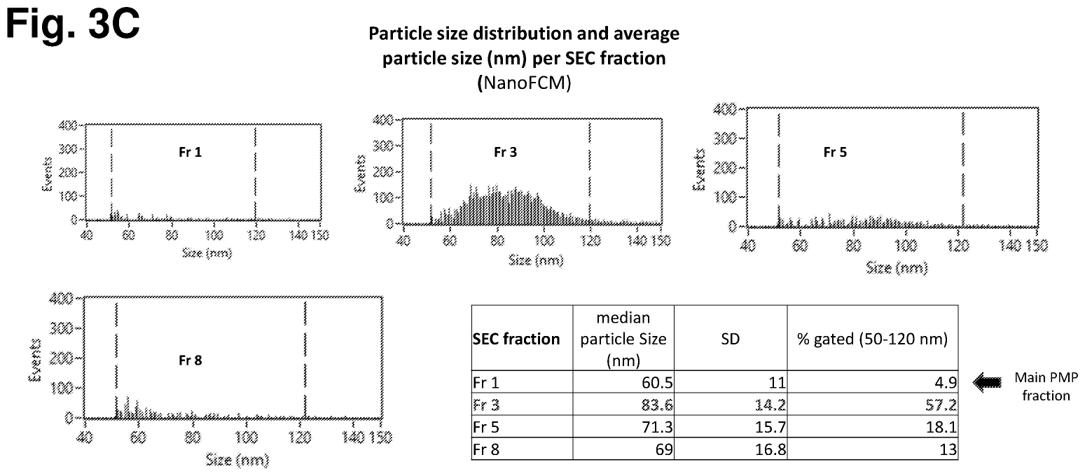

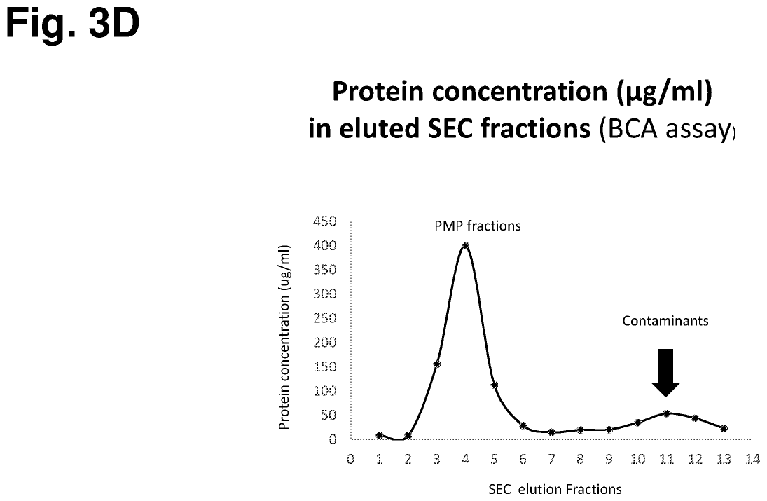

Juice sacs were isolated from a red grapefruit, as described in Example 14a, and were gently pressed against a tea strainer mesh to collect 28 ml juice. The workflow for grapefruit PMP production using UC and SEC is depicted in FIG. 3A. Briefly, juice was subjected to differential centrifugation at 1000×g for 10 minutes, 3000×g for 20 minutes, and 10,000×g for 40 minutes to remove large debris. 28 ml of cleared juice was ultracentrifuged on a Sorvall™ MX 120 Plus Micro-Ultracentrifuge at 100,000×g for 60 minutes at 4° C. using a S50-ST (4×7 mL) swing bucket rotor to obtain a crude PMP pellet which was resuspended in MES buffer (20 mM MES, NaCl, pH 6). After washing the pellets twice with MES buffer, the final pellet was resuspended in 1 ml PBS, pH 7.4. Next, we used size exclusion chromatography to elute the PMP-containing fractions. SEC elution fractions were analyzed by nano-flow cytometry using a NanoFCM to determine PMP size and concentration using concentration and size standards provided by the manufacturer. In addition, absorbance at 280 nm (SpectraMax®) and protein concentration (Pierce™ BCA assay, ThermoFisher) were determined on SEC fractions to identify in which fractions PMPs are eluted (FIGS. 3B-3D). SEC fractions 2-4 were identified as the PMP-containing fractions. Analysis of earlier- and later-eluting fractions indicated that SEC fraction 3 is the main PMP-containing fraction, with a concentration of 2.83×10¹¹ PMPs/mL (57.2% of all particles in the 50-120 nm size range), with a median size of 83.6 nm+/−14.2 nm (SD). While the late elution fractions 8-13 had a very low concentration of particles as shown by NanoFCM, protein contaminants were detected in these fractions by BCA analysis.

Our data shows that TFF and SEC can be used to isolate purified PMPs from late-eluting contaminants, and that a combination of the analysis methods used here can identify PMP fractions from late-eluting contaminants.

### Example 17: Scaled PMP Production Using Tangential Flow Filtration and Size Exclusion Chromatography Combined with EDTA/Dialysis to Reduce Contaminants

This example describes the scaled production of PMPs from fruits by using Tangential Flow Filtration (TFF) and Size Exclusion Chromatography (SEC), combined with an EDTA incubation to reduce the formation of pectin macromolecules, and overnight dialysis to reduce contaminants. In this example, grapefruit is used as a model fruit.

a) Production of Grapefruit PMPs Using TFF and SEC

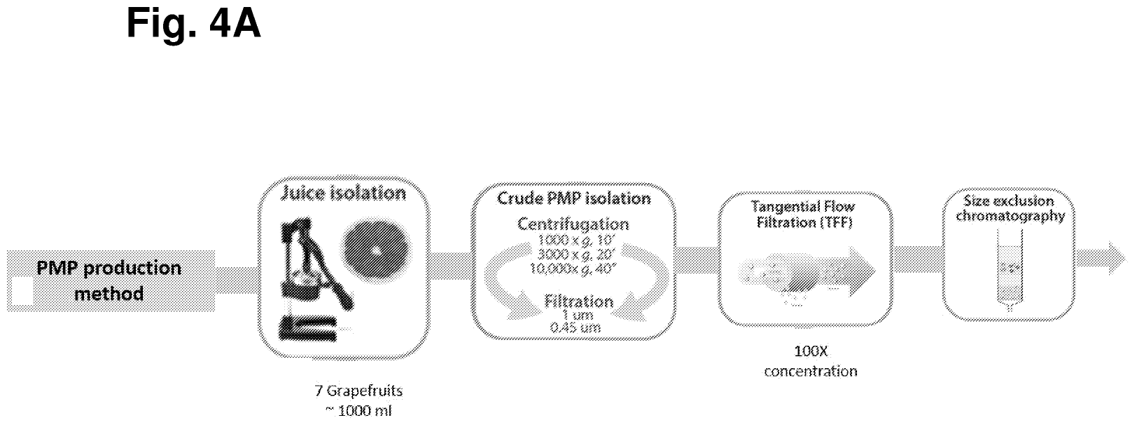

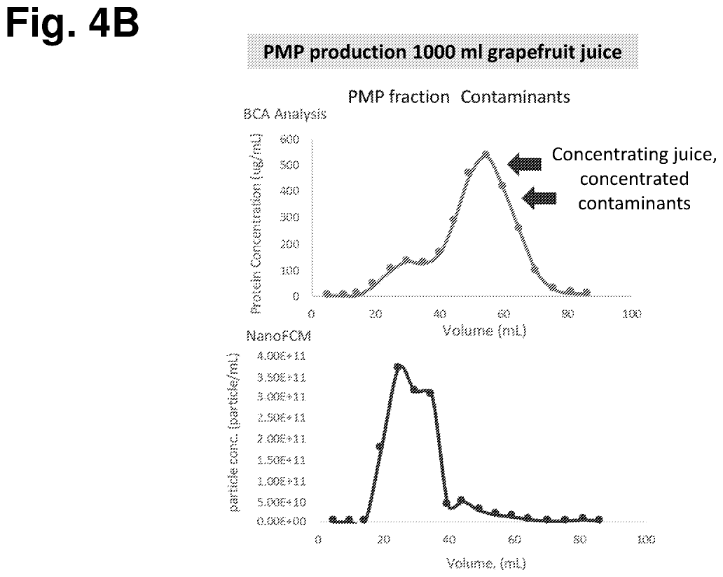

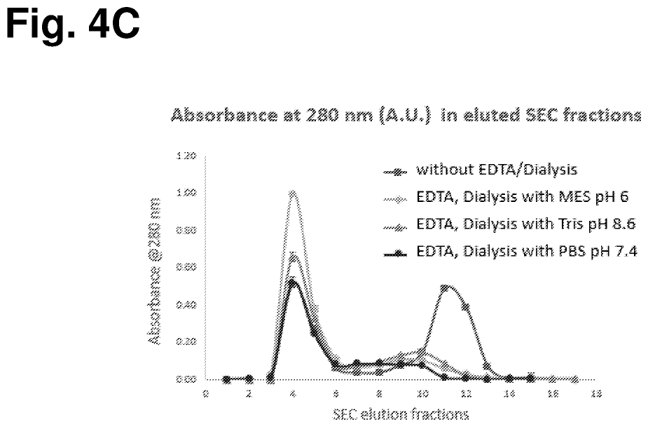

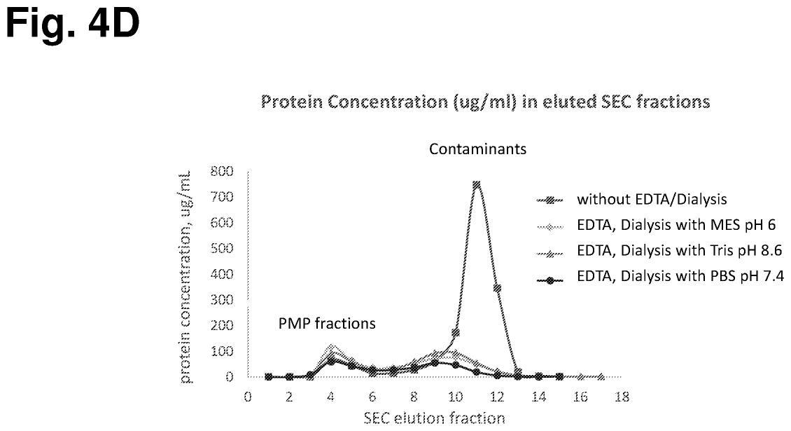

Red grapefruits were obtained from a local Whole Foods Market®, and 1000 ml juice was isolated using a juice press. The workflow for grapefruit PMP production using TFF and SEC is depicted in FIG. 4A. Juice was subjected to differential centrifugation at 1000×g for 10 minutes, 3000×g for 20 minutes, and 10,000×g for 40 minutes to remove large debris. Cleared grapefruit juice was concentrated and washed once using a TFF (5 nm pore size) to 2 mL (100×). Next, we used size exclusion chromatography to elute the PMP-containing fractions. SEC elution fractions were analyzed by nano-flow cytometry using a NanoFCM to determine PMP concentration using concentration and size standards provided by the manufacturer. In addition, protein concentration (Piercer™ BCA assay, ThermoFisher) was determined for SEC fractions to identify the fractions in which PMPs are eluted. The scaled production from 1 liter of juice (100× concentrated) also concentrated a high amount of contaminants in the late SEC fractions as can be detected by BCA assay (FIG. 4B, top panel). The overall total PMP yield (FIG. 4B, bottom panel) was lower in the scaled production when compared to single grapefruit isolations, which may indicate loss of PMPs.

b) Reducing Contaminants by EDTA Incubation and Dialysis

Red grapefruits were obtained from a local Whole Foods Market®, and 800 ml juice was isolated using a juice press. Juice was subjected to differential centrifugation at 1000×g for 10 minutes, 3000×g for 20 minutes, and 10,000×g for 40 minutes to remove large debris, and filtered through a 1 μm and 0.45 μm filter to remove large particles. Cleared grapefruit juice was split into 4 different treatment groups containing 125 ml juice each. Treatment Group 1 was processed as described in Example 17a, concentrated and washed (PBS) to a final concentration of 63×, and subjected to SEC. Prior to TFF, 475 ml juice was incubated with a final concentration of 50 mM EDTA, pH 7.15 for 1.5 hrs at RT to chelate iron and reduce the formation of pectin macromolecules. Afterwards, juice was split in three treatment groups that underwent TFF concentration with either a PBS (without calcium/magnesium) pH 7.4, MES pH 6, or Tris pH 8.6 wash to a final juice concentration of 63×. Next, samples were dialyzed in the same wash buffer overnight at 4° C. using a 300 kDa membrane and subjected to SEC. Compared to the high contaminant peak in the late elution fractions of the TFF only control, EDTA incubation followed by overnight dialysis strongly reduced contaminants, as shown by absorbance at 280 nm (FIG. 4C) and BCA protein analysis (FIG. 4D), which is sensitive to the presence of sugars and pectins. There was no difference in the dialysis buffers used (PBS without calcium/magnesium pH 7.4, MES pH 6, Tris pH 8.6).

Our data indicates that incubation with EDTA followed by dialysis reduces the amount of co-purified contaminants, facilitating scaled PMP production.

### Example 18: PMP Stability

This example demonstrates that PMPs are stable at different environmental conditions. In this example, grapefruit and lemon PMPs are used as model PMPs.

a) Production of Grapefruit PMPs Using TFF Combined with SEC

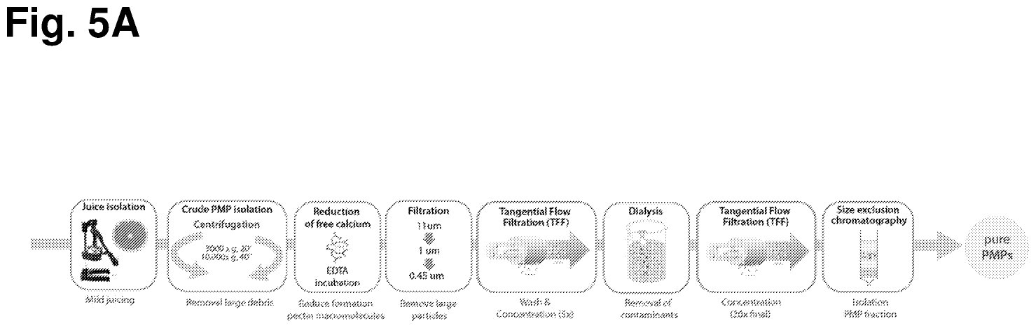

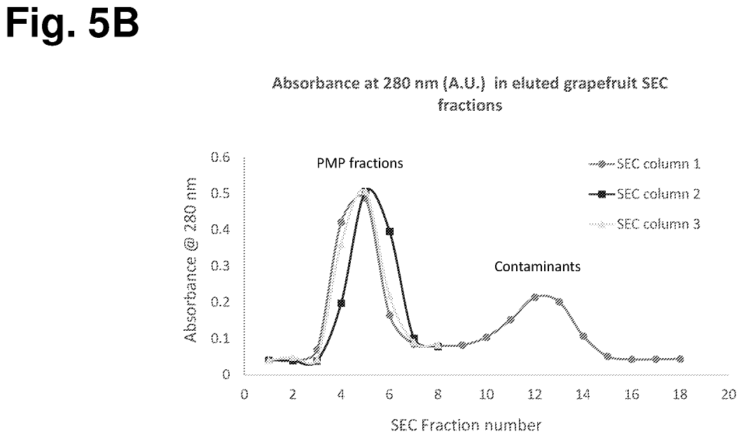

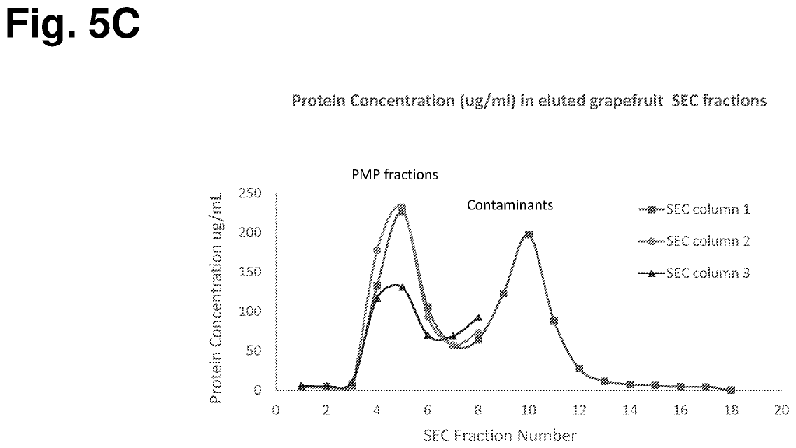

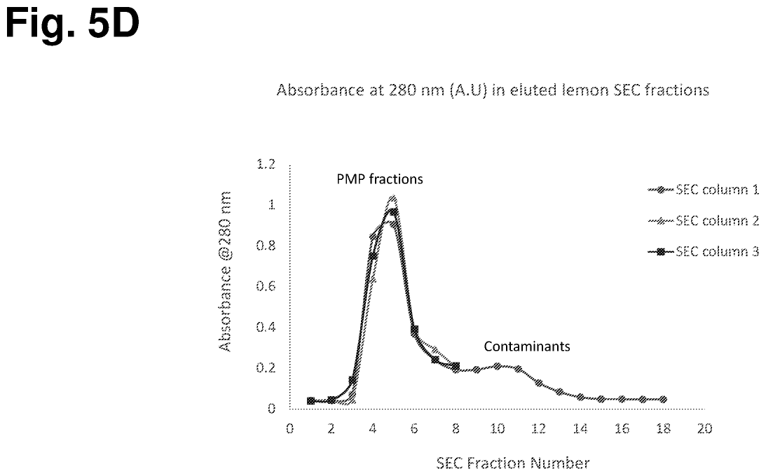

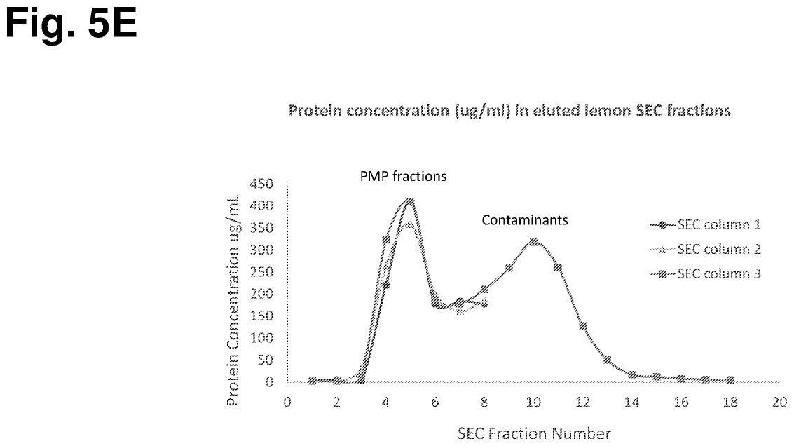

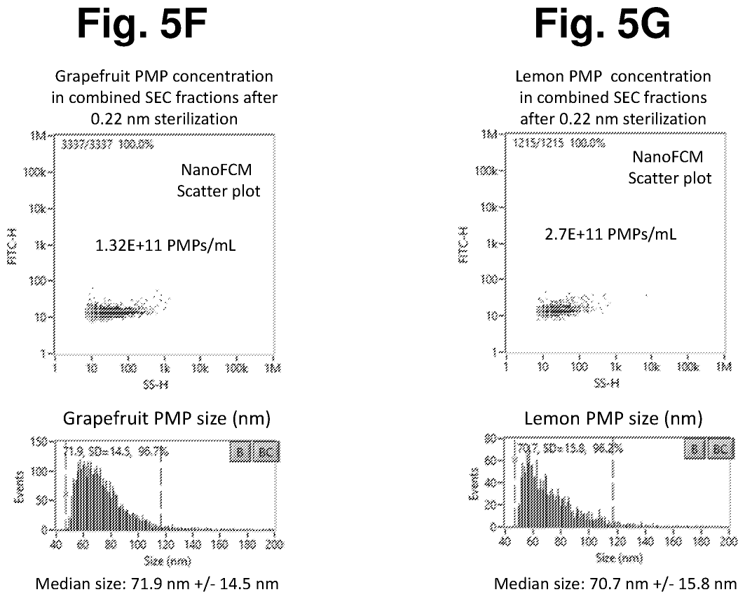

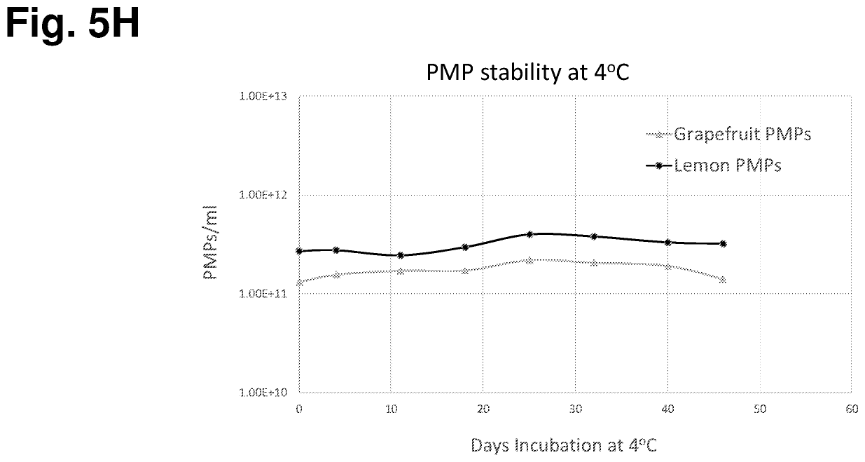

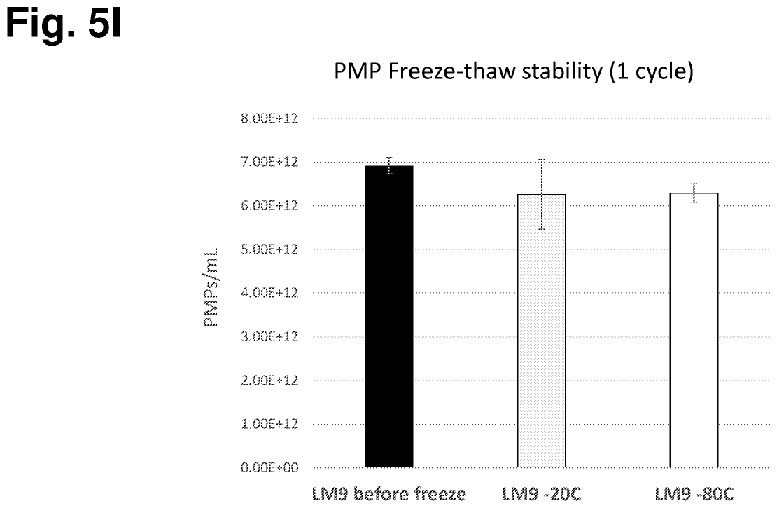

Red organic grapefruits (Florida) were obtained from a local Whole Foods Market®. The PMP production workflow is depicted in FIG. 5A. One liter of grapefruit juice was collected using a juice press, and was subsequently centrifuged at 3000×g for 20 minutes, followed by 10,000×g for 40 minutes to remove large debris. Next, 500 mM EDTA pH 8.6 was added to a final concentration of 50 mM EDTA, pH 7, and the solution was incubated for 30 minutes to chelate calcium and prevent the formation of pectin macromolecules. Subsequently the juice was passaged through 11 μm, 1 μm and 0.45 μm filters to remove large particles. Filtered juice was concentrated and washed (500 ml PBS) by Tangential Flow Filtration (TFF) (pore size 5 nm) to 400 ml (2.5×) and dialyzed overnight in PBS pH 7.4 (with one medium exchange) using a 300 kDa dialysis membrane to remove contaminants. Subsequently, the dialyzed juice was further concentrated by TFF to a final concentration of 50 ml (20×). Next, we used size exclusion chromatography to elute the PMP-containing fractions, which were analyzed by absorbance at 280 nm (SpectraMax®) and a protein concentration assay (Piercer™ BCA assay, ThermoFisher) to verify the PMP-containing fractions and late fractions containing contaminants (FIGS. 5B and 5C). SEC fractions 4-6 contained purified PMPs (fractions 8-14 contained contaminants), were pooled together, and were filter sterilized by sequential filtration using 0.8 μm, 0.45 μm and 0.22 pin syringe filters. The final PMP concentration (1.32×10¹¹ PMPs/mL) and median PMP size (71.9 nm+/−14.5 nm) in the combined sterilized PMP-containing fractions were determined by NanoFCM using concentration and size standards provided by the manufacturer (FIG. 5F).

b) Production of Lemon PMPs Using TFF Combined with SEC

Lemons were obtained from a local Whole Foods Market®. One liter of lemon juice was collected using a juice press, and was subsequently centrifuged at 3000 g for 20 minutes, followed by 10,000 g for 40 minutes to remove large debris. Next, 500 mM EDTA pH 8.6 was added to a final concentration of 50 mM EDTA, pH 7, and the solution was incubated for 30 minutes to chelate calcium and prevent the formation of pectin macromolecules. Subsequently the juice was passaged through a coffee filter, 1 μm and 0.45 μm filters to remove large particles. Filtered juice was concentrated by Tangential Flow Filtration (TFF) (5 nm pore size) to 400 ml (2.5× concentrated) and dialyzed overnight in PBS pH 7.4 using a 300 kDa dialysis membrane to remove contaminants. Subsequently, the dialyzed juice was further concentrated by TFF to a final concentration of 50 ml (20×). Next, we used size exclusion chromatography to elute the PMP-containing fractions, which were analyzed by absorbance at 280 nm (SpectraMax®) and a protein concentration assay (Piercer™ BCA assay, ThermoFisher) to verify the PMP-containing fractions and late fractions containing contaminants (FIGS. 5D and 5E). SEC fractions 4-6 contained purified PMPs (fractions 8-14 contained contaminants), were pooled together, and were filter sterilized by sequential filtration using 0.8 pm, 0.45 pm and 0.22 μm syringe filters. The final PMP concentration (2.7×10¹¹ PMPs/mL) and median PMP size (70.7 nm+/−15.8 nm) in the combined sterilized PMP-containing fractions were determined by NanoFCM, using concentration and size standards provided by the manufacturer (FIG. 5G).

c) Stability of Grapefruit and Lemon PMPs at 4° C.

Grapefruit and lemon PMPs were produced as described in Examples 18a and 18b. The stability of PMPs was assessed by measurement of concentration of total PMPs (PMP/ml) in the sample over time using NanoFCM. The stability study was carried out at 4° C. for 46 days in the dark. Aliquots of PMPs were stored at 4° C. and analyzed by NanoFCM on predetermined days. The concentrations of total PMPs in the sample were analyzed (FIG. 5H). The relative measured PMP concentration of lemon and grapefruit PMPs between the start and endpoint of the experiment at 46 days was 119% and 107%, respectively. Our data indicate that PMPs are stable for at least 46 days at 4° C.

d) Freeze-Thaw Stability of Lemon PMPs

To determine the freeze-thaw stability of PMPs, lemon PMPs were produced from organic lemons purchased at a local Whole Foods Market®. One liter of lemon juice was collected using a juice press, and was subsequently centrifuged at 3000 g for 20 minutes, followed by 10,000 g for 40 minutes to remove large debris. Next, 500 mM EDTA pH 8.6 was added to final concentration of 50 mM EDTA, pH 7.5 and incubated for 30 minutes to chelate calcium and prevent the formation of pectin macromolecules. Subsequently, the juice was passaged through 11 μm, 1 μm and 0.45 μm filters to remove large particles. Filtered juice was concentrated and washed with 400 ml PBS, pH 7.4 by Tangential Flow Filtration (TFF) to a final volume of 400 ml (2.5× concentrated) and dialyzed overnight in PBS pH 7.4 using a 300 kDa dialysis membrane to remove contaminants. Subsequently, the dialyzed juice was further concentrated by TFF to a final concentration of 60 ml (˜17×). Next, we used size exclusion chromatography to elute the PMP-containing fractions, which were analyzed by absorbance at 280 nm (SpectraMax®) and a protein concentration assay (Piercer™ BCA assay, ThermoFisher) to verify the PMP-containing fractions and late fractions containing contaminants. SEC fractions 4-6 contained purified PMPs (fractions 8-14 contained contaminants), were pooled together, and were filter sterilized by sequential filtration using 0.8 μm, 0.45 μm and 0.22 μm syringe filters. The final PMP concentration (6.92×10¹² PMPs/mL) in the combined sterilized PMP containing fractions was determined by NanoFCM, using concentration and size standards provided by the manufacturer.

Lemon PMPs were frozen at −20° C. or −80° C. for one week, thawed at room temperature, and the concentration was measured by NanoFCM (FIG. 5I). The data indicate that lemon PMPs are stable after 1 freeze-thaw cycle after storage for one week at −20° C. or −80° C.

### Example 19: PMP Production from Plant Cell Culture Medium

This example demonstrates that PMPs can be produced from plant cell culture. In this example, the Zea mays Black Mexican Sweet (BMS) cell line is used as a model plant cell line.

a) Production of Zea mays BMS cell line PMPs The Zea mays Black Mexican sweet (BMS) cell line was purchased from the ABRC and was grown in Murashige and Skoog basal medium pH 5.8, containing 4.3 g/L Murashige and Skoog Basal Salt Mixture (Sigma M5524), 2% sucrose (S0389, Millipore Sigma), 1×MS vitamin solution (M3900, Millipore Sigma), 2 mg/L 2,4-dichlorophenoxyacetic acid (D7299, Millipore Sigma) and 250 ug/L thiamine HCL (V-014, Millipore Sigma), at 24° C. with agitation (110 rpm), and was passaged 20% volume/volume every 7 days.

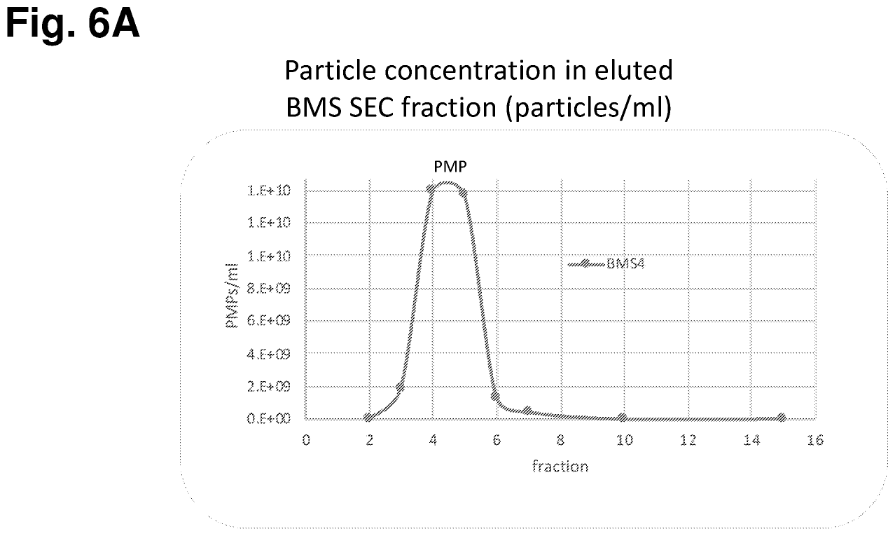

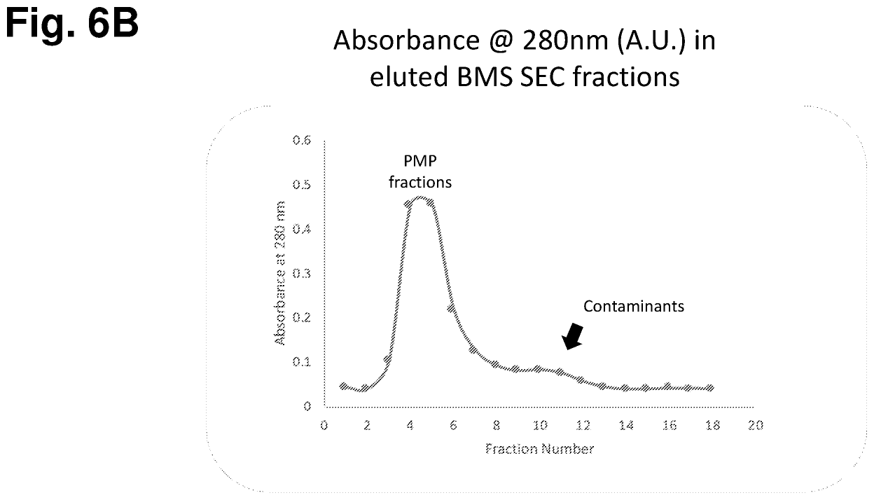

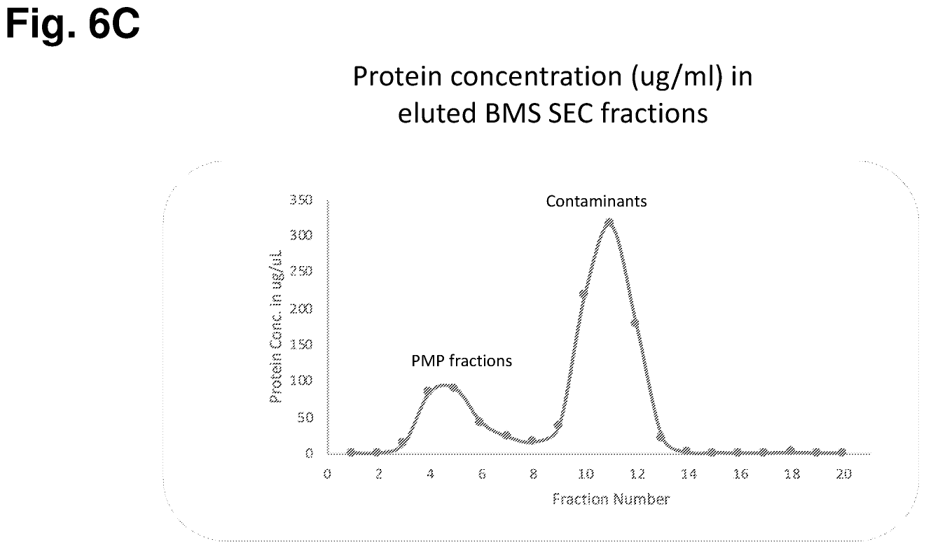

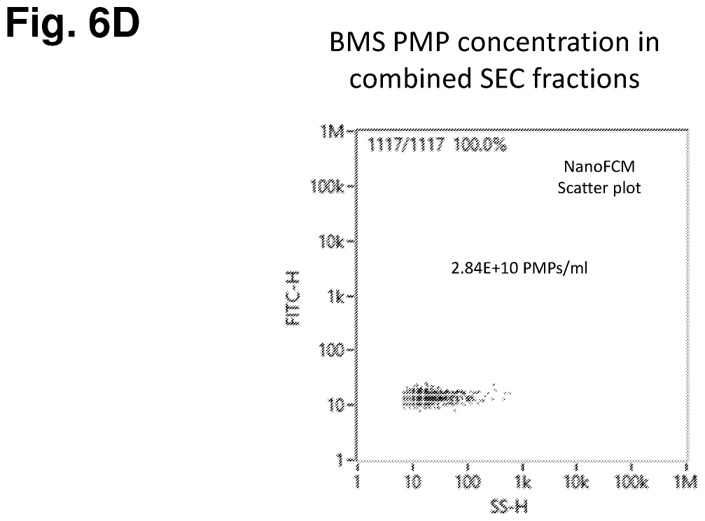

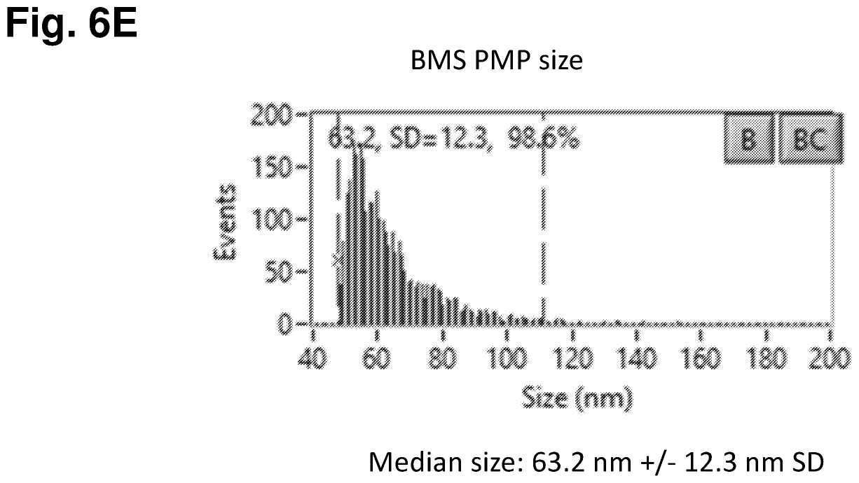

Three days after passaging, 160 ml BMS cells was collected and spun down at 500×g for 5 min to remove cells, and 10,000×g for 40 min to remove large debris. Medium was passed through a 0.45 μm filter to remove large particles, and filtered medium was concentrated and washed (100 ml MES buffer, 20 mM MES, 100 mM NaCL, pH 6) by TFF (5 nm pore size) to 4 mL (40×). Next, we used size exclusion chromatography to elute the PMP-containing fractions, which were analyzed by NanoFCM for PMP concentration, by absorbance at 280 nm (SpectraMax®), and by a protein concentration assay (Piercer™ BCA assay, ThermoFisher) to verify the PMP-containing fractions and late fractions containing contaminants (FIGS. 6A-6C). SEC fractions 4-6 contained purified PMPs (fractions 9-13 contained contaminants), and were pooled together. The final PMP concentration (2.84×10¹⁰ PMPs/ml) and median PMP size (63.2 nm+/−12.3 nm SD) in the combined PMP containing fractions were determined by NanoFCM, using concentration and size standards provided by the manufacturer (FIGS. 6D-6E).

These data show that PMPs can be isolated, purified, and concentrated from plant liquid culture media.

### Example 20: Uptake of PMPs by Bacteria and Fungi

This example demonstrates the ability of PMPs to associate with and be taken up by bacteria and fungi. In this example, grapefruit and lemon PMPs are used as a model PMP, Escherichia coli and Pseudomonas aeruginosa are used as model pathogenic bacteria, and the yeast Saccharomyces cerevisiae is used as a model pathogenic fungus.

a) Labeling of Grapefruit and Lemon PMPs with DyLight 800 NHS Ester

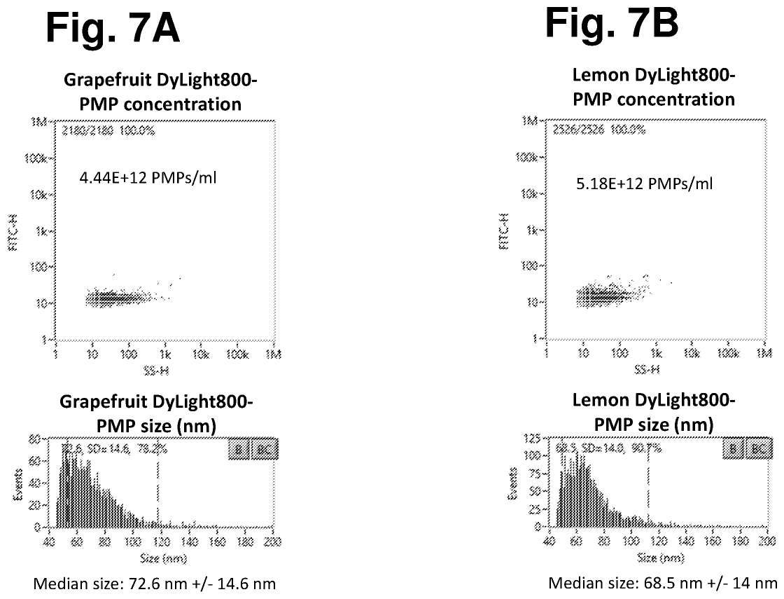

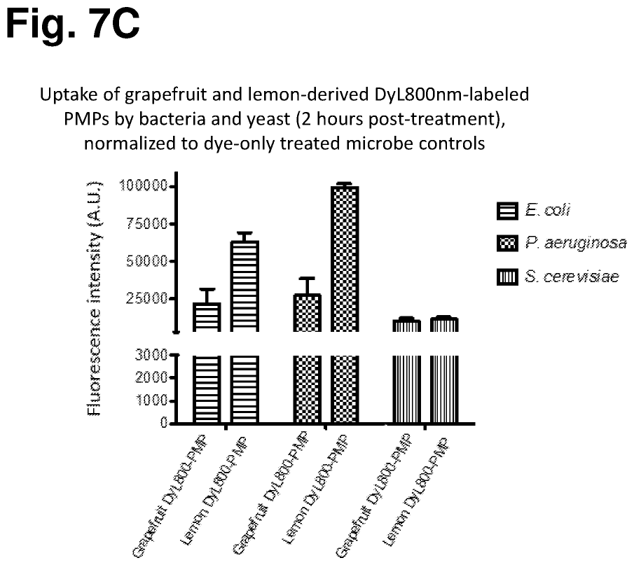

Grapefruit and lemon PMPs were produced as described in Examples 18a and 18b. PMPs were labeled with the DyLight 800 NHS Ester (Life Technologies, #46421) covalent membrane dye (DyL800). Briefly, DyL800 was dissolved in DMSO to a final concentration of 10 mg/ml, and 200 μI of PMPs were mixed with 5 μI dye and incubated for 1 h at room temperature on a shaker. Labeled PMPs were washed 2-3 times by ultracentrifuge at 100,000×g for 1 hr at 4° C., and pellets were resuspended with 1.5 ml UltraPure water. To control for the presence of potential dye aggregates, a dye-only control sample was prepared according to the same procedure, adding 200 μI of UltraPure water instead of PMPs. The final DyL800-labeled PMP pellet and DyL800 dye-only control were resuspended in a minimal amount of UltraPure water and characterized by NanoFCM. The final concentration of grapefruit DyL800-labeled PMPs was 4.44×10¹² PMPs/ml, with a median DyL800-PMP size of 72.6 nm+/−14.6 nm (FIG. 7A), and the final concentration of lemon DyL800-labeled PMPs was 5.18×10¹² PMPs/ml with an average DyL800-PMP size of 68.5 nm+/−14 nm (FIG. 7B).

b. Uptake of DyL800-Labeled Grapefruit and Lemon PMPs by Yeast

Saccharomyces cerevisiae (ATCC, #9763) was grown on yeast extract peptone dextrose broth (YPD) and maintained at 30° C. To determine whether PMPs can be taken up by yeast, a fresh 5 ml yeast culture was grown overnight at 30° C., and cells were pelleted at 1500×g for 5 min and resuspended in 10 ml water. Yeast cells were washed once with 10 ml water, resuspended in 10 ml water, and incubated for 2h at 30° C. with shaking to nutrient starve the cells. Next, 95 ul of yeast cells were mixed with either 5 ul water (negative control), DyL800 dye only control (dye aggregate control), or DyL800-PMPs to a final concentration of 5×10¹⁰ DyL800-PMPs/ml in a 1.5 ml tube. Samples were incubated for 2h at 30° C. with shaking. Next, treated cells were washed with 1 ml wash buffer (water supplemented with 0.5% Triton X-100), incubated for 5 min, and spun down at 1500×g for 5 min. The supernatant was removed and the yeast cells were washed an additional 3 times to remove PMPs that are not taken up by the cells and a final time with water to remove the detergent. Yeast cells were resuspended in 100 ul water and transferred to a clear bottom 96 well plate, and the relative fluorescence intensity (A.U.) at 800 nm excitation was measured on an Odyssey® CLx scanner (Li-Cor).

To assess DyL800-PMP uptake by yeast, samples were normalized to the DyL800 dye only control, and the grapefruit and lemon DyL800-PMP relative fluorescence intensities were compared. Our data indicates that Saccharomyces cerevisiae takes up PMPs, and no uptake difference was observed between lemon and grapefruit DyL800-PMPs (FIG. 7C).

c) Uptake of DyL800-Labeled Grapefruit and Lemon PMPs by Bacteria

Bacteria and yeast strains were maintained as indicated by the supplier: E. coli (Ec, ATCC, #25922) was grown on Trypticase Soy Agar/broth at 37° C. and Pseudomonas aeruginosa (Pa, ATCC) was grown on Tryptic soy Agar/broth with 50 mg/ml rifampicin at 37° C.

To determine whether PMPs can be taken up by bacteria, fresh 5 ml bacterial cultures were grown overnight, and cells were pelleted at 3000×g for 5 min, resuspended in 5 ml 10 mM MgCl₂, washed once with 5 ml 10 mM MgCl₂, and resuspended in 5 ml 10 mM MgCl₂. Cells were incubated for 2 h at 37° C. (Ec) or 30° C. (Pa) in a shaking incubator at ˜200 rpm to nutrient starve the cells. The OD600 was measured and cell densities were adjusted to ˜10×10⁹ CFU/ml. Next, 95 ul of bacterial cells were mixed with either 5 ul water (negative control), DyL800 dye only control (dye aggregate control), or DyL800-PMPs at a final concentration of 5×10¹⁰ DyL800-PMPs/ml in a 1.5 ml tube. Samples were incubated for 2h at 30° C. with shaking. Next, treated cells were washed with 1 ml wash buffer (10 mM MgCl₂ with 0.5% Triton X-100), incubated for 5 min, and spun down at 3000×g for 5 min. The supernatant was removed and the yeast cells were washed an additional 3 times to remove PMPs that are not taken up by the cells, and once more with 1 ml 10 mM MgCl₂ to remove detergent. Bacterial cells were resuspended in 100 ul 10 mM MgCl₂ and transferred to a clear bottom 96 well plate, and the relative fluorescence intensity (A.U.) at 800 nm excitation was measured on an Odyssey® CLx scanner (Li-Cor).

To assess DyL800-PMP uptake by bacteria, samples were normalized to the DyL800 dye only control, and the grapefruit and lemon DyL800-PMP relative fluorescence intensities were compared. Our data indicates that all bacteria species tested take up PMPs (FIG. 7C). In general, lemon PMPs were preferentially taken up (higher signal intensity than grapefruit PMPs). E. coli and P. aeruginosa displayed the highest DyL800-PMP uptake.

### Example 21: Uptake of PMPs by Insect Cells

This example demonstrates the ability of PMPs to associate with and be taken up by insect cells. In this example, sf9 Spodoptera frugiperda (insect) cells and S2 Drosophila melanogaster (insect) cell lines are used as model insect cells, and lemon PMPs are used as model PMPs.

a) Production of Lemon PMPs

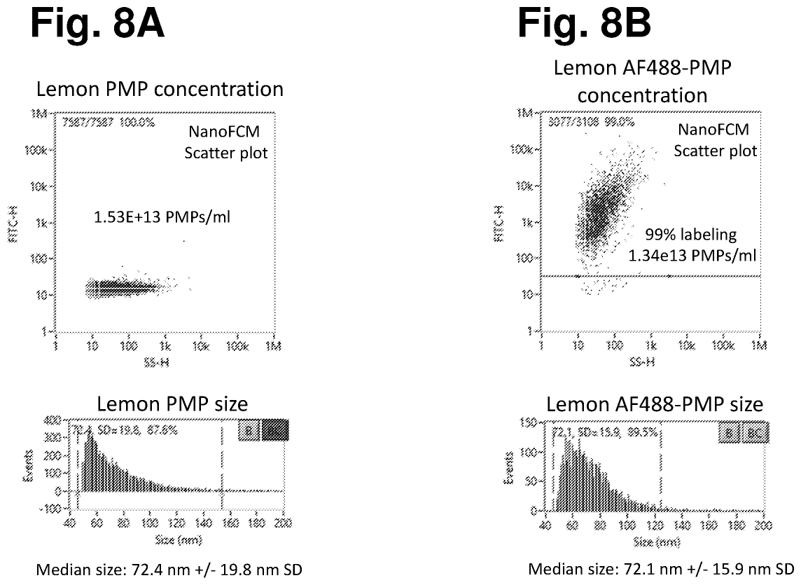

Lemons were obtained from a local Whole Foods Market®. Lemon juice (3.3 L) was collected using a juice press, pH adjusted to pH4 with NaOH, and incubated with 0.5 U/ml pectinase (Sigma, 17389) to remove pectin contaminants. Juice was incubated for one hour at room temperature with stirring, and stored overnight at 4 C, and subsequently centrifuged at 3000 g for 20 minutes, followed by 10,000 g for 40 minutes to remove large debris. Next, the processed juice was incubated with 500 mM EDTA pH8.6, to a final concentration of 50 mM EDTA, pH7.5 for 30 minutes at room temperature to chelate calcium and prevent the formation of pectin macromolecules. Subsequently, the EDTA-treated juice was passaged through an 11 pm, 1 μm and 0.45 pm filter to remove large particles. Filtered juice was washed (300 ml PBS during TFF procedure) and concentrated 2× to a total volume of 1350 ml by Tangential Flow Filtration (TFF), and dialyzed overnight using a 300 kDa dialysis membrane. Subsequently, the dialyzed juice was further washed (500 ml PMS during TFF procedure) and concentrated by TFF to a final concentration of 160 ml (˜20×). Next, we used size exclusion chromatography to elute the PMP-containing fractions, and analyzed the 280 nm absorbance (SpectraMax®) to determine the PMP-containing fractions from late elution fractions containing contaminants. SEC fractions 4-7 containing purified PMPs were pooled together, filter sterilized by sequential filtration using 0.85 μm, 0.4 μm and 0.22 μm syringe filters, and concentrated further by pelleting PMPs for 1.5 hrs at 40,000×g and finally the pellet is resuspended in Ultrapure water. The final PMP concentration (1.53×10¹³ PMPs/ml) and median PMP size (72.4 nm+/−19.8 nm SD) (FIG. 8A) were determined by nano-flow cytometry (NanoFCM) using concentration and size standards provided by the manufacturer, and PMP protein concentration (12.317 mg/ml) was determined using a Pierce™ BCA assay (ThermoFisher) according to the manufacturer's instructions.

b) Labeling of Lemon PMPs with Alexa Fluor 488 NHS Ester

Lemon PMPs were labeled with the Alexa Fluor 488 NHS Ester (Life Technologies, covalent membrane dye (AF488). Briefly, AF488 was dissolved in DMSO to a final concentration of 10 mg/ml, 200 μI of PMPs (1.53×10¹³ PMPs/ml) were mixed with 5 μI dye, incubated for 1 h at room temperature on a shaker, and labeled PMPs were washed 2-3 times by ultracentrifuge at 100,000 xg for 1 hr at 4° C. and pellets were resuspended with 1.5 ml UltraPure water. To control for the presence of potential dye aggregates, a dye-only control sample was prepared according to the same procedure, adding 200 ul of UltraPure water instead of PMPs. The final AF488-labeled PMP pellet and AF488 dye-only control were resuspended in a minimal amount of UltraPure water and characterized by NanoFCM. The final concentration of AF488-labeled PMPs was 1.33×10¹³ PMPs/ml with a median AF488-PMP size of 72.1 nm+/−15.9 nm SD, and a labeling efficiency of 99% was achieved (FIG. 8B).

c) Treatment of Insect Cells with Lemon AF488-PMPs

Lemon PMPs were produced and labeled as described in Examples 21a and 21 b. The sf9 Spodoptera frugiperda cell line was obtained from ThermoFisher Scientific (#B82501), and maintained in TNM-FH insect medium (Sigma Aldrich, T1032) supplemented with 10% heat inactivated fetal bovine serum. The S2 Drosophila melanogaster cell line was obtained from the ATCC (#CRL-1963) and maintained in Schneider's Drosophila medium (Gibco/ThermoFisher Scientific #21720024) supplemented with 10% heat inactivated fetal bovine serum. Both cell lines were grown at 26° C. For PMP treatment, S2/Sf9 cells were seeded at 50% confluency on sterile 0.01% poly-1-lysine-coated glass coverslips in a 24 well plate in 2 ml of complete medium, and allowed to adhere to the coverslip overnight. Next, cells were treated by adding 10 ul AF488 dye only (dye aggregate control), lemon PMPs (PMP only control), or AF488-PMPs to duplicate samples, which were incubated for 2h at 26° C. The final concentration was 1.33×10¹¹ PMPs/AF488-PMPs per well. The cells were then washed twice with 1 ml PBS, and fixed for 15 min with 4% formaldehyde in PBS. Cells were subsequently permeabilized with PBS+0.02% triton X-100 for 15 min, and nuclei were stained with a 1:1000 DAPI solution for 30 min. Cells were washed once with PBS and coverslips were mounted on a glass slides with ProLong™ Gold Antifade (ThermoFisher Scientific) to reduce photobleaching. The resin was set overnight and the cells were examined on an Olympus epifluorescence microscope using a 100× objective, and Z-stack images of 10 um with 0.25 um increments were taken. Similar results were obtained for both S2 D. melanogaster and S9 L. frugiperda cells. While no green foci were observed in the AF488 dye only control, and the PMP only control, nearly all insect cells treated with AF488-PMPs displayed green foci within the insect cells. There was a strong signal in the cytoplasm with several bright larger foci indicative on endosomal compartments. Due to bleed through of DAPI in the 488 channel, it was not possible to assess for the presence of AF488-PMP signal in the nucleus. For sf9 cells, 94.4% (n=38) of the examined cells displayed green foci, while this was not observed in the control samples AF488 dye only (n=68) or PMP only (n=42) controls.

Our data indicate that PMPs can associate with insect cell membranes, and can be efficiently taken up by insect cells.

### Example 22: Loading of PMPs with a Small Molecule

This example demonstrates loading of PMPs with a model small molecule for the purpose of delivering an agent using different PMP sources and encapsulation methods. In this example, doxorubicin is used as a model small molecule, and lemon and grapefruit PMPs are used as model PMPs.

We show that PMPs can be efficiently loaded with doxorubicin, and that loaded PMPs are stable for at least 8 weeks at 4° C.

a) Production of Grapefruit PMPs Using TFF Combined with SEC

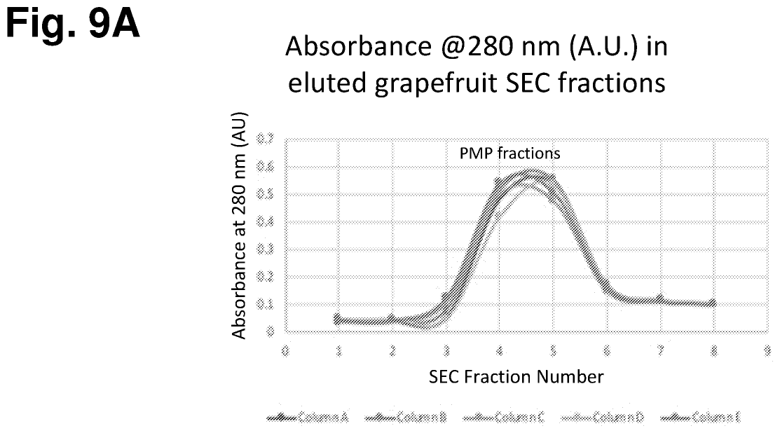

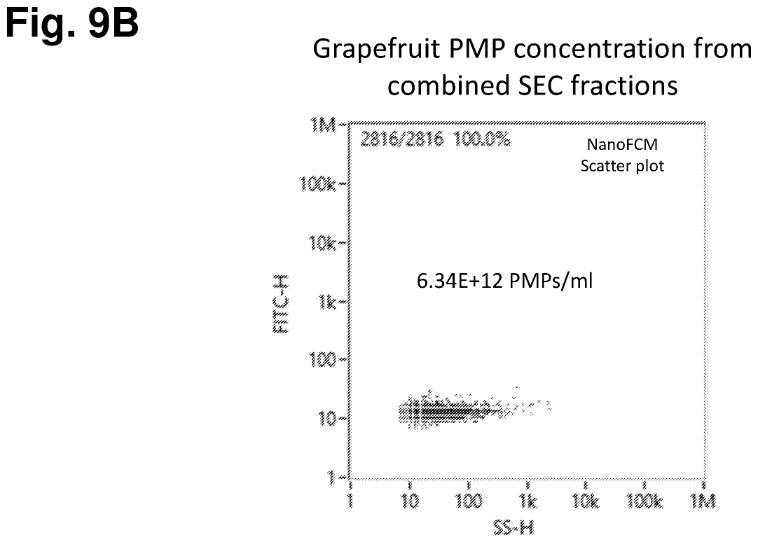

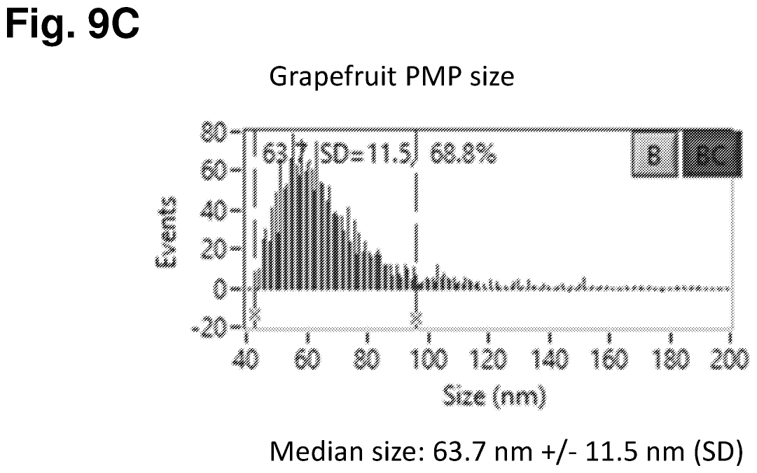

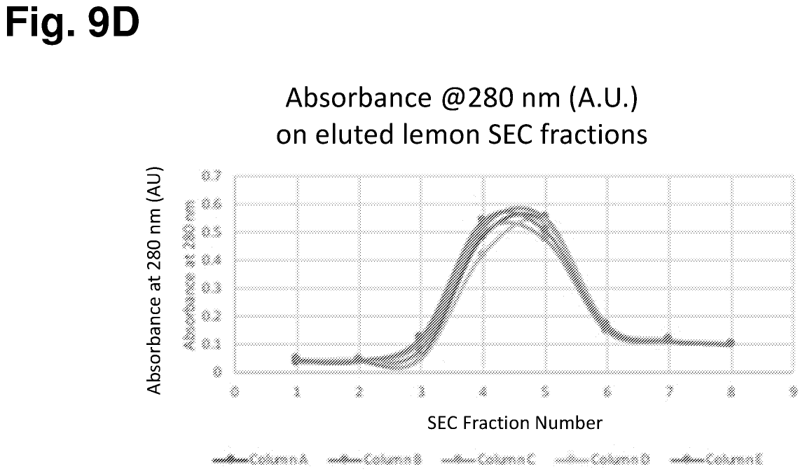

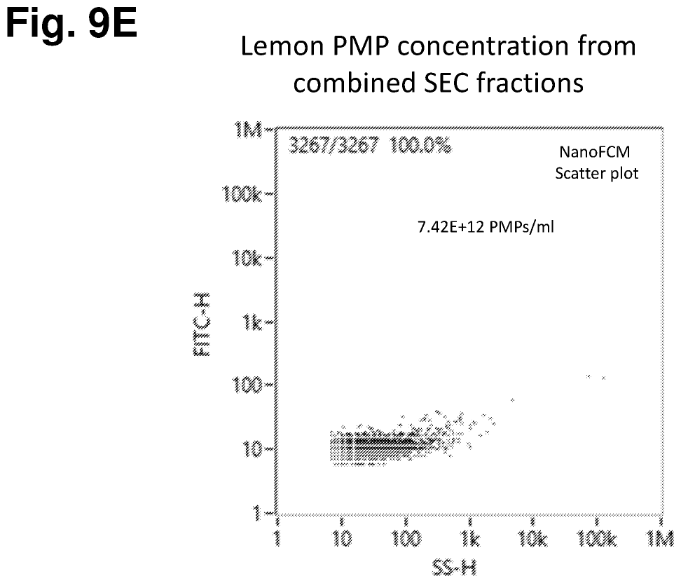

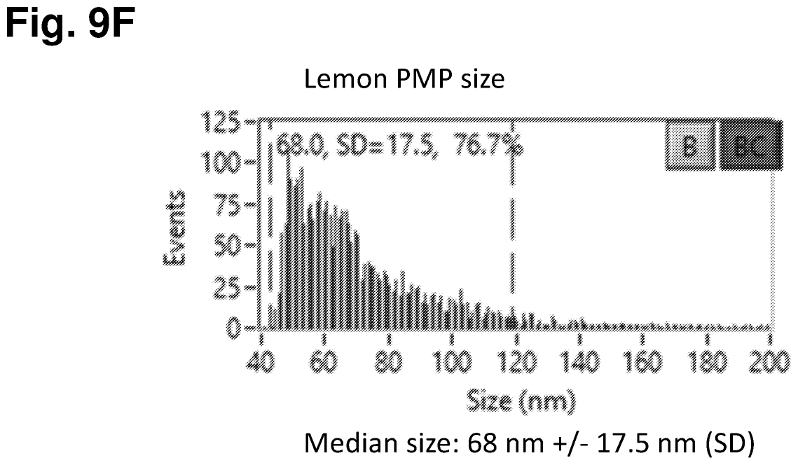

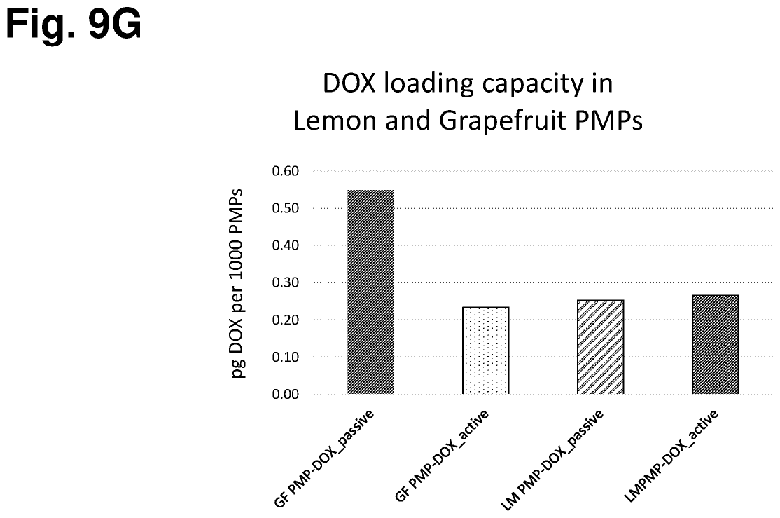

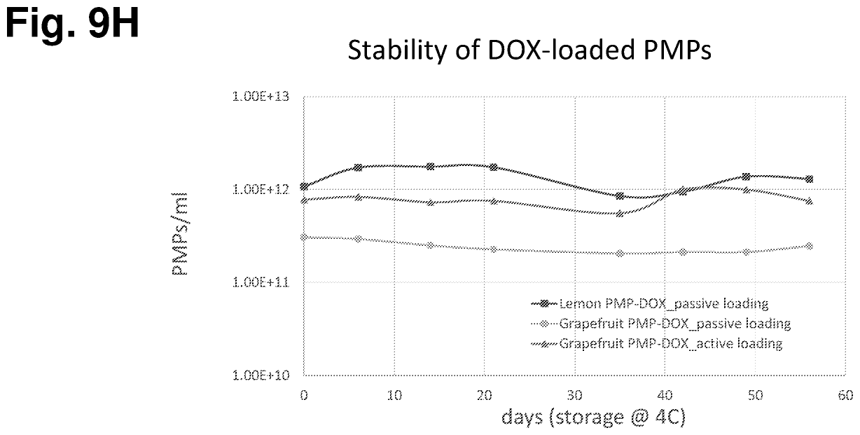

White grapefruits (Florida) were obtained from a local Whole Foods Market®. One liter of grapefruit juice was collected using a juice press, and was subsequently centrifuged at 3000×g for 20 minutes, followed by 10,000×g for 40 minutes to remove large debris. Next, 500 mM EDTA pH8.6 was added to final concentration of 50 mM EDTA, pH7 and incubated for 30 minutes to chelate calcium and prevent the formation of pectin macromolecules. Subsequently the juice was passaged through a coffee filter and 1 μm and 0.45 pm filters to remove large particles. Filtered juice was concentrated by Tangential Flow Filtration (TFF, 5 nm pore size) to 400 ml and dialyzed overnight in PBS pH 7.4 using a 300 kDa dialysis membrane to remove contaminants. Subsequently, the dialyzed juice was further concentrated by TFF to a final concentration of 50 ml (20×). Next, we used size exclusion chromatography to elute the PMP-containing fractions, which were analyzed by 280 nm absorbance (SpectraMax®) to verify the PMP-containing fractions and late fractions containing contaminants (FIG. 9A). SEC fractions 4-6 containing purified PMPs were pooled together, and concentrated further by pelleting PMPs for 1.5 hrs at 40,000×g and resuspending the pellet in Ultrapure water. The final PMP concentration (6.34×10¹² PMPs/ml) and median PMP size (63.7 nm+/−11.5 nm (SD)) were determined by NanoFCM, using concentration and size standards provided by the manufacturer (FIGS. 9B and 9C).

b) Production of Lemon PMPs Using TFF Combined with SEC

Lemons were obtained from a local Whole Foods Market®. One liter of lemon juice was collected using a juice press, and was subsequently centrifuged at 3000 g for 20 minutes, followed by 10,000 g for 40 minutes to remove large debris. Next, 500 mM EDTA pH8.6 was added to final concentration of 50 mM EDTA, pH7 and incubated for 30 minutes to chelate calcium and prevent the formation of pectin macromolecules. Subsequently the juice was passaged through a coffee filter, 1 um and 0.45 um filters to remove large particles. Filtered juice was concentrated by Tangential Flow Filtration (TFF, 5 nm pore size) to 400 ml and dialyzed overnight in PBS pH 7.4 using a 300 kDa dialysis membrane to remove contaminants. Subsequently, the dialyzed juice was further concentrated by TFF to a final concentration of 50 ml (20×). Next, we used size exclusion chromatography to elute the PMP-containing fractions, which were analyzed by 280 nm absorbance (SpectraMax®) to verify the PMP-containing fractions and late fractions containing contaminants (FIG. 9D). SEC fractions 4-6 containing purified PMPs were pooled together, and concentrated further by pelleting PMPs for 1.5 hrs at 40,000×g and resuspending the pellet in Ultrapure water. Final PMP concentration (7.42×10¹² PMPs/ml) and median PMP size (68 nm+/−17.5 nm (SD)) were determined by NanoFCM, using concentration and size standards provided by the manufacturer (FIGS. 9E and 9F).

c) Passive Loading of Doxorubicin in Lemon and Grapefruit PMPs

Grapefruit (Example 22a) and lemon (Example 22b) PMPs were used for loading doxorubicin (DOX). A stock solution of doxorubicin (DOX, Sigma PHR1789) was prepared at a concentration of 10 mg/mL in Ultrapure water (UltraPure™ DNase/RNase-Free Distilled Water, ThermoFisher, 10977023), filter sterilized (0.22 pm), and stored at 4° C. 0.5 mL of PMPs were mixed with 0.25 mL of DOX solution. The final DOX concentration in the mixture was 3.3 mg/mL. The initial particle concentration for grapefruit (GF) PMPs was 9.8×10¹² PMPs/mL and for lemon (LM) PMPs was 1.8×10¹³ PMPs/mL. The mixture was agitated for 4 hours at 25° C., 100 rpm, in the dark. Then the mixture was diluted 3.3 times with UltraPure water (the final concentration of DOX in the mixture was 1 mg/ml) and split into two equals parts (1.25 mL for passive loading, and 1.25 mL for active loading (Example 22c/). Both samples were incubated for an additional 23h at 25° C., 100 rpm, in the dark. All steps were carried out under sterile conditions.

For passive loading of DOX, to remove unloaded or weakly bound DOX, the sample was purified by ultracentrifugation. The mixture was split into 6 equal parts (200 uL each) and sterile water (1.3 mL) was added. Samples were spun down (40,000×g, 1.5 h, 4° C.) in 1.5 mL ultracentrifuge tubes. The PMP-DOX pellets were resuspended in sterile water and spun down twice. Samples were kept at 4° C. for three days. Prior to use, DOX-loaded PMPs were washed one more time by ultracentrifugation (40,000×g, 1.5 h, 4° C.). The final pellet was resuspended in sterile UltraPure water and stored at 4° C. until further use. The concentration of DOX in PMPs was determined by a SpectraMax spectrophotometer (Ex/Em=485/550 nm) and concentration of the total number of particles was determined by nano-flow cytometry (NanoFCM).

d) Active Loading of Doxorubicin in Lemon and Grapefruit PMPs

Grapefruit (Example 22a) and lemon (Example 22b) PMPs were used for loading doxorubicin (DOX). A stock solution of doxorubicin (DOX, Sigma PHR1789) was prepared at a concentration of 10 mg/mL in UltraPure water (ThermoFisher, 10977023), sterilized (0.22 um), and stored at 4° C. 0.5 mL of PMPs were mixed with 0.25 mL of DOX solution. The final DOX concentration in the mixture was 3.3 mg/mL. The initial particle concentration for grapefruit (GF) PMPs was 9.8×10¹² PMPs/mL and for lemon (LM) PMPs was 1.8×10¹³ PMPs/mL. The mixture was agitated for 4 hours at 25° C., 100 rpm, in the dark. Then the mixture was diluted 3.3 times with UltraPure water (the final concentration of DOX in the mixture was 1 mg/ml) and split into two equals parts (1.25 mL for passive loading (Example 22c), and 1.25 mL for active loading). Both samples were incubated for additional 23h at 25° C., 100 rpm, in the dark. All steps were carried out under sterile conditions.

After incubation at 25° C. for a day, the mixture was kept at 4° C. for 4 days. Then the mixture was sonicated for 30 min in a sonication bath (Branson 2800) at 42° C., vortexed, and sonicated once more for 20 min. Next, the mixture was diluted two times with sterile water and extruded using an Avanti Mini Extruder (Avanti Lipids). To reduce the number of lipid bilayers and overall particle size, the DOX-loaded PMPs were extruded in a decreasing stepwise fashion: 800 nm, 400 nm and 200 nm for grapefruit (GF) PMPs; and 800 nm, 400 nm for lemon (LM) PMPs. To remove unloaded or weakly bound DOX, the samples were washed using an ultracentrifugation approach. Specifically, the sample (1.5 mL) was diluted with sterile UltraPure water (6.5 mL total) and was spun down twice at 40,000×g for 1 h at 4° C. in 7 mL ultracentrifuge tubes. The final pellet was resuspended in sterile UltraPure water and kept at 4° C. until further use.

e) Determination of the Loading Capacity of DOX-Loaded PMPs Prepared by Passive and Active Loading

To assess the loading capacity of DOX in PMPs, DOX concentration was assessed by fluorescence intensity measurement (Ex/Em=485/550 nm) using a SpectraMax® spectrophotometer. A calibration curve of free DOX from 0 to 83.3 ug/mL was used. To dissociate DOX-loaded PMPs and DOX complexes (π-π stacking), samples and standards were incubated with 1% SDS at 37° C. for 30 min prior to fluorescence measurements. Loading capacity (pg DOX per 1000 particles) was calculated as concentration of DOX (pg/mL) divided by the total concentration of PMPs (PMPs/mL) (FIG. 9G). The loading capacity for passively loaded PMPs was 0.55 μg DOX (GF PMP-DOX) and 0.25 μg DOX (LM PMP-DOX) for 1000 PMPs. The loading capacity for actively loaded PMPs was 0.23 μg DOX (GF PMP-DOX) and 0.27 μg DOX (LM PMP-DOX) for 1000 PMPs.

f) Stability of Doxorubicin-Loaded Grapefruit and Lemon PMPs

The stability of DOX-loaded PMPs was assessed by measurement of concentration of total PMPs (PMP/ml) in the sample over time using NanoFCM. The stability study was carried out at 4° C. for eight weeks in the dark. Aliquots of PMP-DOX were stored at 4° C. and analyzed by NanoFCM on predetermined days. The particle size of PMP-DOX did not change significantly. Thus, for passively loaded GF PMPs the range of average particle sizes was 70-80 nm over two months. Concentrations of total PMPs in the sample were analyzed (FIG. 9H). The range of concentrations for passively loaded GF PMPs was from 2.06×10¹¹ to 3.06×10¹¹ PMPs/ml, for actively loaded GF PMPs was from 5.55×10¹¹ to 9.97×10¹¹ PMPs/ml, and for passively loaded LM PMPs was from 8.52×10¹¹ to 1.76×10¹² PMPs/ml over eight weeks at 4° C. Our data indicate that DOX-loaded PMPs are stable for 8 weeks at 4° C.

### Example 23: Treatment of Bacteria and Fungi with Small Molecule-Loaded PMPs

This example demonstrates the ability of PMPs to be loaded with a small molecule with the purpose of decreasing the fitness of pathogenic bacteria and fungi. In this example, grapefruit PMPs are used as a model PMP, E. coli and P. aeruginosa are used as model pathogenic bacteria, the yeast S. cerevisiae is used as a model pathogenic fungus, and doxorubicin is used as a model small molecule. Doxorubicin is a cytotoxic anthracycline antibiotic isolated from cultures of Streptomyces peucetius var. caesius. Doxorubicin interacts with DNA by intercalation and inhibits both DNA replication and RNA transcription. Doxorubicin has been shown to have antibiotic activity (Westrnan et al., Chem Biol, 19(10): 1255-1264, 2012.)

a) Production of Grapefruit PMPs Using TFF Combined with SEC

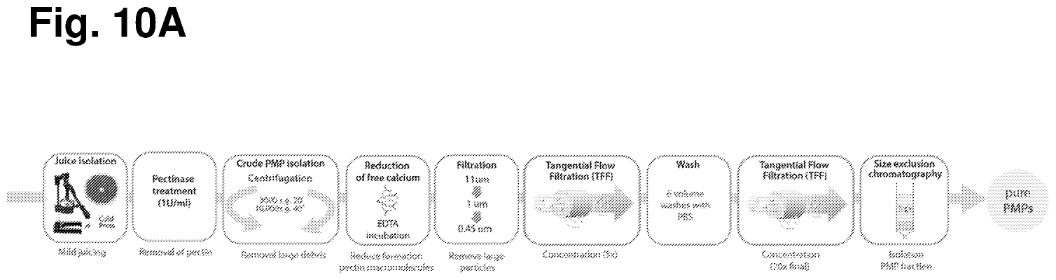

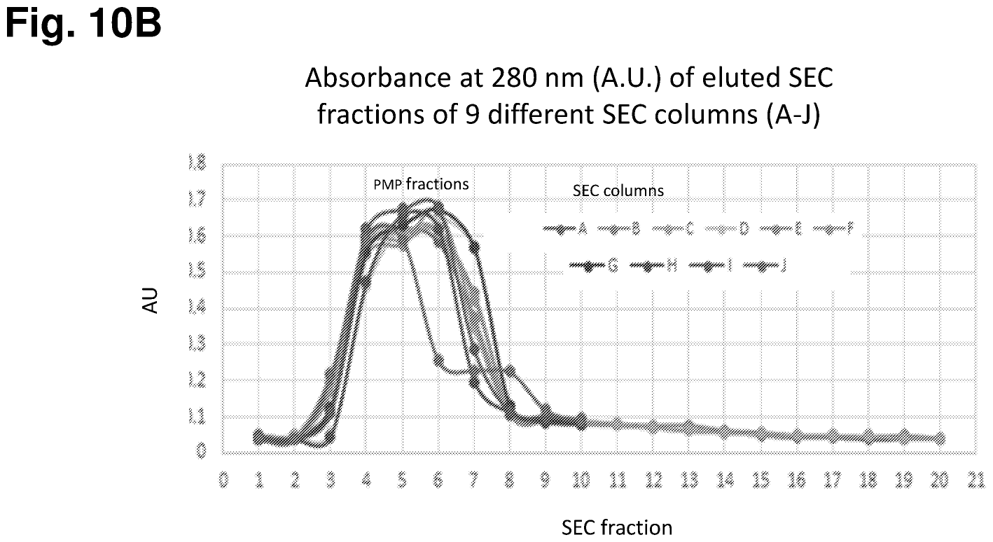

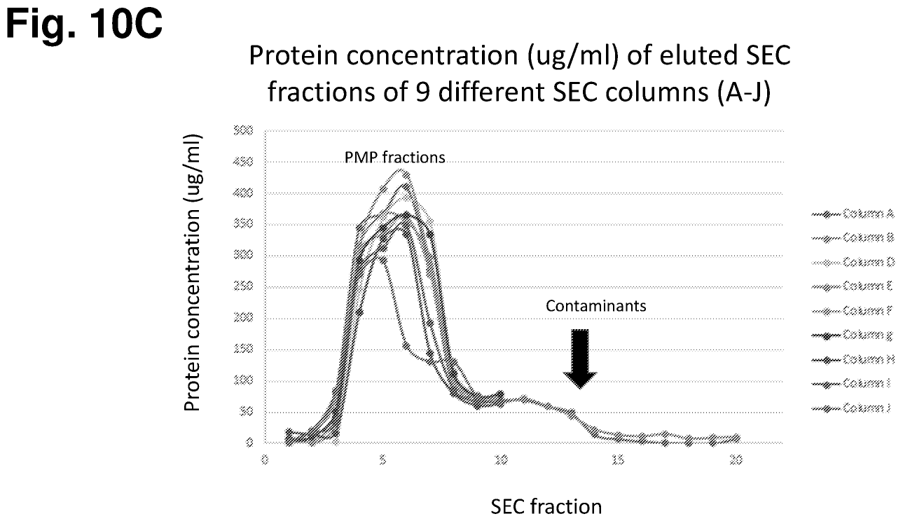

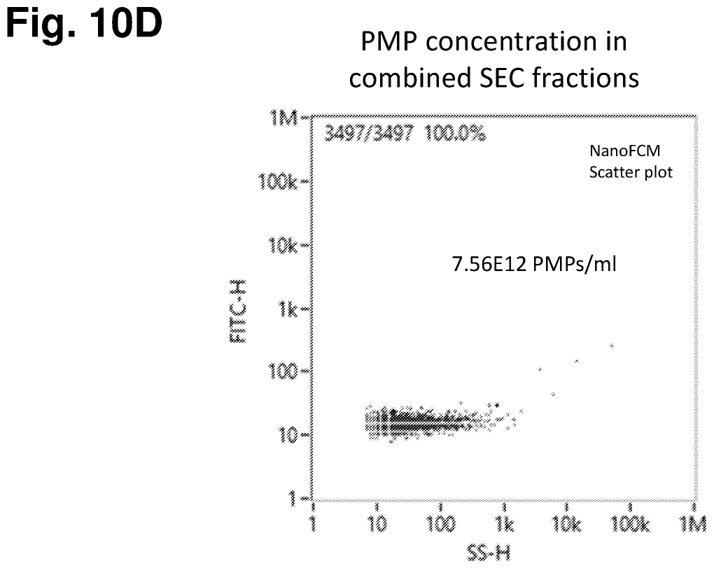

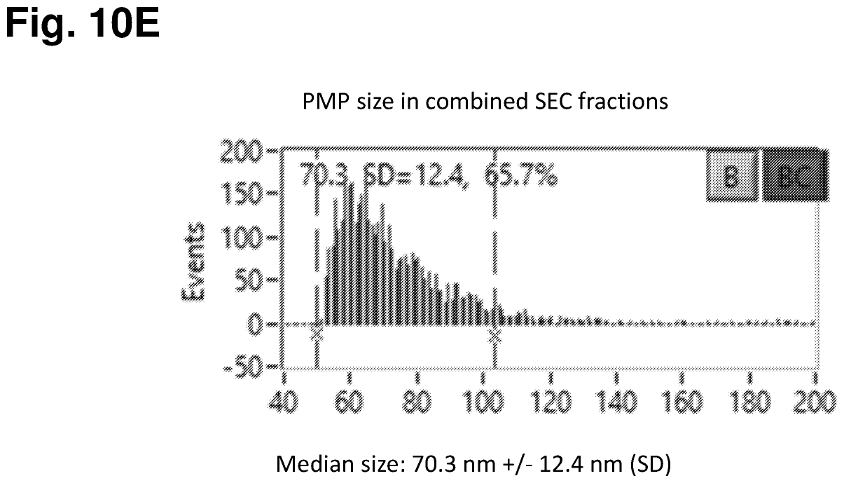

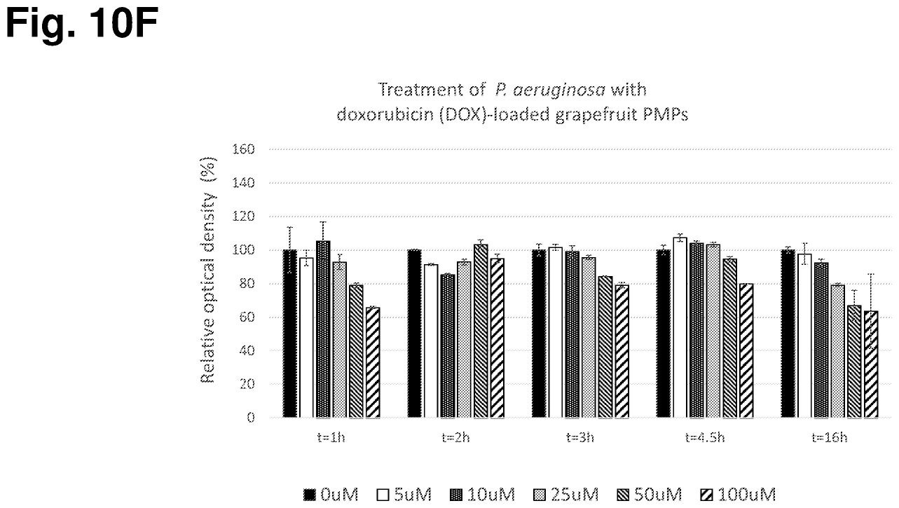

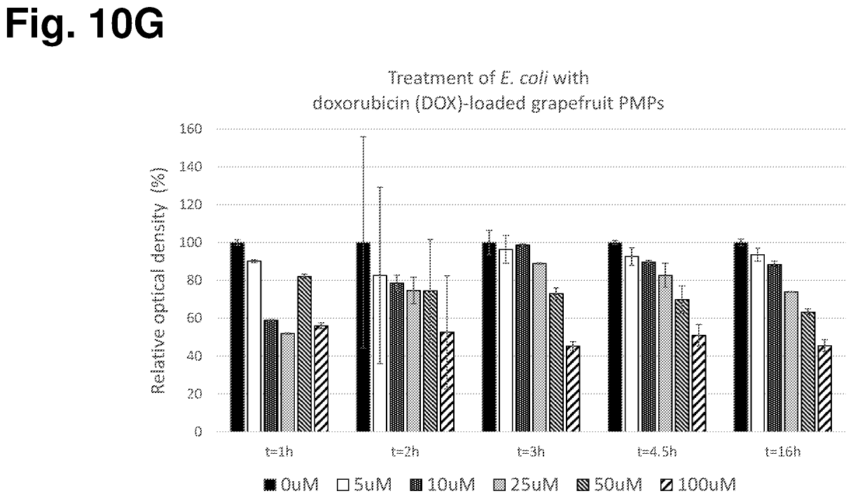

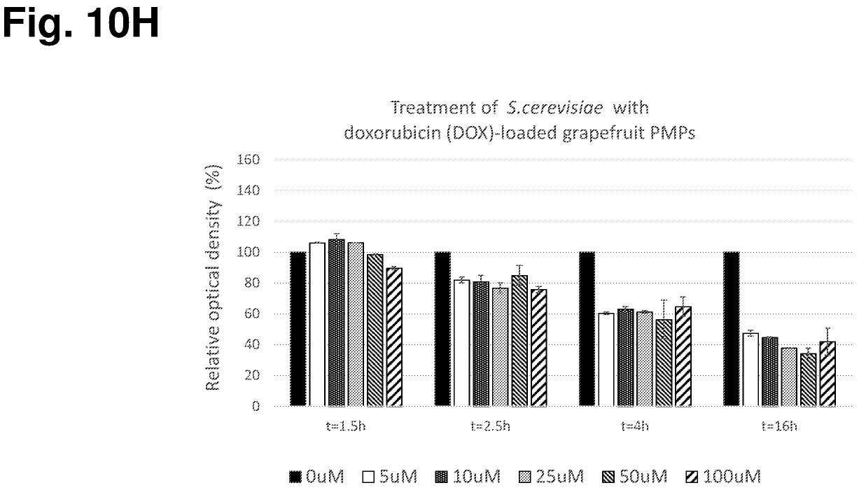

Red organic grapefruits were obtained from a local Whole Foods Market®. An overview of the PMP production workflow is given in FIG. 10A. Four liters of grapefruit juice were collected using a juice press, pH adjusted to pH4 with NaOH, incubated with 1 U/ml pectinase (Sigma, 17389) to remove pectin contaminants, and subsequently centrifuged at 3,000 g for 20 minutes, followed by 10,000 g for 40 minutes to remove large debris. Next, the processed juice was incubated with 500 mM EDTA pH8.6, to a final concentration of 50 mM EDTA, pH7.7 for 30 minutes to chelate calcium and prevent the formation of pectin macromolecules. Subsequently, the EDTA-treated juice was passaged through an 11 μm, 1 μm and 0.45 μm filter to remove large particles. Filtered juice was washed and concentrated by Tangential Flow Filtration (TFF) using a 300 kDa TFF. Juice was concentrated 5×, followed by a 6 volume exchange wash with PBS, and further filtrated to a final concentration 198 mL (20×). Next, we used size exclusion chromatography to elute the PMP-containing fractions, which were analyzed by absorbance at 280 nm (SpectraMax®) and protein concentration (Piercer™ BCA assay, ThermoFisher) to verify the PMP-containing fractions and late fractions containing contaminants (FIGS. 10B and 10C). SEC fractions 3-7 contained purified PMPs (fractions 9-12 contained contaminants), were pooled together, were filter sterilized by sequential filtration using 0.8 μm, 0.45 μm and 0.22 μm syringe filters, and were concentrated further by pelleting PMPs for 1.5 hrs at 40,000×g and resuspending the pellet in 4 ml UltraPure™ DNase/RNase-Free Distilled Water (ThermoFisher, 10977023). Final PMP concentration (7.56×10¹² PMPs/ml) and average PMP size (70.3 nm+/−12.4 nm SD) were determined by NanoFCM, using concentration and size standards provided by the manufacturer (FIGS. 10D and 10E). The produced grapefruit PMPs were used for loading doxorubicin.

b) Loading of Doxorubicin in Grapefruit PMPs

Grapefruit PMPs produced in Example 23a were used for loading doxorubicin (DOX). A stock solution of doxorubicin (Sigma PHR1789) was prepared at a concentration of 10 mg/mL in UltraPure water and filter sterilized (0.22 μm). Sterile grapefruit PMPs (3 mL at particle concentration of 7.56×10¹² PMPs/ml) were mixed with the 1.29 mL of DOX solution. The final DOX concentration in the mixture was 3 mg/mL. The mixture was sonicated for 20 min in a sonication bath (Branson 2800) with temperature rising to 40° C. and kept an additional 15 minutes in the bath without sonication. The mixture was agitated for 4 hours at 24° C., 100 rpm, in the dark. Next, the mixture was extruded using Avanti Mini Extruder (Avanti Lipids). To reduce the number of lipid bilayers and overall particle size, the DOX-loaded PMPs were extruded in a decreasing stepwise fashion: 800 nm, 400 nm and 200 nm. The extruded sample was filter sterilized by subsequent passage through a 0.8 μm and 0.45 μm filter (Millipore, diameter 13 mm) in a TC hood. To remove unloaded or weakly-bound DOX, the sample was purified using an ultracentrifugation approach. Specifically, the sample was spun down at 100,000×g for 1h at 4° C. in 1.5 mL ultracentrifuge tubes. The supernatant was collected for further analysis and stored at 4° C. The pellet was resuspended in sterile water and ultracentrifuged under the same conditions. This step was repeated four times. The final pellet was resuspended in sterile UltraPure water and kept at 4° C. until further use.

Next, the concentration of particles and the loading capacity of PMPs was determined. The total number of PMPs in the sample (4.76×10¹² PMP/ml) and the median particle size (72.8 nm+/−21 nm SD) were determined using a NanoFCM. The DOX concentration was assessed by fluorescence intensity measurement (Ex/Em=485/550 nm) using a SpectraMax® spectrophotometer. A calibration curve of free DOX from 0 to 50 ug/mL was prepared in sterile water. To dissociate DOX-loaded PMPs and DOX complexes (π-π stacking), samples and standards were incubated with 1% SDS at 37° C. for 45 min prior to fluorescence measurements. The loading capacity (pg DOX per 1000 particles) was calculated as the concentration of DOX (pg/ml) divided by the total number of PMPs (PMPs/ml). The PMP-DOX loading capacity was 1.2 μg DOX per 1000 PMPs. However, it should be noted that the loading efficiency (the % of DOX-loaded PMPs compared to the total number of PMPs) could not be assessed as the DOX fluorescence spectrum could not be detected on the NanoFCM.

Our results indicate that PMPs can be efficiently loaded with a small molecule.

c) Treatment of Bacteria and Yeast with Dox-Loaded Grapefruit PMPs

To establish that PMPs can deliver a cytotoxic agent, several microbe species were treated with Doxorubicin-loaded grapefruit PMPs (PMP-DOX) from Example 23b.

Bacteria and yeast strains were maintained as indicated by the supplier: E. coli (ATCC, #25922) was grown on Trypticase Soy Agar/broth at 37° C., Pseudomonas aeruginosa (ATCC) was grown on Tryptic soy Agar/broth with 50 mg/ml rifampicin at 37° C., and Saccharomyces cerevisiae (ATCC, #9763) was grown on yeast extract peptone dextrose broth (YPD) and maintained at 30° C. Prior to treatment, fresh one day cultures were grown overnight, the OD (600 nm) was adjusted to 0.1 OD with medium prior to use, and bacteria/yeast were transferred to a 96 well plate for treatment (duplicate samples, 100 μl/well). Bacteria/yeast were treated with a 50 μl PMP-DOX solution in Ultrapure water to an effective DOX concentration of 0 (negative control), 5 μm, 10 μM, 25 μM, 50 μM and 100 μM (final volume per well was 150 μl). Plates were covered with aluminum foil, and incubated at 37° C. (E. coli, P. aeruginosa), or 30° C. (S. cerevisiae) and agitated at 220 rpm.

A kinetic Absorbance measurement at 600 nm was performed on a SpectraMax® spectrophotometer to monitor the OD of the cultures at t=0h, t=1h, t=2h, t=3h, t=4.5h, t=16h (E. coli, P. aeruginosa) or t=0.5h, t=1.5h, t=2.5h, t=3.5h, t=4h, t=16h (S. cerevisiae). Since doxorubicin has a broad fluorescence spectrum that partially bleeds into the 600 nm absorbance at a high DOX concentration, all OD values per treatment dose were first normalized to the OD of the first time point at that dose (t=0 for E. coli, P. aeruginosa, t=0.5 for S. cerevisiae). To compare the cytotoxic effect of PMP-DOX treatment on different bacterial and yeast strains, within each treatment group the relative OD was determined as compared to the untreated control (set to 100%). All microbe species tested showed a varying degree of cytotoxixity induced by PMP-DOX (FIGS. 10E-101), which was dose dependent except in S. cerevisiae. S. cerevisiae was the most sensitive to PMP-DOX, already showing a cytotoxic response after 2.5 hrs of treatment, and reaching an 1050 at the lowest effective dose tested (5 uM), 16 hours post-treatment, which is 10× more sensitive than any other microbe tested in this series. From 3 hours after treatment, E. coli reached an 1050 only for 100 M. P. aeruginosa was the least sensitive to PMP-DOX, showing a maximum growth reduction of 37% at effective DOX dosages of 50 and 100 μM. We also tested free doxorubicin and found that using the same dosages, cytotoxicity is induced earlier than with PMP-DOX delivery. This indicates that the small doxorubicin molecule readily diffuses into the unicellular organisms, compared to lipid membrane PMPs which, to release their cargo, need to cross the microbial cell wall and fuse with target cell membranes either directly with the plasma membrane or with the endosomal membrane after endocytic uptake.

Our data shows that PMPs loaded with a small molecule can negatively impact the fitness of a variety of bacteria and yeast.

### Example 24: Treatment of a Microbe with Protein Loaded PMPs

This example demonstrates that PMPs can be exogenously loaded with a protein, PMPs can protect their cargo from degradation, and PMPs can deliver their functional cargo to an organism. In this example, grapefruit PMPs are used as model PMP, Pseudomonas aeruginosa bacteria is used as a model organism, and luciferase protein is used as a model protein.

While protein and peptide-based drugs have great potential to impact the fitness of a wide variety pathogenic bacteria and fungi that are resistant or hard to treat, their deployment has been unsuccessful due to their instability and formulation challenges.

a) Loading of Luciferase Protein into Grapefruit PMPs

Grapefruit PMPs were produced as described in Example 10a. Luciferase (Luc) protein was purchased from LSBio (cat. no. LS-G5533-150) and dissolved in PBS, pH7.4 to a final concentration of 300 μg/mL. Filter-sterilized PMPs were loaded with luciferase protein by electroporation, using a protocol adapted from Rachael W. Sirianni and Bahareh Behkam (eds.), Targeted Drug Delivery: Methods and Protocols, Methods in Molecular Biology, vol. 1831. PMPs alone (PMP control), luciferase protein alone (protein control), or PMP+luciferase protein (protein-loaded PMPs), were mixed with 4.8× electroporation buffer (100% Optiprep (Sigma, D1556) in UltraPure water) to have a final 21% Optiprep concentration in the reaction mix (see Table 3). Protein control was made by mixing luciferase protein with UltraPure water instead of Optiprep (protein control), as the final PMP-Luc pellet was diluted in water. Samples were transferred into chilled cuvettes and electroporated at 0.400 kV, 125 pF (0.125 mF), resistance low 100Ω-high 600Ω with two pulses (4-10 ms) using a Biorad GenePulser®. The reaction was put on ice for 10 minutes, and transferred to a pre-ice chilled 1.5 ml ultracentrifuge tube. All samples containing PMPs were washed 3 times by adding 1.4 ml ultrapure water, followed by ultracentrifugation (100,000×g for 1.5 h at 4° C.). The final pellet was resuspended in a minimal volume of UltraPure water (50 μL) and kept at 4° C. until use. After electroporation, samples containing luciferase protein only were not washed by centrifugation and were stored at 4° C. until use.

To determine the PMP loading capacity, one microliter of Luciferase-loaded PMPs (PMP-Luc) and one microliter of unloaded PMPs were used. To determine the amount of Luciferase protein loaded in the PMPs, a Luciferase protein (LSBio, LS-G5533-150) standard curve was made (10, 30, 100, 300, and 1000 ng). Luciferase activity in all samples and standards was assayed using the ONE-Glo™ luciferase assay kit (Promega, E6110) and measuring luminescence using a SpectraMax® spectrophotometer. The amount of luciferase protein loaded in PMPs was determined using a standard curve of Luciferase protein (LSBio, LS-G5533-150) and normalized to the luminescence in the unloaded PMP sample. The loading capacity (ng luciferase protein per 1 E+9 particles) was calculated as the luciferase protein concentration (ng) divided by the number of loaded PMPs (PMP-Luc). The PMP-Luc loading capacity was 2.76 ng Luciferase protein/1×10⁹ PMPs.

Our results indicate that PMPs can be loaded with a model protein that remains active after encapsulation.

TABLE 3
Luciferase protein loading stragety using electroporation.

| | Luciferase PMP | Luciferase | PMP |
|---|---|---|---|
| | (protein-loaded PMPs) | (protein control) | (PMP control) |
| Luciferase protein (300 μg/mL | 25 | 25 | 0 |
| (μL) | | | |
| Optiprep 100% (μL) | 14.7 | 0 | 14.7 |
| UltraPure water (μL) | 10.3 | 45 | 35.3 |
| PMP GF (PMP stock | 20 | 0 | 20 |
| concentration = 7.56 × 10¹² | | | |
| PMP/mL) | | | |
| Final volume | 70 | 70 | 70 |

Note:
25 μL luciferase is equivalent to 7.5 μg luciferase protein.

b) Treatment of Pseudomonas aeruginosa with Luciferase Protein-Loaded Grapefruit PMPs

Pseudomonas aeruginosa (ATCC) was grown overnight at 30° C. in tryptic soy broth supplemented with 50 ug/ml Rifampicin, according to the supplier's instructions. Pseudomonas aeruginosa cells (total volume of 5 ml) were collected by centrifugation at 3,000×g for 5 min. Cells were washed twice with 10 ml 10 mM MgCl₂ and resuspended in 5 ml 10 mM MgCl₂. The OD600 was measured and adjusted to 0.5.

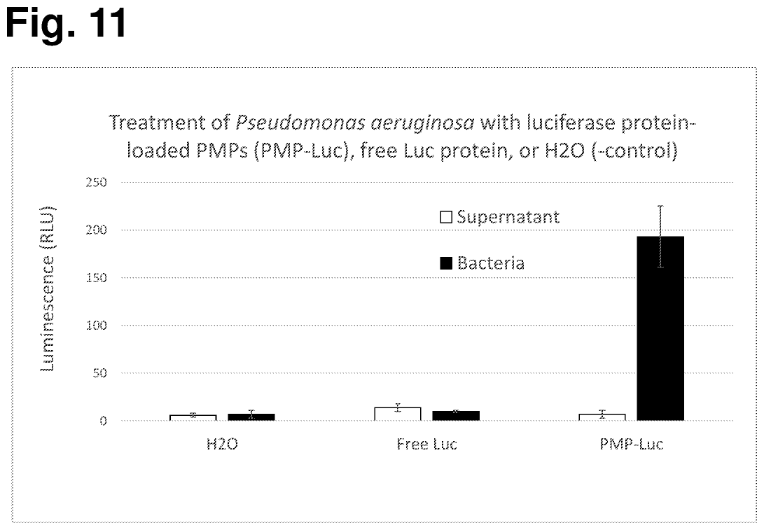

Treatments were performed in duplicate in 1.5 ml Eppendorf tubes, containing 50 μl of the resuspended Pseudomonas aeruginosa cells supplemented with either 3 ng of PMP-Luc (diluted in Ultrapure water), 3 ng free luciferase protein (protein only control; diluted in Ultrapure water), or Ultrapure water (negative control). Ultrapure water was added to 75 μl in all samples. Samples were mixed and incubated at room temperature for 2 h and covered with aluminum foil. Samples were next centrifuged at 6,000×g for 5 min, and 70 μl of the supernatant was collected and saved for luciferase detection. The bacterial pellet was subsequently washed three times with 500 μI 10 mM MgCl₂ containing 0.5% Triton X-100 to remove/burst PMPs that were not taken up. A final wash with 1 ml 10 mM MgCl₂ was performed to remove residual Triton X-100. 970 μI of the supernatant was removed (leaving the pellet in 30 ul wash buffer) and 20 μl 10 mM MgCl₂ and 25 μl Ultrapure water were added to resuspend the Pseudomonas aeruginosa pellets. Luciferase protein was measured by luminescence using the ONE-Glo™ luciferase assay kit (Promega, E6110), according to the manufacturer's instructions. Samples (bacterial pellet and supernatant samples) were incubated for 10 minutes, and luminescence was measured on a SpectraMax® spectrophotometer. Pseudomonas aeruginosa treated with Luciferase protein-loaded grapefruit PMPs had a 19.3 fold higher luciferase expression than treatment with free luciferase protein alone or the Ultrapure water control (negative control), indicating that PMPs are able to efficiently deliver their protein cargo into bacteria (FIG. 11). In addition, PMPs appear to protect luciferase protein from degradation, as free luciferase protein levels in both the supernatant and bacterial pellets are very low. Considering the treatment dose was 3 ng luciferase protein, based on the luciferase protein standard curve, free luciferase protein in supernatant or bacterial pellets after 2 hours of RT incubation in water corresponds to <0.1 ng luciferase protein, indicating protein degradation.

Our data shows that PMPs can deliver a protein cargo into organisms, and that PMPs can protect their cargo from degradation by the environment.

### Other Embodiments

Some embodiments of the invention are within the following numbered paragraphs.
1. A pathogen control composition comprising a plurality of PMPs, wherein each of the plurality of PMPs comprises a heterologous pathogen control agent and wherein the composition is formulated for delivery to an agricultural or veterinary animal pathogen or a vector thereof.
2. The pathogen control composition of paragraph 1, wherein the heterologous pathogen control agent is an antibacterial agent, an antifungal agent, a virucidal agent, an anti-viral agent, an insecticidal agent, a nematicidal agent, an antiparasitic agent, or an insect repellent.
3. The pathogen control composition of paragraph 2, wherein the antibacterial agent is doxorubicin. 4. The pathogen control composition of paragraph 2, wherein the antibacterial agent is an antibiotic.
5. The pathogen control composition of paragraph 4, wherein the antibiotic is vancomycin.
6. The pathogen control composition of paragraph 4, wherein the antibiotic is a penicillin, a cephalosporin, a monobactam, a carbapenem, a macrolide, an aminoglycoside, a quinolone, a sulfonamide, a tetracycline, a glycopeptide, a lipoglycopeptide, an oxazolidinone, a rifamycin, a tuberactinomycin, chloramphenicol, metronidazole, tinidazole, nitrofurantoin, teicoplanin, telavancin, linezolid, cycloserine 2, bacitracin, polymyxin B, viomycin, or capreomycin.
7. The pathogen control composition of paragraph 2, wherein the antifungal agent is an allylamine, an imidazole, a triazole, a thiazole, a polyene, or an echinocandin.
8. The pathogen control composition of paragraph 2, wherein the insecticidal agent is a chloronicotinyl, a neonicotinoid, a carbamate, an organophosphate, a pyrethroid, an oxadiazine, a spinosyn, a cyclodiene, an organochlorine, a fiprole, a mectin, a diacylhydrazine, a benzoylurea, an organotin, a pyrrole, a dinitroterpenol, a METI, a tetronic acid, a tetramic acid, or a pthalamide.
9. The pathogen control composition of paragraph 1, wherein the heterologous pathogen control agent is a small molecule, a nucleic acid, or a polypeptide.
10. The pathogen control composition of paragraph 9, wherein the small molecule is an antibiotic or a secondary metabolite.
11. The pathogen control composition of paragraph 9, wherein the nucleic acid is an inhibitory RNA.
12. The pathogen control composition of any one of paragraphs 1-11, wherein the heterologous pathogen control agent is encapsulated by each of the plurality of PMPs.
13. The pathogen control composition of any one of paragraphs 1-11, wherein the heterologous pathogen control agent is embedded on the surface of each of the plurality of PMPs.
14. The pathogen control composition of any one of paragraphs 1-11, wherein the heterologous pathogen control agent is conjugated to the surface of each of the plurality of PMPs.
15. The pathogen control composition of any one of paragraphs 1-14, wherein each of the plurality of PMPs further comprises an additional pathogen control agent.
16. The pathogen control composition of any one of paragraphs 1-15, wherein the pathogen is a bacterium, a fungus, a parasitic insect, a parasitic nematode, or a parasitic protozoan.
17. The pathogen control composition of paragraph 16, wherein the bacterium is a Pseudomonas species, an Escherichia species, a Streptococcus species, a Pneumococcus species, a Shigella species, a Salmonella species, or a Campylobacter species.
18. The pathogen control composition of paragraph 17, wherein the Pseudomonas species is Pseudomonas aeruginosa.
19. The pathogen control composition of paragraph 17, wherein the Escherichia species is Escherichia coli.
20. The pathogen control composition of paragraph 16, wherein the fungus is a Saccharomyces species or a Candida species.
21. The pathogen control composition of paragraph 16, wherein the parasitic insect is a Cimex species.
22. The pathogen control composition of paragraph 16, wherein the parasitic nematode is a Heligmosomoides species.
23. The pathogen control composition of paragraph 16, wherein the parasitic protozoan is a Trichomonas species.
24. The pathogen control composition of paragraph 1, wherein the vector is an insect.
25. The pathogen control composition of paragraph 24, wherein the vector is a mosquito, a tick, a mite, or a louse.
26. The pathogen control composition of any one of paragraphs 1-25, wherein the composition is stable for at least one day at room temperature, and/or stable for at least one week at 4° C.
27. The pathogen control composition of any one of paragraphs 1-26, wherein the PMPs are stable for at least 24 hours, 48 hours, seven days, or 30 days at 4° C.
28. The pathogen control composition of paragraph 27, wherein the PMPs are stable at a temperature of at least 20° C., 24° C., or 37° C.
29. The pathogen control composition of any one of paragraphs 1-23 or 26-28, wherein the plurality of PMPs in the composition is at a concentration effective to decrease the fitness of an animal pathogen.
30. The pathogen control composition of any one of paragraphs 1-15 or 24-28, wherein the plurality of PMPs in the composition is at a concentration effective to decrease the fitness of an animal pathogen vector.
31. The pathogen control composition of any one of paragraphs 1-23 or 26-30, wherein the plurality of PMPs in the composition is at a concentration effective to treat an infection in an animal infected with a pathogen.
32. The pathogen control composition of any one of paragraphs 1-23 or 26-30, wherein the plurality of PMPs in the composition is at a concentration effective to prevent an infection in an animal at risk of an infection with a pathogen.
33. The pathogen control composition of any one of paragraphs 1-32, wherein the plurality of PMPs in the composition is at a concentration of at least 0.01 ng, 0.1 ng, 1 ng, 2 ng, 3 ng, 4 ng, 5 ng, 10 ng, 50 ng, 100 ng, 250 ng, 500 ng, 750 ng, 1 μg, 10 μg, 50 μg, 100 μg, or 250 μg PMP protein/ml.
34. The pathogen control composition of any one of paragraphs 1-33, wherein the composition comprises an agriculturally acceptable carrier.
35. The pathogen control composition of any one of paragraphs 1-34, wherein the composition comprises a pharmaceutically acceptable carrier.
36. The pathogen control composition of any one of paragraphs 1-35, wherein the composition is formulated to stabilize the PMPs.
37. The pathogen control composition of any one of paragraphs 1-36, wherein the composition is formulated as a liquid, a solid, an aerosol, a paste, a gel, or a gas composition.
38. The pathogen control composition of any one of paragraphs 1-37, wherein the composition comprises at least 5% PMPs.
39. A pathogen control composition comprising a plurality of PMPs, wherein the PMPs are isolated from a plant by a process which comprises the steps of:
   (a) providing an initial sample from a plant, or a part thereof, wherein the plant or part thereof comprises EVs;
   (b) isolating a crude PMP fraction from the initial sample, wherein the crude PMP fraction has a decreased level of at least one contaminant or undesired component from the plant or part thereof relative to the level in the initial sample;
   (c) purifying the crude PMP fraction, thereby producing a plurality of pure PMPs, wherein the plurality of pure PMPs have a decreased level of at least one contaminant or undesired component from the plant or part thereof relative to the level in the crude EV fraction;
   (d) loading the plurality of PMPs of step (c) with a pathogen control agent; and
   (e) formulating the PMPs of step (d) for delivery to an agricultural or veterinary animal pathogen or a vector thereof.
40. An animal pathogen comprising the pathogen control composition of any one of paragraphs 1-39.
41. An animal pathogen vector comprising the pathogen control composition of any one of paragraphs 1-40.
42. A method of delivering a pathogen control composition to an animal comprising administering to the animal the composition of any one of paragraphs 1-39.
43. A method of treating an infection in an animal in need thereof, the method comprising administering to the animal an effective amount of the composition of any one of paragraphs 1-39.
44. A method of preventing an infection in an animal at risk thereof, the method comprising administering to the animal an effective amount of the composition of any one of paragraphs 1-39, wherein the method decreases the likelihood of the infection in the animal relative to an untreated animal.
45. The method of any one of paragraphs 42-44, wherein the infection is caused by a pathogen, and the pathogen is a bacterium, a fungus, a virus, a parasitic insect, a parasitic nematode, or a parasitic protozoan.
46. The method of paragraph 45, wherein the bacterium is a Pseudomonas species, an Escherichia species, a Streptococcus species, a Pneumococcus species, a Shigella species, a Salmonella species, or a Campylobacter species.
47. The method of paragraph 45, wherein the fungus is a Saccharomyces species or a Candida species.
48. The method of paragraph 45, wherein the parasitic insect is a Cimex species.
49. The method of paragraph 45, wherein the parasitic nematode is a Heligmosomoides species.
50. The method of paragraph 45, wherein the parasitic protozoan is a Trichomonas species.
51. The method of any one of paragraphs 42-50, wherein the pathogen control composition is administered to the animal orally, intravenously, or subcutaneously.
52. A method of delivering a pathogen control composition to a pathogen comprising contacting the pathogen with the composition of any one of paragraphs 1-39.
53. A method of decreasing the fitness of a pathogen, the method comprising delivering to the pathogen the composition of any one of paragraphs 1-39, wherein the method decreases the fitness of the pathogen relative to an untreated pathogen.
54. The method of paragraph 52 or 53, wherein the method comprises delivering the composition to at least one habitat where the pathogen grows, lives, reproduces, feeds, or infests.
55. The method of any one of paragraphs 52-54, wherein the composition is delivered as a pathogen comestible composition for ingestion by the pathogen.
56. The method of any one of paragraphs 52-55, wherein the pathogen is a bacterium, a fungus, a parasitic insect, a parasitic nematode, or a parasitic protozoan.
57. The method of paragraph 56, wherein the bacterium is a Pseudomonas species, an Escherichia species, a Streptococcus species, a Pneumococcus species, a Shigella species, a Salmonella species, or a Campylobacter species.
58. The method of paragraph 56, wherein the fungus is a Saccharomyces species or a Candida species.
59. The method of paragraph 56, wherein the parasitic insect is a Cimex species.
60. The method of paragraph 56, wherein the parasitic nematode is a Heligmosomoides species.
61. The method of paragraph 56, wherein the parasitic protozoan is a Trichomonas species.
62. The method of any one of paragraphs 52-61, wherein the composition is delivered as a liquid, a solid, an aerosol, a paste, a gel, or a gas.
63. A method of decreasing the fitness of an animal pathogen vector, the method comprising delivering to the vector an effective amount of the composition of any one of paragraphs 1-39, wherein the method decreases the fitness of the vector relative to an untreated vector.
64. The method of paragraph 63, wherein the method comprises delivering the composition to at least one habitat where the vector grows, lives, reproduces, feeds, or infests.
65. The method of paragraph 63 or 64, wherein the composition is delivered as a comestible composition for ingestion by the vector.
66. The method of any one of paragraphs 63-65, wherein the vector is an insect.
67. The method of paragraph 66, wherein the insect is a mosquito, a tick, a mite, or a louse.
68. The method of any one of paragraphs 63-67, wherein the composition is delivered as a liquid, a solid, an aerosol, a paste, a gel, or a gas.
69. A method of treating an animal having a fungal infection, wherein the method comprises administering to the animal an effective amount of a pathogen control composition comprising a plurality of PMPs.
70. A method of treating an animal having a fungal infection, wherein the method comprises administering to the animal an effective amount of a pathogen control composition comprising a plurality of PMPs, and wherein the plurality of PMPs comprises an antifungal agent.
71. The method of paragraph 70, wherein the antifungal agent is a nucleic acid that inhibits expression of a gene in a fungus that causes the fungal infection.
72. The method of paragraph 71, wherein the gene is Enhanced Filamentous Growth Protein (EFG1).
73. The method of any one of paragraphs 70-72, wherein the fungal infection is caused by Candida albicans.
74. The method of any one of paragraphs 70-73, wherein the composition comprises a PMP derived from Arabidopsis.
75. The method of any one of paragraphs 70-74, wherein the method decreases or substantially eliminates the fungal infection.
76. A method of treating an animal having a bacterial infection, wherein the method comprises administering to the animal an effective amount of a pathogen control composition comprising a plurality of PMPs.
77. A method of treating an animal having a bacterial infection, wherein the method comprises administering to the animal an effective amount of a pathogen control composition comprising a plurality of PMPs, and wherein the plurality of PMPs comprises an antibacterial agent.
78. The method of paragraph 77, wherein the antibacterial agent is Amphotericin B.
79. The method of paragraph 77 or 78, wherein the bacterium is a Pseudomonas species, an Escherichia species, a Streptococcus species, a Pneumococcus species, a Shigella species, a Salmonella species, or a Campylobacter species.
80. The method of any one of paragraphs 77-79, wherein the composition comprises a PMP derived from Arabidopsis.
81. The method of any one of paragraphs 77-80, wherein the method decreases or substantially eliminates the bacterial infection.
82. The method of any one of paragraphs 69-81, wherein the animal is a veterinary animal, or a livestock animal.
83. A method of decreasing the fitness of a parasitic insect, wherein the method comprises delivering to the parasitic insect a pathogen control composition comprising a plurality of PMPs.
84. A method of decreasing the fitness of a parasitic insect, wherein the method comprises delivering to the parasitic insect a pathogen control composition comprising a plurality of PMPs, and wherein the plurality of PMPs comprise an insecticidal agent.
85. The method of paragraph 84, wherein the insecticidal agent is a peptide nucleic acid. 86. The method of any one of paragraphs 83-85, wherein the parasitic insect is a bedbug. 87. The method of any one of paragraphs 83-86, wherein the method decreases the fitness of the parasitic insect relative to an untreated parasitic insect.
88. A method of decreasing the fitness of a parasitic nematode, wherein the method comprises delivering to the parasitic nematode a pathogen control composition comprising a plurality of PMPs.
89. A method of decreasing the fitness of a parasitic nematode, wherein the method comprises delivering to the parasitic nematode a pathogen control composition comprising a plurality of PMPs, and wherein the plurality of PMPs comprises a nematicidal agent.
90. The method of paragraph 88 or 89, wherein the parasitic nematode is Heligmosomoides polygyrus.
91. The method of any one of paragraphs 88-90, wherein the method decreases the fitness of the parasitic nematode relative to an untreated parasitic nematode.
92. A method of decreasing the fitness of a parasitic protozoan, wherein the method comprises delivering to the parasitic protozoan a pathogen control composition comprising a plurality of PMPs.
93. A method of decreasing the fitness of a parasitic protozoan, wherein the method comprises delivering to the parasitic protozoan a pathogen control composition comprising a plurality of PMPs, and wherein the plurality of PMPs comprises an antiparasitic agent.
94. The method of paragraph 92 or 93, wherein the parasitic protozoan is T. vaginalis.
95. The method of any one of paragraphs 92-94, wherein the method decreases the fitness of the parasitic protozoan relative to an untreated parasitic protozoan.
96. A method of decreasing the fitness of an insect vector of an animal pathogen, wherein the method comprises delivering to the vector a pathogen control composition comprising a plurality of PMPs.
97. A method of decreasing the fitness of an insect vector of an animal pathogen, wherein the method comprises delivering to the vector a pathogen control composition comprising a plurality of PMPs, and wherein the plurality of PMPs comprises an insecticidal agent.
98. The method of paragraph 96 or 97, wherein the method decreases the fitness of the vector relative to an untreated vector.
99. The method of any one of paragraphs 96-98, wherein the insect is a mosquito, tick, mite, or louse.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference. Other embodiments are within the claims.

### APPENDIX

TABLE 1
Plant EV-Markers

| Example Species | Accession No. | Protein Name |
|---|---|---|
| Arabidopsis thaliana | C0LGG8 | Probable LRR receptor-like serine/threonine-protein kinase |
| | | At1g53430 (EC 2.7.11.1) |
| Arabidopsis thaliana | F4HQT8 | Uncharacterized protein |
| Arabidopsis thaliana | F4HWU0 | Protein kinase superfamily protein |
| Arabidopsis thaliana | F4I082 | Bifunctional inhibitor/lipid-transfer protein/seed storage 2S |
| | | albumin superfamily protein |
| Arabidopsis thaliana | F4I3M3 | Kinase with tetratricopeptide repeat domain-containing |
| | | protein |
| Arabidopsis thaliana | F4IB62 | Leucine-rich repeat protein kinase family protein |
| Arabidopsis thaliana | O03042 | Ribulose bisphosphate carboxylase large chain (RuBisCO |
| | | large subunit) (EC 4.1.1.39) |
| Arabidopsis thaliana | O03986 | Heat shock protein 90-4 (AtHSP90.4) (AtHsp90-4) (Heat |
| | | shock protein 81-4) (Hsp81-4) |
| Arabidopsis thaliana | O04023 | Protein SRC2 homolog (AtSRC2) |
| Arabidopsis thaliana | O04309 | Jacalin-related lectin 35 (JA-responsive protein 1) |
| | | (Myrosinase-binding protein-like At3g16470) |
| Arabidopsis thaliana | O04314 | PYK10-binding protein 1 (Jacalin-related lectin 30) (Jasmonic |
| | | acid-induced protein) |
| Arabidopsis thaliana | O04922 | Probable glutathione peroxidase 2 (EC 1.11.1.9) |
| Arabidopsis thaliana | O22126 | Fasciclin-like arabinogalactan protein 8 (AtAGP8) |
| Arabidopsis thaliana | O23179 | Patatin-like protein 1 (AtPLP1 (EC 3.1.1.—) (Patatin-related |
| | | phospholipase A IIgamma) (pPLAIIg) (Phospholipase A IVA) |
| | | (AtPLAIVA) |
| Arabidopsis thaliana | O23207 | Probable NAD(P)H dehydrogenase (quinone) FQR1-like 2 |
| | | (EC 1.6.5.2) |
| Arabidopsis thaliana | O23255 | Adenosylhomocysteinase 1 (AdoHcyase 1) (EC 3.3.1.1) |
| | | (Protein EMBRYO DEFECTIVE 1395) (Protein |
| | | HOMOLOGY-DEPENDENT GENE SILENCING 1) |
| | | (S-adenosyl-L-homocysteine hydrolase 1) |
| | | (SAH hydrolase 1) |
| Arabidopsis thaliana | O23482 | Oligopeptide transporter 3 (AtOPT3) |
| Arabidopsis thaliana | O23654 | V-type proton ATPase catalytic subunit A (V-ATPase subunit |
| | | A) (EC 3.6.3.14) (V-ATPase 69 kDa subunit) (Vacuolar H(+)- |
| | | ATPase subunit A) (Vacuolar proton pump subunit alpha) |
| Arabidopsis thaliana | O48788 | Probable inactive receptor kinase At2g26730 |
| Arabidopsis thaliana | O48963 | Phototropin-1 (EC 2.7.11.1) (Non-phototropic hypocotyl |
| | | protein 1) (Root phototropism protein 1) |
| Arabidopsis thaliana | O49195 | Vegetative storage protein 1 |
| Arabidopsis thaliana | O50008 | 5-methyltetrahydropteroyltriglutamate--homocysteine |
| | | methyltransferase 1 (EC 2.1.1.14) (Cobalamin-independent |
| | | methionine synthase 1) (AtMS1) (Vitamin-B12-independent |
| | | methionine synthase 1) |
| Arabidopsis thaliana | O64696 | Putative uncharacterized protein At2g34510 |
| Arabidopsis thaliana | O65572 | Carotenoid 9,10(9′,10′)-cleavage dioxygenase 1 (EC |
| | | 1.14.99.n4) (AtCCD1) (Neoxanthin cleavage enzyme NC1) |
| | | (AtNCED1) |
| Arabidopsis thaliana | O65660 | PLAT domain-containing protein 1 (AtPLAT1) (PLAT domain |
| | | protein 1) |
| Arabidopsis thaliana | O65719 | Heat shock 70 kDa protein 3 (Heat shock cognate 70 kDa |
| | | protein 3) (Heat shock cognate protein 70-3) (AtHsc70-3) |
| | | (Heat shock protein 70-3) (AtHsp70-3) |
| Arabidopsis thaliana | O80517 | Uclacyanin-2 (Blue copper-binding protein II) (BCB II) |
| | | (Phytocyanin 2) (Uclacyanin-II) |
| Arabidopsis thaliana | O80576 | At2g44060 (Late embryogenesis abundant protein, group 2) |
| | | (Similar to late embryogenesis abundant proteins) |
| Arabidopsis thaliana | O80725 | ABC transporter B family member 4 (ABC transporter |
| | | ABCB.4) (AtABCB4) (Multidrug resistance protein 4) |
| | | (P-glycoprotein 4) |
| Arabidopsis thaliana | O80837 | Remorin (DNA-binding protein) |
| Arabidopsis thaliana | O80852 | Glutathione S-transferase F9 (AtGSTF9) (EC 2.5.1.18) |
| | | (AtGSTF7) (GST class-phi member 9) |
| Arabidopsis thaliana | O80858 | Expressed protein (Putative uncharacterized protein |
| | | At2g30930) (Putative uncharacterized protein At2g30930; |
| | | F7F1.14) |
| Arabidopsis thaliana | O80939 | L-type lectin-domain containing receptor kinase IV.1 |
| | | (Arabidopsis thaliana lectin-receptor kinase e) (AthlecRK-e) |
| | | (LecRK-IV.1) (EC 2.7.11.1) (Lectin Receptor Kinase 1) |
| Arabidopsis thaliana | O80948 | Jacalin-related lectin 23 (Myrosinase-binding protein-like |
| | | At2g39330) |
| Arabidopsis thaliana | O82628 | V-type proton ATPase subunit G1 (V-ATPase subunit G1) |
| | | (Vacuolar H(+)-ATPase subunit G isoform 1) (Vacuolar |
| | | proton pump subunit G1) |
| Arabidopsis thaliana | P10795 | Ribulose bisphosphate carboxylase small chain 1A, |
| | | chloroplastic (RuBisCO small subunit 1A) (EC 4.1.1.39) |
| Arabidopsis thaliana | P10896 | Ribulose bisphosphate carboxylase/oxygenase activase, |
| | | chloroplastic (RA) (RuBisCO activase) |
| Arabidopsis thaliana | P17094 | 60S ribosomal protein L3-1 (Protein EMBRYO DEFECTIVE |
| | | 2207) |
| Arabidopsis thaliana | P19456 | ATPase 2, plasma membrane-type (EC 3.6.3.6) (Proton |
| | | pump 2) |
| Arabidopsis thaliana | P20649 | ATPase 1, plasma membrane-type (EC 3.6.3.6) (Proton |
| | | pump 1) |
| Arabidopsis thaliana | P22953 | Probable mediator of RNA polymerase II transcription subunit |
| | | 37e (Heat shock 70 kDa protein 1) (Heat shock cognate 70 |
| | | kDa protein 1) (Heat shock cognate protein 70-1) (AtHsc70-1) |
| | | (Heat shock protein 70-1) (AtHsp70-1) (Protein |
| | | EARLY-RESPONSIVE TO DEHYDRATION 2) |
| Arabidopsis thaliana | P23586 | Sugar transport protein 1 (Glucose transporter) (Hexose |
| | | transporter 1) |
| Arabidopsis thaliana | P24636 | Tubulin beta-4 chain (Beta-4-tubulin) |
| Arabidopsis thaliana | P25696 | Bifunctional enolase 2/transcriptional activator (EC 4.2.1.11) |
| | | (2-phospho-D-glycerate hydro-lyase 2) (2-phosphoglycerate |
| | | dehydratase 2) (LOW EXPRESSION OF OSMOTICALLY |
| | | RESPONSIVE GENES 1) |
| Arabidopsis thaliana | P25856 | Glyceraldehyde-3-phosphate dehydrogenase GAPA1, |
| | | chloroplastic (EC 1.2.1.13) (NADP-dependent |
| | | glyceraldehydephosphate dehydrogenase A subunit 1) |
| Arabidopsis thaliana | P28186 | Ras-related protein RABE1c (AtRABE1c) (Ras-related |
| | | protein Ara-3) (Ras-related protein Rab8A) (AtRab8A) |
| Arabidopsis thaliana | P30302 | Aquaporin PIP2-3 (Plasma membrane intrinsic protein 2-3) |
| | | (AtPIP2; 3) (Plasma membrane intrinsic protein 2c) (PIP2c) |
| | | (RD28-PIP) (TMP2C) (Water stress-induced tonoplast |
| | | intrinsic protein) (WSI-TIP) [Cleaved into: Aquaporin PIP2-3, |
| | | N-terminally processed] |
| Arabidopsis thaliana | P31414 | Pyrophosphate-energized vacuolar membrane proton pump |
| | | 1 (EC 3.6.1.1) (Pyrophosphate-energized inorganic |
| | | pyrophosphatase 1) (H(+)-PPase 1) (Vacuolar proton |
| | | pyrophosphatase 1) (Vacuolar proton pyrophosphatase 3) |
| Arabidopsis thaliana | P32961 | Nitrilase 1 (EC 3.5.5.1) |
| Arabidopsis thaliana | P38666 | 60S ribosomal protein L24-2 (Protein SHORT VALVE 1) |
| Arabidopsis thaliana | P39207 | Nucleoside diphosphate kinase 1 (EC 2.7.4.6) (Nucleoside |
| | | diphosphate kinase I) (NDK I) (NDP kinase I) (NDPK I) |
| Arabidopsis thaliana | P42643 | 14-3-3-like protein GF14 chi (General regulatory factor 1) |
| Arabidopsis thaliana | P42737 | Beta carbonic anhydrase 2, chloroplastic (AtbCA2) |
| | | (AtbetaCA2) (EC 4.2.1.1) (Beta carbonate dehydratase 2) |
| Arabidopsis thaliana | P42759 | Dehydrin ERD10 (Low-temperature-induced protein LTI45) |
| Arabidopsis thaliana | P42761 | Glutathione S-transferase F10 (AtGSTF10) (EC 2.5.1.18) |
| | | (AtGSTF4) (GST class-phi member 10) (Protein EARLY |
| | | RESPONSE TO DEHYDRATION 13) |
| Arabidopsis thaliana | P42763 | Dehydrin ERD14 |
| Arabidopsis thaliana | P42791 | 60S ribosomal protein L18-2 |
| Arabidopsis thaliana | P43286 | Aquaporin PIP2-1 (Plasma membrane intrinsic protein 2-1) |
| | | (AtPIP2; 1) (Plasma membrane intrinsic protein 2a) (PIP2a) |
| | | [Cleaved into: Aquaporin PIP2-1, N-terminally processed] |
| Arabidopsis thaliana | P46286 | 60S ribosomal protein L8-1 (60S ribosomal protein L2) |
| | | (Protein EMBRYO DEFECTIVE 2296) |
| Arabidopsis thaliana | P46422 | Glutathione S-transferase F2 (AtGSTF2) (EC 2.5.1.18) (24 |
| | | kDa auxin-binding protein) (AtPM24) (GST class-phi member |
| | | 2) |
| Arabidopsis thaliana | P47998 | Cysteine synthase 1 (EC 2.5.1.47) (At.OAS.5-8) (Beta-substituted |
| | | Ala synthase 1; 1) (ARAth-Bsas1; 1) (CSase A) |
| | | (AtCS-A) (Cys-3A) (O-acetylserine (thiol)-lyase 1) (OAS-TL |
| | | A) (O-acetylserine sulfhydrylase) (Protein ONSET OF LEAF |
| | | DEATH 3) |
| Arabidopsis thaliana | P48347 | 14-3-3-like protein GF14 epsilon (General regulatory factor |
| | | 10) |
| Arabidopsis thaliana | P48491 | Triosephosphate isomerase, cytosolic (TIM) (Triose-phosphate |
| | | isomerase) (EC 5.3.1.1) |
| Arabidopsis thaliana | P50318 | Phosphoglycerate kinase 2, chloroplastic (EC 2.7.2.3) |
| Arabidopsis thaliana | P53492 | Actin-7 (Actin-2) |
| Arabidopsis thaliana | P54144 | Ammonium transporter 1 member 1 (AtAMT1; 1) |
| Arabidopsis thaliana | P92963 | Ras-related protein RABB1c (AtRABB1c) (Ras-related |
| | | protein Rab2A) (AtRab2A) |
| Arabidopsis thaliana | P93004 | Aquaporin PIP2-7 (Plasma membrane intrinsic protein 2-7) |
| | | (AtPIP2; 7) (Plasma membrane intrinsic protein 3) (Salt |
| | | stress-induced major intrinsic protein) [Cleaved into: |
| | | Aquaporin PIP2-7, N-terminally processed] |
| Arabidopsis thaliana | P93025 | Phototropin-2 (EC 2.7.11.1) (Defective in chloroplast |
| | | avoidance protein 1) (Non-phototropic hypocotyl 1-like |
| | | protein 1) (AtKin7) (NPH1-like protein 1) |
| Arabidopsis thaliana | P93819 | Malate dehydrogenase 1, cytoplasmic (EC 1.1.1.37) |
| | | (Cytosolic NAD-dependent malate dehydrogenase 1) |
| | | (cNAD-MDH1) (Cytosolic malate dehydrogenase 1) |
| | | (Cytosolic MDH1) |
| Arabidopsis thaliana | Q03250 | Glycine-rich RNA-binding protein 7 (AtGR-RBP7) (AtRBG7) |
| | | (Glycine-rich protein 7) (AtGRP7) (Protein COLD, |
| | | CIRCADIAN RHYTHM, AND RNA BINDING 2) (Protein |
| | | CCR2) |
| Arabidopsis thaliana | Q05431 | L-ascorbate peroxidase 1, cytosolic (AP) (AtAPx01) (EC |
| | | 1.11.1.11) |
| Arabidopsis thaliana | Q06611 | Aquaporin PIP1-2 (AtPIP1; 2) (Plasma membrane intrinsic |
| | | protein 1b) (PIP1b) (Transmembrane protein A) (AthH2) |
| | | (TMP-A) |
| Arabidopsis thaliana | Q07488 | Blue copper protein (Blue copper-binding protein) (AtBCB) |
| | | (Phytocyanin 1) (Stellacyanin) |
| Arabidopsis thaliana | Q0WLB5 | Clathrin heavy chain 2 |
| Arabidopsis thaliana | Q0WNJ6 | Clathrin heavy chain 1 |
| Arabidopsis thaliana | Q1ECE0 | Vesicle-associated protein 4-1 (Plant VAP homolog 4-1) |
| | | (AtPVA41) (Protein MEMBRANE-ASSOCIATED MANNITOL-INDUCED) |
| | | (AtMAMI) (VAMP-associated protein 4-1) |
| Arabidopsis thaliana | Q38882 | Phospholipase D alpha 1 (AtPLDalpha1) (PLD alpha 1) (EC |
| | | 3.1.4.4) (Choline phosphatase 1) (PLDalpha) |
| | | (Phosphatidylcholine-hydrolyzing phospholipase D 1) |
| Arabidopsis thaliana | Q38900 | Peptidyl-prolyl cis-trans isomerase CYP19-1 (PPIase CYP19-1) |
| | | (EC 5.2.1.8) (Cyclophilin of 19 kDa 1) (Rotamase |
| | | cyclophilin-3) |
| Arabidopsis thaliana | Q39033 | Phosphoinositide phospholipase C 2 (EC 3.1.4.11) |
| | | (Phosphoinositide phospholipase PLC2) (AtPLC2) (PI-PLC2) |
| Arabidopsis thaliana | Q39085 | Delta(24)-sterol reductase (EC 1.3.1.72) (Cell elongation |
| | | protein DIMINUTO) (Cell elongation protein Dwarf1) (Protein |
| | | CABBAGE1) (Protein ENHANCED VERY-LOW-FLUENCE |
| | | RESPONSE 1) |
| Arabidopsis thaliana | Q39228 | Sugar transport protein 4 (Hexose transporter 4) |
| Arabidopsis thaliana | Q39241 | Thioredoxin H5 (AtTrxh5) (Protein LOCUS OF |
| | | INSENSITIVITY TO VICTORIN 1) (Thioredoxin 5) (AtTRX5) |
| Arabidopsis thaliana | Q39258 | V-type proton ATPase subunit E1 (V-ATPase subunit E1) |
| | | (Protein EMBRYO DEFECTIVE 2448) (Vacuolar |
| | | H(+)-ATPase subunit E isoform 1) (Vacuolar proton pump subunit |
| | | E1) |
| Arabidopsis thaliana | Q42112 | 60S acidic ribosomal protein P0-2 |
| Arabidopsis thaliana | Q42403 | Thioredoxin H3 (AtTrxh3) (Thioredoxin 3) (AtTRX3) |
| Arabidopsis thaliana | Q42479 | Calcium-dependent protein kinase 3 (EC 2.7.11.1) |
| | | (Calcium-dependent protein kinase isoform CDPK6) (AtCDPK6) |
| Arabidopsis thaliana | Q42547 | Catalase-3 (EC 1.11.1.6) |
| Arabidopsis thaliana | Q56WH1 | Tubulin alpha-3 chain |
| Arabidopsis thaliana | Q56WK6 | Patellin-1 |
| Arabidopsis thaliana | Q56X75 | CASP-like protein 4D2 (AtCASPL4D2) |
| Arabidopsis thaliana | Q56ZI2 | Patellin-2 |
| Arabidopsis thaliana | Q7Y208 | Glycerophosphodiester phosphodiesterase GDPDL1 (EC |
| | | 3.1.4.46) (Glycerophosphodiester phosphodiesterase-like 1) |
| | | (ATGDPDL1) (Glycerophosphodiesterase-like 3) (Protein |
| | | SHV3-LIKE 2) |
| Arabidopsis thaliana | Q84VZ5 | Uncharacterized GPI-anchored protein At5g19240 |
| Arabidopsis thaliana | Q84WU7 | Eukaryotic aspartyl protease family protein (Putative |
| | | uncharacterized protein At3g51330) |
| Arabidopsis thaliana | Q8GUL8 | Uncharacterized GPI-anchored protein At5g19230 |
| Arabidopsis thaliana | Q8GYA4 | Cysteine-rich receptor-like protein kinase 10 (Cysteine-rich |
| | | RLK10) (EC 2.7.11.—) (Receptor-like protein kinase 4) |
| Arabidopsis thaliana | Q8GYN5 | RPM1-interacting protein 4 |
| Arabidopsis thaliana | Q8GZ99 | At5g49760 (Leucine-rich repeat protein kinase family protein) |
| | | (Leucine-rich repeat receptor-like protein kinase) (Putative |
| | | receptor protein kinase) |
| Arabidopsis thaliana | Q8L636 | Sodium/calcium exchanger NCL (Na(+)/Ca(2+)-exchange |
| | | protein NCL) (Protein NCX-like) (AtNCL) |
| Arabidopsis thaliana | Q8L7S1 | At1g45200 (At1g45200/At1g45200) (Triacylglycerol |
| | | lipase-like 1) |
| Arabidopsis thaliana | Q8LAA6 | Probable aquaporin PIP1-5 (AtPIP1; 5) (Plasma membrane |
| | | intrinsic protein 1d) (PIP1d) |
| Arabidopsis thaliana | Q8LCP6 | Endoglucanase 10 (EC 3.2.1.4) (Endo-1,4-beta glucanase |
| | | 10) |
| Arabidopsis thaliana | Q8RWV0 | Transketolase-1, chloroplastic (TK) (EC 2.2.1.1) |
| Arabidopsis thaliana | Q8S8Q6 | Tetraspanin-8 |
| Arabidopsis thaliana | Q8VZG8 | MDIS1-interacting receptor like kinase 2 (AtMIK2) (Probable |
| | | LRR receptor-like serine/threonine-protein kinase |
| | | At4g08850) (EC 2.7.11.1) |
| Arabidopsis thaliana | Q8VZU2 | Syntaxin-132 (AtSYP132) |
| Arabidopsis thaliana | Q8W4E2 | V-type proton ATPase subunit B3 (V-ATPase subunit B3) |
| | | (Vacuolar H(+)-ATPase subunit B isoform 3) (Vacuolar |
| | | proton pump subunit B3) |
| Arabidopsis thaliana | Q8W4S4 | V-type proton ATPase subunit a3 (V-ATPase subunit a3) |
| | | (V-type proton ATPase 95 kDa subunit a isoform 3) (V-ATPase |
| | | 95 kDa isoform a3) (Vacuolar H(+)-ATPase subunit a isoform |
| | | 3) (Vacuolar proton pump subunit a3) (Vacuolar proton |
| | | translocating ATPase 95 kDa subunit a isoform 3) |
| Arabidopsis thaliana | Q93VG5 | 40S ribosomal protein S8-1 |
| Arabidopsis thaliana | Q93XY5 | Tetraspanin-18 (TOM2A homologous protein 2) |
| Arabidopsis thaliana | Q93YS4 | ABC transporter G family member 22 (ABC transporter |
| | | ABCG.22) (AtABCG22) (White-brown complex homolog |
| | | protein 23) (AtWBC23) |
| Arabidopsis thaliana | Q93Z08 | Glucan endo-1,3-beta-glucosidase 6 (EC 3.2.1.39) |
| | | ((1−>3)-beta-glucan endohydrolase 6) |
| | | ((1−>3)-beta-glucanase 6) (Beta-1,3-endoglucanase |
| | | 6) (Beta-1,3-glucanase 6) |
| Arabidopsis thaliana | Q940M8 | 3-oxo-5-alpha-steroid 4-dehydrogenase (DUF1295) |
| | | (At1g73650/F25P22_7) |
| Arabidopsis thaliana | Q944A7 | Probable serine/threonine-protein kinase At4g35230 (EC |
| | | 2.7.11.1) |
| Arabidopsis thaliana | Q944G5 | Protein NRT1/PTR FAMILY 2.10 (AtNPF2.10) (Protein |
| | | GLUCOSINOLATE TRANSPORTER-1) |
| Arabidopsis thaliana | Q94AZ2 | Sugar transport protein 13 (Hexose transporter 13) |
| | | (Multicopy suppressor of snf4 deficiency protein 1) |
| Arabidopsis thaliana | Q94BT2 | Auxin-induced in root cultures protein 12 |
| Arabidopsis thaliana | Q94CE4 | Beta carbonic anhydrase 4 (AtbCA4) (AtbetaCA4) (EC |
| | | 4.2.1.1) (Beta carbonate dehydratase 4) |
| Arabidopsis thaliana | Q94KI8 | Two pore calcium channel protein 1 (Calcium channel protein |
| | | 1) (AtCCH1) (Fatty acid oxygenation up-regulated protein 2) |
| | | (Voltage-dependent calcium channel protein TPC1) (AtTPC1) |
| Arabidopsis thaliana | Q96262 | Plasma membrane-associated cation-binding protein 1 |
| | | (AtPCAP1) (Microtubule-destabilizing protein 25) |
| Arabidopsis thaliana | Q9C5Y0 | Phospholipase D delta (AtPLDdelta) (PLD delta) (EC 3.1.4.4) |
| Arabidopsis thaliana | Q9C7F7 | Non-specific lipid transfer protein GPI-anchored 1 |
| | | (AtLTPG-1) (Protein LTP-GPI-ANCHORED 1) |
| Arabidopsis thaliana | Q9C821 | Proline-rich receptor-like protein kinase PERK15 (EC |
| | | 2.7.11.1) (Proline-rich extensin-like receptor kinase 15) |
| | | (AtPERK15) |
| Arabidopsis thaliana | Q9C8G5 | CSC1-like protein ERD4 (Protein EARLY-RESPONSIVE TO |
| | | DEHYDRATION STRESS 4) |
| Arabidopsis thaliana | Q9C9C5 | 60S ribosomal protein L6-3 |
| Arabidopsis thaliana | Q9CAR7 | Hypersensitive-induced response protein 2 (AtHIR2) |
| Arabidopsis thaliana | Q9FFH6 | Fasciclin-like arabinogalactan protein 13 |
| Arabidopsis thaliana | Q9FGT8 | Temperature-induced lipocalin-1 (AtTIL1) |
| Arabidopsis thaliana | Q9FJ62 | Glycerophosphodiester phosphodiesterase GDPDL4 (EC |
| | | 3.1.4.46) (Glycerophosphodiester phosphodiesterase-like 4) |
| | | (ATGDPDL4) (Glycerophosphodiesterase-like 1) (Protein |
| | | SHV3-LIKE 1) |
| Arabidopsis thaliana | Q9FK68 | Ras-related protein RABA1c (AtRABA1c) |
| Arabidopsis thaliana | Q9FKS8 | Lysine histidine transporter 1 |
| Arabidopsis thaliana | Q9FM65 | Fasciclin-like arabinogalactan protein 1 |
| Arabidopsis thaliana | Q9FNH6 | NDR1/HIN1-like protein 3 |
| Arabidopsis thaliana | Q9FRL3 | Sugar transporter ERD6-like 6 |
| Arabidopsis thaliana | Q9FWR4 | Glutathione S-transferase DHAR1, mitochondrial (EC |
| | | 2.5.1.18) (Chloride intracellular channel homolog 1) (CLIC |
| | | homolog 1) (Glutathione-dependent dehydroascorbate |
| | | reductase 1) (AtDHAR1) (GSH-dependent dehydroascorbate |
| | | reductase 1) (mtDHAR) |
| Arabidopsis thaliana | Q9FX54 | Glyceraldehyde-3-phosphate dehydrogenase GAPC2, |
| | | cytosolic (EC 1.2.1.12) (NAD-dependent |
| | | glyceraldehydephosphate dehydrogenase C subunit 2) |
| Arabidopsis thaliana | Q9LE22 | Probable calcium-binding protein CML27 (Calmodulin-like |
| | | protein 27) |
| Arabidopsis thaliana | Q9LEX1 | At3g61050 (CaLB protein) (Calcium-dependent lipid-binding |
| | | (CaLB domain) family protein) |
| Arabidopsis thaliana | Q9LF79 | Calcium-transporting ATPase 8, plasma membrane-type (EC |
| | | 3.6.3.8) (Ca(2+)-ATPase isoform 8) |
| Arabidopsis thaliana | Q9LJG3 | GDSL esterase/lipase ESM1 (EC 3.1.1.—) (Extracellular lipase |
| | | ESM1) (Protein EPITHIOSPECIFIER MODIFIER 1) |
| | | (AtESM1) |
| Arabidopsis thaliana | Q9LJI5 | V-type proton ATPase subunit d1 (V-ATPase subunit d1) |
| | | (Vacuolar H(+)-ATPase subunit d isoform 1) (Vacuolar proton |
| | | pump subunit d1) |
| Arabidopsis thaliana | Q9LME4 | Probable protein phosphatase 2C 9 (AtPP2C09) (EC |
| | | 3.1.3.16) (Phytochrome-associated protein phosphatase 2C) |
| | | (PAPP2C) |
| Arabidopsis thaliana | Q9LNP3 | At1g17620/F11A6_23 (F1L3.32) (Late embryogenesis |
| | | abundant (LEA) hydroxyproline-rich glycoprotein family) |
| | | (Putative uncharacterized protein At1g17620) |
| Arabidopsis thaliana | Q9LNW1 | Ras-related protein RABA2b (AtRABA2b) |
| Arabidopsis thaliana | Q9LQU2 | Protein PLANT CADMIUM RESISTANCE 1 (AtPCR1) |
| Arabidopsis thaliana | Q9LQU4 | Protein PLANT CADMIUM RESISTANCE 2 (AtPCR2) |
| Arabidopsis thaliana | Q9LR30 | Glutamate--glyoxylate aminotransferase 1 (AtGGT2) (EC |
| | | 2.6.1.4) (Alanine aminotransferase GGT1) (EC 2.6.1.2) |
| | | (Alanine--glyoxylate aminotransferase GGT1) (EC 2.6.1.44) |
| | | (Alanine-2-oxoglutarate aminotransferase 1) (EC 2.6.1.—) |
| Arabidopsis thaliana | Q9LSI9 | Inactive LRR receptor-like serine/threonine-protein kinase |
| | | BIR2 (Protein BAK1-INTERACTING RECEPTOR-LIKE |
| | | KINASE 2) |
| Arabidopsis thaliana | Q9LSQ5 | NAD(P)H dehydrogenase (quinone) FQR1 (EC 1.6.5.2) |
| | | (Flavodoxin-like quinone reductase 1) |
| Arabidopsis thaliana | Q9LUT0 | Protein kinase superfamily protein (Putative uncharacterized |
| | | protein At3g17410) (Serine/threonine protein kinase-like |
| | | protein) |
| Arabidopsis thaliana | Q9LV48 | Proline-rich receptor-like protein kinase PERK1 (EC 2.7.11.1) |
| | | (Proline-rich extensin-like receptor kinase 1) (AtPERK1) |
| Arabidopsis thaliana | Q9LX65 | V-type proton ATPase subunit H (V-ATPase subunit H) |
| | | (Vacuolar H(+)-ATPase subunit H) (Vacuolar proton pump |
| | | subunit H) |
| Arabidopsis thaliana | Q9LYG3 | NADP-dependent malic enzyme 2 (AtNADP-ME2) |
| | | (NADP-malic enzyme 2) (EC 1.1.1.40) |
| Arabidopsis thaliana | Q9M088 | Glucan endo-1,3-beta-glucosidase 5 (EC 3.2.1.39) |
| | | ((1−>3)-beta-glucan endohydrolase 5) |
| | | ((1−>3)-beta-glucanase 5) (Beta-1,3-endoglucanase |
| | | 5) (Beta-1,3-glucanase 5) |
| Arabidopsis thaliana | Q9M2D8 | Uncharacterized protein At3g61260 |
| Arabidopsis thaliana | Q9M386 | Late embryogenesis abundant (LEA) hydroxyproline-rich |
| | | glycoprotein family (Putative uncharacterized protein |
| | | At3g54200) (Putative uncharacterized protein F24B22.160) |
| Arabidopsis thaliana | Q9M390 | Protein NRT1/PTR FAMILY 8.1 (AtNPF8.1) (Peptide |
| | | transporter PTR1) |
| Arabidopsis thaliana | Q9M5P2 | Secretory carrier-associated membrane protein 3 (AtSC3) |
| | | (Secretory carrier membrane protein 3) |
| Arabidopsis thaliana | Q9M8T0 | Probable inactive receptor kinase At3g02880 |
| Arabidopsis thaliana | Q9SDS7 | V-type proton ATPase subunit C (V-ATPase subunit C) |
| | | (Vacuolar H(+)-ATPase subunit C) (Vacuolar proton pump |
| | | subunit C) |
| Arabidopsis thaliana | Q9SEL6 | Vesicle transport v-SNARE 11 (AtVTI11) (Protein SHOOT |
| | | GRAVITROPISM 4) (Vesicle soluble NSF attachment protein |
| | | receptor VTI1a) (AtVTI1a) (Vesicle transport v-SNARE |
| | | protein VTI1a) |
| Arabidopsis thaliana | Q9SF29 | Syntaxin-71 (AtSYP71) |
| Arabidopsis thaliana | Q9SF85 | Adenosine kinase 1 (AK 1) (EC 2.7.1.20) (Adenosine |
| | | 5′-phosphotransferase 1) |
| Arabidopsis thaliana | Q9SIE7 | PLAT domain-containing protein 2 (AtPLAT2) (PLAT domain |
| | | protein 2) |
| Arabidopsis thaliana | Q9SIM4 | 60S ribosomal protein L14-1 |
| Arabidopsis thaliana | Q9SIU8 | Probable protein phosphatase 2C 20 (AtPP2C20) (EC |
| | | 3.1.3.16) (AtPPC3; 1.2) |
| Arabidopsis thaliana | Q9SJ81 | Fasciclin-like arabinogalactan protein 7 |
| Arabidopsis thaliana | Q9SKB2 | Leucine-rich repeat receptor-like serine/threonine/tyrosine-protein |
| | | kinase SOBIR1 (EC 2.7.10.1) (EC 2.7.11.1) (Protein |
| | | EVERSHED) (Protein SUPPRESSOR OF BIR1-1) |
| Arabidopsis thaliana | Q9SKR2 | Synaptotagmin-1 (NTMC2T1.1) (Synaptotagmin A) |
| Arabidopsis thaliana | Q9SLF7 | 60S acidic ribosomal protein P2-2 |
| Arabidopsis thaliana | Q9SPE6 | Alpha-soluble NSF attachment protein 2 (Alpha-SNAP2) |
| | | (N-ethylmaleimide-sensitive factor attachment protein alpha 2) |
| Arabidopsis thaliana | Q9SRH6 | Hypersensitive-induced response protein 3 (AtHIR3) |
| Arabidopsis thaliana | Q9SRY5 | Glutathione S-transferase F7 (EC 2.5.1.18) (AtGSTF8) (GST |
| | | class-phi member 7) (Glutathione S-transferase 11) |
| Arabidopsis thaliana | Q9SRZ6 | Cytosolic isocitrate dehydrogenase [NADP] (EC 1.1.1.42) |
| Arabidopsis thaliana | Q9SSK5 | MLP-like protein 43 |
| Arabidopsis thaliana | Q9SU13 | Fasciclin-like arabinogalactan protein 2 |
| Arabidopsis thaliana | Q9SU40 | Monocopper oxidase-like protein SKU5 (Skewed roots) |
| Arabidopsis thaliana | Q9SUR6 | Cystine lyase CORI3 (EC 4.4.1.35) (Protein CORONATINE |
| | | INDUCED 3) (Protein JASMONIC ACID RESPONSIVE 2) |
| | | (Tyrosine aminotransferase CORI3) |
| Arabidopsis thaliana | Q9SVC2 | Syntaxin-122 (AtSYP122) (Synt4) |
| Arabidopsis thaliana | Q9SVF0 | Putative uncharacterized protein AT4g38350 (Putative |
| | | uncharacterized protein F22I13.120) |
| Arabidopsis thaliana | Q9SW40 | Major facilitator superfamily protein (Putative uncharacterized |
| | | protein AT4g34950) (Putative uncharacterized protein |
| | | T11I11.190) |
| Arabidopsis thaliana | Q9SYT0 | Annexin D1 (AnnAt1) (Annexin A1) |
| Arabidopsis thaliana | Q9SZ11 | Glycerophosphodiester phosphodiesterase GDPDL3 (EC |
| | | 3.1.4.46) (Glycerophosphodiester phosphodiesterase-like 3) |
| | | (ATGDPDL3) (Glycerophosphodiesterase-like 2) (Protein |
| | | MUTANT ROOT HAIR 5) (Protein SHAVEN 3) |
| Arabidopsis thaliana | Q9SZN1 | V-type proton ATPase subunit B2 (V-ATPase subunit B2) |
| | | (Vacuolar H(+)-ATPase subunit B isoform 2) (Vacuolar |
| | | proton pump subunit B2) |
| Arabidopsis thaliana | Q9SZP6 | AT4g38690/F20M13_250 (PLC-like phosphodiesterases |
| | | superfamily protein) (Putative uncharacterized protein |
| | | AT4g38690) (Putative uncharacterized protein F20M13.250) |
| Arabidopsis thaliana | Q9SZR1 | Calcium-transporting ATPase 10, plasma membrane-type |
| | | (EC 3.6.3.8) (Ca(2+)-ATPase isoform 10) |
| Arabidopsis thaliana | Q9T053 | Phospholipase D gamma 1 (AtPLDgamma1) (PLD gamma 1) |
| | | (EC 3.1.4.4) (Choline phosphatase) (Lecithinase D) |
| | | (Lipophosphodiesterase II) |
| Arabidopsis thaliana | Q9T076 | Early nodulin-like protein 2 (Phytocyanin-like protein) |
| Arabidopsis thaliana | Q9T0A0 | Long chain acyl-CoA synthetase 4 (EC 6.2.1.3) |
| Arabidopsis thaliana | Q9T0G4 | Putative uncharacterized protein AT4g10060 (Putative |
| | | uncharacterized protein T5L19.190) |
| Arabidopsis thaliana | Q9XEE2 | Annexin D2 (AnnAt2) |
| Arabidopsis thaliana | Q9XGM1 | V-type proton ATPase subunit D (V-ATPase subunit D) |
| | | (Vacuolar H(+)-ATPase subunit D) (Vacuolar proton pump |
| | | subunit D) |
| Arabidopsis thaliana | Q9XI93 | At1g13930/F16A14.27 (F16A14.14) (F7A19.2 protein) |
| | | (Oleosin-B3-like protein) |
| Arabidopsis thaliana | Q9XIE2 | ABC transporter G family member 36 (ABC transporter |
| | | ABCG.36) (AtABCG36) (Pleiotropic drug resistance protein |
| | | 8) (Protein PENETRATION 3) |
| Arabidopsis thaliana | Q9ZPZ4 | Putative uncharacterized protein (Putative uncharacterized |
| | | protein At1g09310) (T31J12.3 protein) |
| Arabidopsis thaliana | Q9ZQX4 | V-type proton ATPase subunit F (V-ATPase subunit F) |
| | | (V-ATPase 14 kDa subunit) (Vacuolar H(+)-ATPase subunit F) |
| | | (Vacuolar proton pump subunit F) |
| Arabidopsis thaliana | Q9ZSA2 | Calcium-dependent protein kinase 21 (EC 2.7.11.1) |
| Arabidopsis thaliana | Q9ZSD4 | Syntaxin-121 (AtSYP121) (Syntaxin-related protein At-Syr1) |
| Arabidopsis thaliana | Q9ZV07 | Probable aquaporin PIP2-6 (Plasma membrane intrinsic |
| | | protein 2-6) (AtPIP2; 6) (Plasma membrane intrinsic protein |
| | | 2e) (PIP2e) [Cleaved into: Probable aquaporin PIP2-6, |
| | | N-terminally processed] |
| Arabidopsis thaliana | Q9ZVF3 | MLP-like protein 328 |
| Arabidopsis thaliana | Q9ZWA8 | Fasciclin-like arabinogalactan protein 9 |
| Arabidopsis thaliana | Q9ZSD4 | SYR1, Syntaxin Related Protein 1, also known as SYP121, |
| | | PENETRATION1/PEN1 (Protein PENETRATION 1) |
| Citrus lemon | A1ECK0 | Putative glutaredoxin |
| Citrus lemon | A9YVC9 | Pyrophosphate--fructose 6-phosphate 1-phosphotransferase |
| | | subunit beta (PFP) (EC 2.7.1.90) (6-phosphofructokinase, |
| | | pyrophosphate dependent) (PPi-PFK) |
| | | (Pyrophosphate-dependent 6-phosphofructose-1-kinase) |
| Citrus lemon | B2YGY1 | Glycosyltransferase (EC 2.4.1.—) |
| Citrus lemon | B6DZD3 | Glutathione S-transferase Tau2 (Glutathione transferase |
| | | Tau2) |
| Citrus lemon | C3VIC2 | Translation elongation factor |
| Citrus lemon | C8CPS0 | Importin subunit alpha |
| Citrus lemon | D3JWB5 | Flavanone 3-hydroxylase |
| Citrus lemon | E0ADY2 | Putative caffeic acid O-methyltransferase |
| Citrus lemon | E5DK62 | ATP synthase subunit alpha (Fragment) |
| Citrus lemon | E9M5S3 | Putative L-galactose-1-phosphate phosphatase |
| Citrus lemon | F1CGQ9 | Heat shock protein 90 |
| Citrus lemon | F8WL79 | Aminopeptidase (EC 3.4.11.—) |
| Citrus lemon | F8WL86 | Heat shock protein |
| Citrus lemon | K9JG59 | Abscisic acid stress ripening-related protein |
| Citrus lemon | Q000W4 | Fe(III)-chelate reductase |
| Citrus lemon | Q39538 | Heat shock protein (Fragment) |
| Citrus lemon | Q5UEN6 | Putative signal recognition particle protein |
| Citrus lemon | Q8GV08 | Dehydrin |
| Citrus lemon | Q8L893 | Cytosolic phosphoglucomutase (Fragment) |
| Citrus lemon | Q8S990 | Polygalacturonase-inhibiting protein |
| Citrus lemon | Q8W3U6 | Polygalacturonase-inhibitor protein |
| Citrus lemon | Q93XL8 | Dehydrin COR15 |
| Citrus lemon | Q941Q1 | Non-symbiotic hemoglobin class 1 |
| Citrus lemon | Q9MBF3 | Glycine-rich RNA-binding protein |
| Citrus lemon | Q9SP55 | V-type proton ATPase subunit G (V-ATPase subunit G) |
| | | (Vacuolar proton pump subunit G) |
| Citrus lemon | Q9THJ8 | Ribulose bisphosphate carboxylase large chain (EC 4.1.1.39) |
| | | (Fragment) |
| Citrus lemon | Q9ZST2 | Pyrophosphate--fructose 6-phosphate 1-phosphotransferase |
| | | subunit alpha (PFP) (6-phosphofructokinase, pyrophosphate |
| | | dependent) (PPi-PFK) (Pyrophosphate-dependent |
| | | 6-phosphofructose-1-kinase) |
| Citrus lemon | Q9ZWH6 | Polygalacturonase inhibitor |
| Citrus lemon | S5DXI9 | Nucleocapsid protein |
| Citrus lemon | S5NFC6 | GTP cyclohydrolase |
| Citrus lemon | V4RG42 | Uncharacterized protein |
| Citrus lemon | V4RGP4 | Uncharacterized protein |
| Citrus lemon | V4RHN8 | Uncharacterized protein |
| Citrus lemon | V4RJ07 | Uncharacterized protein |
| Citrus lemon | V4RJK9 | Adenosylhomocysteinase (EC 3.3.1.1) |
| Citrus lemon | V4RJM1 | Uncharacterized protein |
| Citrus lemon | V4RJX1 | 40S ribosomal protein S6 |
| Citrus lemon | V4RLB2 | Uncharacterized protein |
| Citrus lemon | V4RMX8 | Uncharacterized protein |
| Citrus lemon | V4RNA5 | Uncharacterized protein |
| Citrus lemon | V4RP81 | Glycosyltransferase (EC 2.4.1.—) |
| Citrus lemon | V4RPZ5 | Adenylyl cyclase-associated protein |
| Citrus lemon | V4RTN9 | Histone H4 |
| Citrus lemon | V4RUZ4 | Phosphoserine aminotransferase (EC 2.6.1.52) |
| Citrus lemon | V4RVF6 | Uncharacterized protein |
| Citrus lemon | V4RXD4 | Uncharacterized protein |
| Citrus lemon | V4RXG2 | Uncharacterized protein |
| Citrus lemon | V4RYA0 | Uncharacterized protein |
| Citrus lemon | V4RYE3 | Uncharacterized protein |
| Citrus lemon | V4RYH3 | Uncharacterized protein |
| Citrus lemon | V4RYX8 | Uncharacterized protein |
| Citrus lemon | V4RZ12 | Coatomer subunit beta′ |
| Citrus lemon | V4RZ89 | Uncharacterized protein |
| Citrus lemon | V4RZE3 | Uncharacterized protein |
| Citrus lemon | V4RZF3 | 1,2-dihydroxy-3-keto-5-methylthiopentene dioxygenase (EC |
| | | 1.13.11.54) (Acireductone dioxygenase (Fe(2+)-requiring)) |
| | | (ARD) (Fe-ARD) |
| Citrus lemon | V4RZM7 | Uncharacterized protein |
| Citrus lemon | V4RZX6 | Uncharacterized protein |
| Citrus lemon | V4S1V0 | Uncharacterized protein |
| Citrus lemon | V4S2B6 | Uncharacterized protein |
| Citrus lemon | V4S2N1 | Uncharacterized protein |
| Citrus lemon | V4S2S5 | Uncharacterized protein (Fragment) |
| Citrus lemon | V4S346 | Uncharacterized protein |
| Citrus lemon | V4S3T8 | Uncharacterized protein |
| Citrus lemon | V4S409 | Cyanate hydratase (Cyanase) (EC 4.2.1.104) (Cyanate |
| | | hydrolase) (Cyanate lyase) |
| Citrus lemon | V4S4E4 | Histone H2B |
| Citrus lemon | V4S4F6 | Flavin-containing monooxygenase (EC 1.—.—.—) |
| Citrus lemon | V4S4J1 | Uncharacterized protein |
| Citrus lemon | V4S4K9 | Uncharacterized protein |
| Citrus lemon | V4S535 | Proteasome subunit alpha type (EC 3.4.25.1) |
| Citrus lemon | V4S5A8 | Isocitrate dehydrogenase [NADP] (EC 1.1.1.42) |
| Citrus lemon | V4S5G8 | Uncharacterized protein |
| Citrus lemon | V4S5I6 | Uncharacterized protein |
| Citrus lemon | V4S5N4 | Uncharacterized protein (Fragment) |
| Citrus lemon | V4S5Q3 | Uncharacterized protein |
| Citrus lemon | V4S5X8 | Uncharacterized protein |
| Citrus lemon | V4S5Y1 | Uncharacterized protein |
| Citrus lemon | V4S6P4 | Calcium-transporting ATPase (EC 3.6.3.8) |
| Citrus lemon | V4S6W0 | Uncharacterized protein |
| Citrus lemon | V4S6W7 | Uncharacterized protein (Fragment) |
| Citrus lemon | V4S6Y4 | Uncharacterized protein |
| Citrus lemon | V4S773 | Ribosomal protein L19 |
| Citrus lemon | V4S7U0 | Uncharacterized protein |
| Citrus lemon | V4S7U5 | Uncharacterized protein |
| Citrus lemon | V4S7W4 | Pyruvate kinase (EC 2.7.1.40) |
| Citrus lemon | V4S885 | Uncharacterized protein |
| Citrus lemon | V4S8T3 | Peptidyl-prolyl cis-trans isomerase (PPIase) (EC 5.2.1.8) |
| Citrus lemon | V4S920 | Uncharacterized protein |
| Citrus lemon | V4S999 | Uncharacterized protein |
| Citrus lemon | V4S9G5 | Phosphoglycerate kinase (EC 2.7.2.3) |
| Citrus lemon | V4S9Q6 | Beta-amylase (EC 3.2.1.2) |
| Citrus lemon | V4SA44 | Serine/threonine-protein phosphatase (EC 3.1.3.16) |
| Citrus lemon | V4SAE0 | Alpha-1,4 glucan phosphorylase (EC 2.4.1.1) |
| Citrus lemon | V4SAF6 | Uncharacterized protein |
| Citrus lemon | V4SAI9 | Eukaryotic translation initiation factor 3 subunit M (eIF3m) |
| Citrus lemon | V4SAJ5 | Ribosomal protein |
| Citrus lemon | V4SAR3 | Uncharacterized protein |
| Citrus lemon | V4SB37 | Uncharacterized protein |
| Citrus lemon | V4SBI0 | Elongation factor 1-alpha |
| Citrus lemon | V4SBI8 | D-3-phosphoglycerate dehydrogenase (EC 1.1.1.95) |
| Citrus lemon | V4SBL9 | Polyadenylate-binding protein (PABP) |
| Citrus lemon | V4SBR1 | S-formylglutathione hydrolase (EC 3.1.2.12) |
| Citrus lemon | V4SBR6 | Uncharacterized protein |
| Citrus lemon | V4SCG7 | Uncharacterized protein |
| Citrus lemon | V4SCJ2 | Uncharacterized protein |
| Citrus lemon | V4SCQ6 | Peptidyl-prolyl cis-trans isomerase (PPIase) (EC 5.2.1.8) |
| Citrus lemon | V4SDJ8 | Uncharacterized protein |
| Citrus lemon | V4SE41 | Protein DETOXIFICATION (Multidrug and toxic compound |
| | | extrusion protein) |
| Citrus lemon | V4SE90 | Uncharacterized protein |
| Citrus lemon | V4SED1 | Succinate dehydrogenase [ubiquinone] flavoprotein subunit, |
| | | mitochondrial (EC 1.3.5.1) |
| Citrus lemon | V4SEI1 | Uncharacterized protein |
| Citrus lemon | V4SEN9 | Uncharacterized protein |
| Citrus lemon | V4SEX8 | Uncharacterized protein |
| Citrus lemon | V4SF31 | Uncharacterized protein |
| Citrus lemon | V4SF69 | 40S ribosomal protein S24 |
| Citrus lemon | V4SF76 | Cysteine synthase (EC 2.5.1.47) |
| Citrus lemon | V4SFK3 | Uncharacterized protein |
| Citrus lemon | V4SFL4 | Uncharacterized protein |
| Citrus lemon | V4SFW2 | Uncharacterized protein |
| Citrus lemon | V4SGC9 | Uncharacterized protein |
| Citrus lemon | V4SGJ4 | Uncharacterized protein |
| Citrus lemon | V4SGN4 | Uncharacterized protein |
| Citrus lemon | V4SGV6 | Uncharacterized protein |
| Citrus lemon | V4SGV7 | Uncharacterized protein |
| Citrus lemon | V4SHH1 | Plasma membrane ATPase (EC 3.6.3.6) (Fragment) |
| Citrus lemon | V4SHI2 | Uncharacterized protein |
| Citrus lemon | V4SHJ3 | Uncharacterized protein |
| Citrus lemon | V4SI86 | Uncharacterized protein |
| Citrus lemon | V4SI88 | Uncharacterized protein |
| Citrus lemon | V4SIA2 | Uncharacterized protein |
| Citrus lemon | V4SIC1 | Phospholipase D (EC 3.1.4.4) |
| Citrus lemon | V4SJ14 | Uncharacterized protein |
| Citrus lemon | V4SJ48 | Uncharacterized protein |
| Citrus lemon | V4SJ69 | Uncharacterized protein |
| Citrus lemon | V4SJD9 | Uncharacterized protein |
| Citrus lemon | V4SJS7 | Uncharacterized protein |
| Citrus lemon | V4SJT5 | Uncharacterized protein |
| Citrus lemon | V4SKA2 | Uncharacterized protein |
| Citrus lemon | V4SKG4 | Glucose-6-phosphate isomerase (EC 5.3.1.9) |
| Citrus lemon | V4SKJ1 | Uncharacterized protein |
| Citrus lemon | V4SL90 | Uncharacterized protein |
| Citrus lemon | V4SLC6 | Proteasome subunit beta type (EC 3.4.25.1) |
| Citrus lemon | V4SLI7 | Uncharacterized protein |
| Citrus lemon | V4SLQ6 | Uncharacterized protein |
| Citrus lemon | V4SMD8 | Uncharacterized protein |
| Citrus lemon | V4SMN7 | Uncharacterized protein |
| Citrus lemon | V4SMV5 | Uncharacterized protein |
| Citrus lemon | V4SN00 | Uncharacterized protein |
| Citrus lemon | V4SNA9 | Uncharacterized protein |
| Citrus lemon | V4SNC1 | Uncharacterized protein |
| Citrus lemon | V4SNC4 | Aconitate hydratase (Aconitase) (EC 4.2.1.3) |
| Citrus lemon | V4SNZ3 | Uncharacterized protein |
| Citrus lemon | V4SP86 | Uncharacterized protein |
| Citrus lemon | V4SPM1 | 40S ribosomal protein S12 |
| Citrus lemon | V4SPW4 | 40S ribosomal protein S4 |
| Citrus lemon | V4SQ71 | Uncharacterized protein |
| Citrus lemon | V4SQ89 | Uncharacterized protein |
| Citrus lemon | V4SQ92 | Uncharacterized protein |
| Citrus lemon | V4SQC7 | Peroxidase (EC 1.11.1.7) |
| Citrus lemon | V4SQG3 | Uncharacterized protein |
| Citrus lemon | V4SR15 | Uncharacterized protein |
| Citrus lemon | V4SRN3 | Transmembrane 9 superfamily member |
| Citrus lemon | V4SS09 | Uncharacterized protein |
| Citrus lemon | V4SS11 | Uncharacterized protein |
| Citrus lemon | V4SS50 | Uncharacterized protein |
| Citrus lemon | V4SSB6 | Uncharacterized protein |
| Citrus lemon | V4SSB8 | Proteasome subunit alpha type (EC 3.4.25.1) |
| Citrus lemon | V4SSL7 | Uncharacterized protein |
| Citrus lemon | V4SSQ1 | Uncharacterized protein |
| Citrus lemon | V4SST6 | Uncharacterized protein |
| Citrus lemon | V4SSW9 | Uncharacterized protein |
| Citrus lemon | V4SSX5 | Uncharacterized protein |
| Citrus lemon | V4SU82 | Uncharacterized protein |
| Citrus lemon | V4SUD3 | Uncharacterized protein |
| Citrus lemon | V4SUL7 | Uncharacterized protein |
| Citrus lemon | V4SUP3 | Uncharacterized protein |
| Citrus lemon | V4SUT4 | UDP-glucose 6-dehydrogenase (EC 1.1.1.22) |
| Citrus lemon | V4SUY5 | Uncharacterized protein |
| Citrus lemon | V4SV60 | Serine/threonine-protein phosphatase (EC 3.1.3.16) |
| Citrus lemon | V4SV61 | Uncharacterized protein |
| Citrus lemon | V4SVI5 | Proteasome subunit alpha type (EC 3.4.25.1) |
| Citrus lemon | V4SVI6 | Uncharacterized protein |
| Citrus lemon | V4SW04 | Uncharacterized protein (Fragment) |
| Citrus lemon | V4SWD9 | Uncharacterized protein |
| Citrus lemon | V4SWJ0 | 40S ribosomal protein S3a |
| Citrus lemon | V4SWQ9 | Uncharacterized protein |
| Citrus lemon | V4SWR9 | Uncharacterized protein |
| Citrus lemon | V4SWU9 | Fructose-bisphosphate aldolase (EC 4.1.2.13) |
| Citrus lemon | V4SX11 | Uncharacterized protein |
| Citrus lemon | V4SX99 | Uncharacterized protein |
| Citrus lemon | V4SXC7 | Proteasome subunit alpha type (EC 3.4.25.1) |
| Citrus lemon | V4SXQ5 | Uncharacterized protein |
| Citrus lemon | V4SXW1 | Beta-adaptin-like protein |
| Citrus lemon | V4SXY9 | Uncharacterized protein |
| Citrus lemon | V4SY74 | Uncharacterized protein |
| Citrus lemon | V4SY90 | Uncharacterized protein |
| Citrus lemon | V4SY93 | Uncharacterized protein |
| Citrus lemon | V4SYH9 | Uncharacterized protein |
| Citrus lemon | V4SYK6 | Uncharacterized protein |
| Citrus lemon | V4SZ03 | Uncharacterized protein |
| Citrus lemon | V4SZ73 | Uncharacterized protein |
| Citrus lemon | V4SZI9 | Uncharacterized protein |
| Citrus lemon | V4SZX7 | Uncharacterized protein |
| Citrus lemon | V4T057 | Ribosomal protein L15 |
| Citrus lemon | V4T0V5 | Eukaryotic translation initiation factor 3 subunit A (eIF3a) |
| | | (Eukaryotic translation initiation factor 3 subunit 10) |
| Citrus lemon | V4T0Y1 | Uncharacterized protein |
| Citrus lemon | V4T1Q6 | Uncharacterized protein |
| Citrus lemon | V4T1U7 | Uncharacterized protein |
| Citrus lemon | V4T2D9 | Uncharacterized protein |
| Citrus lemon | V4T2M6 | Tubulin beta chain |
| Citrus lemon | V4T3G2 | Uncharacterized protein |
| Citrus lemon | V4T3P3 | 6-phosphogluconate dehydrogenase, decarboxylating (EC |
| | | 1.1.1.44) |
| Citrus lemon | V4T3V9 | Uncharacterized protein |
| Citrus lemon | V4T3Y6 | Uncharacterized protein |
| Citrus lemon | V4T4H3 | Uncharacterized protein |
| Citrus lemon | V4T4I7 | Uncharacterized protein |
| Citrus lemon | V4T4M7 | Superoxide dismutase [Cu—Zn] (EC 1.15.1.1) |
| Citrus lemon | V4T539 | Uncharacterized protein |
| Citrus lemon | V4T541 | Uncharacterized protein |
| Citrus lemon | V4T576 | Uncharacterized protein |
| Citrus lemon | V4T5E1 | Uncharacterized protein |
| Citrus lemon | V4T5I3 | Uncharacterized protein |
| Citrus lemon | V4T5W7 | Uncharacterized protein |
| Citrus lemon | V4T6T5 | 60S acidic ribosomal protein P0 |
| Citrus lemon | V4T722 | Uncharacterized protein |
| Citrus lemon | V4T785 | Uncharacterized protein |
| Citrus lemon | V4T7E2 | Uncharacterized protein |
| Citrus lemon | V4T7I7 | Uncharacterized protein |
| Citrus lemon | V4T7N0 | Proteasome subunit beta type (EC 3.4.25.1) |
| Citrus lemon | V4T7N4 | Uncharacterized protein |
| Citrus lemon | V4T7T2 | Uncharacterized protein |
| Citrus lemon | V4T7W5 | Uncharacterized protein |
| Citrus lemon | V4T825 | Uncharacterized protein |
| Citrus lemon | V4T846 | Uncharacterized protein |
| Citrus lemon | V4T8E9 | S-acyltransferase (EC 2.3.1.225) (Palmitoyltransferase) |
| Citrus lemon | V4T8G2 | Uncharacterized protein |
| Citrus lemon | V4T8G9 | Chorismate synthase (EC 4.2.3.5) |
| Citrus lemon | V4T8Y6 | Uncharacterized protein |
| Citrus lemon | V4T8Y8 | Uncharacterized protein |
| Citrus lemon | V4T939 | Carboxypeptidase (EC 3.4.16.—) |
| Citrus lemon | V4T957 | Uncharacterized protein |
| Citrus lemon | V4T998 | Uncharacterized protein |
| Citrus lemon | V4T9B9 | Uncharacterized protein |
| Citrus lemon | V4T9Y7 | Uncharacterized protein |
| Citrus lemon | V4TA70 | Uncharacterized protein |
| Citrus lemon | V4TAF6 | Uncharacterized protein |
| Citrus lemon | V4TB09 | Uncharacterized protein |
| Citrus lemon | V4TB32 | Uncharacterized protein |
| Citrus lemon | V4TB89 | Uncharacterized protein |
| Citrus lemon | V4TBN7 | Phosphoinositide phospholipase C (EC 3.1.4.11) |
| Citrus lemon | V4TBQ3 | Uncharacterized protein |
| Citrus lemon | V4TBS4 | Uncharacterized protein |
| Citrus lemon | V4TBU3 | Uncharacterized protein |
| Citrus lemon | V4TCA6 | Uncharacterized protein |
| Citrus lemon | V4TCL3 | Uncharacterized protein |
| Citrus lemon | V4TCS5 | Pectate lyase (EC 4.2.2.2) |
| Citrus lemon | V4TD99 | Uncharacterized protein |
| Citrus lemon | V4TDB5 | Uncharacterized protein |
| Citrus lemon | V4TDI2 | Uncharacterized protein |
| Citrus lemon | V4TDY3 | Serine/threonine-protein kinase (EC 2.7.11.1) |
| Citrus lemon | V4TE72 | Uncharacterized protein |
| Citrus lemon | V4TE95 | Uncharacterized protein |
| Citrus lemon | V4TEC0 | Uncharacterized protein |
| Citrus lemon | V4TED8 | Uncharacterized protein |
| Citrus lemon | V4TES4 | Uncharacterized protein |
| Citrus lemon | V4TEY9 | Uncharacterized protein |
| Citrus lemon | V4TF24 | Proteasome subunit alpha type (EC 3.4.25.1) |
| Citrus lemon | V4TF52 | Uricase (EC 1.7.3.3) (Urate oxidase) |
| Citrus lemon | V4TFV8 | Catalase (EC 1.11.1.6) |
| Citrus lemon | V4TGU1 | Uncharacterized protein |
| Citrus lemon | V4TH28 | Uncharacterized protein |
| Citrus lemon | V4TH78 | Reticulon-like protein |
| Citrus lemon | V4THM9 | Uncharacterized protein |
| Citrus lemon | V4TIU2 | Ribulose-phosphate 3-epimerase (EC 5.1.3.1) |
| Citrus lemon | V4TIW6 | Uncharacterized protein |
| Citrus lemon | V4TIY6 | Uncharacterized protein |
| Citrus lemon | V4TIZ5 | Uncharacterized protein |
| Citrus lemon | V4TJ75 | Uncharacterized protein |
| Citrus lemon | V4TJC3 | Uncharacterized protein |
| Citrus lemon | V4TJQ9 | Uncharacterized protein |
| Citrus lemon | V4TK29 | NEDD8-activating enzyme E1 regulatory subunit |
| Citrus lemon | V4TL04 | Uncharacterized protein |
| Citrus lemon | V4TLL5 | Uncharacterized protein |
| Citrus lemon | V4TLP6 | Uncharacterized protein |
| Citrus lemon | V4TM00 | Uncharacterized protein |
| Citrus lemon | V4TM19 | Uncharacterized protein |
| Citrus lemon | V4TMB7 | Uncharacterized protein (Fragment) |
| Citrus lemon | V4TMD1 | Uncharacterized protein |
| Citrus lemon | V4TMD6 | Uncharacterized protein |
| Citrus lemon | V4TMV4 | Uncharacterized protein |
| Citrus lemon | V4TN30 | Uncharacterized protein |
| Citrus lemon | V4TN38 | Uncharacterized protein |
| Citrus lemon | V4TNY8 | Uncharacterized protein |
| Citrus lemon | V4TP87 | Carbonic anhydrase (EC 4.2.1.1) (Carbonate dehydratase) |
| Citrus lemon | V4TPM1 | Homoserine dehydrogenase (HDH) (EC 1.1.1.3) |
| Citrus lemon | V4TQB6 | Uncharacterized protein |
| Citrus lemon | V4TQM7 | Uncharacterized protein |
| Citrus lemon | V4TQR2 | Uncharacterized protein |
| Citrus lemon | V4TQV9 | Uncharacterized protein |
| Citrus lemon | V4TS21 | Proteasome subunit beta type (EC 3.4.25.1) |
| Citrus lemon | V4TS28 | Annexin |
| Citrus lemon | V4TSD8 | Uncharacterized protein (Fragment) |
| Citrus lemon | V4TSF8 | Uncharacterized protein |
| Citrus lemon | V4TSI9 | Uncharacterized protein |
| Citrus lemon | V4TT89 | Uncharacterized protein |
| Citrus lemon | V4TTA0 | Uncharacterized protein |
| Citrus lemon | V4TTR8 | Uncharacterized protein |
| Citrus lemon | V4TTV4 | Uncharacterized protein |
| Citrus lemon | V4TTZ7 | Uncharacterized protein |
| Citrus lemon | V4TU54 | Uncharacterized protein |
| Citrus lemon | V4TVB6 | Uncharacterized protein |
| Citrus lemon | V4TVG1 | Eukaryotic translation initiation factor 5A (eIF-5A) |
| Citrus lemon | V4TVJ4 | Profilin |
| Citrus lemon | V4TVM6 | Uncharacterized protein |
| Citrus lemon | V4TVM9 | Uncharacterized protein |
| Citrus lemon | V4TVP7 | Uncharacterized protein |
| Citrus lemon | V4TVT8 | Uncharacterized protein |
| Citrus lemon | V4TW14 | Uncharacterized protein |
| Citrus lemon | V4TWG9 | T-complex protein 1 subunit delta |
| Citrus lemon | V4TWU1 | Probable bifunctional methylthioribulose-1-phosphate |
| | | dehydratase/enolase-phosphatase E1 [Includes: Enolase-phosphatase |
| | | E1 (EC 3.1.3.77) (2,3-diketo-5-methylthio-1-phosphopentane |
| | | phosphatase); Methylthioribulose-1-phosphate dehydratase |
| | | (MTRu-1-P dehydratase) (EC 4.2.1.109)] |
| Citrus lemon | V4TWX8 | Uncharacterized protein |
| Citrus lemon | V4TXH0 | Glutamate decarboxylase (EC 4.1.1.15) |
| Citrus lemon | V4TXK9 | Uncharacterized protein |
| Citrus lemon | V4TXU9 | Thiamine thiazole synthase, chloroplastic (Thiazole |
| | | biosynthetic enzyme) |
| Citrus lemon | V4TY40 | Uncharacterized protein |
| Citrus lemon | V4TYJ6 | Uncharacterized protein |
| Citrus lemon | V4TYP5 | 60S ribosomal protein L13 |
| Citrus lemon | V4TYP6 | Uncharacterized protein |
| Citrus lemon | V4TYR6 | Uncharacterized protein |
| Citrus lemon | V4TYZ8 | Tubulin alpha chain |
| Citrus lemon | V4TZ91 | Guanosine nucleotide diphosphate dissociation inhibitor |
| Citrus lemon | V4TZA8 | Uncharacterized protein |
| Citrus lemon | V4TZJ1 | Uncharacterized protein |
| Citrus lemon | V4TZK5 | Uncharacterized protein |
| Citrus lemon | V4TZP2 | Uncharacterized protein |
| Citrus lemon | V4TZT8 | Uncharacterized protein |
| Citrus lemon | V4TZU3 | Mitogen-activated protein kinase (EC 2.7.11.24) |
| Citrus lemon | V4TZU5 | Dihydrolipoyl dehydrogenase (EC 1.8.1.4) |
| Citrus lemon | V4TZZ0 | Uncharacterized protein |
| Citrus lemon | V4U003 | Eukaryotic translation initiation factor 3 subunit K (eIF3k) |
| | | (eIF-3 p25) |
| Citrus lemon | V4U068 | Uncharacterized protein |
| Citrus lemon | V4U088 | Uncharacterized protein |
| Citrus lemon | V4U0J7 | Uncharacterized protein |
| Citrus lemon | V4U133 | Uncharacterized protein |
| Citrus lemon | V4U1A8 | Uncharacterized protein |
| Citrus lemon | V4U1K1 | Xylose isomerase (EC 5.3.1.5) |
| Citrus lemon | V4U1M1 | Uncharacterized protein |
| Citrus lemon | V4U1V0 | Uncharacterized protein |
| Citrus lemon | V4U1X7 | Uncharacterized protein |
| Citrus lemon | V4U1X9 | Proteasome subunit beta type (EC 3.4.25.1) |
| Citrus lemon | V4U251 | Uncharacterized protein |
| Citrus lemon | V4U283 | Uncharacterized protein |
| Citrus lemon | V4U2E4 | Uncharacterized protein |
| Citrus lemon | V4U2F7 | Uncharacterized protein |
| Citrus lemon | V4U2H8 | Uncharacterized protein |
| Citrus lemon | V4U2L0 | Malate dehydrogenase (EC 1.1.1.37) |
| Citrus lemon | V4U2L2 | Uncharacterized protein |
| Citrus lemon | V4U2W4 | V-type proton ATPase subunit C |
| Citrus lemon | V4U3L2 | Uncharacterized protein |
| Citrus lemon | V4U3W8 | Uncharacterized protein |
| Citrus lemon | V4U412 | Uncharacterized protein |
| Citrus lemon | V4U4K2 | Uncharacterized protein |
| Citrus lemon | V4U4M4 | Uncharacterized protein |
| Citrus lemon | V4U4N5 | Eukaryotic translation initiation factor 6 (eIF-6) |
| Citrus lemon | V4U4S9 | Uncharacterized protein |
| Citrus lemon | V4U4X3 | Serine hydroxymethyltransferase (EC 2.1.2.1) |
| Citrus lemon | V4U4Z9 | Uncharacterized protein |
| Citrus lemon | V4U500 | Uncharacterized protein |
| Citrus lemon | V4U5B0 | Eukaryotic translation initiation factor 3 subunit E (eIF3e) |
| | | (Eukaryotic translation initiation factor 3 subunit 6) |
| Citrus lemon | V4U5B8 | Glutathione peroxidase |
| Citrus lemon | V4U5R5 | Citrate synthase |
| Citrus lemon | V4U5Y8 | Uncharacterized protein |
| Citrus lemon | V4U6I5 | ATP synthase subunit beta (EC 3.6.3.14) |
| Citrus lemon | V4U6Q8 | Uncharacterized protein |
| Citrus lemon | V4U706 | Uncharacterized protein |
| Citrus lemon | V4U717 | Uncharacterized protein |
| Citrus lemon | V4U726 | Uncharacterized protein |
| Citrus lemon | V4U729 | Uncharacterized protein |
| Citrus lemon | V4U734 | Serine/threonine-protein phosphatase (EC 3.1.3.16) |
| Citrus lemon | V4U7G7 | Uncharacterized protein |
| Citrus lemon | V4U7H5 | Uncharacterized protein |
| Citrus lemon | V4U7R1 | Potassium transporter |
| Citrus lemon | V4U7R7 | Mitogen-activated protein kinase (EC 2.7.11.24) |
| Citrus lemon | V4U833 | Malic enzyme |
| Citrus lemon | V4U840 | Uncharacterized protein |
| Citrus lemon | V4U8C3 | Uncharacterized protein |
| Citrus lemon | V4U8J1 | 3-phosphoshikimate 1-carboxyvinyltransferase (EC 2.5.1.19) |
| Citrus lemon | V4U8J8 | T-complex protein 1 subunit gamma |
| Citrus lemon | V4U995 | Uncharacterized protein |
| Citrus lemon | V4U999 | Uncharacterized protein |
| Citrus lemon | V4U9C7 | Eukaryotic translation initiation factor 3 subunit D (eIF3d) |
| | | (Eukaryotic translation initiation factor 3 subunit 7) (eIF-3-zeta) |
| Citrus lemon | V4U9G8 | Proline iminopeptidase (EC 3.4.11.5) |
| Citrus lemon | V4U9L1 | Uncharacterized protein |
| Citrus lemon | V4UA63 | Phytochrome |
| Citrus lemon | V4UAC8 | Uncharacterized protein |
| Citrus lemon | V4UAR4 | Uncharacterized protein |
| Citrus lemon | V4UB30 | Uncharacterized protein |
| Citrus lemon | V4UBK8 | V-type proton ATPase subunit a |
| Citrus lemon | V4UBL3 | Coatomer subunit alpha |
| Citrus lemon | V4UBL5 | Uncharacterized protein (Fragment) |
| Citrus lemon | V4UBM0 | Uncharacterized protein |
| Citrus lemon | V4UBZ8 | Aspartate aminotransferase (EC 2.6.1.1) |
| Citrus lemon | V4UC72 | Uncharacterized protein |
| Citrus lemon | V4UC97 | Beta-glucosidase (EC 3.2.1.21) |
| Citrus lemon | V4UCE2 | Uncharacterized protein |
| Citrus lemon | V4UCT9 | Acetyl-coenzyme A synthetase (EC 6.2.1.1) |
| Citrus lemon | V4UCZ1 | Uncharacterized protein |
| Citrus lemon | V4UE34 | Uncharacterized protein |
| Citrus lemon | V4UE78 | Uncharacterized protein |
| Citrus lemon | V4UER3 | Uncharacterized protein |
| Citrus lemon | V4UET6 | Uncharacterized protein |
| Citrus lemon | V4UEZ6 | Uncharacterized protein |
| Citrus lemon | V4UFD0 | Uncharacterized protein |
| Citrus lemon | V4UFG8 | Uncharacterized protein |
| Citrus lemon | V4UFK1 | Uncharacterized protein |
| Citrus lemon | V4UG68 | Eukaryotic translation initiation factor 3 subunit I (eIF3i) |
| Citrus lemon | V4UGB0 | Uncharacterized protein |
| Citrus lemon | V4UGH4 | Uncharacterized protein |
| Citrus lemon | V4UGL9 | Uncharacterized protein |
| Citrus lemon | V4UGQ0 | Ubiquitinyl hydrolase 1 (EC 3.4.19.12) |
| Citrus lemon | V4UH00 | Uncharacterized protein |
| Citrus lemon | V4UH48 | Uncharacterized protein |
| Citrus lemon | V4UH77 | Proteasome subunit alpha type (EC 3.4.25.1) |
| Citrus lemon | V4UHD8 | Uncharacterized protein |
| Citrus lemon | V4UHD9 | Uncharacterized protein |
| Citrus lemon | V4UHF1 | Uncharacterized protein |
| Citrus lemon | V4UHZ5 | Uncharacterized protein |
| Citrus lemon | V4UI07 | 40S ribosomal protein S8 |
| Citrus lemon | V4UI34 | Eukaryotic translation initiation factor 3 subunit L (eIF3l) |
| Citrus lemon | V4UIF1 | Uncharacterized protein |
| Citrus lemon | V4UIN5 | Uncharacterized protein |
| Citrus lemon | V4UIX8 | Uncharacterized protein |
| Citrus lemon | V4UJ12 | Uncharacterized protein |
| Citrus lemon | V4UJ42 | Uncharacterized protein |
| Citrus lemon | V4UJ63 | Uncharacterized protein |
| Citrus lemon | V4UJB7 | Uncharacterized protein (Fragment) |
| Citrus lemon | V4UJC4 | Uncharacterized protein |
| Citrus lemon | V4UJX0 | Phosphotransferase (EC 2.7.1.—) |
| Citrus lemon | V4UJY5 | Uncharacterized protein |
| Citrus lemon | V4UK18 | Uncharacterized protein |
| Citrus lemon | V4UK52 | Uncharacterized protein |
| Citrus lemon | V4UKM9 | Uncharacterized protein |
| Citrus lemon | V4UKS4 | Uncharacterized protein |
| Citrus lemon | V4UKV6 | 40S ribosomal protein SA |
| Citrus lemon | V4UL30 | Pyrophosphate--fructose 6-phosphate 1-phosphotransferase |
| | | subunit beta (PFP) (EC 2.7.1.90) (6-phosphofructokinase, |
| | | pyrophosphate dependent) (PPi-PFK) |
| | | (Pyrophosphate-dependent 6-phosphofructose-1-kinase) |
| Citrus lemon | V4UL39 | Uncharacterized protein |
| Citrus lemon | V4ULH9 | Uncharacterized protein |
| Citrus lemon | V4ULL2 | Uncharacterized protein |
| Citrus lemon | V4ULS0 | Uncharacterized protein |
| Citrus lemon | V4UMU7 | Uncharacterized protein |
| Citrus lemon | V4UN36 | Uncharacterized protein |
| Citrus lemon | V4UNT5 | Uncharacterized protein |
| Citrus lemon | V4UNW1 | Uncharacterized protein |
| Citrus lemon | V4UP89 | Uncharacterized protein |
| Citrus lemon | V4UPE4 | Uncharacterized protein |
| Citrus lemon | V4UPF7 | Uncharacterized protein |
| Citrus lemon | V4UPK0 | Uncharacterized protein |
| Citrus lemon | V4UPX5 | Uncharacterized protein |
| Citrus lemon | V4UQ58 | Uncharacterized protein |
| Citrus lemon | V4UQF6 | Uncharacterized protein |
| Citrus lemon | V4UR21 | Uncharacterized protein |
| Citrus lemon | V4UR80 | Uncharacterized protein |
| Citrus lemon | V4URK3 | Uncharacterized protein |
| Citrus lemon | V4URT3 | Uncharacterized protein |
| Citrus lemon | V4US96 | Uncharacterized protein |
| Citrus lemon | V4USQ8 | Uncharacterized protein |
| Citrus lemon | V4UT16 | Uncharacterized protein |
| Citrus lemon | V4UTC6 | Uncharacterized protein |
| Citrus lemon | V4UTC8 | Uncharacterized protein |
| Citrus lemon | V4UTP6 | Uncharacterized protein |
| Citrus lemon | V4UTY0 | Proteasome subunit alpha type (EC 3.4.25.1) |
| Citrus lemon | V4UU96 | Uncharacterized protein |
| Citrus lemon | V4UUB6 | Uncharacterized protein |
| Citrus lemon | V4UUJ9 | Aminopeptidase (EC 3.4.11.—) |
| Citrus lemon | V4UUK6 | Uncharacterized protein |
| Citrus lemon | V4UV09 | Uncharacterized protein |
| Citrus lemon | V4UV83 | Lysine--tRNA ligase (EC 6.1.1.6) (Lysyl-tRNA synthetase) |
| Citrus lemon | V4UVJ5 | Diacylglycerol kinase (DAG kinase) (EC 2.7.1.107) |
| Citrus lemon | V4UW03 | Uncharacterized protein |
| Citrus lemon | V4UW04 | Uncharacterized protein |
| Citrus lemon | V4UWR1 | Uncharacterized protein |
| Citrus lemon | V4UWV8 | Uncharacterized protein |
| Citrus lemon | V4UX36 | Uncharacterized protein |
| Citrus lemon | V4V003 | Uncharacterized protein |
| Citrus lemon | V4V0J0 | 40S ribosomal protein S26 |
| Citrus lemon | V4V1P8 | Uncharacterized protein |
| Citrus lemon | V4V4V0 | Uncharacterized protein |
| Citrus lemon | V4V5T8 | Ubiquitin-fold modifier 1 |
| Citrus lemon | V4V600 | Uncharacterized protein |
| Citrus lemon | V4V622 | Aldehyde dehydrogenase |
| Citrus lemon | V4V6W1 | Uncharacterized protein |
| Citrus lemon | V4V6Z2 | Uncharacterized protein |
| Citrus lemon | V4V738 | Uncharacterized protein |
| Citrus lemon | V4V8H5 | Vacuolar protein sorting-associated protein 35 |
| Citrus lemon | V4V9P6 | Eukaryotic translation initiation factor 3 subunit F (eIF3f) |
| | | (eIF-3-epsilon) |
| Citrus lemon | V4V9V7 | Clathrin heavy chain |
| Citrus lemon | V4V9X3 | Uncharacterized protein |
| Citrus lemon | V4VAA3 | Superoxide dismutase (EC 1.15.1.1) |
| Citrus lemon | V4VAF3 | Uncharacterized protein |
| Citrus lemon | V4VBQ0 | Uncharacterized protein (Fragment) |
| Citrus lemon | V4VCL1 | Proteasome subunit beta type (EC 3.4.25.1) |
| Citrus lemon | V4VCZ9 | Uncharacterized protein |
| Citrus lemon | V4VDK1 | Peptidylprolyl isomerase (EC 5.2.1.8) |
| Citrus lemon | V4VEA1 | Uncharacterized protein |
| Citrus lemon | V4VEB3 | Alanine--tRNA ligase (EC 6.1.1.7) (Alanyl-tRNA synthetase) |
| | | (AlaRS) |
| Citrus lemon | V4VEE3 | Glutamine synthetase (EC 6.3.1.2) |
| Citrus lemon | V4VFM3 | Uncharacterized protein |
| Citrus lemon | V4VFN5 | Proteasome subunit beta type (EC 3.4.25.1) |
| Citrus lemon | V4VGD6 | Uncharacterized protein |
| Citrus lemon | V4VGL9 | Uncharacterized protein |
| Citrus lemon | V4VHI6 | Uncharacterized protein |
| Citrus lemon | V4VIP4 | Uncharacterized protein |
| Citrus lemon | V4VJT4 | Uncharacterized protein |
| Citrus lemon | V4VK14 | Uncharacterized protein |
| Citrus lemon | V4VKI5 | Protein-L-isoaspartate O-methyltransferase (EC 2.1.1.77) |
| Citrus lemon | V4VKP2 | Glyceraldehyde-3-phosphate dehydrogenase (EC 1.2.1.—) |
| Citrus lemon | V4VL73 | Acyl-coenzyme A oxidase |
| Citrus lemon | V4VLL7 | Uncharacterized protein |
| Citrus lemon | V4VN43 | Uncharacterized protein (Fragment) |
| Citrus lemon | V4VQH3 | Methylenetetrahydrofolate reductase (EC 1.5.1.20) |
| Citrus lemon | V4VTC9 | Uncharacterized protein (Fragment) |
| Citrus lemon | V4VTT4 | Uncharacterized protein |
| Citrus lemon | V4VTY7 | Uncharacterized protein |
| Citrus lemon | V4VU14 | Uncharacterized protein |
| Citrus lemon | V4VU32 | Uncharacterized protein |
| Citrus lemon | V4VUK6 | S-(hydroxymethyl)glutathione dehydrogenase (EC 1.1.1.284) |
| Citrus lemon | V4VVR8 | Uncharacterized protein |
| Citrus lemon | V4VXE2 | Uncharacterized protein |
| Citrus lemon | V4VY37 | Phosphomannomutase (EC 5.4.2.8) |
| Citrus lemon | V4VYC0 | Uncharacterized protein |
| Citrus lemon | V4VYV1 | Uncharacterized protein |
| Citrus lemon | V4VZ80 | Uncharacterized protein |
| Citrus lemon | V4VZJ7 | Uncharacterized protein |
| Citrus lemon | V4W2P2 | Alpha-mannosidase (EC 3.2.1.—) |
| Citrus lemon | V4W2Z9 | Chloride channel protein |
| Citrus lemon | V4W378 | Uncharacterized protein |
| Citrus lemon | V4W4G3 | Uncharacterized protein |
| Citrus lemon | V4W5F1 | Uncharacterized protein |
| Citrus lemon | V4W5N8 | Uncharacterized protein |
| Citrus lemon | V4W5U2 | Uncharacterized protein |
| Citrus lemon | V4W6G1 | Uncharacterized protein |
| Citrus lemon | V4W730 | Uncharacterized protein |
| Citrus lemon | V4W7J4 | Obg-like ATPase 1 |
| Citrus lemon | V4W7L5 | Uncharacterized protein |
| Citrus lemon | V4W8C5 | Uncharacterized protein |
| Citrus lemon | V4W8C9 | Uncharacterized protein |
| Citrus lemon | V4W8D3 | Uncharacterized protein |
| Citrus lemon | V4W951 | Uncharacterized protein |
| Citrus lemon | V4W9F6 | 60S ribosomal protein L18a |
| Citrus lemon | V4W9G2 | Uncharacterized protein (Fragment) |
| Citrus lemon | V4W9L3 | Uncharacterized protein |
| Citrus lemon | V4W9Y8 | Uncharacterized protein |
| Citrus lemon | V4WAP9 | Coatomer subunit beta (Beta-coat protein) |
| Citrus lemon | V4WBK6 | Cytochrome b-c1 complex subunit 7 |
| Citrus lemon | V4WC15 | Malic enzyme |
| Citrus lemon | V4WC19 | Uncharacterized protein |
| Citrus lemon | V4WC74 | Uncharacterized protein |
| Citrus lemon | V4WC86 | Serine/threonine-protein phosphatase 2A 55 kDa regulatory |
| | | subunit B |
| Citrus lemon | V4WCS4 | GTP-binding nuclear protein |
| Citrus lemon | V4WD80 | Aspartate aminotransferase (EC 2.6.1.1) |
| Citrus lemon | V4WDK0 | Uncharacterized protein |
| Citrus lemon | V4WDK3 | ATP-dependent 6-phosphofructokinase (ATP-PFK) |
| | | (Phosphofructokinase) (EC 2.7.1.11) (Phosphohexokinase) |
| Citrus lemon | V4WE00 | Uncharacterized protein |
| Citrus lemon | V4WEE3 | Uncharacterized protein |
| Citrus lemon | V4WEN2 | Uncharacterized protein |
| Citrus lemon | V4WG97 | Autophagy-related protein |
| Citrus lemon | V4WGV2 | Uncharacterized protein |
| Citrus lemon | V4WGW5 | Uridine kinase (EC 2.7.1.48) |
| Citrus lemon | V4WHD4 | Uncharacterized protein |
| Citrus lemon | V4WHF8 | Sucrose synthase (EC 2.4.1.13) |
| Citrus lemon | V4WHK2 | Pectinesterase (EC 3.1.1.11) |
| Citrus lemon | V4WHQ4 | Uncharacterized protein |
| Citrus lemon | V4WHT6 | Uncharacterized protein |
| Citrus lemon | V4WJ93 | Uncharacterized protein |
| Citrus lemon | V4WJA9 | Uncharacterized protein |
| Citrus lemon | V4WJB1 | Uncharacterized protein |
| Citrus lemon | V9HXG3 | Protein disulfide-isomerase (EC 5.3.4.1) |
| Citrus lemon | W8Q8K1 | Putative inorganic pyrophosphatase |
| Citrus lemon | W8QJL0 | Putative isopentenyl pyrophosphate isomerase |

| Grape | Accession Number | Identified Proteins |
|---|---|---|
| Grape | A5C5K3 (+2) | Adenosylhomocysteinase |
| Grape | Q9M6B5 | Alcohol dehydrogenase 6 |
| Grape | A3FA65 (+1) | Aquaporin PIP1; 3 |
| Grape | Q0MX13 (+2) | Aquaporin PIP2; 2 |
| Grape | A3FA69 (+4) | Aquaporin PIP2; 4 |
| Grape | A5AFS1 (+2) | Elongation factor 1-alpha |
| Grape | UPI0001985702 | elongation factor 2 |
| Grape | D7T227 | Enolase |
| Grape | D7TJ12 | Enolase |
| Grape | A5B118 (+1) | Fructose-bisphosphate aldolase |
| Grape | E0CQ39 | Glucose-6-phosphate isomerase |
| Grape | D7TW04 | Glutathione peroxidase |
| Grape | A1YW90 (+3) | Glutathione S-transferase |
| Grape | A5BEW0 | Histone H4 |
| Grape | UPI00015C9A6A | HSC70-1 (heat shock cognate 70 kDa protein 1); ATP |
| | | binding isoform 1 |
| Grape | D7FBC0 (+1) | Malate dehydrogenase |
| Grape | D7TBH4 | Malic enzyme |
| Grape | A5ATB7 (+1) | Methylenetetrahydrofolate reductase |
| Grape | A5JPK7 (+1) | Monodehydroascorbate reductase |
| Grape | A5AKD8 | Peptidyl-prolyl cis-trans isomerase |
| Grape | A5BQN6 | Peptidyl-prolyl cis-trans isomerase |
| Grape | A5CAF6 | Phosphoglycerate kinase |
| Grape | Q09VU3 (+1) | Phospholipase D |
| Grape | D7SK33 | Phosphorylase |
| Grape | A5AQ89 | Profilin |
| Grape | C5DB50 (+2) | Putative 2,3-bisphosphoglycerate-independent |
| | | phosphoglycerate mutase |
| Grape | D7TIZ5 | Pyruvate kinase |
| Grape | A5BV65 | Triosephosphate isomerase |
| Grapefruit | G8Z362 (+1) | (E)-beta-farnesene synthase |
| Grapefruit | Q5CD81 | (E)-beta-ocimene synthase |
| Grapefruit | D0UZK1 (+2) | 1,2 rhamnosyltransferase |
| Grapefruit | A7ISD3 | 1,6-rhamnosyltransferase |
| Grapefruit | Q80H98 | 280 kDa protein |
| Grapefruit | Q15GA4 (+2) | 286 kDa polyprotein |
| Grapefruit | D7NHW9 | 2-phospho-D-glycerate hydrolase |
| Grapefruit | D0EAL9 | 349 kDa polyprotein |
| Grapefruit | Q9DTG5 | 349-kDa polyprotein |
| Grapefruit | O22297 | Acidic cellulase |
| Grapefruit | Q8H986 | Acidic class I chitinase |
| Grapefruit | D3GQL0 | Aconitate hydratase 1 |
| Grapefruit | K7N8A0 | Actin |
| Grapefruit | A8W8Y0 | Alcohol acyl transferase |
| Grapefruit | Q84V85 | Allene oxide synthase |
| Grapefruit | F8WL79 | Aminopeptidase |
| Grapefruit | Q09MG5 | Apocytochrome f |
| Grapefruit | J7EIR8 | Ascorbate peroxidase |
| Grapefruit | B9VRH6 | Ascorbate peroxidase |
| Grapefruit | G9I820 | Auxin-response factor |
| Grapefruit | J7ICW8 | Beta-amylase |
| Grapefruit | Q8L5Q9 | Beta-galactosidase |
| Grapefruit | A7BG60 | Beta-pinene synthase |
| Grapefruit | C0KLD1 | Beta-tubulin |
| Grapefruit | Q91QZ1 | Capsid protein |
| Grapefruit | Q3SAK9 | Capsid protein |
| Grapefruit | D2U833 | Cation chloride cotransporter |
| Grapefruit | C3VPJ0 (+3) | Chaicone synthase |
| Grapefruit | D5LM39 | Chloride channel protein |
| Grapefruit | Q9M4U0 | Cinnamate 4-hydroxylase CYP73 |
| Grapefruit | Q39627 | Citrin |
| Grapefruit | G2XKD3 | Coat protein |
| Grapefruit | Q3L2I6 | Coat protein |
| Grapefruit | D5FV16 | CRT/DRE binding factor |
| Grapefruit | Q8H6S5 | CTV.2 |
| Grapefruit | Q8H6Q8 | CTV.20 |
| Grapefruit | Q8H6Q7 | CTV.22 |
| Grapefruit | Q1I1D7 | Cytochrome P450 |
| Grapefruit | Q7Y045 | Dehydrin |
| Grapefruit | F8WLD2 | DNA excision repair protein |
| Grapefruit | Q09MI8 | DNA-directed RNA polymerase subunit beta″ |
| Grapefruit | D2WKC9 | Ethylene response 1 |
| Grapefruit | D2WKD2 | Ethylene response sensor 1 |
| Grapefruit | D7PVG7 | Ethylene-insensitive 3-like 1 protein |
| Grapefruit | G3CHK8 | Eukaryotic translation initiation factor 3 subunit E |
| Grapefruit | A9NJG4 (+3) | Fatty acid hydroperoxide lyase |
| Grapefruit | B8Y9B5 | F-box family protein |
| Grapefruit | Q000W4 | Fe(III)-chelate reductase |
| Grapefruit | Q6Q3H4 | Fructokinase |
| Grapefruit | F8WL95 | Gag-pol polyprotein |
| Grapefruit | Q8L5K4 | Gamma-terpinene synthase, chloroplastic |
| Grapefruit | Q9SP43 | Glucose-1-phosphate adenylyltransferase |
| Grapefruit | Q3HM93 | Glutathione S-transferase |
| Grapefruit | D0VEW6 | GRAS family transcription factor |
| Grapefruit | F8WL87 | Heat shock protein |
| Grapefruit | H9NHK0 | Hsp90 |
| Grapefruit | Q8H6R4 | Jp18 |
| Grapefruit | G3CHK6 | Leucine-rich repeat family protein |
| Grapefruit | B2YGX9 (+1) | Limonoid UDP-glucosyltransferase |
| Grapefruit | Q05KK0 | MADS-box protein |
| Grapefruit | F8WLB4 | Mechanosensitive ion channel domain-containing protein |
| Grapefruit | Q5CD82 | Monoterpene synthase |
| Grapefruit | F8WLC4 | MYB transcription factor |
| Grapefruit | A5YWA9 | NAC domain protein |
| Grapefruit | Q09MC9 | NAD(P)H-quinone oxidoreductase subunit 5, chloroplastic |
| Grapefruit | Q8H6R9 | NBS-LRR type disease resistance protein |
| Grapefruit | Q8H6S0 | NBS-LRR type disease resistance protein |
| Grapefruit | Q8H6R6 | NBS-LRR type disease resistance protein |
| Grapefruit | J9WR93 | p1a |
| Grapefruit | Q1X8V8 | P23 |
| Grapefruit | E7DSS0 (+4) | P23 |
| Grapefruit | G0Z9I6 | p27 |
| Grapefruit | I3XHN0 | p33 |
| Grapefruit | B8YDL3 | p33 protein |
| Grapefruit | B9VB22 | p33 protein |
| Grapefruit | P87587 | P346 |
| Grapefruit | B9VB56 | p349 protein |
| Grapefruit | I3RWW7 | p349 protein |
| Grapefruit | B9VB20 | p349 protein |
| Grapefruit | Q9WID7 | p349 protein |
| Grapefruit | Q2XP16 | P353 |
| Grapefruit | O04886 (+1) | Pectinesterase 1 |
| Grapefruit | F8WL74 | Peptidyl-prolyl cis-trans isomerase |
| Grapefruit | Q0ZA67 | Peroxidase |
| Grapefruit | F1CT41 | Phosphoenolpyruvate carboxylase |
| Grapefruit | B1PBV7 (+2) | Phytoene synthase |
| Grapefruit | Q9ZWQ8 | Plastid-lipid-associated protein, chloroplastic |
| Grapefruit | Q94FM1 | Pol polyprotein |
| Grapefruit | Q94FM0 | Pol polyprotein |
| Grapefruit | G9I825 | Poly C-binding protein |
| Grapefruit | O64460 (+7) | Polygalacturonase inhibitor |
| Grapefruit | I3XHM8 | Polyprotein |
| Grapefruit | C0STR9 | Polyprotein |
| Grapefruit | H6U1F0 | Polyprotein |
| Grapefruit | B8QHP8 | Polyprotein |
| Grapefruit | I3V6C0 | Polyprotein |
| Grapefruit | C0STS0 | Polyprotein |
| Grapefruit | K0FGH5 | Polyprotein |
| Grapefruit | Q3HWZ1 | Polyprotein |
| Grapefruit | F8WLA5 | PPR containing protein |
| Grapefruit | Q06652 (+1) | Probable phospholipid hydroperoxide glutathione |
| | | peroxidase |
| Grapefruit | P84177 | Profilin |
| Grapefruit | Q09MB4 | Protein ycf2 |
| Grapefruit | A8C183 | PSI reaction center subunit II |
| Grapefruit | A5JVP6 | Putative 2b protein |
| Grapefruit | D0EFM2 | Putative eukaryotic translation initiation factor 1 |
| Grapefruit | Q18L98 | Putative gag-pol polyprotein |
| Grapefruit | B5AMI9 | Putative movement protein |
| Grapefruit | A1ECK5 | Putative multiple stress-responsive zinc-finger protein |
| Grapefruit | B5AMJ0 | Putative replicase polyprotein |
| Grapefruit | I7CYN5 | Putative RNA-dependent RNA polymerase |
| Grapefruit | Q8RVR2 | Putative terpene synthase |
| Grapefruit | B5TE89 | Putative uncharacterized protein |
| Grapefruit | Q8JVF3 | Putative uncharacterized protein |
| Grapefruit | F8WLB0 | Putative uncharacterized protein ORF43 |
| Grapefruit | A5JVP4 | Putative viral replicase |
| Grapefruit | M1JAW3 | Replicase |
| Grapefruit | H6VXK8 | Replicase polyprotein |
| Grapefruit | J9UF50 (+1) | Replicase protein 1a |
| Grapefruit | J9RV45 | Replicase protein 2a |
| Grapefruit | Q5EGG5 | Replicase-associated polyprotein |
| Grapefruit | G9I823 | RNA recognition motif protein 1 |
| Grapefruit | J7EPC0 | RNA-dependent RNA polymerase |
| Grapefruit | Q6DN67 | RNA-directed RNA polymerase L |
| Grapefruit | A9CQM4 | SEPALLATA1 homolog |
| Grapefruit | Q9SLS2 | Sucrose synthase |
| Grapefruit | Q9SLV8 (+1) | Sucrose synthase |
| Grapefruit | Q38JC1 | Temperature-induced lipocalin |
| Grapefruit | D0ELH6 | Tetratricopeptide domain-containing thioredoxin |
| Grapefruit | D2KU75 | Thaumatin-like protein |
| Grapefruit | C3VIC2 | Translation elongation factor |
| Grapefruit | D5LY07 | Ubiquitin/ribosomal fusion protein |
| Grapefruit | C6KI43 | UDP-glucosyltransferase family 1 protein |
| Grapefruit | A0FKR1 | Vacuolar citrate/H+ symporter |
| Grapefruit | Q944C8 | Vacuolar invertase |
| Grapefruit | Q9MB46 | V-type proton ATPase subunit E |
| Grapefruit | F8WL82 | WD-40 repeat family protein |
| Helianthuus annuus | HanXRQChr03g0080391 | Hsp90 |
| Helianthuus annuus | HanXRQChr13g0408351 | Hsp90 |
| Helianthuus annuus | HanXRQChr13g0408441 | Hsp90 |
| Helianthuus annuus | HanXRQChr14g0462551 | Hsp90 |
| Helianthuus annuus | HanXRQChr02g0044471 | Hsp70 |
| Helianthuus annuus | HanXRQChr02g0044481 | Hsp70 |
| Helianthuus annuus | HanXRQChr05g0132631 | Hsp70 |
| Helianthuus annuus | HanXRQChr05g0134631 | Hsp70 |
| Helianthuus annuus | HanXRQChr05g0134801 | Hsp70 |
| Helianthuus annuus | HanXRQChr10g0299441 | glutathione S-transferase |
| Helianthuus annuus | HanXRQChr16g0516291 | glutathione S-transferase |
| Helianthuus annuus | HanXRQChr03g0091431 | lactate/malate dehydrogenase |
| Helianthuus annuus | HanXRQChr13g0421951 | lactate/malate dehydrogenase |
| Helianthuus annuus | HanXRQChr10g0304821 | lactate/malate dehydrogenase |
| Helianthuus annuus | HanXRQChr12g0373491 | lactate/malate dehydrogenase |
| Helianthuus annuus | HanXRQChr01g0031071 | small GTPase superfamily, Rab type |
| Helianthuus annuus | HanXRQChr01g0031091 | small GTPase superfamily, Rab type |
| Helianthuus annuus | HanXRQChr02g0050791 | small GTPase superfamily, Rab type |
| Helianthuus annuus | HanXRQChr11g0353711 | small GTPase superfamily, Rab type |
| Helianthuus annuus | HanXRQChr13g0402771 | small GTPase superfamily, Rab type |
| Helianthuus annuus | HanXRQChr07g0190171 | isocitrate/isopropylmalate dehydrogenase |
| Helianthuus annuus | HanXRQChr16g0532251 | isocitrate/isopropylmalate dehydrogenase |
| Helianthuus annuus | HanXRQChr03g0079131 | phosphoenolpyruvate carboxylase |
| Helianthuus annuus | HanXRQChr15g0495261 | phosphoenolpyruvate carboxylase |
| Helianthuus annuus | HanXRQChr13g0388931 | phosphoenolpyruvate carboxylase |
| Helianthuus annuus | HanXRQChr14g0442731 | phosphoenolpyruvate carboxylase |
| Helianthuus annuus | HanXRQChr15g0482381 | UTP--glucose-1-phosphate uridylyltransferase |
| Helianthuus annuus | HanXRQChr16g0532261 | UTP--glucose-1-phosphate uridylyltransferase |
| Helianthuus annuus | HanXRQChr05g0135591 | tubulin |
| Helianthuus annuus | HanXRQChr06g0178921 | tubulin |
| Helianthuus annuus | HanXRQChr08g0237071 | tubulin |
| Helianthuus annuus | HanXRQChr11g0337991 | tubulin |
| Helianthuus annuus | HanXRQChr13g0407921 | tubulin |
| Helianthuus annuus | HanXRQChr05g0145191 | tubulin |
| Helianthuus annuus | HanXRQChr07g0187021 | tubulin |
| Helianthuus annuus | HanXRQChr07g0189811 | tubulin |
| Helianthuus annuus | HanXRQChr09g0253681 | tubulin |
| Helianthuus annuus | HanXRQChr10g0288911 | tubulin |
| Helianthuus annuus | HanXRQChr11g0322631 | tubulin |
| Helianthuus annuus | HanXRQChr12g0367231 | tubulin |
| Helianthuus annuus | HanXRQChr13g0386681 | tubulin |
| Helianthuus annuus | HanXRQChr13g0393261 | tubulin |
| Helianthuus annuus | HanXRQChr12g0371591 | ubiquitin |
| Helianthuus annuus | HanXRQChr12g0383641 | ubiquitin |
| Helianthuus annuus | HanXRQChr17g0569881 | ubiquitin |
| Helianthuus annuus | HanXRQChr06g0171511 | photosystem II HCF136, stability/assembly factor |
| Helianthuus annuus | HanXRQChr17g0544921 | photosystem II HCF136, stability/assembly factor |
| Helianthuus annuus | HanXRQChr16g0526461 | proteasome B-type subunit |
| Helianthuus annuus | HanXRQChr17g0565551 | proteasome B-type subunit |
| Helianthuus annuus | HanXRQChr05g0149801 | proteasome B-type subunit |
| Helianthuus annuus | HanXRQChr09g0241421 | proteasome B-type subunit |
| Helianthuus annuus | HanXRQChr11g0353161 | proteasome B-type subunit |
| Helianthuus annuus | HanXRQChr16g0506311 | proteinase inhibitor family I3 (Kunitz) |
| Helianthuus annuus | HanXRQChr16g0506331 | proteinase inhibitor family I3 (Kunitz) |
| Helianthuus annuus | HanXRQChr09g0265401 | metallopeptidase (M10 family) |
| Helianthuus annuus | HanXRQChr09g0265411 | metallopeptidase (M10 family) |
| Helianthuus annuus | HanXRQChr05g0154561 | ATPase, AAA-type |
| Helianthuus annuus | HanXRQChr08g0235061 | ATPase, AAA-type |
| Helianthuus annuus | HanXRQChr09g0273921 | ATPase, AAA-type |
| Helianthuus annuus | HanXRQChr16g0498881 | ATPase, AAA-type |
| Helianthuus annuus | HanXRQChr02g0058711 | oxoacid dehydrogenase acyltransferase |
| Helianthuus annuus | HanXRQChr08g0214191 | oxoacid dehydrogenase acyltransferase |
| Helianthuus annuus | HanXRQChr08g0208631 | small GTPase superfamily, SAR1-type |
| Helianthuus annuus | HanXRQChr11g0331441 | small GTPase superfamily, SAR1-type |
| Helianthuus annuus | HanXRQChr12g0371571 | small GTPase superfamily, SAR1-type |
| Helianthuus annuus | HanXRQChr12g0383571 | small GTPase superfamily, SAR1-type |
| Helianthuus annuus | HanXRQChr14g0446771 | small GTPase superfamily, SAR1-type |
| Helianthuus annuus | HanXRQChr17g0539461 | small GTPase superfamily, SAR1-type |
| Helianthuus annuus | HanXRQChr17g0548271 | small GTPase superfamily, SAR1-type |
| Helianthuus annuus | HanXRQChr17g0569871 | small GTPase superfamily, SAR1-type |
| Helianthuus annuus | HanXRQChr10g0311201 | ATPase, V1 complex, subunit A |
| Helianthuus annuus | HanXRQChr12g0359711 | ATPase, V1 complex, subunit A |
| Helianthuus annuus | HanXRQChr04g0124671 | fructose-1,6-bisphosphatase |
| Helianthuus annuus | HanXRQChr06g0176631 | fructose-1,6-bisphosphatase |
| Helianthuus annuus | HanXRQCPg0579861 | photosystem II PsbD/D2, reaction centre |
| Helianthuus annuus | HanXRQChr00c0439g0574731 | photosystem II PsbD/D2, reaction centre |
| Helianthuus annuus | HanXRQChr04g0099321 | photosystem II PsbD/D2, reaction centre |
| Helianthuus annuus | HanXRQChr08g0210231 | photosystem II PsbD/D2, reaction centre |
| Helianthuus annuus | HanXRQChr11g0326671 | photosystem II PsbD/D2, reaction centre |
| Helianthuus annuus | HanXRQChr17g0549121 | photosystem II PsbD/D2, reaction centre |
| Helianthuus annuus | HanXRQCPg0579731 | photosystem II protein D1 |
| Helianthuus annuus | HanXRQChr00c0126g0571821 | photosystem II protein D1 |
| Helianthuus annuus | HanXRQChr00c0165g0572191 | photosystem II protein D1 |
| Helianthuus annuus | HanXRQChr00c0368g0574171 | photosystem II protein D1 |
| Helianthuus annuus | HanXRQChr00c0454g0574931 | photosystem II protein D1 |
| Helianthuus annuus | HanXRQChr00c0524g0575441 | photosystem II protein D1 |
| Helianthuus annuus | HanXRQChr00c0572g0575941 | photosystem II protein D1 |
| Helianthuus annuus | HanXRQChr09g0257281 | photosystem II protein D1 |
| Helianthuus annuus | HanXRQChr11g0326571 | photosystem II protein D1 |
| Helianthuus annuus | HanXRQChr11g0327051 | photosystem II protein D1 |
| Helianthuus annuus | HanXRQChr16g0503941 | photosystem II protein D1 |
| Helianthuus annuus | HanXRQCPg0580061 | photosystem II cytochrome b559 |
| Helianthuus annuus | HanXRQChr01g0020331 | photosystem II cytochrome b559 |
| Helianthuus annuus | HanXRQChr10g0283581 | photosystem II cytochrome b559 |
| Helianthuus annuus | HanXRQChr10g0284271 | photosystem II cytochrome b559 |
| Helianthuus annuus | HanXRQChr10g0289291 | photosystem II cytochrome b559 |
| Helianthuus annuus | HanXRQChr10g0318171 | photosystem II cytochrome b559 |
| Helianthuus annuus | HanXRQChr11g0326851 | photosystem II cytochrome b559 |
| Helianthuus annuus | HanXRQChr16g0529011 | photosystem II cytochrome b559 |
| Helianthuus annuus | HanXRQChr08g0219051 | chlorophyll A-B binding protein |
| Helianthuus annuus | HanXRQChr12g0370841 | chlorophyll A-B binding protein |
| Helianthuus annuus | HanXRQChr02g0053151 | chlorophyll A-B binding protein |
| Helianthuus annuus | HanXRQChr02g0053161 | chlorophyll A-B binding protein |
| Helianthuus annuus | HanXRQCPg0580051 | cytochrome f |
| Helianthuus annuus | HanXRQChr01g0020341 | cytochrome f |
| Helianthuus annuus | HanXRQChr10g0283571 | cytochrome f |
| Helianthuus annuus | HanXRQChr10g0284261 | cytochrome f |
| Helianthuus annuus | HanXRQChr10g0289281 | cytochrome f |
| Helianthuus annuus | HanXRQChr10g0318181 | cytochrome f |
| Helianthuus annuus | HanXRQChr11g0326841 | cytochrome f |
| Helianthuus annuus | HanXRQChr15g0497521 | cytochrome f |
| Helianthuus annuus | HanXRQChr06g0163851 | ribosomal protein |
| Helianthuus annuus | HanXRQChr09g0252071 | ribosomal protein |
| Helianthuus annuus | HanXRQChr12g0374041 | ribosomal protein |
| Helianthuus annuus | HanXRQChr04g0128141 | ribosomal protein |
| Helianthuus annuus | HanXRQChr05g0163131 | ribosomal protein |
| Helianthuus annuus | HanXRQChr03g0076971 | ribosomal protein |
| Helianthuus annuus | HanXRQChr05g0159851 | ribosomal protein |
| Helianthuus annuus | HanXRQChr05g0159971 | ribosomal protein |
| Helianthuus annuus | HanXRQChr11g0324631 | ribosomal protein |
| Helianthuus annuus | HanXRQChr13g0408051 | ribosomal protein |
| Helianthuus annuus | HanXRQChr03g0089331 | ribosomal protein |
| Helianthuus annuus | HanXRQChr13g0419951 | ribosomal protein |
| Helianthuus annuus | HanXRQChr15g0497041 | ribosomal protein |
| Helianthuus annuus | HanXRQChr16g0499761 | ribosomal protein |
| Helianthuus annuus | HanXRQChr04g0106961 | ribosomal protein |
| Helianthuus annuus | HanXRQChr06g0175811 | ribosomal protein |
| Helianthuus annuus | HanXRQChr04g0122771 | ribosomal protein |
| Helianthuus annuus | HanXRQChr09g0245691 | ribosomal protein |
| Helianthuus annuus | HanXRQChr16g0520021 | ribosomal protein |
| Helianthuus annuus | HanXRQChr03g0060471 | ribosomal protein |
| Helianthuus annuus | HanXRQChr14g0429531 | ribosomal protein |
| Helianthuus annuus | HanXRQChr06g0171911 | ribosomal protein |
| Helianthuus annuus | HanXRQChr15g0479091 | ribosomal protein |
| Helianthuus annuus | HanXRQChr15g0479101 | ribosomal protein |
| Helianthuus annuus | HanXRQChr17g0543641 | ribosomal protein |
| Helianthuus annuus | HanXRQChr17g0543661 | ribosomal protein |
| Helianthuus annuus | HanXRQChr04g0105831 | ribosomal protein |
| Helianthuus annuus | HanXRQChr09g0258341 | ribosomal protein |
| Helianthuus annuus | HanXRQChr10g0287141 | ribosomal protein |
| Helianthuus annuus | HanXRQChr15g0463911 | ribosomal protein |
| Helianthuus annuus | HanXRQChr03g0076171 | ribosomal protein |
| Helianthuus annuus | HanXRQChr05g0159291 | ribosomal protein |
| Helianthuus annuus | HanXRQChr13g0407551 | ribosomal protein |
| Helianthuus annuus | HanXRQChr12g0380701 | ribosomal protein |
| Helianthuus annuus | HanXRQChr15g0477271 | ribosomal protein |
| Helianthuus annuus | HanXRQChr17g0545211 | ribosomal protein |
| Helianthuus annuus | HanXRQChr17g0570741 | ribosomal protein |
| Helianthuus annuus | HanXRQChr17g0570761 | ribosomal protein |
| Helianthuus annuus | HanXRQChr02g0044021 | ribosomal protein |
| Helianthuus annuus | HanXRQChr05g0152871 | ribosomal protein |
| Helianthuus annuus | HanXRQChr01g0012781 | ribosomal protein |
| Helianthuus annuus | HanXRQChr08g0230861 | ribosomal protein |
| Helianthuus annuus | HanXRQChr13g0391831 | ribosomal protein |
| Helianthuus annuus | HanXRQChr11g0337791 | bifunctional trypsin/alpha-amylase inhibitor |
| Helianthuus annuus | HanXRQChr10g0312371 | 2-oxoacid dehydrogenase acyltransferase |
| Helianthuus annuus | HanXRQChr09g0276191 | acid phosphatase (class B) |
| Helianthuus annuus | HanXRQChr05g0142271 | aldose-1-epimerase |
| Helianthuus annuus | HanXRQChr14g0439791 | alpha-D-phosphohexomutase |
| Helianthuus annuus | HanXRQChr09g0251071 | alpha-L-fucosidase |
| Helianthuus annuus | HanXRQChr05g0147371 | annexin |
| Helianthuus annuus | HanXRQChr09g0247561 | Asp protease (Peptidase family A1) |
| Helianthuus annuus | HanXRQChr13g0409681 | berberine-bridge enzyme (S)-reticulin: oxygen oxido-reductase |
| Helianthuus annuus | HanXRQChr10g0295971 | beta-hydroxyacyl-(acyl-carrier-protein) dehydratase |
| Helianthuus annuus | HanXRQChr13g0412571 | carbohydrate esterase family 13 - CE13 (pectin acylesterase - PAE) |
| Helianthuus annuus | HanXRQChr12g0360101 | carbohydrate esterase family 8 - CE8 (pectin methylesterase - PME) |
| Helianthuus annuus | HanXRQChr01g0019231 | carbonic anhydrase |
| Helianthuus annuus | HanXRQChr02g0036611 | cellular retinaldehyde binding/alpha-tocopherol transport |
| Helianthuus annuus | HanXRQChr10g0313581 | chaperonin Cpn60 |
| Helianthuus annuus | HanXRQChr09g0251791 | chlathrin |
| Helianthuus annuus | HanXRQChr11g0329811 | chlorophyll A-B binding protein |
| Helianthuus annuus | HanXRQChr13g0398861 | cobalamin (vitamin B12)-independent methionine synthase |
| Helianthuus annuus | HanXRQChr10g0298981 | cyclophilin |
| Helianthuus annuus | HanXRQChr04g0103281 | Cys protease (papain family) |
| Helianthuus annuus | HanXRQChr09g0268361 | cytochrome P450 |
| Helianthuus annuus | HanXRQChr17g0535591 | dirigent protein |
| Helianthuus annuus | HanXRQChr03g0065901 | expansin |
| Helianthuus annuus | HanXRQChr11g0336761 | expressed protein (cupin domain, seed storage protein |
| | | domain) |
| Helianthuus annuus | HanXRQChr10g0280931 | expressed protein (cupin domain, seed storage protein |
| | | domain) |
| Helianthuus annuus | HanXRQChr10g0288971 | expressed protein (cupin domain, seed storage protein |
| | | domain) |
| Helianthuus annuus | HanXRQChr12g0380361 | expressed protein (cupin domain, seed storage protein |
| | | domain) |
| Helianthuus annuus | HanXRQChr09g0254381 | expressed protein (cupin domain, seed storage protein |
| | | domain) |
| Helianthuus annuus | HanXRQChr04g0112711 | expressed protein (cupin domain, seed storage protein |
| | | domain) |
| Helianthuus annuus | HanXRQChr07g0196131 | expressed protein (cupin domain, seed storage protein |
| | | domain) |
| Helianthuus annuus | HanXRQChr10g0301281 | expressed protein (cupin domain, seed storage protein |
| | | domain) |
| Helianthuus annuus | HanXRQChr10g0301931 | expressed protein (cupin domain, seed storage protein |
| | | domain) |
| Helianthuus annuus | HanXRQChr13g0404461 | expressed protein (cupin domain) |
| Helianthuus annuus | HanXRQChr01g0015821 | expressed protein (DUF642) |
| Helianthuus annuus | HanXRQChr03g0065301 | expressed protein (Gnk2-homologous domain, antifungal |
| | | protein of Ginkgo seeds) |
| Helianthuus annuus | HanXRQChr03g0068311 | expressed protein (LRR domains) |
| Helianthuus annuus | HanXRQChr10g0291371 | expressed protein (LRR domains) |
| Helianthuus annuus | HanXRQChr03g0075061 | fasciclin-like arabinogalactan protein (FLA) |
| Helianthuus annuus | HanXRQChr08g0221961 | ferritin |
| Helianthuus annuus | HanXRQChr09g0257521 | FMN-dependent dehydrogenase |
| Helianthuus annuus | HanXRQChr14g0441641 | fructose-bisphosphate aldolase |
| Helianthuus annuus | HanXRQChr10g0312621 | germin |
| Helianthuus annuus | HanXRQChr09g0244271 | glucose-methanol-choline oxidoreductase |
| Helianthuus annuus | HanXRQChr03g0061571 | glutamate synthase |
| Helianthuus annuus | HanXRQChr05g0144801 | glyceraldehyde 3-phosphate dehydrogenase |
| Helianthuus annuus | HanXRQChr17g0550211 | glycerophosphoryl diester phosphodiesterase |
| Helianthuus annuus | HanXRQChr06g0175391 | glycoside hydrolase family 16 - GH16 (endoxyloglucan |
| | | transferase) |
| Helianthuus annuus | HanXRQChr11g0351571 | glycoside hydrolase family 17 - GH17 (beta-1,3-glucosidase) |
| Helianthuus annuus | HanXRQChr05g0141461 | glycoside hydrolase family 18 - GH18 |
| Helianthuus annuus | HanXRQChr09g0276721 | glycoside hydrolase family 19 - GH19 |
| Helianthuus annuus | HanXRQChr02g0046191 | glycoside hydrolase family 2 - GH2 |
| Helianthuus annuus | HanXRQChr16g0524981 | glycoside hydrolase family 20 - GH20 |
| | | (N-acetyl-beta-glucosaminidase) |
| Helianthuus annuus | HanXRQChr11g0322851 | glycoside hydrolase family 27 - GH27 |
| | | (alpha-galactosidase/melibiase) |
| Helianthuus annuus | HanXRQChr10g0293191 | glycoside hydrolase family 3 - GH3 |
| Helianthuus annuus | HanXRQChr16g0511881 | glycoside hydrolase family 31 - GH31 (alpha-xylosidase) |
| Helianthuus annuus | HanXRQChr14g0461441 | glycoside hydrolase family 32 - GH32 (vacuolar invertase) |
| Helianthuus annuus | HanXRQChr13g0423671 | glycoside hydrolase family 35 - GH35 (beta-galactosidase) |
| Helianthuus annuus | HanXRQChr10g0319301 | glycoside hydrolase family 35 - GH35 (beta-galactosidase) |
| Helianthuus annuus | HanXRQChr09g0256531 | glycoside hydrolase family 38 - GH38 (alpha-mannosidase) |
| Helianthuus annuus | HanXRQChr11g0320901 | glycoside hydrolase family 5 - GH5 (glucan-1,3-beta |
| | | glucosidase) |
| Helianthuus annuus | HanXRQChr05g0130491 | glycoside hydrolase family 51 - GH51 |
| | | (alpha-arabinofuranosidase) |
| Helianthuus annuus | HanXRQChr10g0314191 | glycoside hydrolase family 79 - GH79 |
| | | (endo-beta-glucuronidase/heparanase |
| Helianthuus annuus | HanXRQChr13g0397411 | homologous to A. thaliana PMR5 (Powdery Mildew |
| | | Resistant) (carbohydrate acylation) |
| Helianthuus annuus | HanXRQChr14g0444681 | inhibitor family I3 (Kunitz-P family) |
| Helianthuus annuus | HanXRQChr14g0445181 | lactate/malate dehydrogenase |
| Helianthuus annuus | HanXRQChr17g0564111 | lectin (D-mannose) |
| Helianthuus annuus | HanXRQChr17g0558861 | lectin (PAN-2 domain) |
| Helianthuus annuus | HanXRQChr02g0039251 | lipase acylhydrolase (GDSL family) |
| Helianthuus annuus | HanXRQChr01g0000161 | lipid transfer protein/trypsin-alpha amylase inhibitor |
| Helianthuus annuus | HanXRQChr02g0047121 | mannose-binding lectin |
| Helianthuus annuus | HanXRQChr10g0303361 | mitochondrial carrier protein |
| Helianthuus annuus | HanXRQChr15g0489551 | multicopper oxidase |
| Helianthuus annuus | HanXRQChr05g0135581 | neutral/alkaline nonlysosomal ceramidase |
| Helianthuus annuus | HanXRQChr01g0017621 | nucleoside diphosphate kinase |
| Helianthuus annuus | HanXRQChr10g0295991 | peroxidase |
| Helianthuus annuus | HanXRQChr13g0398251 | peroxiredoxin |
| Helianthuus annuus | HanXRQChr11g0333171 | phosphate-induced (phi) protein 1 |
| Helianthuus annuus | HanXRQChr03g0060421 | phosphodiesterase/nucleotide pyrophosphatase/phosphate |
| | | transferase |
| Helianthuus annuus | HanXRQChr03g0078011 | phosphofructokinase |
| Helianthuus annuus | HanXRQChr13g0408831 | phosphoglycerate kinase |
| Helianthuus annuus | HanXRQChr10g0286701 | phosphoglycerate mutase |
| Helianthuus annuus | HanXRQChr06g0171591 | photosystem II PsbP, oxygen evolving complex |
| Helianthuus annuus | HanXRQChr14g0434951 | plastid lipid-associated protein/fibrillin conserved domain |
| Helianthuus annuus | HanXRQChr05g0146621 | plastocyanin (blue copper binding protein) |
| Helianthuus annuus | HanXRQChr11g0330251 | polyphenol oxidase |
| Helianthuus annuus | HanXRQChr04g0094541 | proteasome A-type subunit |
| Helianthuus annuus | HanXRQChr03g0081271 | proteasome B-type subunit |
| Helianthuus annuus | HanXRQChr12g0356851 | purple acid phosphatase |
| Helianthuus annuus | HanXRQChr15g0485781 | pyridoxal phosphate-dependent transferase |
| Helianthuus annuus | HanXRQChr11g0336791 | ribosomal protein |
| Helianthuus annuus | HanXRQChr11g0330521 | ribosomal protein |
| Helianthuus annuus | HanXRQChr11g0326801 | ribulose bisphosphate carboxylase, large subunit |
| Helianthuus annuus | HanXRQChr16g0523951 | ribulose-1,5-bisphosphate carboxylase small subunit |
| Helianthuus annuus | HanXRQChr01g0022151 | S-adenosyl-L-homocysteine hydrolase |
| Helianthuus annuus | HanXRQChr14g0454811 | S-adenosylmethionine synthetase |
| Helianthuus annuus | HanXRQChr04g0109991 | SCP-like extracellular protein (PR-1) |
| Helianthuus annuus | HanXRQChr03g0072241 | Ser carboxypeptidase (Peptidase family S10) |
| Helianthuus annuus | HanXRQChr12g0377221 | Ser protease (subtilisin) (Peptidase family S8) |
| Helianthuus annuus | HanXRQChr02g0055581 | superoxide dismutase |
| Helianthuus annuus | HanXRQChr15g0493261 | thaumatin (PR5) |
| Helianthuus annuus | HanXRQChr16g0532531 | transketolase |
| Helianthuus annuus | HanXRQChr07g0197421 | translation elongation factor EFTu/EF1A |
| Helianthuus annuus | HanXRQChr06g0173951 | translationally controlled tumour protein |

What is claimed is:

1. A pathogen control composition comprising a plurality of PMPs, wherein each of the plurality of PMPs comprises a heterologous pathogen control agent and wherein the composition is formulated for delivery to an agricultural or veterinary animal pathogen or a vector thereof.

2. The pathogen control composition of claim 1, wherein the heterologous pathogen control agent is an antibacterial agent, an antifungal agent, a virucidal agent, an anti-viral agent, an insecticidal agent, a nematicidal agent, an antiparasitic agent, or an insect repellent.

3. The pathogen control composition of claim 2, wherein the antibacterial agent is doxorubicin.

4. The pathogen control composition of claim 2, wherein the antibacterial agent is an antibiotic.

5. The pathogen control composition of claim 4, wherein the antibiotic is vancomycin.

6. The pathogen control composition of claim 4, wherein the antibiotic is a penicillin, a cephalosporin, a monobactam, a carbapenem, a macrolide, an aminoglycoside, a quinolone, a sulfonamide, a tetracycline, a glycopeptide, a lipoglycopeptide, an oxazolidinone, a rifamycin, a tuberactinomycin, chloramphenicol, metronidazole, tinidazole, nitrofurantoin, teicoplanin, telavancin, linezolid, cycloserine 2, bacitracin, polymyxin B, viomycin, or capreomycin.

7. The pathogen control composition of claim 2, wherein the antifungal agent is an allylamine, an imidazole, a triazole, a thiazole, a polyene, or an echinocandin.

8. The pathogen control composition of claim 2, wherein the insecticidal agent is a chloronicotinyl, a neonicotinoid, a carbamate, an organophosphate, a pyrethroid, an oxadiazine, a spinosyn, a cyclodiene, an organochlorine, a fiprole, a mectin, a diacylhydrazine, a benzoylurea, an organotin, a pyrrole, a dinitroterpenol, a METI, a tetronic acid, a tetramic acid, or a pthalamide.

9. The pathogen control composition of claim 1, wherein the heterologous pathogen control agent is a small molecule, a nucleic acid, or a polypeptide.

10. The pathogen control composition of claim 9, wherein the small molecule is an antibiotic or a secondary metabolite.

11. The pathogen control composition of claim 9, wherein the nucleic acid is an inhibitory RNA.

12. The pathogen control composition of claim 1, wherein the heterologous pathogen control agent is encapsulated by each of the plurality of PMPs.

13. The pathogen control composition of claim 1, wherein the heterologous pathogen control agent is embedded on the surface of each of the plurality of PMPs.

14. The pathogen control composition of claim 1, wherein the heterologous pathogen control agent is conjugated to the surface of each of the plurality of PMPs.

15. The pathogen control composition of claim 1, wherein each of the plurality of PMPs further comprises an additional pathogen control agent.

16. The pathogen control composition of claim 1, wherein the pathogen is a bacterium, a fungus, a parasitic insect, a parasitic nematode, or a parasitic protozoan.

17. The pathogen control composition of claim 16, wherein the bacterium is a Pseudomonas species, an Escherichia species, a Streptococcus species, a Pneumococcus species, a Shigella species, a Salmonella species, or a Campylobacter species.

18. The pathogen control composition of claim 17, wherein the Pseudomonas species is Pseudomonas aeruginosa.

19. The pathogen control composition of claim 17, wherein the Escherichia species is Escherichia coli.

20. The pathogen control composition of claim 16, wherein the fungus is a Saccharomyces species or a Candida species.

21. The pathogen control composition of claim 16, wherein the parasitic insect is a Cimex species.

22. The pathogen control composition of claim 16, wherein the parasitic nematode is a Heligmosomoides species.

23. The pathogen control composition of claim 16, wherein the parasitic protozoan is a Trichomonas species.

24. The pathogen control composition of claim 1, wherein the vector is an insect.

25. The pathogen control composition of claim 24, wherein the vector is a mosquito, a tick, a mite, or a louse.

26. The pathogen control composition of claim 1, wherein the composition is stable for at least one day at room temperature, and/or stable for at least one week at 4° C.

27. The pathogen control composition of claim 1, wherein the PMPs are stable for at least 24 hours, 48 hours, seven days, or 30 days at 4° C.

28. The pathogen control composition of claim 27, wherein the PMPs are stable at a temperature of at least 20° C., 24° C., or 37° C.

29. The pathogen control composition of claim 1, wherein the plurality of PMPs in the composition is at a concentration effective to decrease the fitness of an animal pathogen.

30. The pathogen control composition of claim 1, wherein the plurality of PMPs in the composition is at a concentration effective to decrease the fitness of an animal pathogen vector.

31. The pathogen control composition of claim 1, wherein the plurality of PMPs in the composition is at a concentration effective to treat an infection in an animal infected with a pathogen.

32. The pathogen control composition of claim 1, wherein the plurality of PMPs in the composition is at a concentration effective to prevent an infection in an animal at risk of an infection with a pathogen.

33. The pathogen control composition of claim 1, wherein the plurality of PMPs in the composition is at a concentration of 0.01 ng, 0.1 ng, 1 ng, 2 ng, 3 ng, 4 ng, 5 ng, 10 ng, 50 ng, 100 ng, 250 ng, 500 ng, 750 ng, 1 μg, 10 μg, 50 μg, 100 μg, or 250 μg PMP protein/ml.

34. The pathogen control composition of claim 1, wherein the composition comprises an agriculturally acceptable carrier.

35. The pathogen control composition of claim 1, wherein the composition comprises a pharmaceutically acceptable carrier.

36. The pathogen control composition of claim 1, wherein the composition is formulated to stabilize the PMPs.

37. The pathogen control composition of claim 1, wherein the composition is formulated as a liquid, a solid, an aerosol, a paste, a gel, or a gas composition.

38. The pathogen control composition of claim 1, wherein the composition comprises at least 5% PMPs.

39. A pathogen control composition comprising a plurality of PMPs, wherein the PMPs are isolated from a plant by a process which comprises the steps of:
(a) providing an initial sample from a plant, or a part thereof, wherein the plant or part thereof comprises EVs;
(b) isolating a crude PMP fraction from the initial sample, wherein the crude PMP fraction has a decreased level of at least one contaminant or undesired component from the plant or part thereof relative to the level in the initial sample;
(c) purifying the crude PMP fraction, thereby producing a plurality of pure PMPs, wherein the plurality of pure PMPs have a decreased level of at least one contaminant or undesired component from the plant or part thereof relative to the level in the crude EV fraction;
(d) loading the plurality of PMPs of step (c) with a pathogen control agent; and
(e) formulating the PMPs of step (d) for delivery to an agricultural or veterinary animal pathogen or a vector thereof.

40. An animal pathogen comprising the pathogen control composition of claim 1.

41. An animal pathogen vector comprising the pathogen control composition of claim 1.

42. A method of delivering a pathogen control composition to an animal comprising administering to the animal the composition of claim 1.

43. A method of treating an infection in an animal in need thereof, the method comprising administering to the animal an effective amount of the composition of claim 1.

44. A method of preventing an infection in an animal at risk thereof, the method comprising administering to the animal an effective amount of the composition of claim 1, wherein the method decreases the likelihood of the infection in the animal relative to an untreated animal.

45. The method of claim 42, wherein the infection is caused by a pathogen, and the pathogen is a bacterium, a fungus, a virus, a parasitic insect, a parasitic nematode, or a parasitic protozoan.

46. The method of claim 45, wherein the bacterium is a Pseudomonas species, an Escherichia species, a Streptococcus species, a Pneumococcus species, a Shigella species, a Salmonella species, or a Campylobacter species.

47. The method of claim 45, wherein the fungus is a Saccharomyces species or a Candida species.

48. The method of claim 45, wherein the parasitic insect is a Cimex species.

49. The method of claim 45, wherein the parasitic nematode is a Heligmosomoides species.

50. The method of claim 45, wherein the parasitic protozoan is a Trichomonas species.

51. The method of claim 42, wherein the pathogen control composition is administered to the animal orally, intravenously, or subcutaneously.

52. A method of delivering a pathogen control composition to a pathogen comprising contacting the pathogen with the composition of claim 1.

53. A method of decreasing the fitness of a pathogen, the method comprising delivering to the pathogen the composition of claim 1, wherein the method decreases the fitness of the pathogen relative to an untreated pathogen.

54. The method of claim 52, wherein the method comprises delivering the composition to at least one habitat where the pathogen grows, lives, reproduces, feeds, or infests.

55. The method of claim 52, wherein the composition is delivered as a pathogen comestible composition for ingestion by the pathogen.

56. The method of claim 52, wherein the pathogen is a bacterium, a fungus, a parasitic insect, a parasitic nematode, or a parasitic protozoan.

57. The method of claim 56, wherein the bacterium is a Pseudomonas species, an Escherichia species, a Streptococcus species, a Pneumococcus species, a Shigella species, a Salmonella species, or a Campylobacter species.

58. The method of claim 56, wherein the fungus is a Saccharomyces species or a Candida species.

59. The method of claim 56, wherein the parasitic insect is a Cimex species.

60. The method of claim 56, wherein the parasitic nematode is a Heligmosomoides species.

61. The method of claim 56, wherein the parasitic protozoan is a Trichomonas species.

62. The method of claim 52, wherein the composition is delivered as a liquid, a solid, an aerosol, a paste, a gel, or a gas.

63. A method of decreasing the fitness of an animal pathogen vector, the method comprising delivering to the vector an effective amount of the composition of claim 1, wherein the method decreases the fitness of the vector relative to an untreated vector.

64. The method of claim 63, wherein the method comprises delivering the composition to at least one habitat where the vector grows, lives, reproduces, feeds, or infests.

65. The method of claim 63, wherein the composition is delivered as a comestible composition for ingestion by the vector.

66. The method of claim 63, wherein the vector is an insect.

67. The method of claim 66, wherein the insect is a mosquito, a tick, a mite, or a louse.

68. The method of claim 63, wherein the composition is delivered as a liquid, a solid, an aerosol, a paste, a gel, or a gas.

69. A method of treating an animal having a fungal infection, wherein the method comprises administering to the animal an effective amount of a pathogen control composition comprising a plurality of PMPs.

70. A method of treating an animal having a fungal infection, wherein the method comprises administering to the animal an effective amount of a pathogen control composition comprising a plurality of PMPs, and wherein the plurality of PMPs comprises an antifungal agent.

71. The method of claim 70, wherein the antifungal agent is a nucleic acid that inhibits expression of a gene in a fungus that causes the fungal infection.

72. The method of claim 71, wherein the gene is Enhanced Filamentous Growth Protein (EFG1).

73. The method of claim 70, wherein the fungal infection is caused by Candida albicans.

74. The method of claim 70, wherein the composition comprises a PMP derived from Arabidopsis.

75. The method of claim 70, wherein the method decreases or substantially eliminates the fungal infection.

76. A method of treating an animal having a bacterial infection, wherein the method comprises administering to the animal an effective amount of a pathogen control composition comprising a plurality of PMPs.

77. A method of treating an animal having a bacterial infection, wherein the method comprises administering to the animal an effective amount of a pathogen control composition comprising a plurality of PMPs, and wherein the plurality of PMPs comprises an antibacterial agent.

78. The method of claim 77, wherein the antibacterial agent is Amphotericin B.

79. The method of claim 77, wherein the bacterium is a Pseudomonas species, an Escherichia species, a Streptococcus species, a Pneumococcus species, a Shigella species, a Salmonella species, or a Campylobacter species.

80. The method of claim 77, wherein the composition comprises a PMP derived from Arabidopsis.

81. The method of claim 77, wherein the method decreases or substantially eliminates the bacterial infection.

82. The method of claim 69, wherein the animal is a veterinary animal, or a livestock animal.

83. A method of decreasing the fitness of a parasitic insect, wherein the method comprises delivering to the parasitic insect a pathogen control composition comprising a plurality of PMPs.

84. A method of decreasing the fitness of a parasitic insect, wherein the method comprises delivering to the parasitic insect a pathogen control composition comprising a plurality of PMPs, and wherein the plurality of PMPs comprise an insecticidal agent.

85. The method of claim 84, wherein the insecticidal agent is a peptide nucleic acid.

86. The method of claim 83, wherein the parasitic insect is a bedbug.

87. The method of claim 83, wherein the method decreases the fitness of the parasitic insect relative to an untreated parasitic insect.

88. A method of decreasing the fitness of a parasitic nematode, wherein the method comprises delivering to the parasitic nematode a pathogen control composition comprising a plurality of PMPs.

89. A method of decreasing the fitness of a parasitic nematode, wherein the method comprises delivering to the parasitic nematode a pathogen control composition comprising a plurality of PMPs, and wherein the plurality of PMPs comprises a nematicidal agent.

90. The method of claim 88, wherein the parasitic nematode is Heligmosomoides polygyrus.

91. The method of claim 88, wherein the method decreases the fitness of the parasitic nematode relative to an untreated parasitic nematode.

92. A method of decreasing the fitness of a parasitic protozoan, wherein the method comprises delivering to the parasitic protozoan a pathogen control composition comprising a plurality of PMPs.

93. A method of decreasing the fitness of a parasitic protozoan, wherein the method comprises delivering to the parasitic protozoan a pathogen control composition comprising a plurality of PMPs, and wherein the plurality of PMPs comprises an antiparasitic agent.

94. The method of claim 92, wherein the parasitic protozoan is T. vaginalis.

95. The method of claim 92, wherein the method decreases the fitness of the parasitic protozoan relative to an untreated parasitic protozoan.

96. A method of decreasing the fitness of an insect vector of an animal pathogen, wherein the method comprises delivering to the vector a pathogen control composition comprising a plurality of PMPs.

97. A method of decreasing the fitness of an insect vector of an animal pathogen, wherein the method comprises delivering to the vector a pathogen control composition comprising a plurality of PMPs, and wherein the plurality of PMPs comprises an insecticidal agent.

98. The method of claim 96, wherein the method decreases the fitness of the vector relative to an untreated vector.

99. The method of claim 96, wherein the insect is a mosquito, tick, mite, or louse.


SEQUENCE LISTING (17 sequences)

SEQ ID NO: 1 - DNA, 3 bp, Artificial Sequence
  Synthetic Construct
  misc_feature (1)..(1): n is a, c, g, or t
ngg

SEQ ID NO: 2 - DNA, 6 bp, Artificial Sequence
  Synthetic Construct
  misc_feature (1)..(2): n is a, c, g, or t
nnagaa

SEQ ID NO: 3 - DNA, 5 bp, Artificial Sequence
  Synthetic Construct
  misc_feature (1)..(1): n is a, c, g, or t
  misc_feature (4)..(4): n is a, c, g, or t
nggng

SEQ ID NO: 4 - DNA, 7 bp, Artificial Sequence
  Synthetic Construct
  misc_feature (1)..(3): n is a, c, g, or t
nnngatt

SEQ ID NO: 5 - DNA, 3 bp, Artificial Sequence
  Synthetic Construct
  misc_feature (2)..(3): n is a, c, g, or t
tnn

SEQ ID NO: 6 - DNA, 3 bp, Artificial Sequence
  Synthetic Construct
cta

SEQ ID NO: 7 - RNA, 19 nt, Artificial Sequence
  Synthetic Construct
acauugagcaauuugguuc

SEQ ID NO: 8 - RNA, 19 nt, Artificial Sequence
  Synthetic Construct
gaaccaaauugcucaaugu

SEQ ID NO: 9 - RNA, 18 nt, Artificial Sequence
  Synthetic Construct
auaugcgcaacauugaca

SEQ ID NO: 10 - DNA, 20 bp, Artificial Sequence
  Synthetic Construct
tgccaataatgtgtcggttg

SEQ ID NO: 11 - DNA, 22 bp, Artificial Sequence
  Synthetic Construct
cccatctcttctaccacgtgtc

SEQ ID NO: 12 - DNA, 24 bp, Artificial Sequence
  Synthetic Construct
acggtattgtttccaactgggacg

SEQ ID NO: 13 - DNA, 24 bp, Artificial Sequence
  Synthetic Construct
tggagcttcggtcaacaaaactgg

SEQ ID NO: 14 - DNA, 19 bp, Artificial Sequence
  Synthetic Construct
aggtcgccttgtccaaaac

SEQ ID NO: 15 - DNA, 21 bp, Artificial Sequence
  Synthetic Construct
gcttttagtctcgcctggttc

SEQ ID NO: 16 - DNA, 20 bp, Artificial Sequence
  Synthetic Construct
aggcacggttacatcaaagg

SEQ ID NO: 17 - DNA, 20 bp, Artificial Sequence
  Synthetic Construct
tcgggagcaatgaagagttc